(19) United States
(12) Patent Application Publication
(10) Pub. No.: US 2025/0346859 A1
(43) Pub. Date: Nov. 13, 2025

(54) EXTRACELLULAR VESICLES COMPRISING SMALL INTERFERING RNA

(71) Applicant: City if Hope, Duarte, CA (US)

(72) Inventor: Tristan Scott, Duarte, CA (US)

(21) Appl. No.: 19/203,062

(22) Filed: May 8, 2025

Related U.S. Application Data
(60) Provisional application No. 63/644,421, filed on May 8, 2024.

(51) Int. Cl. C12N 5/0797; C07K 14/005; C07K 14/47; C07K 14/705; C12N 15/113

(52) U.S. Cl. CPC C12N 5/0623; C07K 14/005; C07K 14/47; C07K 14/70596; C12N 15/113; C12N 2310/14; C12N 2310/531; C12N 2760/20222

(57) ABSTRACT

Provided herein, inter alia, are methods for making extracellular vesicles comprising small interfering RNA (siRNA) and cells comprising said extracellular vesicles. Provided herein are stably transfected cells comprising a nucleic acid encoding an Argonaute2 protein and a nucleic acid encoding an shRNA nucleic acid. The stably transfected cells are useful for methods of making extracellular vesicles comprising siRNA.

### CROSS-REFERENCE TO RELATED APPLICATIONS

This application claims priority to U.S. Provisional Application No. 63/644,421, filed May 8, 2024, which is hereby incorporated by reference in its entirety and for all purposes.

### REFERENCE TO AN ELECTRONIC SEQUENCE LISTING

The contents of the electronic sequence listing (048440-898001US_Sequence_Listing_ST26.xml; Size: 7,99,645 bytes; and Date of Creation: May 8, 2025) are hereby incorporated by reference in their entirety.

### BACKGROUND

Extracellular vesicles (EVs) are emerging as pioneering tool for research and biomedical applications. The term extracellular vesicle (EV) is applied to a wide range of cell-derived nanoparticles and these particles are secreted and comprised of (although not limited to) the broadly defined multivesicular body (MVB)-derived vesicles, which contain the small exosome fraction (30-150 nm), the membrane-derived microvesicles (100-1000 nm), and apoptotic bodies. Small EVs are generated within MVBs through the ESC RT pathway and have lipid bilayer membranes and contain various RNA, DNA, and protein payloads in the luminal compartment (5). Traditionally considered a cellular waste product, there is emerging evidence supporting their role in cell-to-cell communication through receptor stimulation by ligands on the EV surface or the delivery of functional payloads; both of which can exert a physiological effect in the recipient cell. EV s themselves are considered important factors in viral, cancer, and inflammatory disorders, contributing significantly to various disease states (6). Furthermore, EVs are thought to cross the blood brain barrier (BBB) (3), largely impermeable to other systemically administered effectors, demonstrating their flexible utility in hard-to-reach areas such as the central nervous system. To fully exploit this technology, methodologies that can load EVs with functional cargo (8) and a means to readily program them with non-immunogenic artificial effector complexes is needed.

Since the discovery that dsRNA triggers degradation of complementary RNA, the application of RNA interference (RNAi) as a research tool has been vital to understand basic biology. RNAi is artificially triggered through three basic dsRNA forms: 1) primary microRNA mimics (miRNAs), 2) short-hairpin RNA (shRNAs), or 3) small-interfering RNA (siRNAs). Each enter the RNAi pathway at various stages and, in the canonical model, converge on the enzyme Dicer that processes the RNA into ˜21 nt dsRNA mature effectors with one strand (the ‘targeting’ strand) loaded into one of four Argonaute proteins (Ago1-4) within the RNA-induced silencing complex (RISC). The RISC is then targeted to a complementary RNA resulting in degradation or suppression.

The delivery of RNAi is vital for its applications, and viral and non-viral nanoparticle platforms have been used. Viral delivery systems are immunogenic, preventing repeat dosing in vivo which limits its therapeutic use. Furthermore, the sustained long-term expression from viral vectors would be unfavorable in scenarios where transient expression may be required. N on-viral synthetic nanoparticles circumvent some of these issues; however, systems like lipids nanoparticles (LNPs) are challenging to alter their tropism to organs other than the liver (2), and some components of nanoparticles can be potently immunogenic (9) and leveraging more native lipid-based particles could solve these issues. Nonetheless, the promise of RNAi-based drugs has been realized in clinic (10), but delivery of the RNAi still hampers translation.

Provided herein, inter alia, are solutions to these and other problems in the art.

### BRIEF SUMMARY

In an aspect is provided a method for making an extracellular vesicle including a small interfering RNA (siRNA) nucleic acid, the method including: i) transfecting a cell with a nucleic acid encoding an argonaute 2 (AGO2) protein and a nucleic acid encoding a short hairpin RNA (shRNA) nucleic acid, and ii) culturing the cell under conditions conducive for the cell to express the AGO2 protein and the shRNA nucleic acid, thereby forming said extracellular vesicle including the siRNA nucleic acid.

In an aspect is provided a method for making an extracellular vesicle (EV) including a small interfering RNA (siRNA) nucleic acid, the method including culturing a cell stably transduced with a nucleic acid encoding an argonaute 2 (AGO2) protein and a nucleic acid encoding a short hairpin RNA (shRNA) nucleic acid under conditions conducive for the cell to express the AGO2 protein and the shRNA nucleic acid, thereby forming the extracellular vesicle including the siRNA nucleic acid.

In an aspect is provided a method for making an extracellular vesicle including a small interfering RNA (siRNA) nucleic acid, the method including: i) transfecting a cell with a nucleic acid encoding an argonaute 2 (AGO2) protein and a nucleic acid encoding a short hairpin RNA (shRNA) nucleic acid, ii) culturing the cell under conditions conducive for the cell to express the AGO2 protein and the shRNA nucleic acid, thereby forming said extracellular vesicle including the siRNA nucleic acid, and iii) isolating the extracellular vesicle including the siRNA nucleic acid from the cell.

In an aspect is provided a method for making an extracellular vesicle (EV) including a small interfering RNA (siRNA) nucleic acid, the method including: i) culturing a cell stably transduced with a nucleic acid encoding an argonaute 2 (AGO2) protein and a nucleic acid encoding a short hairpin RNA (shRNA) nucleic acid under conditions conducive for the cell to express the AGO2 protein and the shRNA nucleic acid, thereby forming the extracellular vesicle including the siRNA nucleic acid, and ii) isolating the extracellular vesicle including the siRNA nucleic acid from the cell.

In an aspect is provided a cell including an argonaute 2 (AGO2) protein and an extracellular vesicle including a small interfering (siRNA) nucleic acid.

In another aspect is a cell stably transfected with a nucleic acid encoding an argonaute 2 (AGO2) protein and a nucleic acid encoding a short hairpin RNA (shRNA) nucleic acid.

### BRIEF DESCRIPTION OF THE DRAWINGS

FIGS. 1A-1B. Exo-shRNA processing is dicer-independent (FIG. 1A) Schematic of Ago2 shRNA processing. Non-canonical shRNAs are processed directly by Ago2, which cleaves the passenger strand. The passenger strand is then trimmed, and the target strand can be loaded into the Ago2 into the RISC complex (FIG. 1B) Knockdown of a reporter vector with a sense and anti-sense target with a dicer-dependent (d) or independent (i) shGFP.

FIGS. 2A-2H. Validation of Exo-shRNAs targeted to GFP. (FIG. 2A) TEM and NTA of purified EVs. (FIG. 2B) EVs were harvested from cells transfected with the Exo-shGFP and the mature effectors detected using RT-qPCR (EVs vs cells). (FIG. 2C) Mature siRNAs detected in EVs transfected with the Exo-shGFP and vectors expressing Ago2, CD63, Ago2-S387A, or CD63-Ago2 fusion. (FIG. 2D) Amplicon sequencing of the RT products from EVs and cells (FIG. 2E). Immunoblotting for FLAG-Ago2 and EV markers (Syntenin and CD81). Calnexin was used as a negative EV marker. (FIG. 2F) EVs were subjected to a protease protection assay in the presence of detergent and proteinase K (PK). TSG101 is luminal control. (FIG. 2G) Exo-shRNA EVs were added to HEK293-GFP cells and knockdown assessed. The EVs were produced with a VSV glycoprotein to assist with fusion. Data sets from left to right represent: Mock, Exo-shGFP, FLAG-Agp2, and ExoshGFP+FLAG-Ago2. (FIG. 2H) EVs packed with two additional Exo-shGFPs (b and c) showed knockdown in reporter cells. a=Exo-shRNA.

FIGS. 3A-3D. RNAi activity is observed with effector enrichment in EVs. (FIG. 3A) EVs were harvested from cells transfected with the Exo-shGFP, Ago2, Ago2+Exo-shGFP and the mature effectors detected using RT-qPCR. (FIG. 3B) The % knockdown of GFP across three independent productions (P) was plotted against relative RNAi enrichment. (FIG. 3C) Schematic of the Exo-shRNA 5′ and 3′ extensions compared to miR-451. (FIG. 3D) Crude EV extracts were prepared from HEK293 supernatant transfected with Cx43 and Nluc-Ago2+Exo-shGFP. GFP control or Mock=untransfected.

FIG. 4 Host factors affecting EV packaging efficiency. RNA levels assessed by RT-qPCR in HEK293 cells transfected with shRNAs to VPS4B or CMPH4C relative to mock control.

FIG. 5. Particles per ml were determined by NTA for EVs harvested from cells transfected with shVPS4B (shV) and shCHMP4C (shC).

FIG. 6. Nluc activity was detected in EVs packaged with CD63-Nluc in the presence of shRNAs to VPS4B (shV) and shCHMP4C (shC), made relative to the physical particle count.

FIGS. 7A-7C. Exo-shRNA EV profiling. (FIG. 7A) EVs generated with the Exo-shGFP with and without an overexpressed VSV-G. (FIG. 7B) Assessing the levels of luciferase activity in various tissues in vivo with implanted HEK293 cells overexpressing CD63-Nluc. (FIG. 7C) Proliferation assays at 72 hrs after treatment demonstrate that the EV transfer does not substantially affect cell proliferation.

FIGS. 8A-8B. EV-mediated delivery of novel non-canonical RNAi effectors. Cartoon of the two models of Exo-shRNA loading into small EVs generated by MVB biogenesis to deliver mature RNAi effectors for target RNA knockdown. (FIG. 8A) Ago2 is a ‘rate limiting’ enzyme that matures the RNAi effectors, which are then loaded into small EVs for transfer, unbound to Ago2. (FIG. 88) “Ago2 RNP transfer model” is when the Ago2 and the mature RNAi effector are loaded in the EVs. The data generated seems to favor the a ‘rate limiting’ model with HEK293 derived EVs.

FIGS. 9A-9B. D icer-independent shRNAs are highly stand selective. Reporter vector with the sense (pSI-GFP-S) and anti-sense (pSI-GFP-AS) target sites in GFP was inserted downstream of Renilla luciferase was transfected into HEK293 cells with a Ago2-dependent shRNA, Agosh-GFPa and Agosh-GFPb. At 48 hrs post-transfection, a dual luciferase assay was performed. The knockdown was normalized to background firefly luciferase levels. A vector that only has a Pol III promoter as an empty vector control (H1) or a vector that expresses an shRNA target to HIV (H1-sh362) were included as negative control. The treatments were made relative to the H1-sh362 set at 100% and the error bars are standard deviation from transfection performed in triplicate. Two different Dicer-independent miR-451 scaffolds modified to target GFP were included for a comparison. (FIG. 9) N on-Canonical shRNAs are strand selective as only the sense target in knockdown, whereas (FIG. 98) Dicersh-GFP can inhibit both sense and anti-sense targets.

FIGS. 11A-10B. Non-conical shRNAs are potent shRNA systems. A reporter vector with the sense target sites in GFP inserted downstream of Renilla luciferase (pSI-GFP-S) was transfected into HEK293 cells with a Ago2-dependent shRNA at reducing ratios to the target vectors (target to shRNA: 1:8, 1:4, 1:2, 1:1, 1:0.5). Two versions of the Ago2-shRNAs were included, (FIG. 10A) Agosh-GFPa and (FIG. 10B) Agosh-GFPb. At 48 hrs pos-transfection, a dual luciferase assay was performed. The knockdown was normalized to background firefly luciferase levels. A vector that only has a Pol III promoter as an empty vector control (H1) or a vector that express an shRNA target to HIV (H1-sh362) were included as negative controls. The treatments were made relative to the H1-sh362 set at 100% and the error bars are standard deviation from transfection performed in triplicate. Two different dicer-independent miR451 scaffolds modified to target GFP were included for a comparison. N on-Canonical Agosh-GFPb is active across all doses and more potent than Agosh-GFPb, miR451-GFP, and Dicersh-GFP. Agosh-GFPb was selected for further evaluation.

FIGS. 11A-11C. Confirmation of shAgo-GFP dicer independence. (FIG. 11A) HEK293 cells were transfected with a vector expressing the Cas13d (CasRx) and a crRNA targeted to Dicer, or a control (empty) crRNA. RNA was extracted and a RT-qPCR was performed. The levels of dicer mRNA knockdown were made relative to the control set at 100% and the error bars are standard deviation from transfections performed in triplicate. (FIG. 11B) A reporter vector with the sense target sites in GFP inserted downstream of Renilla luciferase (pSI-GFP-S) was transfected into HEK293 cells with a Ago2-dependent shRNA (Agosh-GFPb) and the CasRx vector with the Dicer-crRNA or Con-crRNA. The Dicersh-GFP was included as a dicer-dependent control. The levels of knockdown were made relative to a shRNA target to HIV (H1-sh362) control set at 100%. The error bars are standard deviation from transfections performed in triplicate. The experiments were performed twice. (FIG. 1C) HEK293 cells were transfected with the pSI-GFP-S target vector and a series of ‘bulge’ mutants of the Agosh-GFPb. A nucleotide was replaced with an ‘A’ and numbered at the position from the 3′ end. The levels of knockdown were made relative to the H1-sh362 control set at 100%. The error bars are standard deviation from transfections performed in triplicate. The experiments were performed twice. Non-Canonical shRNAs do not lose any activity when dicer mRNA levels was reduced, suggesting they are dicer-independent. The Ago2 cleavage site in the Agosh-GFPb is between the 9th and 10th nucleotide from the 3′ end of the shRNA.

FIGS. 12A-12C. Dicer-independent shRNAs were enriched higher in EVs than previously described miR451-GFP mimic. (FIG. 12A) Schematic of the production set up for generating EVs loaded with matured RNAi effectors. HEK293 cells were triple transfected with a vector expressing the Dicer-independent shRNA target GFP (Agosh-GFPb) and the EVs were purified using differential ultracentrifugation. A miRNA-451 mimic targeted to GFP (U6-miR451-GFP) previously shown to enriched siRNA in EVs was included for a comparison. H1-sh362 targeted to HIV was included as negative detection control. (FIG. 12) Small RNAs were extracted from the EVs, and a RT-qPCR was performed to detect the mature processed RNAi effectors. The levels of processed siRNA for GFP were normalized to the U6snRNA levels. Left and right figures are RT-qPCR from two separate EV productions. (FIG. 12C) Small RNAs were extracted from the EVs from cells transfected with miRNA-451 GFP (U6-miR451-GFP) and miR-16 GFP (U6-miR16-GFP) mimics targeted to GFP. Alternative versions were created to be closer to the wild type miRNA sequence, which were U6-miR451-GFP-2 and U6-miR16-GFPb. RT-qPCR was performed for the mature processed effectors.

FIGS. 13A-13E. The transfer of RNAi by extracellular vesicle with Exo-shRNAs. (FIG. 3A) Schematic of the production set up for generating EVs loaded with matured RNAi effectors. HEK293 cells were triple transfected with a vector expressing the Dicer-independent shRNA target GFP (Agosh-GFPb), a vector expressing a FLAG-tagged Ago2 (Ago2), and a Vesicular stomatitis virus G protein (VSV-G). VSV-G was included as a ‘fusogen’ so the EVs can release the RNAi effectors in the target tissue. The EVs were purified using differential ultracentrifugation. The EVs were produced from HEK293 cells transfected with either an empty vector (PUC19), an Agosh-GFPb only, the Ago2 only, or the Agosh-GFPb and the Ago2 with a VSV-G vector. HEK293 cells transfected with a GFP vector were included as a negative control. (FIG. 13B) Small RNAs were extracted from the EVs, and the RT-qPCR was performed for the mature processed effectors detected using RT-qPCR. The levels of processed siRNA for GFP was normalized to the U6snRNA levels. The levels were made relative to the empty vector (PUC19) control set at 100%. The error bars are standard deviation from three separate EV productions. (FIG. 13C) The EVs were added to HEK293 cells that stably express consistent levels of GFP (HEK293-GFP) and knockdown of GFP was assessed at 72 hrs post-addition by flow cytometry. The EVs produced from HEK293 cells transfected with the empty vector (PUC19) or GFP were negative controls. An untreated (mock) cells was included as a negative control. The error bars are standard deviation from EVs added in triplicate. The experiments were performed twice. (FIG. 13D) Histogram of GFP knockdown compared to mock treated cells. (FIG. 13E) The EVs were quantified, and absolute amounts of EV particles were added to the HEK293-GFP cells and showed dose responsive effects. The Agosh-GFPb can highly enrich mature RNAi effectors in the EV fraction and transfer RNAi to performed knockdown of a HEK293-GFP reporter cell line. The overexpression of Ago2 further enhances RNAi enrichment and knockdown effects and is dose responsive.

FIGS. 14A-14D. Exogenous Ago2 is detected in extracellular fractions. The EVs were produced from HEK293 cells transfected with either an empty vector (PUC19), an Agosh-GFPb only, the FLAG-Ago2 only (Ago2), or the Agosh-GFPb and the Ago2 with a VSV-G vector. (FIG. 14A) Immunoblotting was performed on the small EVs (sEVs) and cell lysates for FLAG-Ago2 or WT Ago2. EV markers for Syntenin-1, ALIX, and CD81 were probed as well as a cell lysate marker, Calnexin (EV negative marker). (FIG. 1B) Representative transmission electron microscope (TEM) image of the purified EVs. (FIG. 14C) Representative Nanotracking analysis (NTA) of the EVs produced from the Agosh-GFPb+Ago2 transfected HEK293 cells showing an average EV size of ˜150 nm. (FIG. 14D) The EVs were subjected to a protease protection assay in the presence of Triton-X detergent and proteinase K (PK). The overexpressed FLAG-Ago2 was detected through the FLAG tag. TSG101 and Syntenin-1 were used as luminal controls. Histone 3 (H3) and VSV-G were included a non-vesicle and surface exposed membrane proteins, respectively. Ago2 can be detected in the EV fractions but is likely not packaged in EVs under these conditions.

FIGS. 15A-15C. Dicer-independent shRNAs processed into mature RNAi effectors are shorter in the extracellular space. (FIG. 15) Small RNA was extracted from the EVs or cells and then a RT-qPCR performed. Primer extension assays were performed with primers to detected the mature RNAi effectors of 18, 19, 20, 21, 22 nucleotide (nts) used. The error bars were standard deviation from experiments performed in duplicate. (FIG. 5B) The RT products were subjected to amplicon sequencing from EVs and cells. The experiments were performed in duplicate. Reads length were counted and ranked. (FIG. 15C) Small RNA seq (smRNA-seq) was performed on duplicate EV (S5 and S6) or cell extracts (S9 and S12). The read lengths of the mature siRNAs were counted and represented as Read per millions (RPM) with units on the left Y-Axis. The ratio of the length of the small siGFP RNAs in EV versus cell is represented as a blue line with units on the right Y-Axis. The dominant RNAi effector length in cells and EVs is 23 nt and 24 nt. However, EVs are enriched for smaller effectors <20nt with a peak at 18 nt.

FIGS. 16A-16B. HEK293 were transfected with no vector control (EVs: S1 and S2, cells: S7 and S8), the FLAG-Ago2 (Ago2) vector only (EVs: S3 and S4, cells: S10 and S11), or the Agosh-GFPb and the Ago2 (U6-Agosh-GFPb+Ago2) (S5 and S6, Cells: S9 and S12), with a VSV-G vector. The EVs were purified using differential ultracentrifugation. The Small RNA s were extracted from the EVs or cells and small RNA-seq (smRNA-seq) was performed. (FIG. 16A) The % RNA-seq composition is shown. (FIG. 16) the endogenous miRNA profile in the EVs is shown. The U6-Agosh-GFPb makes up a significant % of the extracellular reads. The overexpression Ago2 changes the enriched miRNA profile in the EVs, but the overexpression of Agosh-GFPb reduces the miRNA profile. In U6-Agosh-GFPb+Ago2 EVs, miR-25-5p and miR-4521 are significantly upregulated.

FIGS. 17A-17E. The Exo-shRNA is programmable with alternative shRNAs and functional in different cell lines. (FIG. 17A) A reporter vector expressing a GFP-Fluc was transfected into HEK293 cells with Agosh-GFPb, Agosh-GFPb-2, and agosh-GFPb-3, which target three separate sites in GFP. At 48 hrs post-transfection, a dual luciferase assay was performed. The knockdown was normalized to background Renilla luciferase (pRL) levels transfected as a separate vector. A vector that has a Pol III promoter expressing an shRNA target to HIV (H1-sh362) was included as a negative control. The treatments were made relative to the H1-sh362 set at 100% and the error bars are standard deviation from transfections performed in triplicate. (FIG. 17B) The EVs were produced by triple transfection of HEK293 cells with one of three Agosh-RNAs targeted to GFP (Agosh-GFPb, Agosh-GFPb-2, or Agosh-GFPb-3) with the Ago2 and VSV-G vectors. The EVs were purified using differential ultracentrifugation. A HEK293-GFP cell line was treated with the EVs and knockdown of GFP was assessed at 72 hrs post-addition by flow cytometry. Untreated cells were included as a mock control. (FIGS. 17C-17E) The EVs were packaged with the Agosh-GFPb as described and then added to cell lines stably expressing a GFP-Fluc-IRES-Puro which included A549, Huh7, HepG2, U87, SCC154, and Siha cell lines. These cells were transduced with low MOI lentiviral vectors packaged with a GFP-Fluc-IRES-Puromycin and then selected with 1.5 μg/ml puromycin in complete media to enrich for GFP signal. Knockdown of GFP was assessed at 72 hrs post-addition by flow cytometry and represented as (FIG. 17C) % negative GFP, (FIG. 17D) average MFI or (FIG. 17E) relative MFI. For relative MFI, the treatments were made relative to the untreated (mock) control. The error bars represent standard deviation from treatments performed in duplicate. The Exo-shRNA system is programmable with different Agosh-RNA and functions in various cell lines.

FIGS. 18A-18G. The exo-shRNA system can function with SEC purified EV s (FIG. 8A). The EVs were produced by triple transfection of HEK293 cells with the Agosh-GFPb, Ago2 and VSV-G vectors. The EVs were purified using the qEV35 SEC column (iZON). The EVs were quantified by NTA and added in dilution to a HEK293-GFP cell line was treated with the EVs and knockdown of GFP was assessed at 72 hrs post-addition by flow cytometry. Untreated cells were included as a mock control. (FIG. 18B) Representative TEM and (FIG. 19C) NTA performed on the SEC purified EVs. (FIG. 18D) The SEC purified EVs were subjected to a protease protection assay in the presence of Triton-X detergent and proteinase K (PK). TSG101 was used as luminal control. Histone 3 (H3) and VSV-G were included a non-vesicle and surface exposed membrane proteins, respectively. The overexpressed FLAG-Ago2 was detected through the FLAG tag. (FIG. 18E) Fractions (F1, F2, F3, F3, F5) from the SEC purified EVs were collected and analyzed under TEM. Fraction 3 (F3) had the highest EV concentration, but also high levels of aggregated proteins. (FIG. 18F) The EV fractions were added to a HEK293-GFP cell line and knockdown of GFP was assessed at 72 hrs post-addition by flow cytometry. Untreated cells were included as a mock control. (FIG. 19G) Small RNA was extracted from the EVs fractions then a RT-qPCR performed. Primers to the mature RNAi effectors was used and compared to a standard curve to absolute determine copy numbers. The error bars were technical replicates performed in duplicate. The Exo-shRNA EVs are functional when purified by SEC.

FIGS. 19-19D. The exo-shRNA system transfers functional RNAi through small EVs (FIG. 1A) The EVs were produced by triple transfection of HEK293 cells with the Agosh-GFPb, Ago2 and VSV-G vectors. The EVs were purified by differential ultracentrifugation combined with iodixanol density gradient purification (dUC-DG). Five fractions of 1 ml each were combined to collection the EVs (F1-F5 and F6-F10) and protein fractions (F11-15 and F16-20). Remaining fractions were collected which are likely lipid components (F21-F25 and F26-30). Representative TEM images of the fractions are shown. (FIG. 19B) The F1-F5 fraction was measured by NTA. (FIG. 19C) The EV fractions were added to a HEK293-GFP cell line treated with the EVs and knockdown of GFP was assessed at 72 hrs post-addition by flow cytometry. Untreated cells were included as a mock control. (FIG. 19D) Small RNA was extracted from the EV fractions and then a RT-qPCR performed. Primers to the mature RNAi effectors was used and compared to a standard curve to determine absolute copy numbers. The error bars were technical replicates performed in duplicate. The Agosh-GFPb EVs were transferred from the purified EV fractions. Even through the RNAi effectors are in the protein fractions (F11-F15 and F16-F20), these fractions cannot elicit a knockdown effect.

FIGS. 20A-20C. The RNAi effect is not transferred by an Ago2 RNP packaged within the EVs (FIG. 20A) The EVs were produced by triple transfection of HEK293 cells with the Agosh-GFPb, Ago2 and VSV-G vectors. The EVs were purified by differential ultracentrifugation combined with iodixanol density gradient purification (dUC-DG). Five fractions of 1 ml each were combined to collection EV (F1-F5 and F6-F10) (FIG. 20A) and protein fractions (F11-15 and F16-20) (FIG. 2B). Remaining fractions were collected which are likely lipid components (F21-F25 and F26-30) (FIG. 20C). The DG purified EVs were subjected to a protease protection assay in the presence of Triton-X detergent and proteinase K (PK). TSG101 and Syntenin-1 were used as luminal controls. Histone 3 (H3) and VSV-G were included a non-vesicle and surface exposed membrane proteins, respectively. The overexpressed FLAG-Ago2 was detected through the FLAG tag. * represents residual non-specific Proteinase K signal and not syntenin-1 signal. The Exo-shRNA EVs do not contain Ago2 RNP and do not transfer RNP complexes under these conditions.

FIG. 21 The Exo-shRNA RNAi effectors are unbound RNA within EVs. The dUC-DG purified EVs were subjected to a protease and RNA se protection assay. The EVs were exposed to Proteinase K and/or RNA se A in the presence or absence of Triton-X detergent, the small RNA extracted was extracted and a RT-qPCR performed for the mature RNAi effector. The signal was compared to a standard curve to determine absolute copy numbers. The error bars were technical replicates performed in duplicate.

FIGS. 22A-22G. Specific subpopulations of Exo-shRNA EVs are eliciting RNAi transfer likely through VSV-G enrichment. (FIG. 22A) The EVs were produced by triple transfection of HEK293 cells with the Agosh-GFPb, Ago2 and VSV-G vectors. The EVs were purified by differential ultracentrifugation combined with iodixanol density gradient purification. Six fractions of 1 ml each (F1, F2, F3, F4, F5, F6). Representative TEM images of the fractions are shown. (FIGS. 22B-22C). The F1-F5 fraction was measured by NTA with (FIG. 22B) size (nm) and (FIG. 22C) particles per ml. (FIG. 22D) The EV fractions were added to a HEK293-GFP cell line was treated with the EVs and knockdown of GFP was assessed at 72 hrs post-addition by flow cytometry. Untreated cells were included as a mock control. (FIG. 22E) Small RNA was extracted from the EV then a RT-qPCR performed. Primers to the mature RNAi effectors were used and compared to a standard curve to determine absolute copy numbers. The error bars were technical replicates performed in duplicate. (FIG. 22F) Absolute copies of RNAi effector per particles were determined for F1 and F2. (FIG. 22G) Immunoblotting was performed on the small EVs fractions. EV markers for Syntenin-1, CD63, and TSG101 were probed. The densitometry quantification of the bands is shown to the right. Specific EV subpopulations are transferring the RNAi effector, mostly in F2. These EVs are smaller, possibly through VSV-G that is enriched in the small EV fraction. However, both F1 and F2 have high amounts of RNAi effectors per EV. These are specific in the fractions that correspond to high amounts of EV markers.

FIGS. 23A-23B. Cellular factors that can enhance extracellular Nluc-Ago signal. An Ago2 was fused to Nanoluciferase (Nluc-Ago2) and transfected with Agosh-GFPb with a series of overexpression vectors. PUC19 and GFP were included a negative vector controls. The supernatant was collected at 72 hrs post-transfection, centrifuged to removed cells and then microvesicles, and filtered through a 0.2 μm filter. (FIG. 23A, FIG. 23B) Nanoluc levels were detected in the filtered supernatant for factors including (FIG. 23A) VSV-G, PTPN1, CD63, CD63, Cx43, PCT, TRBP, ALIX, HSP90 and (FIG. 23B) KRAS and mutants thereof, HRAS and mutants thereof, NRAS, EGFR and mutants thereof, and AKT3 and mutants thereof. The error bars represent standard deviation from experiments transfected in triplicate. Experiments were performed at least twice. VSV-G, Cx43, HRAS and hyperactive mutants of HRAS and KRAS increase extracellular Nluc-Ago2 signal. The nluc signal is abolished with dominate negative mutants of RAS proteins.

FIGS. 24A-24D. Modification of the RNA sequence does not affect strand selectivity of Ago2-dependent shRNAs. (FIG. 24A, FIG. 24B, FIG. 24C, FIG. 24D) A reporter vector with the sense (pSI-GFP-S) and anti-sense (pSI-GFP-AS) target sites in GFP was inserted downstream of Renilla luciferase and transfected into HEK293 cells with a series of sequence modified Agosh-GFPb. At 48 hrs post-transfection, a dual luciferase assay was performed. The knockdown was normalized to background firefly luciferase levels. A vector that only has a Pol III promoter as an empty vector control (H1 or U1), a U6 prompter expressing a CRISPR RNA (TAR), tRNA targeted to CCR5 (tRNA-CCR5), empty vector (PUC19), or a vector that expresses an shRNA target to HIV (H1-sh362) were included as negative controls. The treatments were made relative to the H1-sh362 or TAR set at 100%. The error bars are standard deviation from transfections performed in triplicate. A Dicer-independent shRNA to GFP (U6-Dicersh-GFP) was included for compassion. Sequence modification or extension at the 3′ and 5′ end of the Agosh-GFPb maintains knockdown of the sense targets only, except tRNA-Agosh-GFPb and U1-Agosh-GFPb may have some knockdown of the anti-sense GFP target.

FIGS. 25A-25B. Ago2 shRNAs transfected into a HEK293-GFP cell line. HEK293-GFP cells were transfected with the Agosh-GFPb and variants, and knockdown of GFP was assessed at 72 hrs later by flow cytometry. An empty vector (PUC19) was included as a mock control. (FIG. 25A) % negative, and (FIG. 25B) MFI are presented. The error bars were generated by transfections performed in triplicate. Expect the U1 expressed shRNAs, all the shRNAs have similar levels of knockdown when transfected into HEK293-GFP cells.

FIGS. 26A-26B. Sequence modified Ago2 shRNAs can enrich extracellular Nluc-Ago signal. An Ago2 was fused to Nanoluciferase (Nluc-Ago2) was transfected with a series of sequence modified Agosh-GFPb vectors. PUC19 was included as a negative control. The supernatant was collected at 72 hrs transfection, centrifuged to removed cells and then microvesicles, and filtered through a 0.2 μm filter. (FIG. 26A) Nanoluc levels were detected in the filtered supernatant. The error bars represent standard deviation from experiments transfected in triplicate. For (FIG. 26A), the experiments were performed at least twice. Sequence modified shRNAs have differential enrichment of Nluc-Ago2 in the extracellular space. (FIG. 26B) U6-Agosh-GFPb-5′A-3′T, U6-Agosh-GFPb-5′A-3′Delta, and U6-Agosh-GFPb-ExoMotif-2 may further increase Nluc-Ago2 signal compared to U6-Agosh-GFPb.

FIG. 27. EV-associated RNA binding proteins (RBPs) do not enrich extracellular Nluc-Ago signal. An Ago2 was fused to Nanoluciferase (Nluc-Ago2) was transfected with sequence modified Agosh-GFPs and RBP that bind EV-enriched RNAs (hRNPA2B1, SYNCRIP, LA, ALYREF, Fus, MEX3C-1). PUC19 and GFP were included as negative controls. The supernatant was collected at 72 hrs transfection, centrifuged to removed cells and then microvesicles, and filtered through a 0.2 μm filter. Nanoluc levels were detected in the filtered supernatant. The error bars represent standard deviation from experiments transfected in triplicate. Sequence modified shRNAs with RBP binding proteins known to enriched RNA in EVs did not further improve extracellular Nluc-Ago2 signal. SYNCRIP reduced Nluc-Ago2 signal.

FIGS. 28A-28B. EVs produced with Agosh-GFP variants can improve knockdown in recipient reporter cells. HEK293 cells were triple transfected with a vector expressing the Dicer-independent shRNA target GFP (Agosh-GFPb) and the (FIG. 28A) Ago-shRNAs expressed off different promoters or (FIG. 28B) shRNA sequence variants, with a vector expressing a FLAG-tagged Ago2 (Ago2) and a Vesicular stomatitis virus G protein (VSV-G). The EVs were purified using differential ultracentrifugation. The purified EV fractions were added to a HEK293-GFP cell line and was treated with the EVs and knockdown of GFP was assessed at 72 hrs post-addition by flow cytometry. The % negative GFP and MFI are presented. U6-Agosh-GFPc, U6-Agosh-GFPb-5′A-3′Delta, U6-Agosh-GFPb-ExoMotif-2, and tRNA lys3-Agosh-GFPb had improved knockdown effects compared to U6-Agosh-GFPb.

FIGS. 29A-29C. An Ago2-dependent shRNA targeted to the HIV-1 promoter can inhibit transgene expression. (FIG. 29A) A reporter vector with the sense (pSI-362-S) and anti-sense (pSI-362-AS) target sites in HIV-1 NF-KB 362 site (362) was inserted downstream of Renilla luciferase and transfected into HEK293 cells with a series of sequence modified Agosh-362 vectors. At 48 hrs post-transfection, a dual luciferase assay was performed. The knockdown was normalized to background firefly luciferase levels. A vector that only has a Pol III expressing a CRISPR RNA (TAR) was included as a negative control. A Dicer-independent shRNA to 362 (H1-Dicersh-362) was included for compassion. The treatments were made relative to the TAR set at 100%. The error bars are standard deviation from transfections performed in triplicate. (FIG. 29B) The U6-Agosh-362 was transfected into a HEK293 with the long terminal repeat (LTR) promoter of HIV-1 driving GFP expression (HEK293-LTR-GFP) and knockdown of GFP was assessed at 72 hrs post-addition by flow cytometry. Untreated cells (mock) or a vector expressing a C R ISPR RNA (TAR) were included as negative controls. The error bars represent standard deviation from experiments transfected in triplicate. (FIG. 29C) EVs were packaged with the U6-Agosh-362 and added to the HEK293-LTR-GFP and knockdown of GFP was assessed at 72 hrs post-addition by flow cytometry. Untreated cells (mock) were included as negative control. The error bars represent standard deviation from experiments treated in triplicate.

FIGS. 30A-30B. D icer-independent shRNAs were targeted to the E6 and E7 of HPV. A reporter vector with the HPV E6-E7 target site inserted downstream of Renilla luciferase was transfected into HEK293 cells with an Ago2-dependent shRNAs targeted to (FIG. 30A) E6 or (FIG. 30B) E7. At 48 hrs post-transfection, a dual luciferase assay was performed. The knockdown was normalized to background firefly luciferase levels. A vector that expresses a shRNA targeted to HIV (H1-sh362) was included as negative control. The treatments were made relative to the H1-sh362 set at 100% and the error bars are standard deviation from transfection performed in triplicate.

FIGS. 31A-31B. Schematic illustrating (FIG. 31A) extracellular vesicle biogenesis within a cell and (FIG. 33B) the endogenous RNAi pathway.

FIGS. 32A-32B. Schmatic illustrating isolation of extracellular vesicles. (FIG. 23A) Differential ultracentrifugation (dUC) process for EV purification. (FIG. 32B) Density gradient purification to separate vesicle and non-vesicle (free protein) components.

FIG. 33. Schematic of a Ago2-luciferase screen to assess extracellular Ago2. Ago2 was fused to Nanoluciferase (Nluc-Ago2) and transfected with the shRNA vectors expressed off different promoters.

FIGS. 34A-34B. Ultra-pure EVs with Agosh-GFP variants maintain improved knockdown in recipient reporter cells. (FIG. 34A) HEK293 cells were triple transfected with a vector expressing the Dicer-independent shRNA target GFP (Agosh-GFPb) and the Ago-shRNAs expressed off different promoters or shRNA sequence variants, with a vector expressing a FLAG-tagged Ago2 (Ago2) and a Vesicular stomatitis virus G protein (VSV-G). The EVs were purified using Iodixanol Density gradient and collected fractions (F1-F5) which contained small EVs. The EVs morphology and size were determined by NTA and TEM. (FIG. 34B) A total of 5E8 total particles (quantified by NTA) were added to a HEK293-GFP cell line and knockdown of GFP was assessed at 72 hrs post-addition by flow cytometry. The % negative GFP and MFI are presented.

FIGS. 35A-35B. EVs produced with Agosh-GFP variants at alternative GFP targets can improve knockdown in recipient reporter cells. HEK293 cells were triple transfected with a vector expressing the (FIG. 35A) Agosh-GFPb-2 or (FIG. 35B) Agosh RNA-GFPb-3 expressed off different promoters or shRNA sequence variants, with a vector expressing a FLAG-tagged Ago2 (Ago2) and a Vesicular stomatitis virus G protein (VSV-G). The EVs were purified using differential ultracentrifugation. The purified EV fractions were added to a HEK293-GFP cell line and knockdown of GFP was assessed at 72 hrs post-addition by flow cytometry. The % negative GFP and MFI are presented.

FIG. 36. A Agosh-GFP expressed of an alternative pol III promoter initiator nucleotide are functional and facilitate knockdown in recipient reporter cells. HEK293 cells were triple transfected with a vector expressing the Dicer-independent shRNA target GFP expressed off a U6 pol III promoter with a Guanine (G) initiator nucleotide (U6-Agosh-GFPb-5′G), with a vector expressing a FLAG-tagged Ago2 (Ago2) and a Vesicular stomatitis virus G protein (VSV-G). The EVs were purified using differential ultracentrifugation. The purified EV fractions were added to a HEK293-GFP cell line and knockdown of GFP was assessed at 72 hrs post-addition by flow cytometry. The U6-Agosh-GFPb-5′G vector was compared to the original U6-Agosh-GFPb vector which utilize an Adenine (A) initiator nucleotide. The U6-Agosh-GFPb-5′G-3′ delta with a variant sequence was also assessed. The % negative GFP and MFI are presented.

FIGS. 37A-27B. Cx43 overexpression with Exo-shRNAs increases enrichment of mature siRNA in EVs (FIG. 37A) HEK293 cells were triple transfected with a vector expressing the Dicer-independent shRNA target GFP (Agosh-GFPb) with a Cx43 vector and FLAG-tagged Ago2 (Ago2). The EVs were purified using Iodixanol Density gradient and collected fractions (F1-F5) which contained small EVs. The EVs size and absolute counts were determined by NTA. (FIG. 37B) The small RNAs was extracted and a RT-qPCR performed for the mature RNAi effector. The signal was compared to a standard curve to determine absolute copy numbers. The error bars were technical replicates performed in duplicate.

FIG. 38. Improved potency with modified Exo-shRNAs is not from mature RNAi effector enrichment. Data shows quantification of mature RNAi effectors in ultra-pure EVs produced with Agosh-GFP variants. HEK293 cells were triple transfected with a vector expressing the Dicer-independent shRNA target GFP (Agosh-GFPb) and the Ago-shRNAs expressed off a Pol III H1 or tRNA lys promoter, with a vector expressing a FLAG-tagged Ago2 (Ago2) and a Vesicular stomatitis virus G protein (VSV-G). The EVs were purified using Iodixanol Density gradient and collected fraction (F1-F5) which contained pure small EVs. Small RNA was extracted from an absolute EV number (quantified by NTA) then a RT-qPCR performed. Primers to the mature RNAi effectors was used and compared to a standard curve to determine absolute copy numbers per μl. The error bars were technical replicates performed in duplicate.

FIGS. 39A-39C. Ago2 shRNAs variants transfected into a HEK293-GFP cell line. HEK293-GFP cells were transfected with the (FIG. 39A) Agosh-GFPb2 and (FIG. 39B) Agosh-GFPb3 and their promoter (tRNAlys) or sequence and motif variants (5′A-3′Delta and Exomotif-2), or (FIG. 39C) Agosh-GFPb with a G transcription initiation nucleotide and its sequence variant (5′A-3′Delta). Knockdown of GFP was assessed at 72 hrs later by flow cytometry. Untreated cells (mock) was included as a negative control. % negative, and MFI are presented. The error bars were generated by transfections performed in duplicate. The results show that shRNAs knockdown when transfected into HEK293-GFP cells did not account for the enhanced knockdown observed when RNAi was transmitted with an EV, suggesting an EV specific mechanism for improved activity.

FIG. 40. An Agosh-GFP expressed off an alternative pol III promoter initiator nucleotide can facilitate knockdown in recipient reporter cells. HEK293 cells were triple transfected with a vector expressing the Dicer-independent shRNA target GFP expressed off a U6 pol III promoter with a Guanine (G) initiator nucleotide (U6-Agosh-GFPb-5′G) and its sequence variant (5′G-3′Delta), with a vector expressing a FLAG-tagged Ago2 (Ago2) and a Vesicular stomatitis virus G protein (VSV-G). The EVs were purified using differential ultracentrifugation. The purified EV fractions were added to a HEK293-GFP cell line in reducing volumes of 10 μl, 5 μl, and 1 μl and knockdown of GFP was assessed at 72 hrs post-addition by flow cytometry. The U6-Agosh-GFPb-5′G EVs were compared to the original U6-Agosh-GFPb which utilizes an Adenine (A) initiator nucleotide. The % negative GFP is presented. The error bars were generated by transfections performed in triplicate. Results show Exo-shRNA with the G transcription initiation nucleotide transfer knockdown similar to an A-initiated Exo-shRNA.

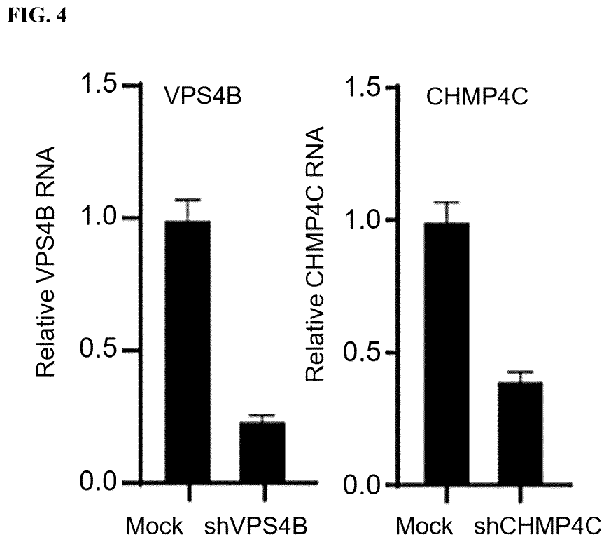

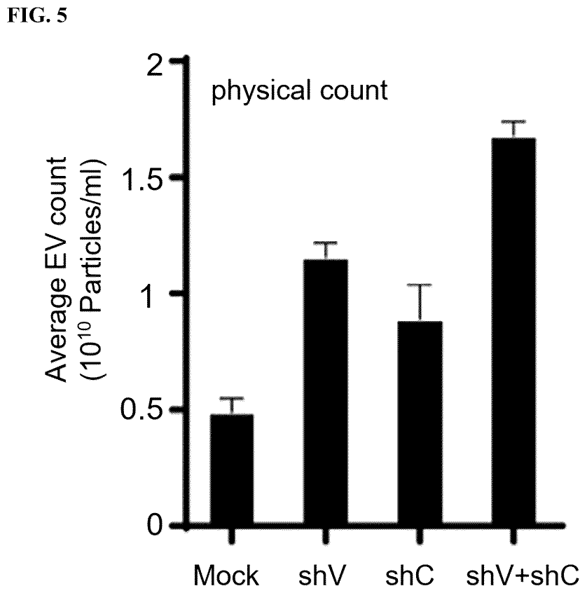

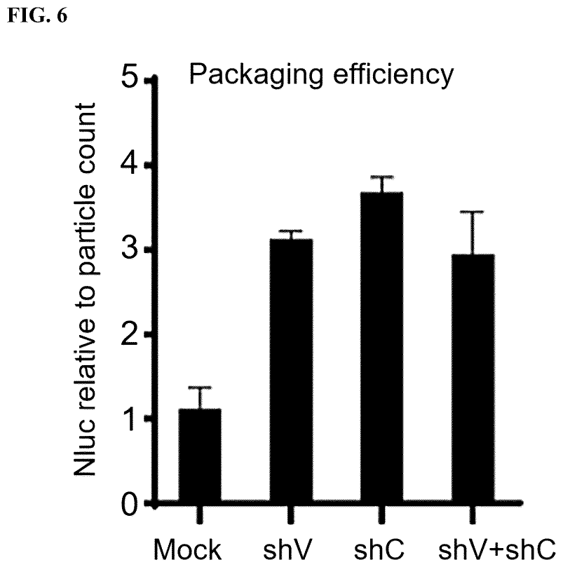

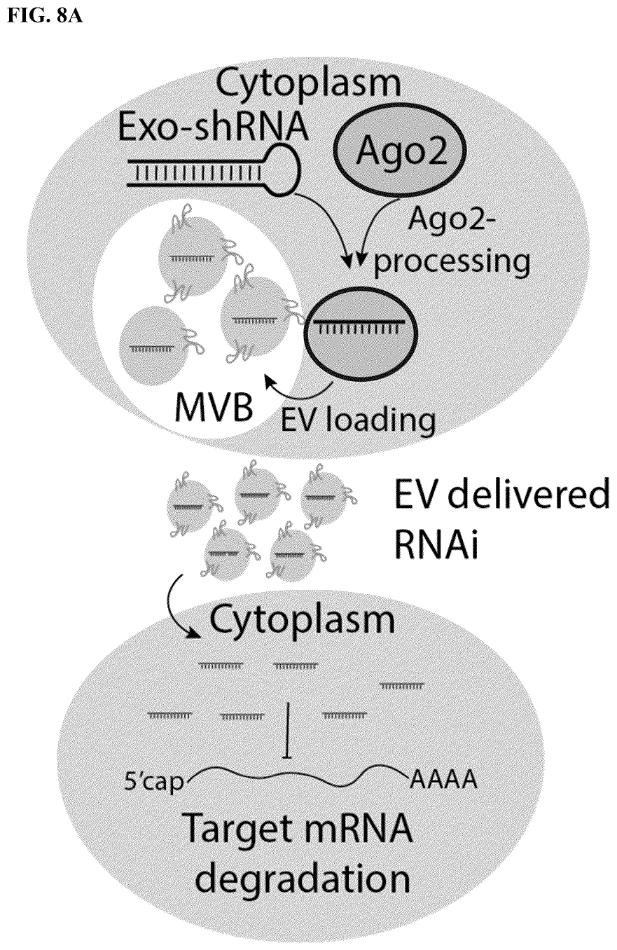

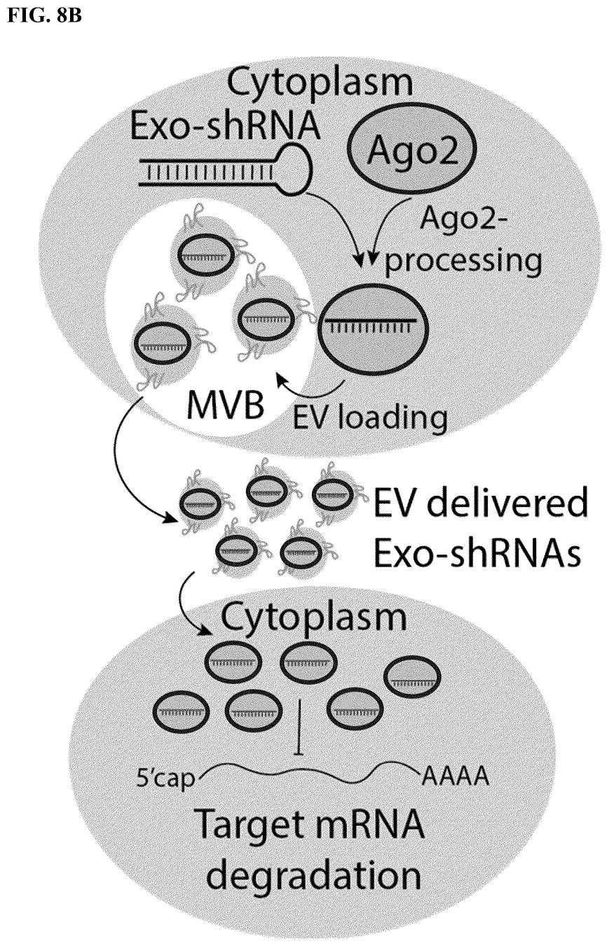

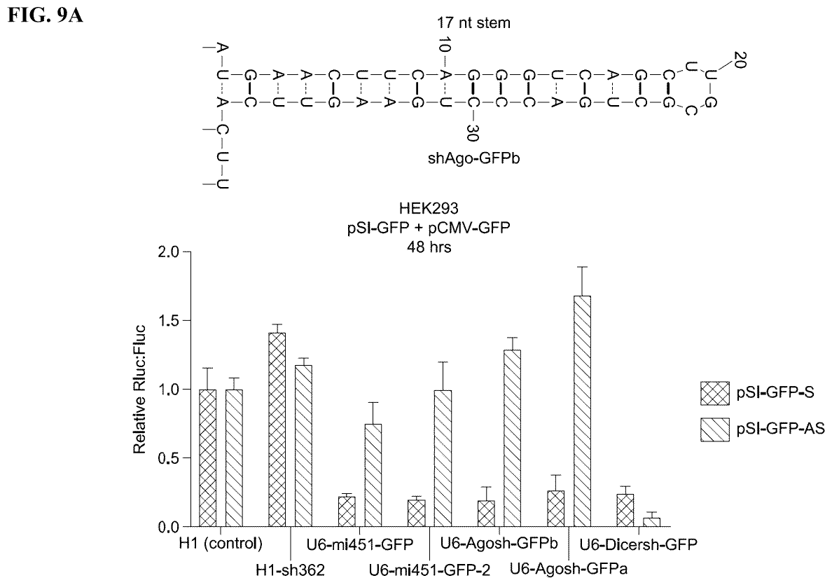

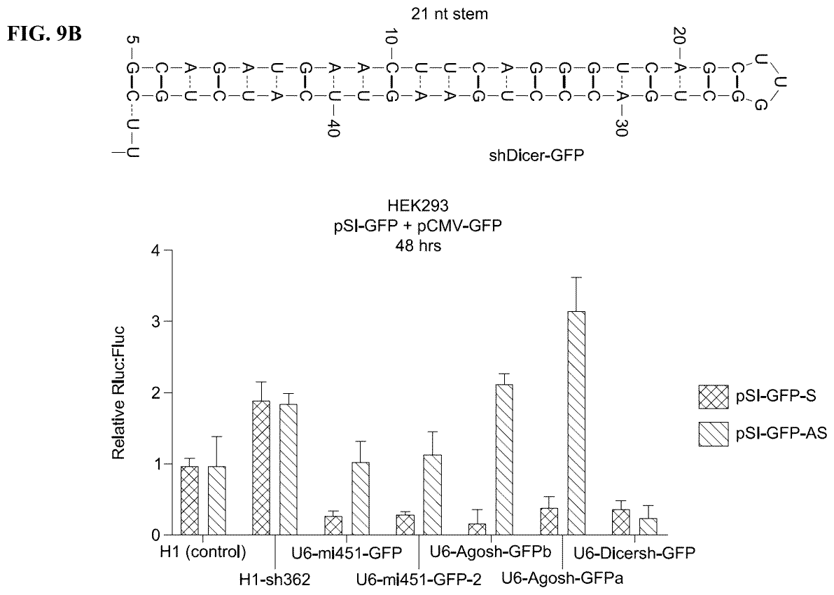

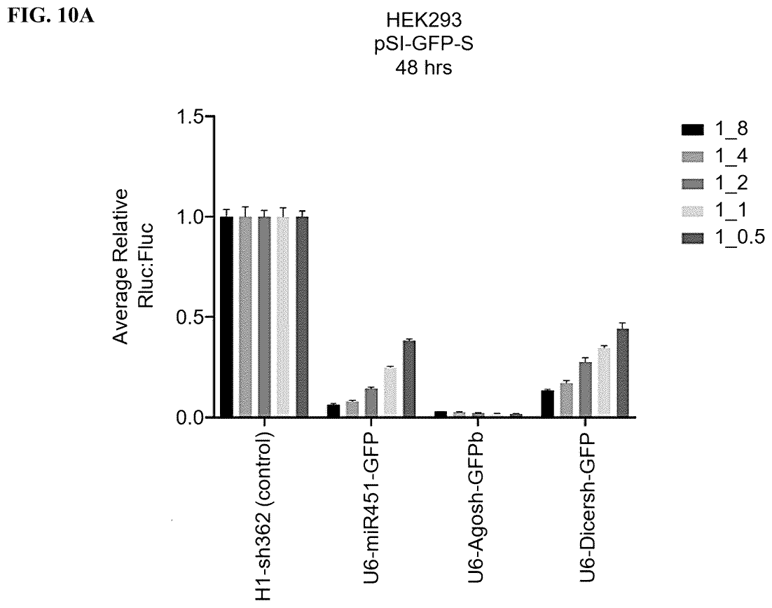

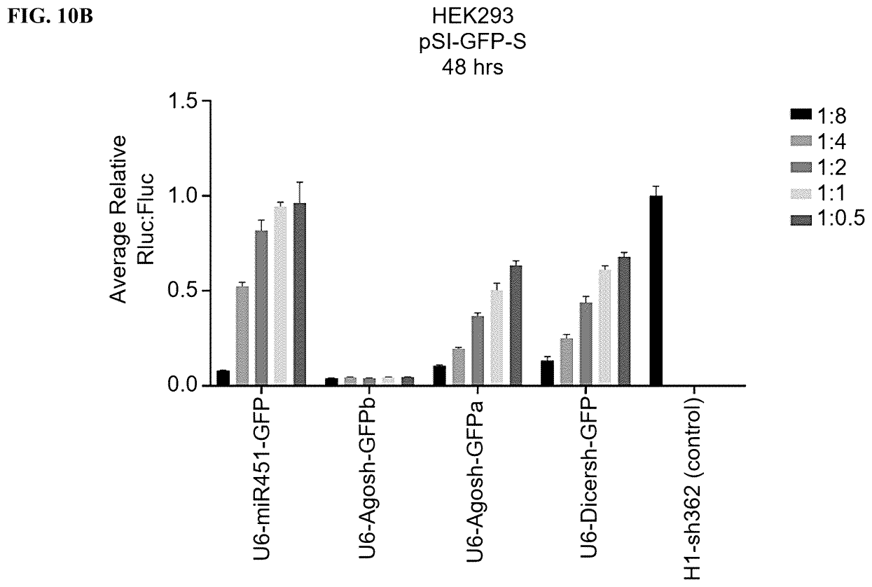

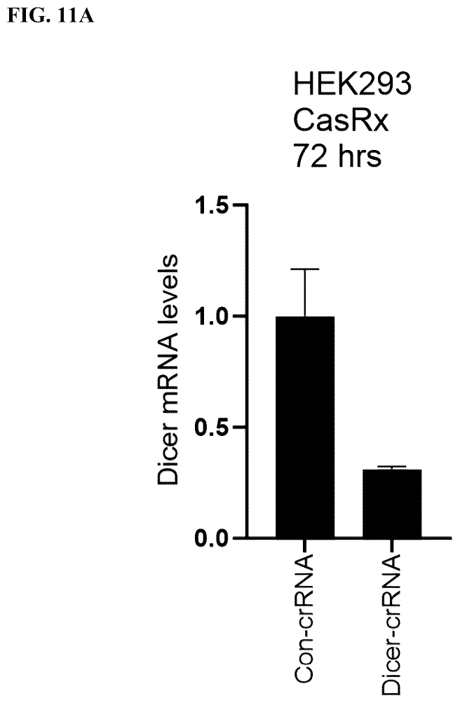

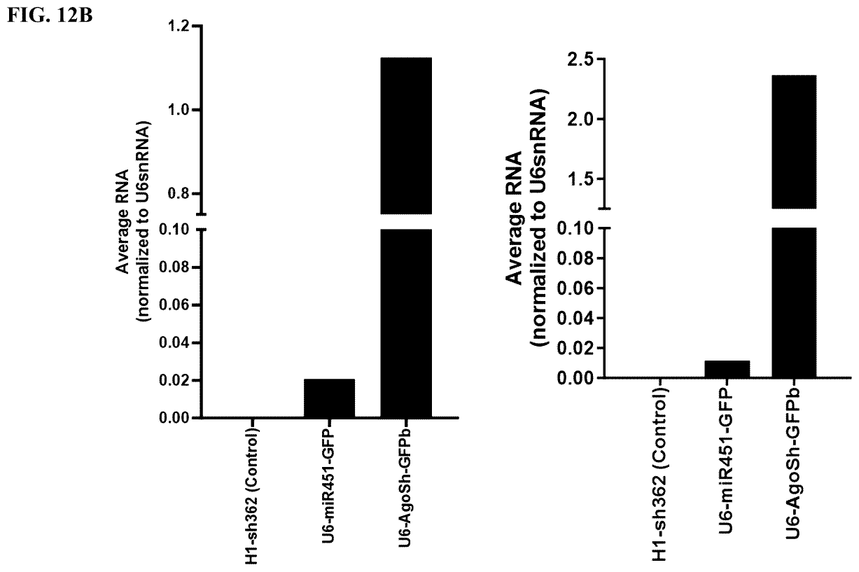

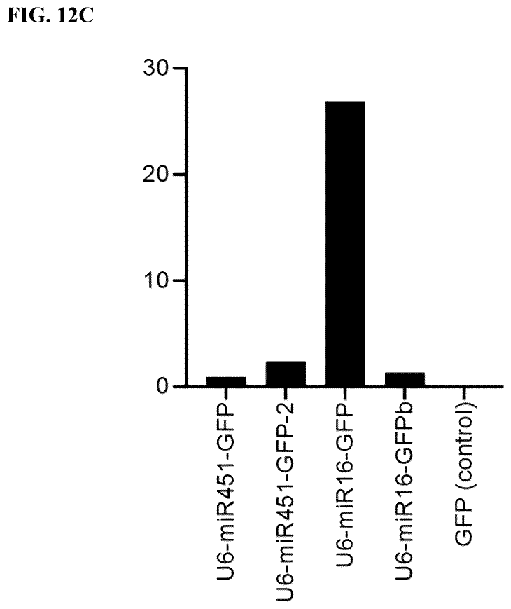

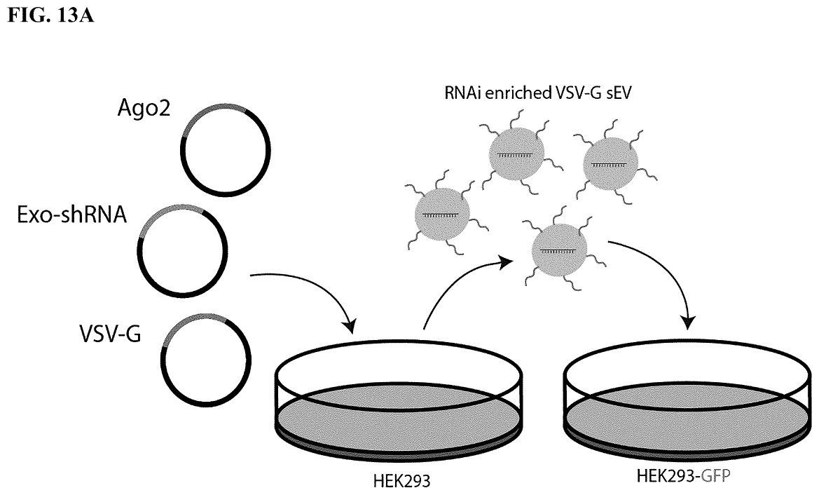

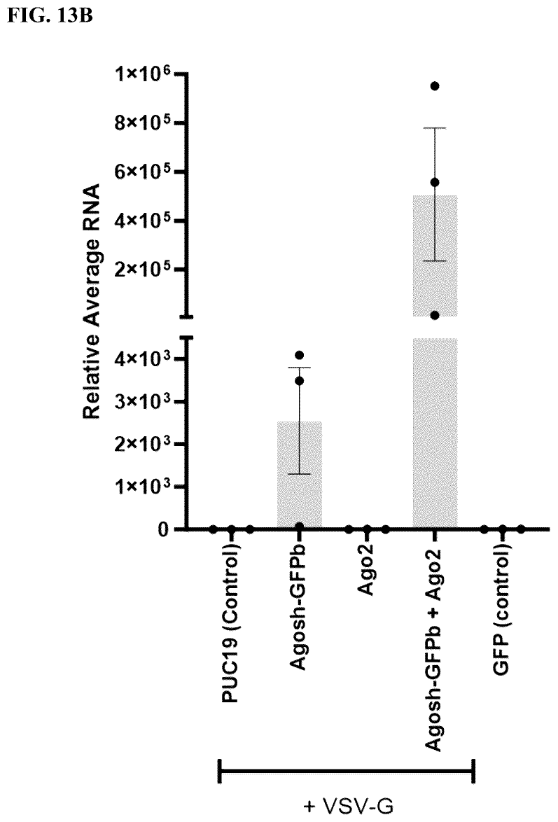

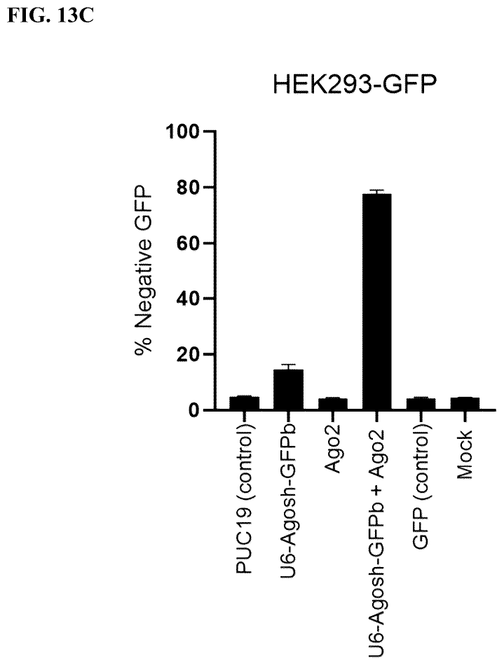

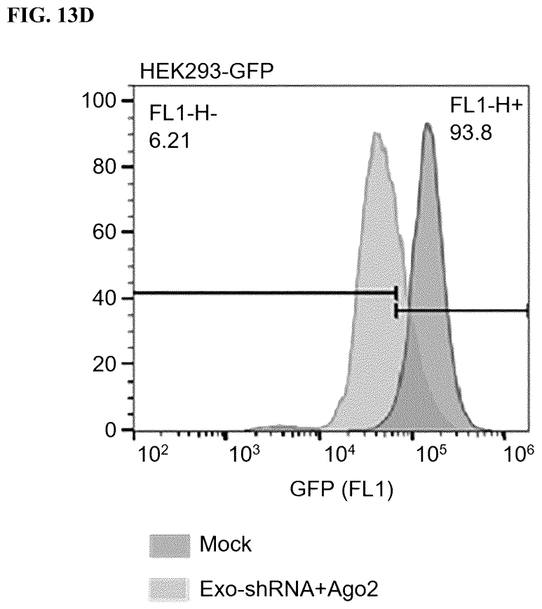

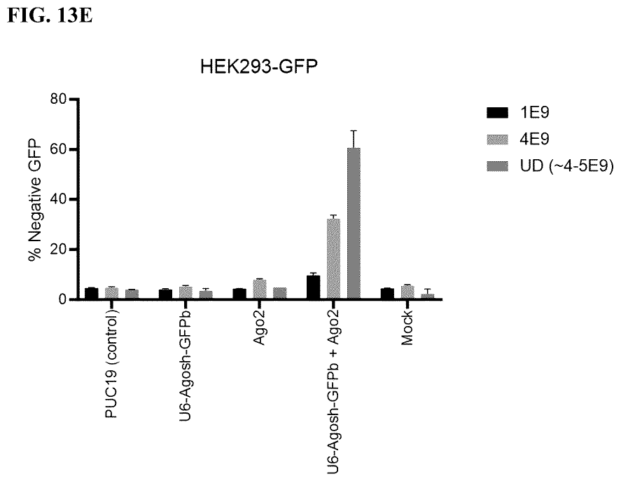

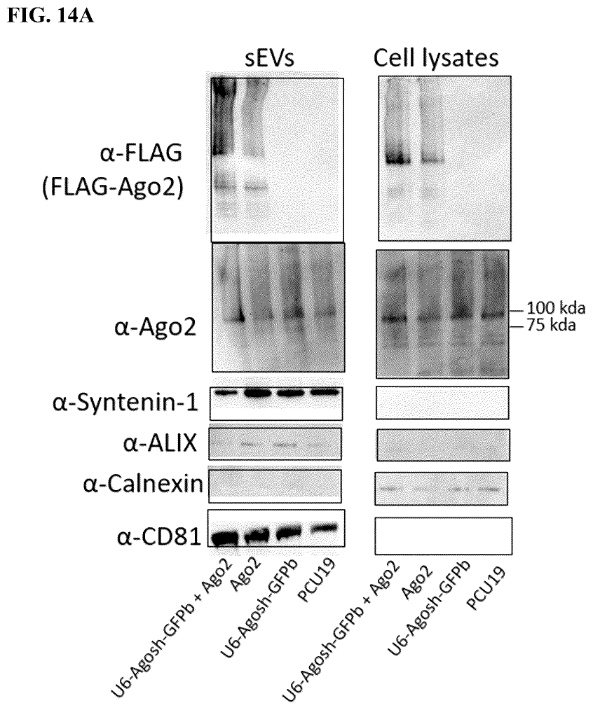

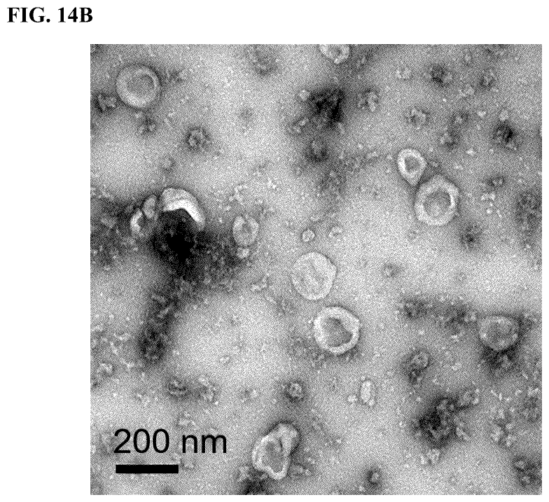

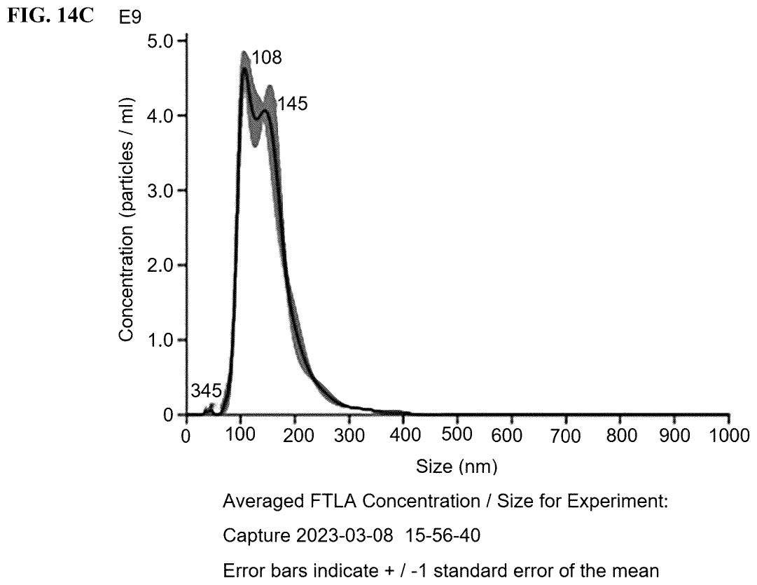

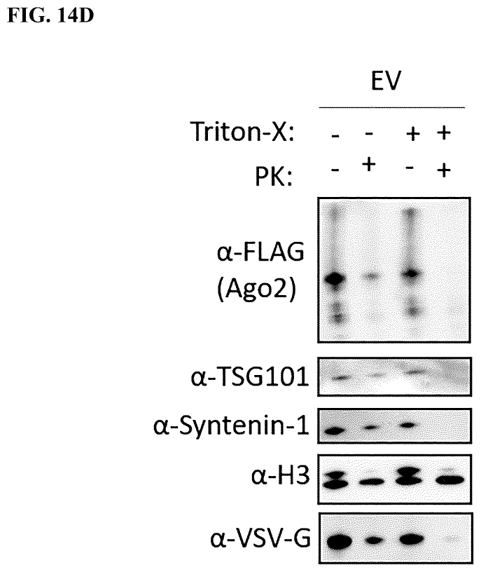

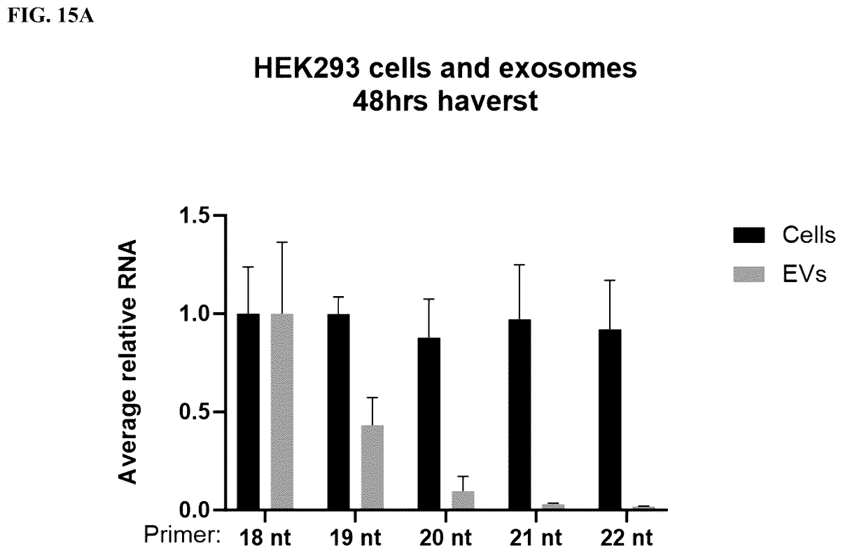

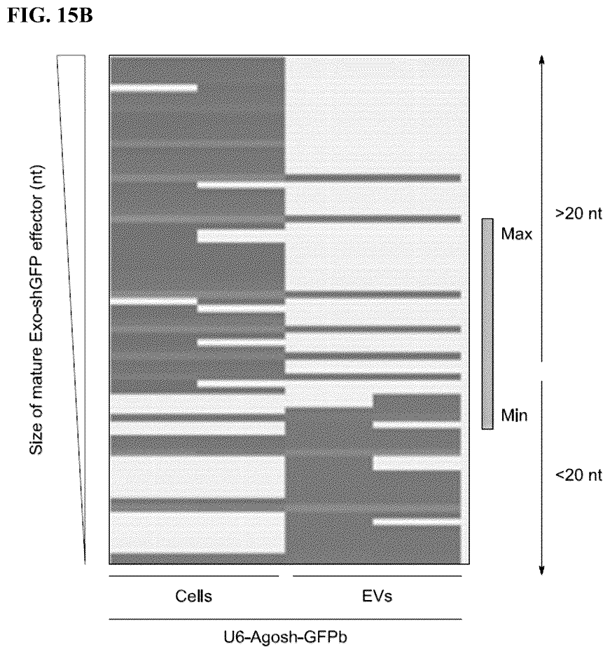

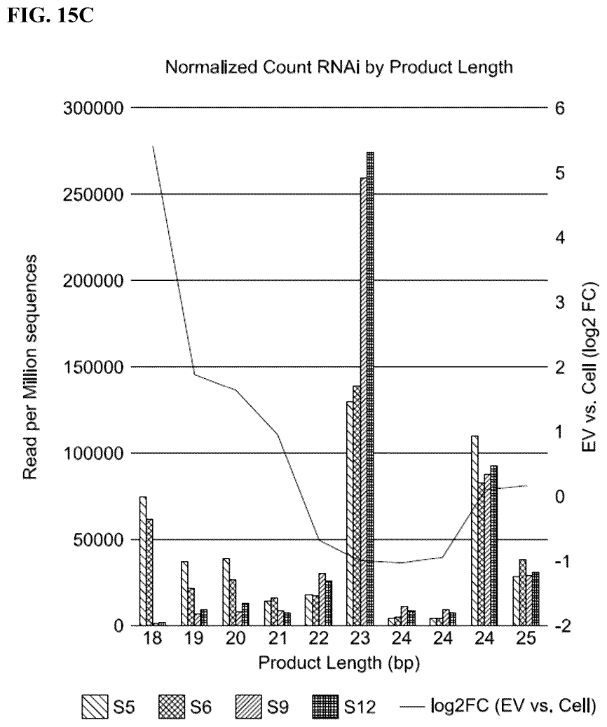

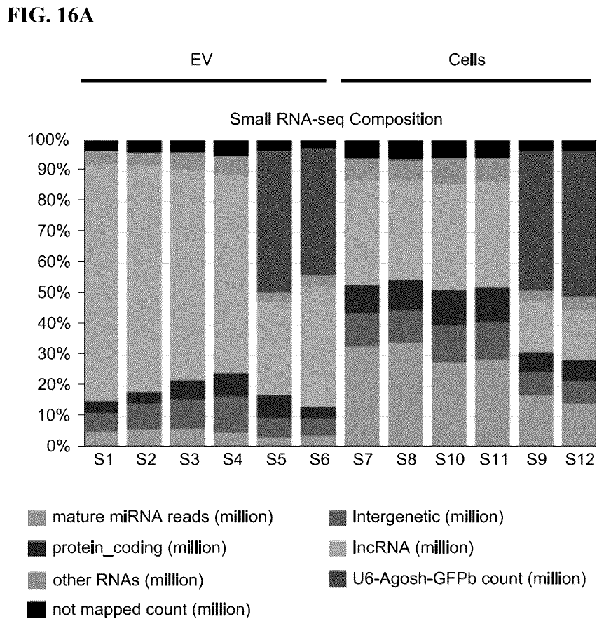

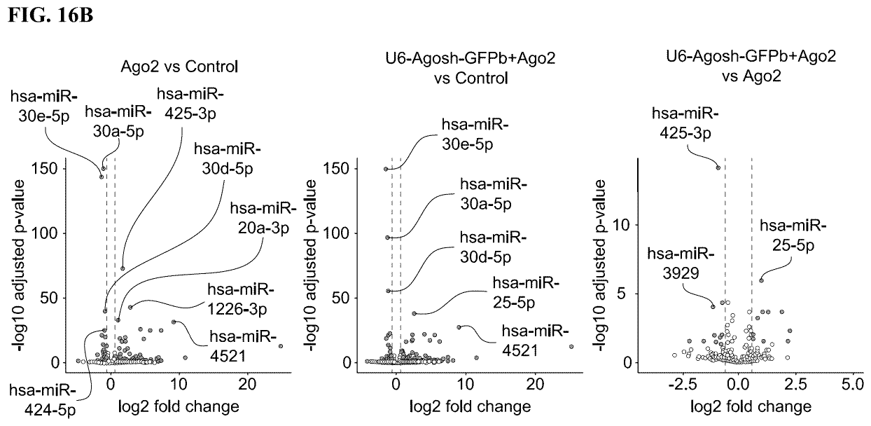

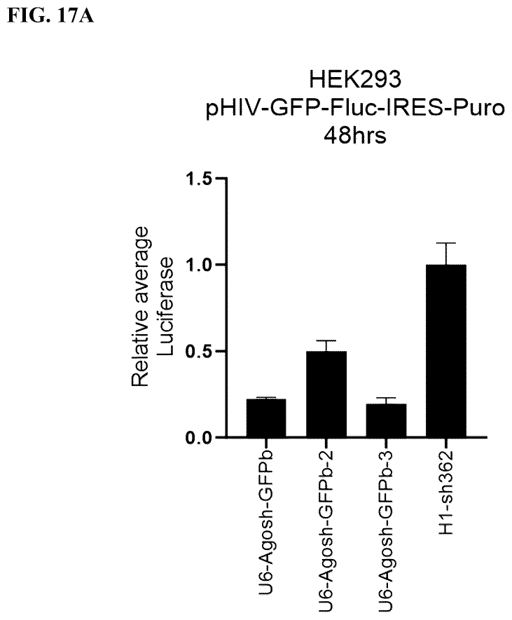

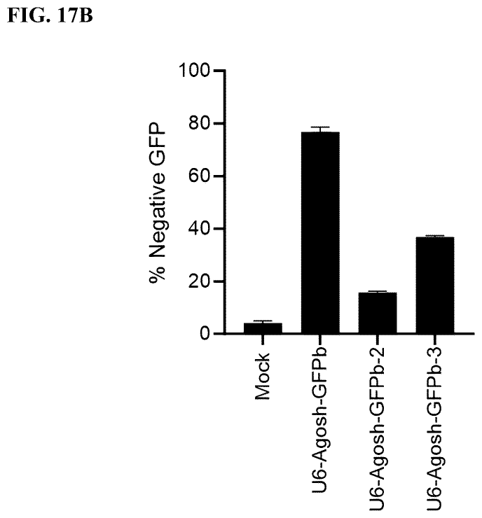

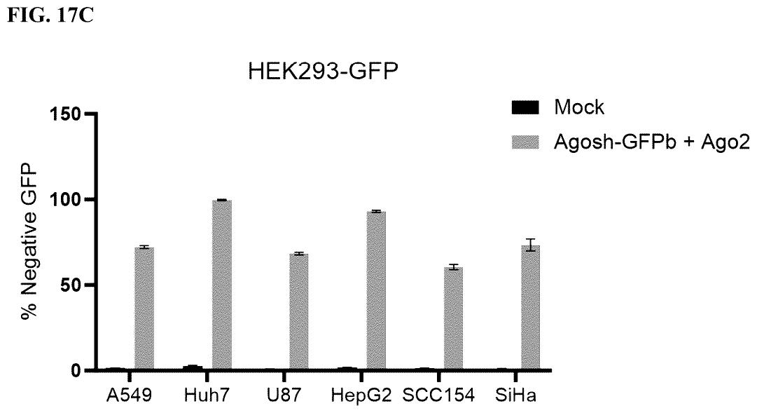

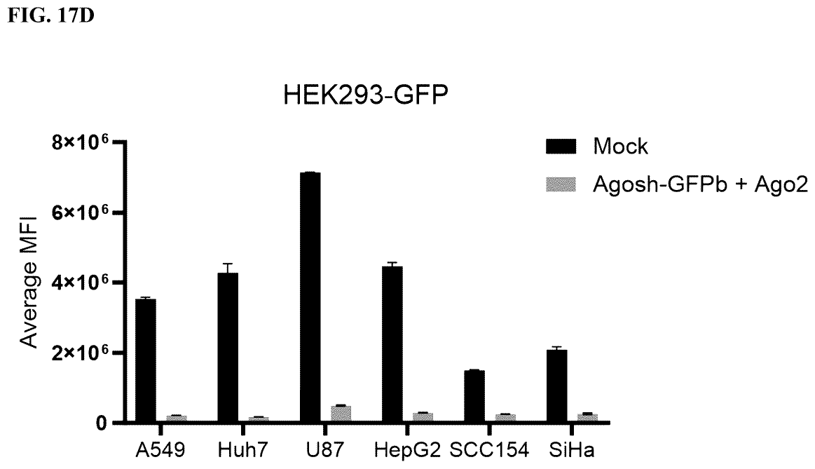

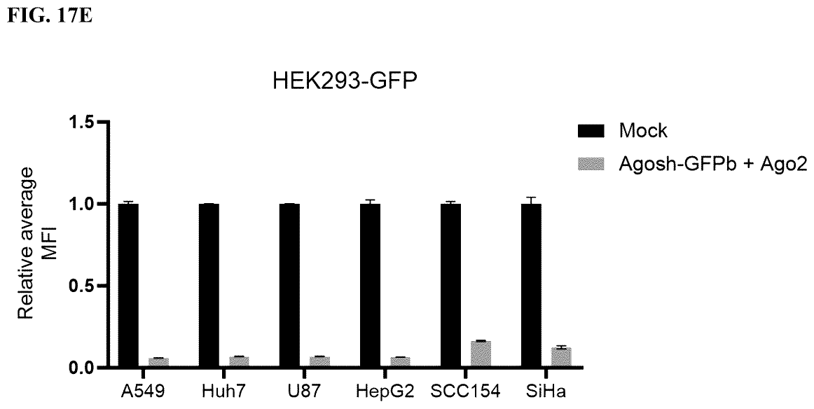

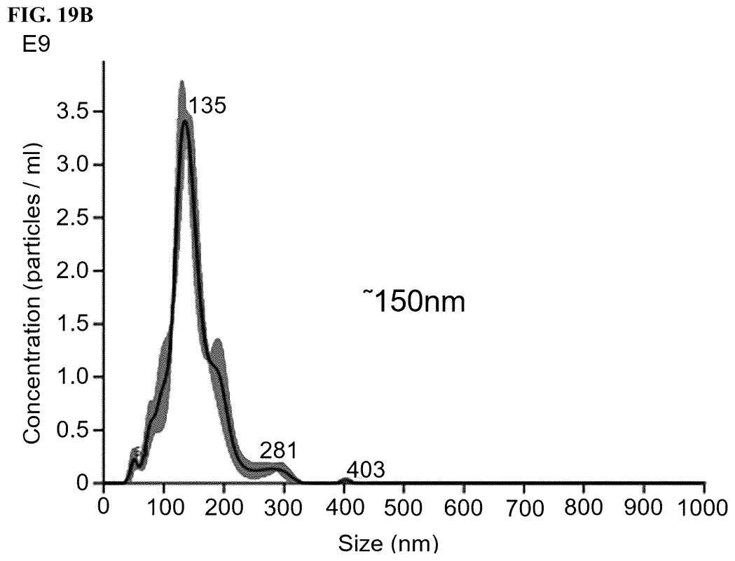

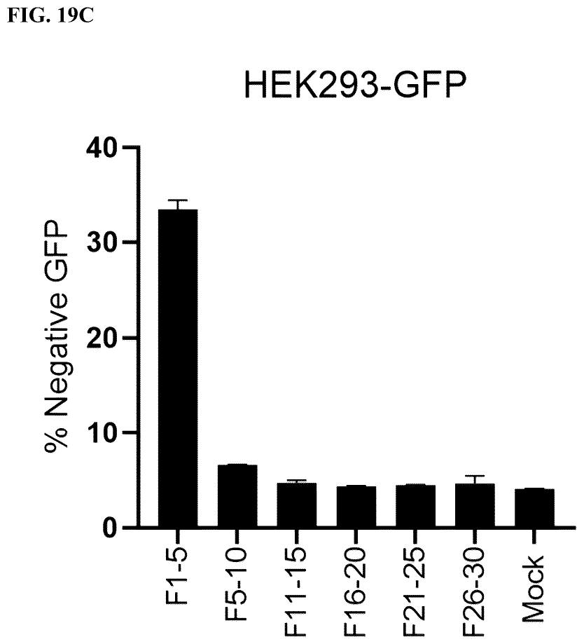

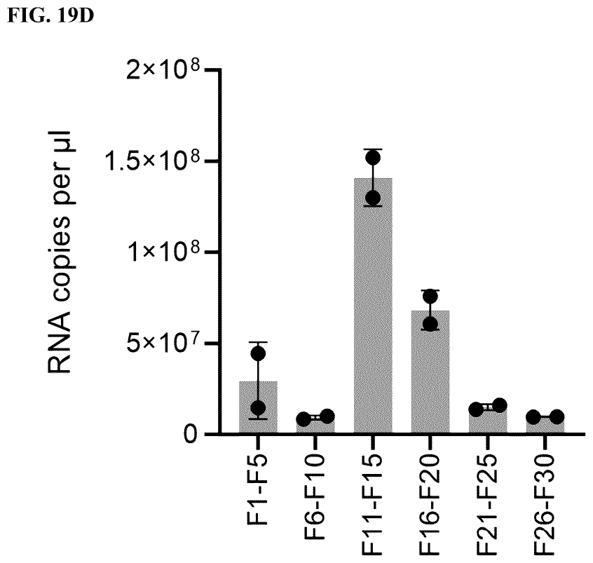

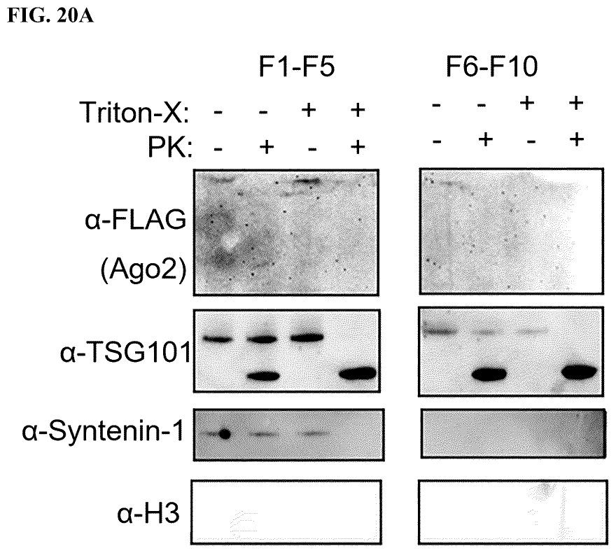

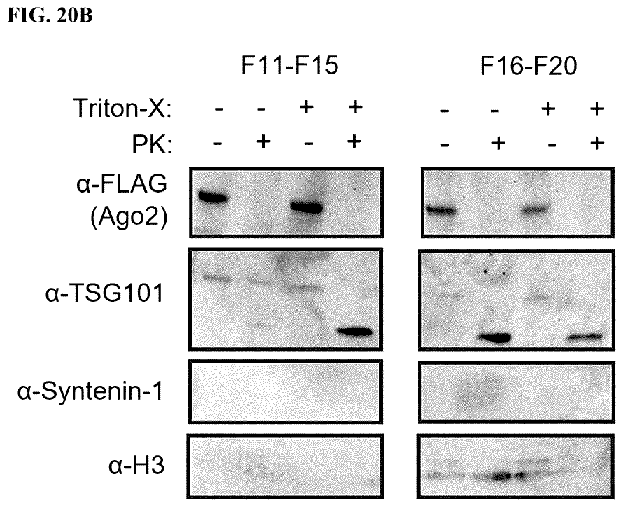

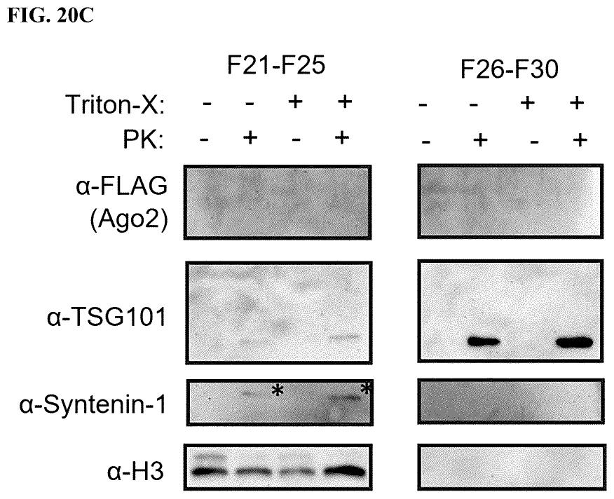

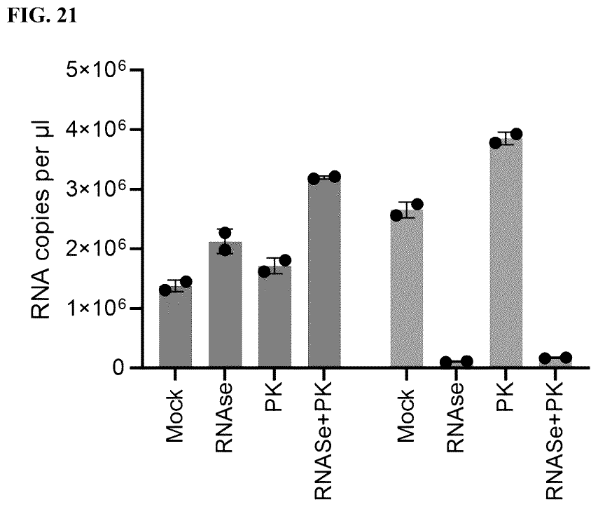

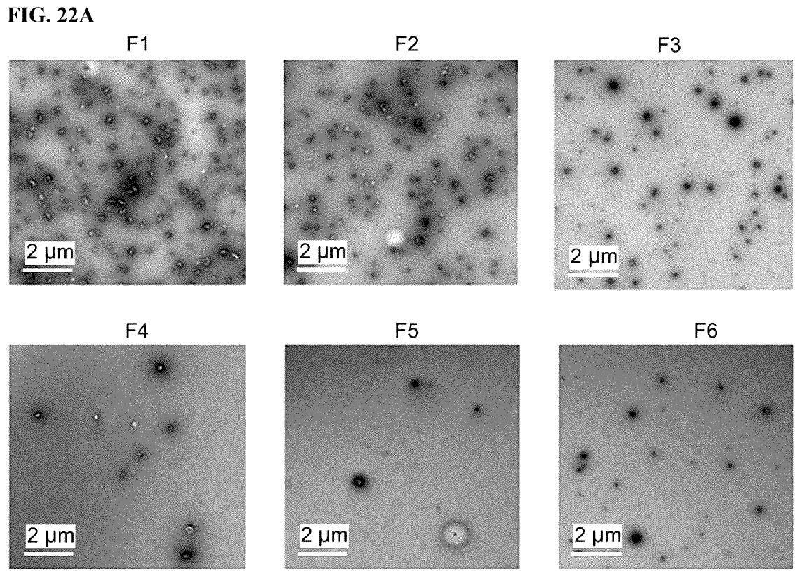

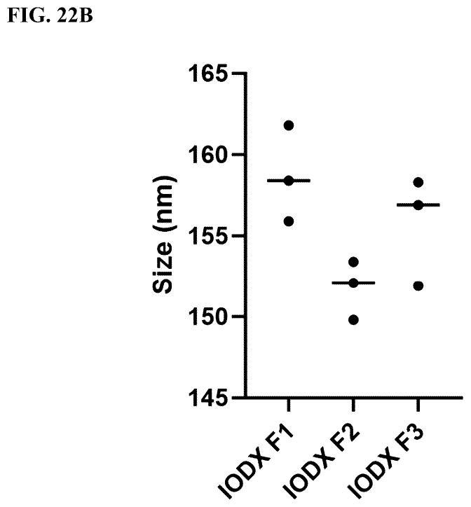

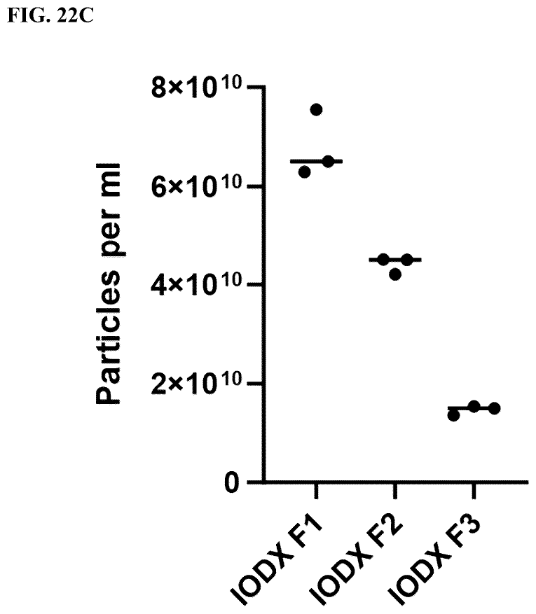

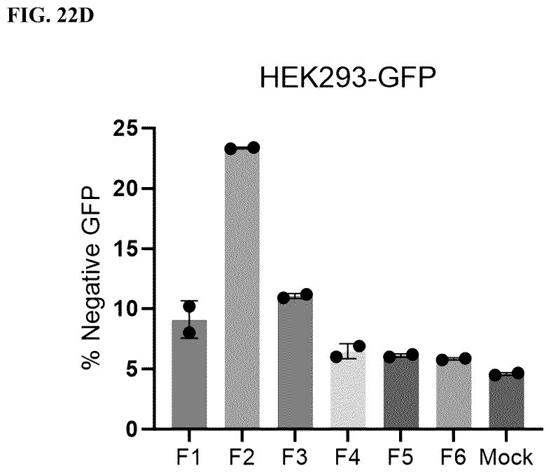

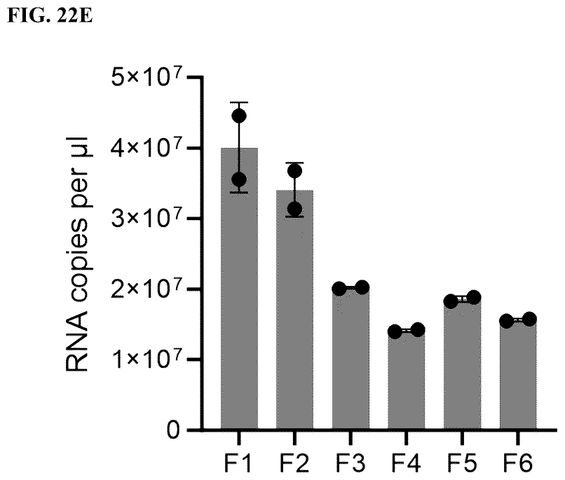

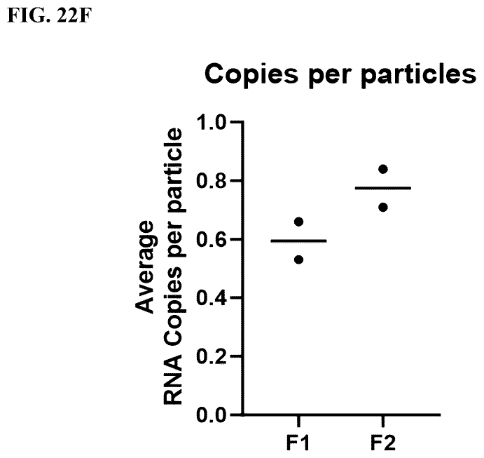

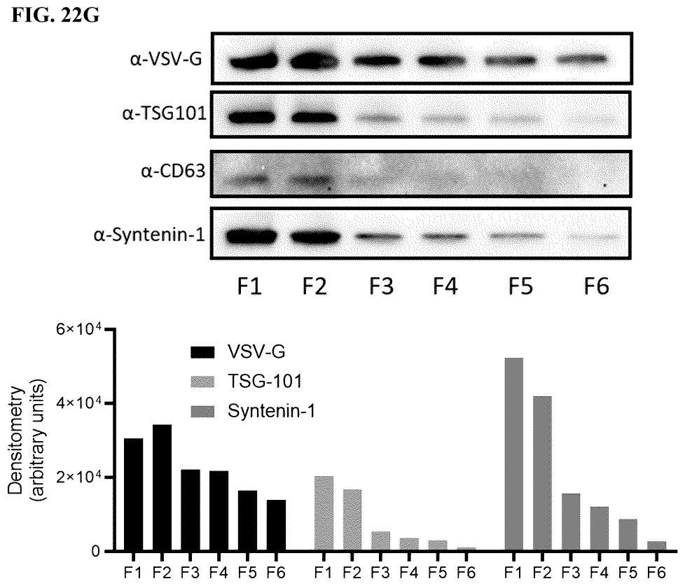

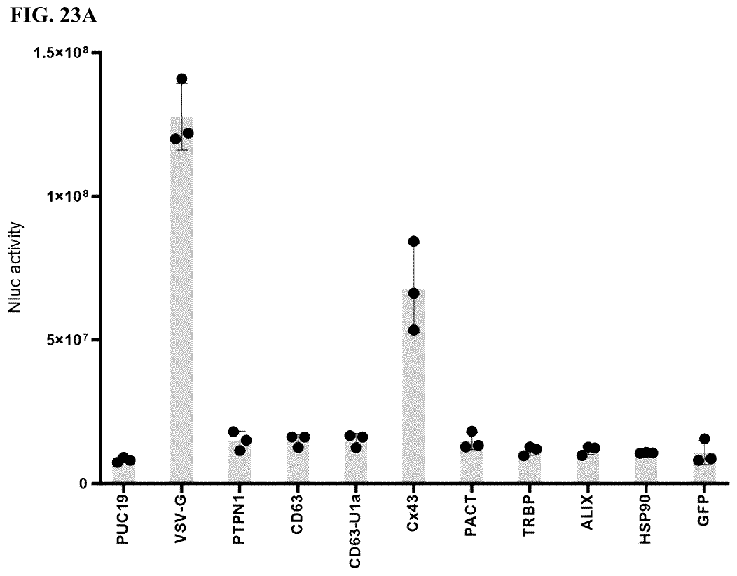

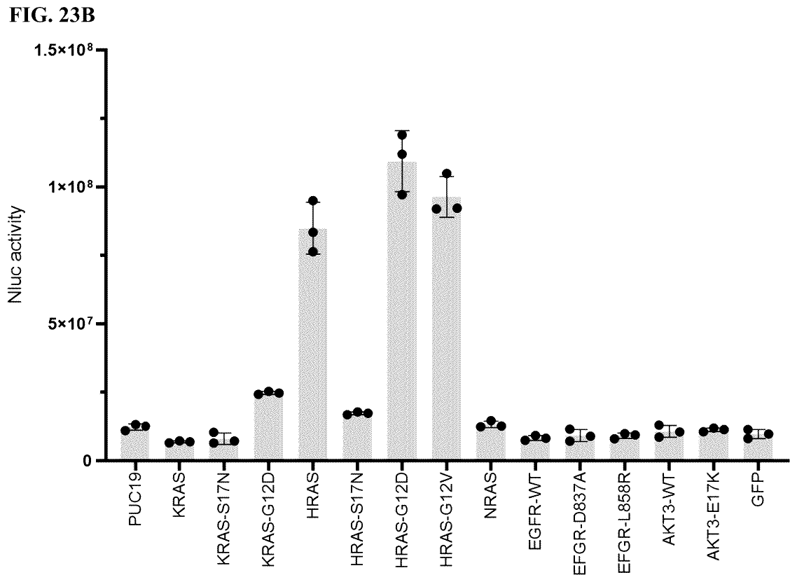

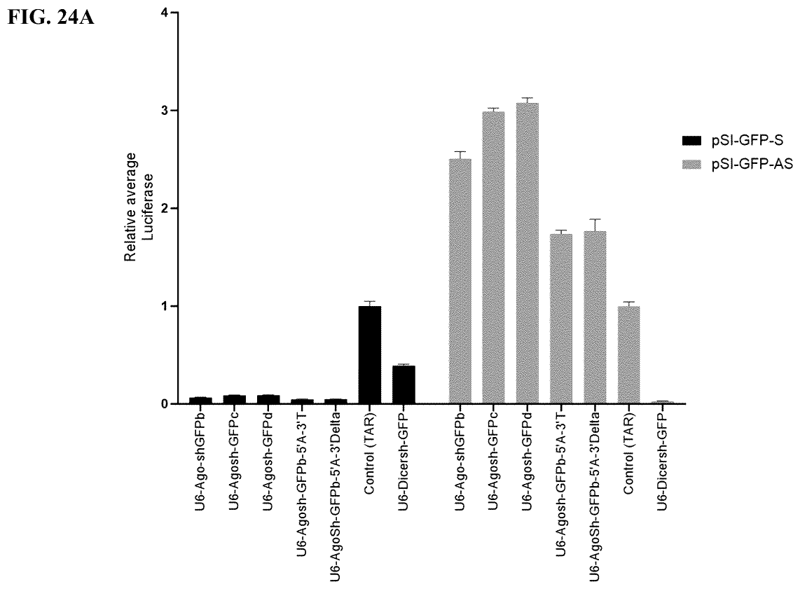

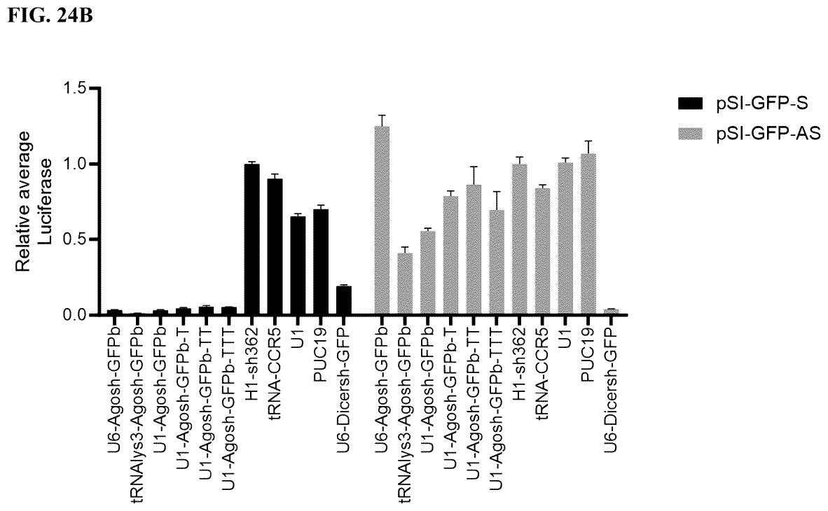

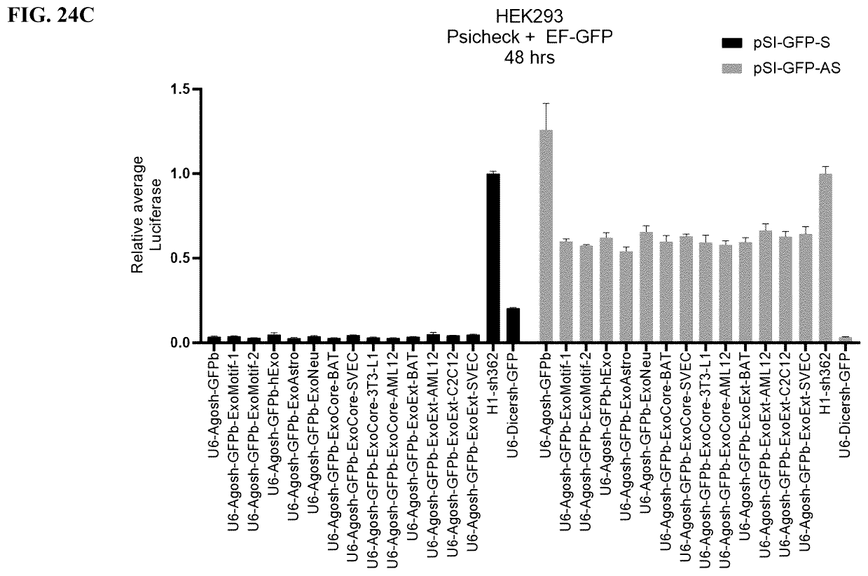

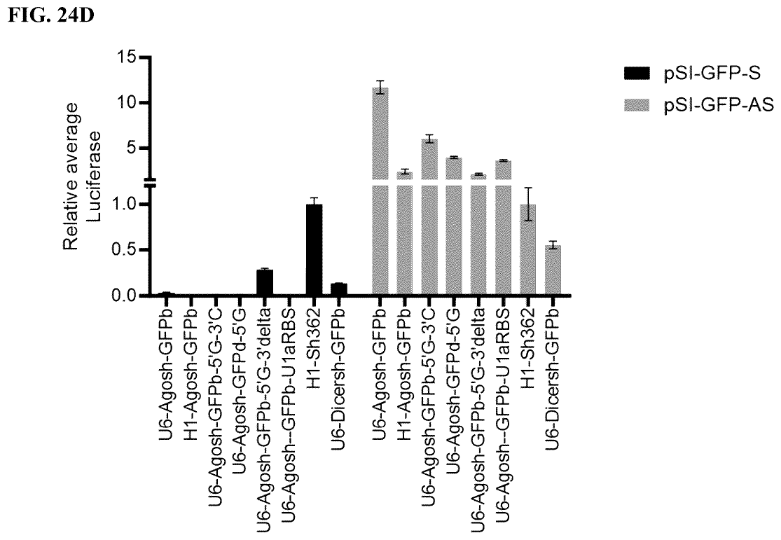

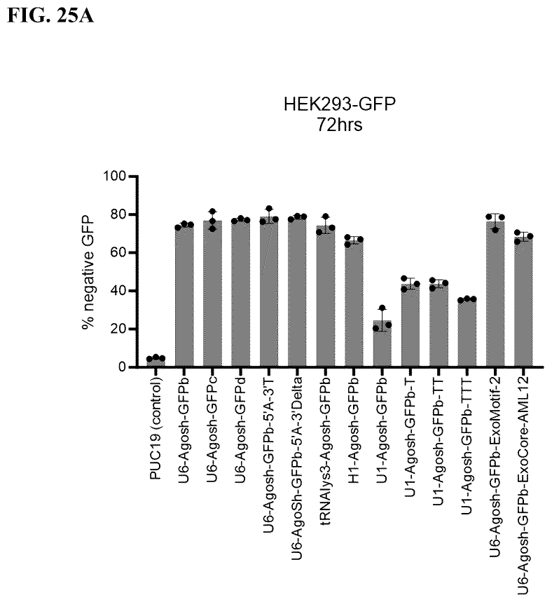

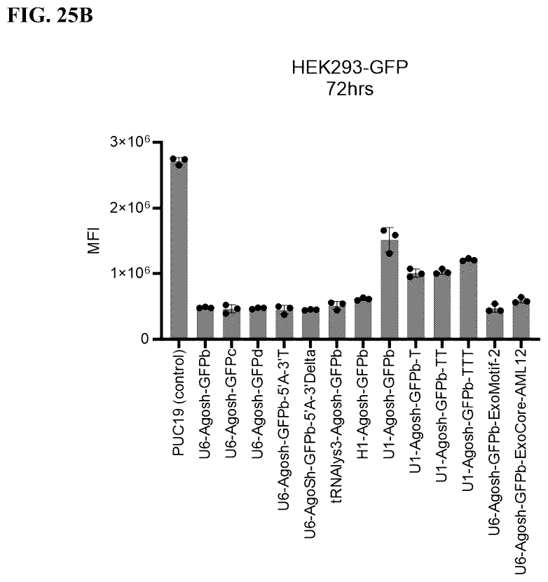

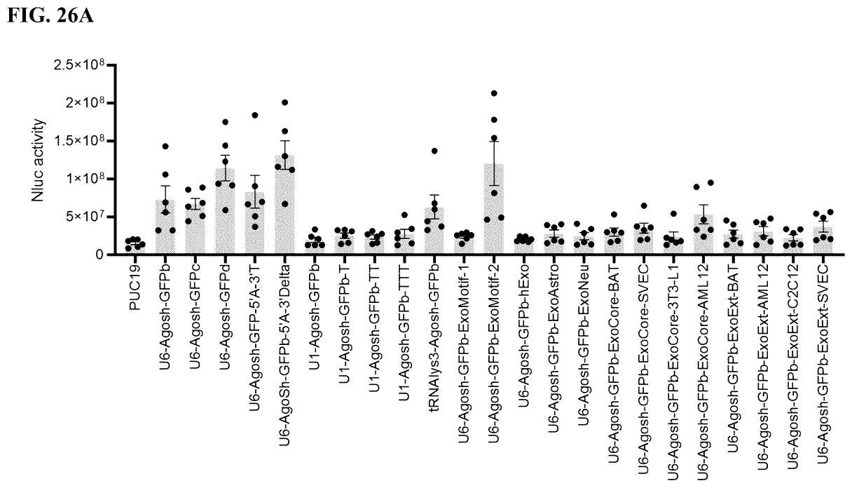

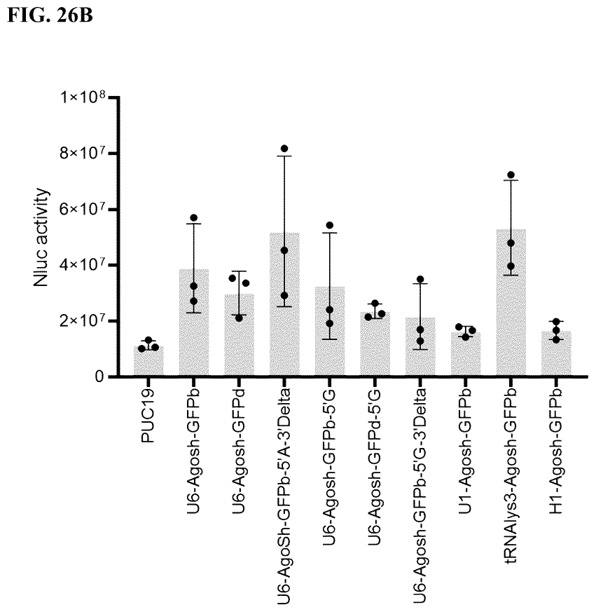

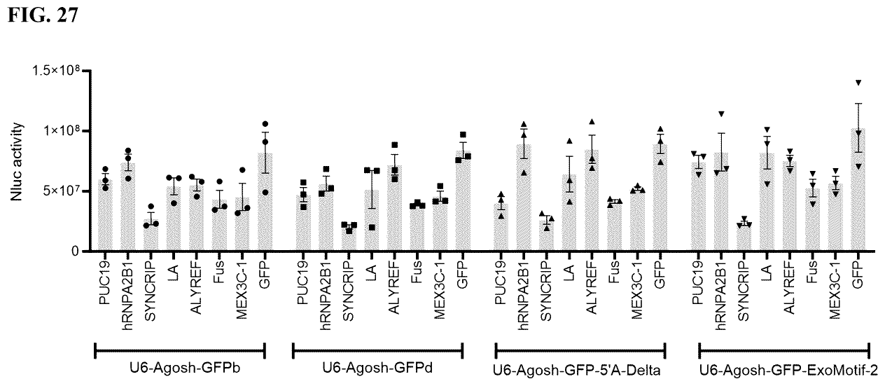

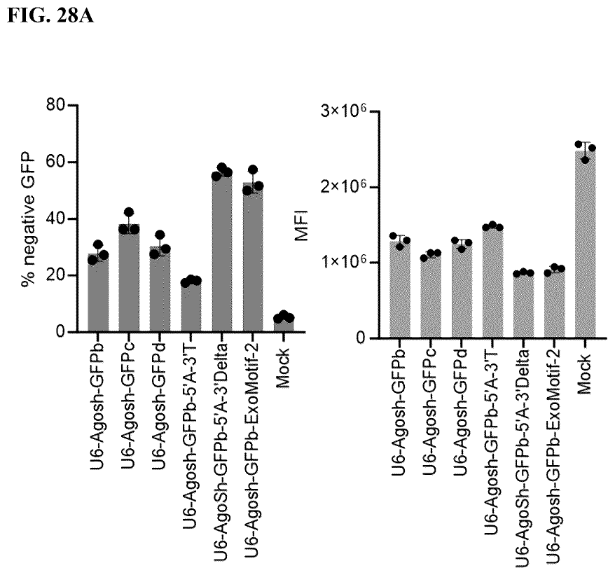

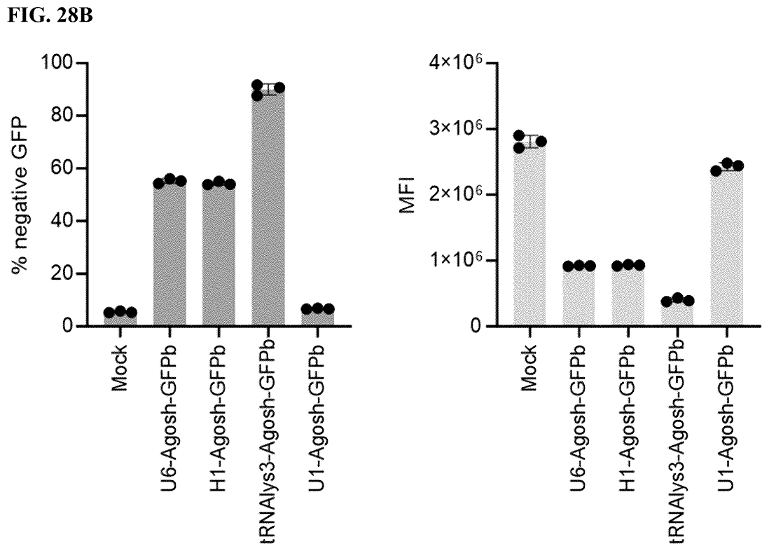

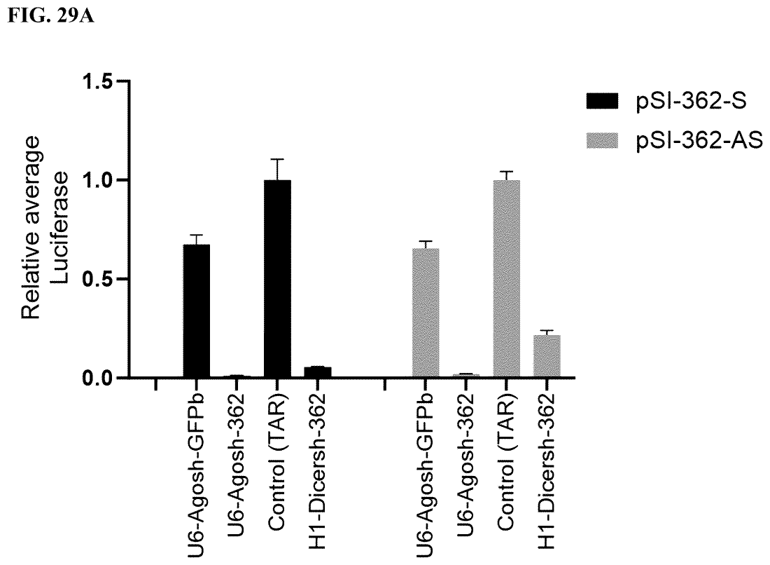

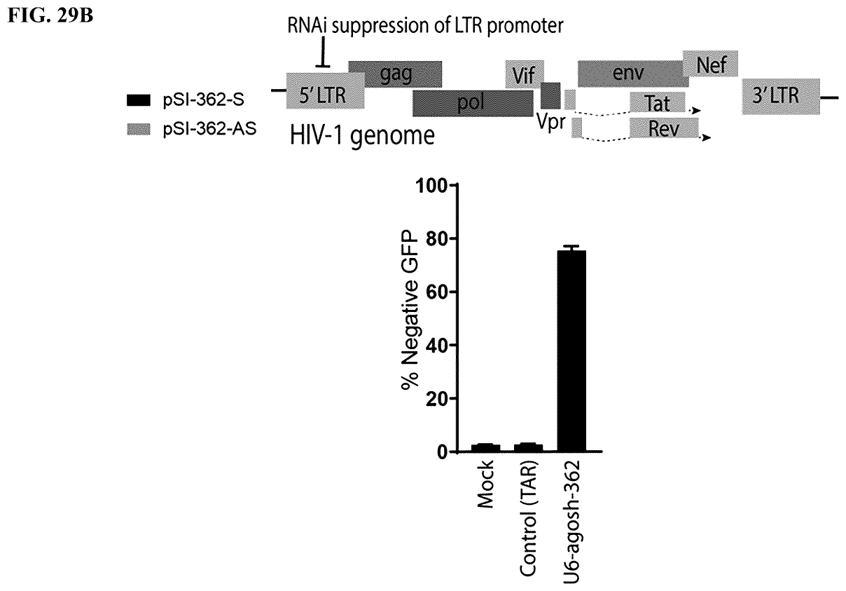

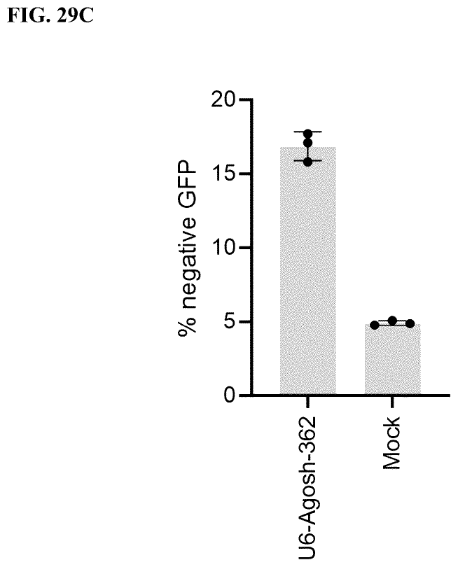

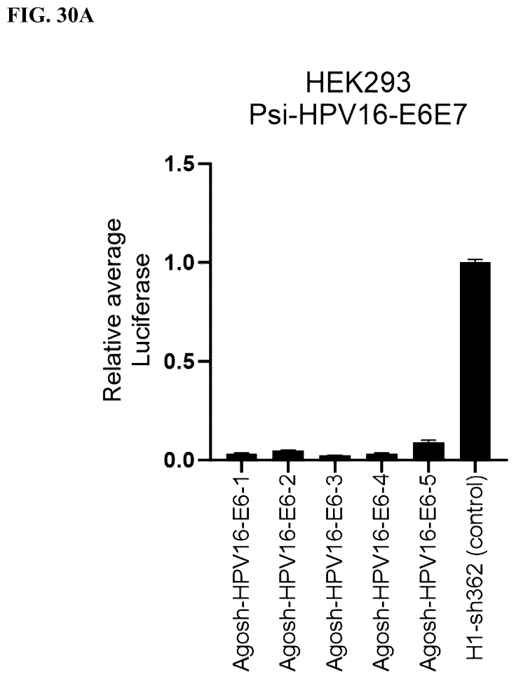

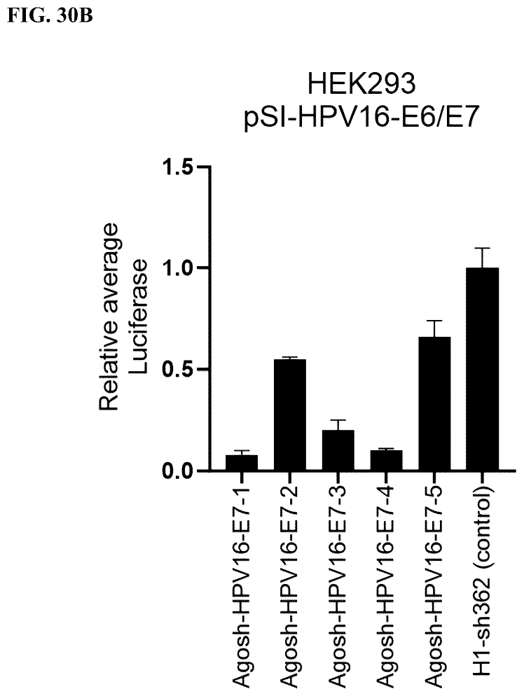

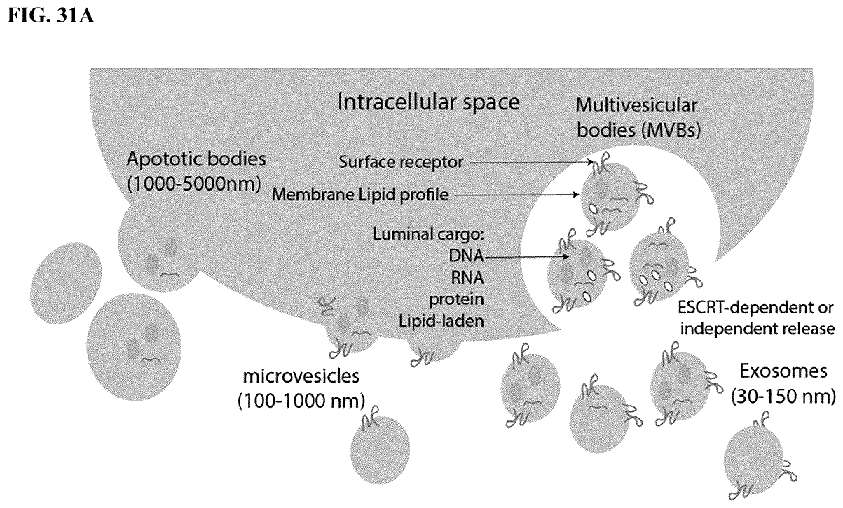

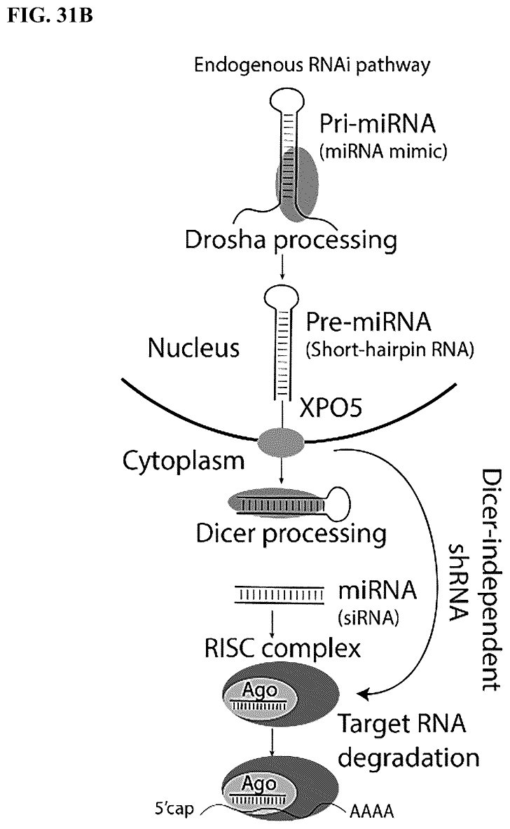

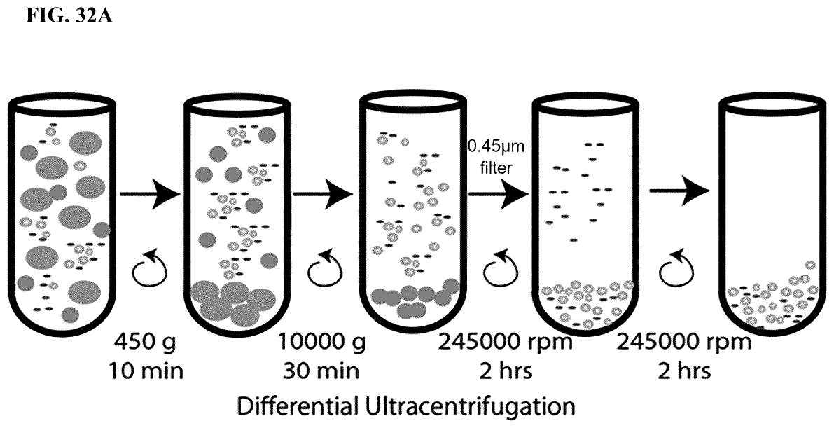

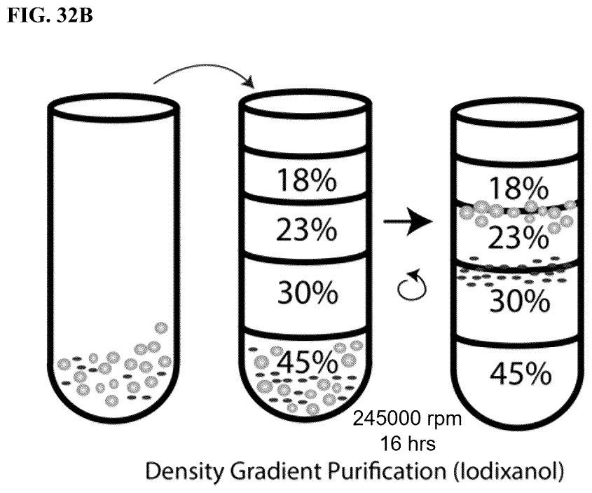

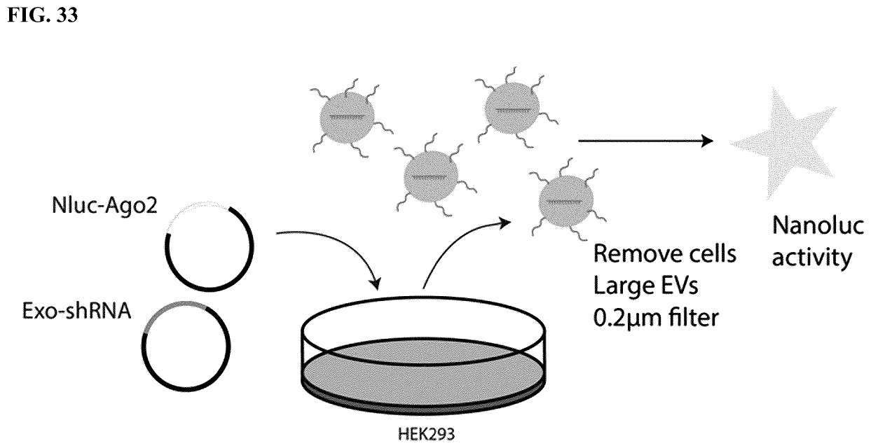

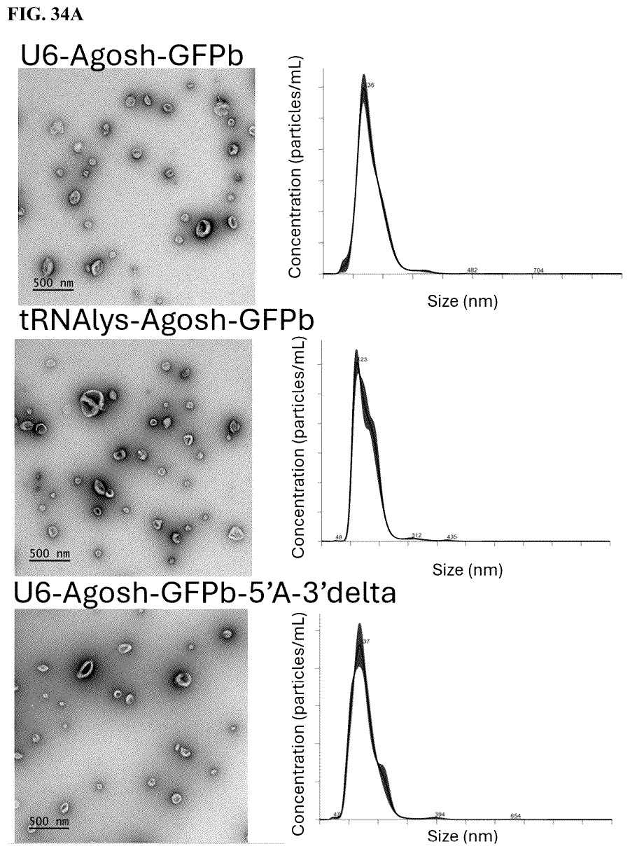

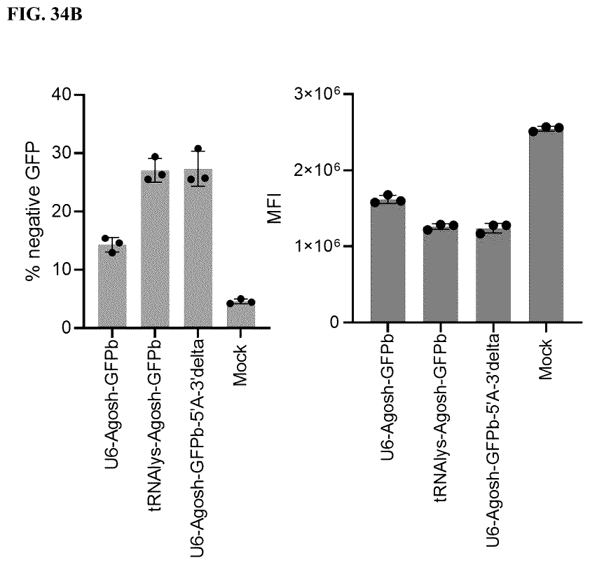

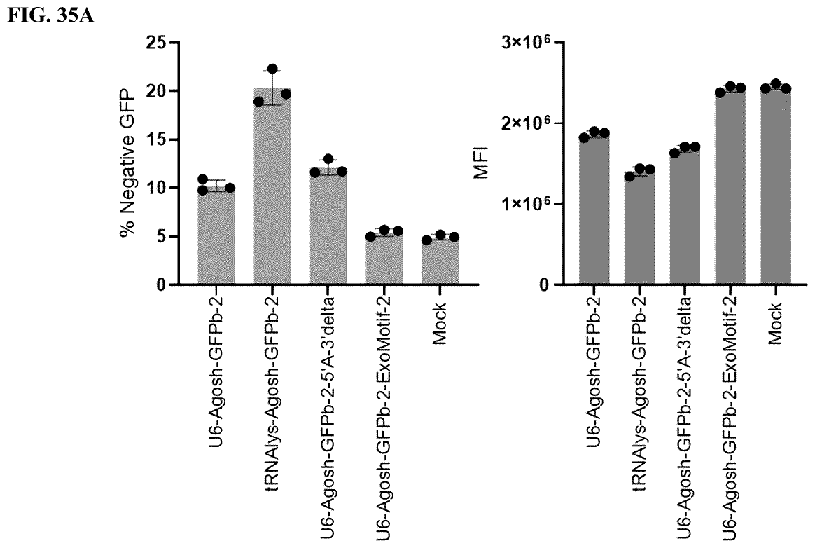

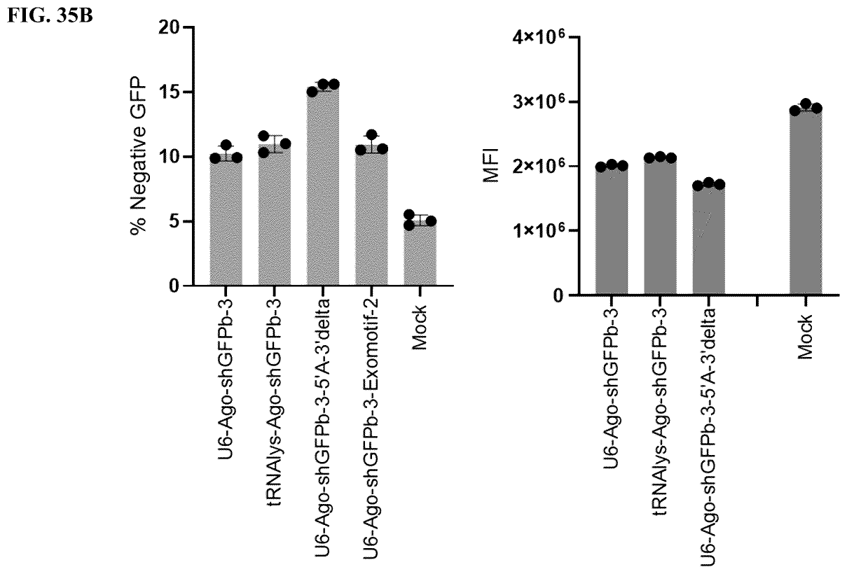

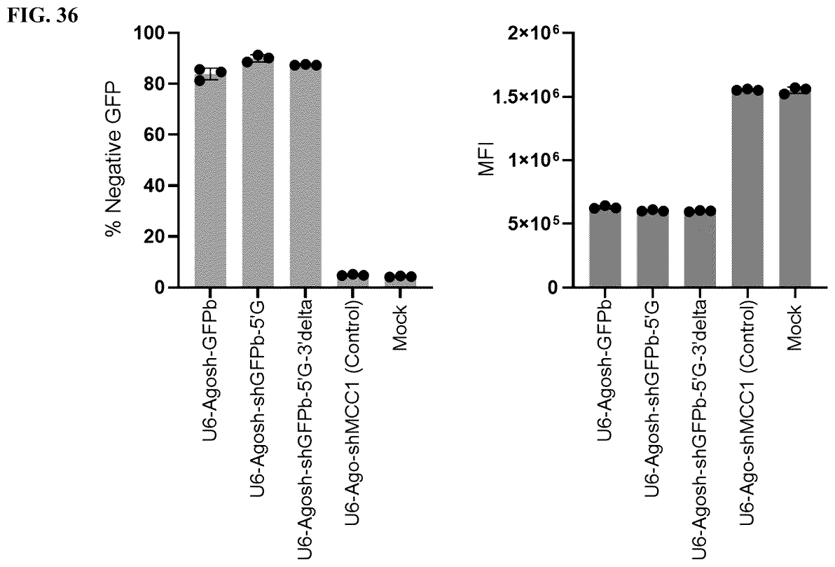

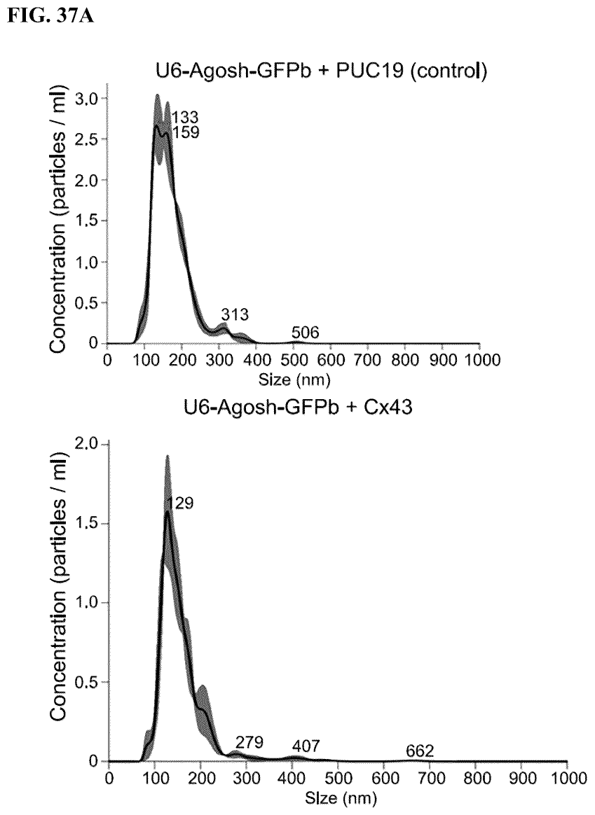

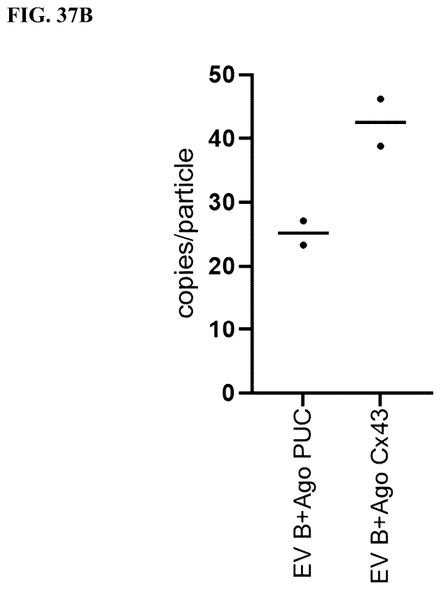

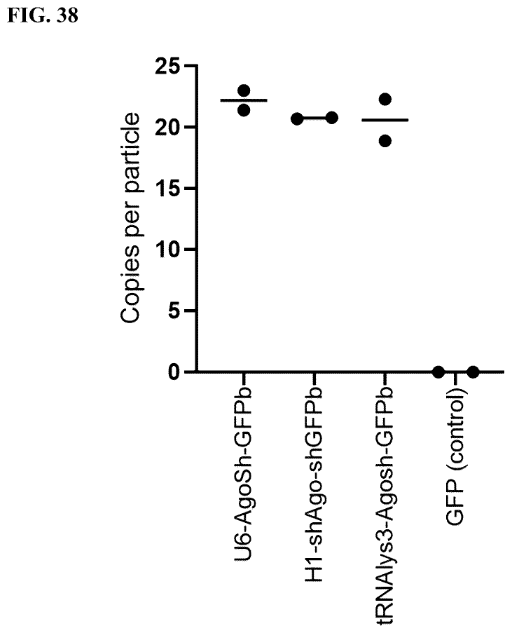

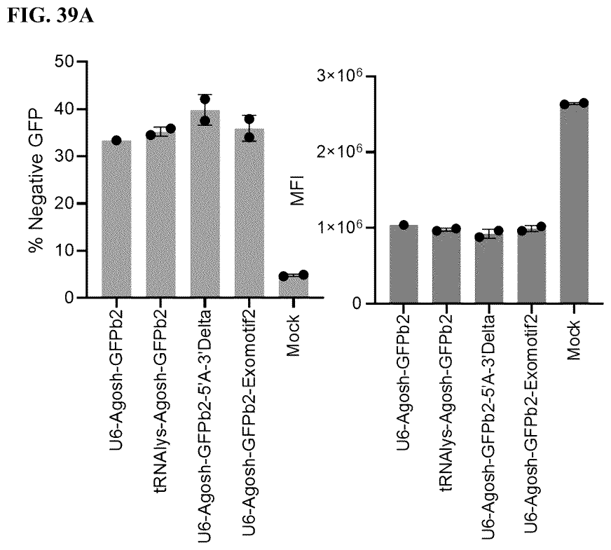

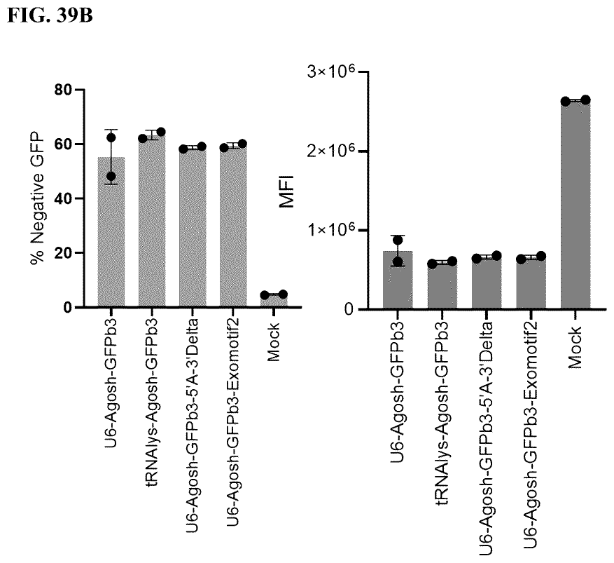

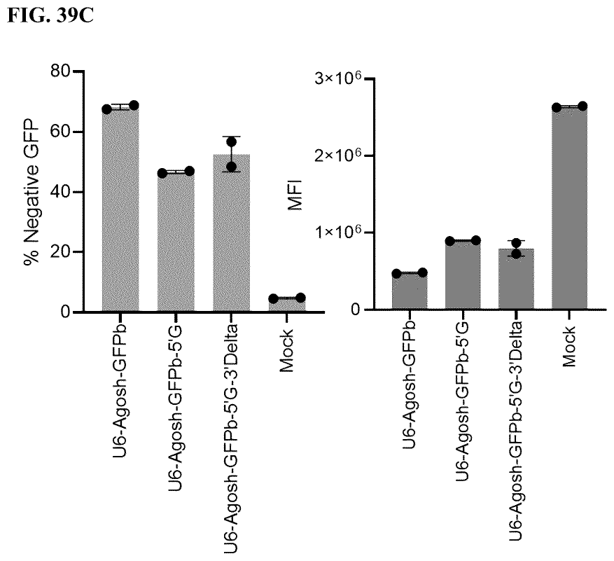

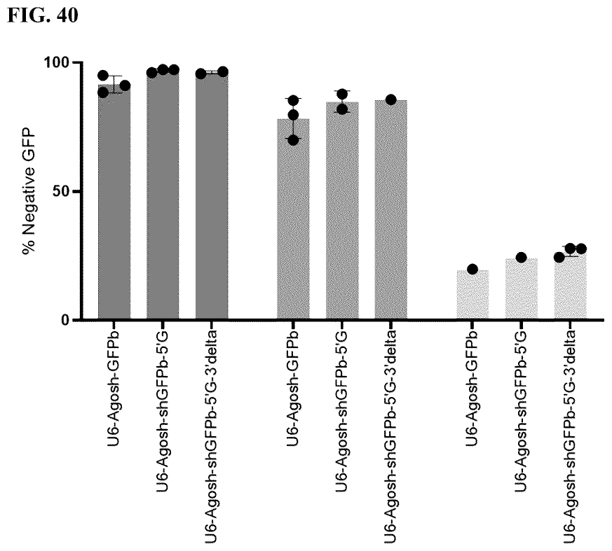

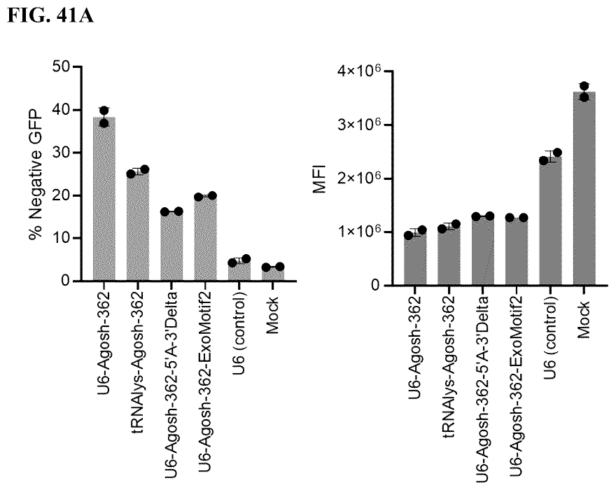

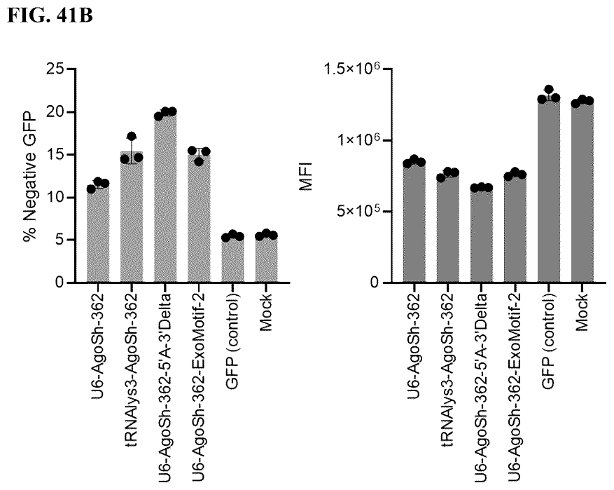

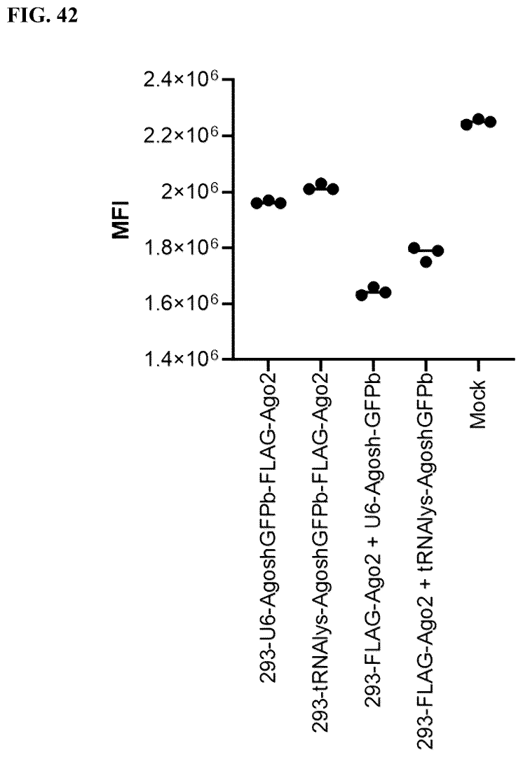

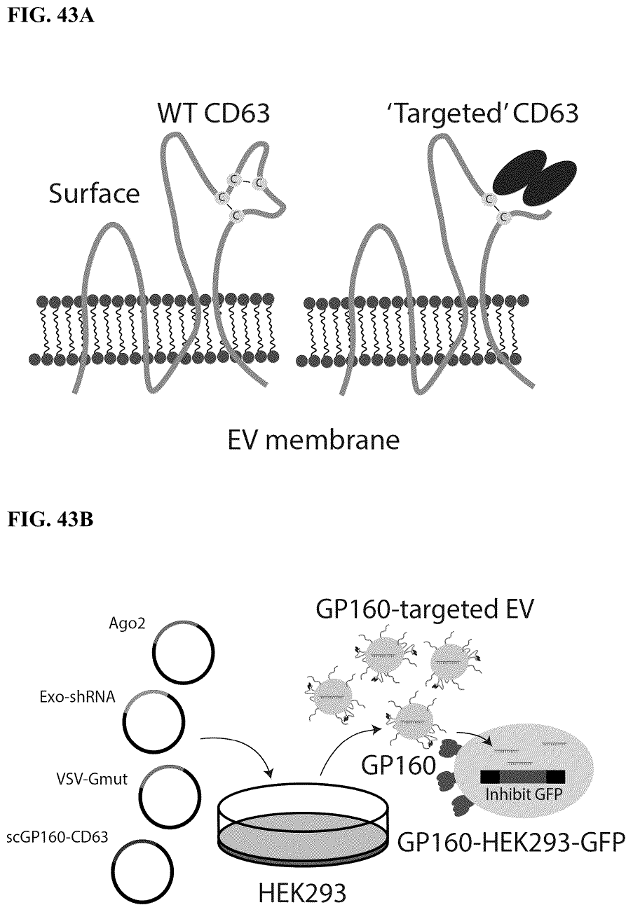

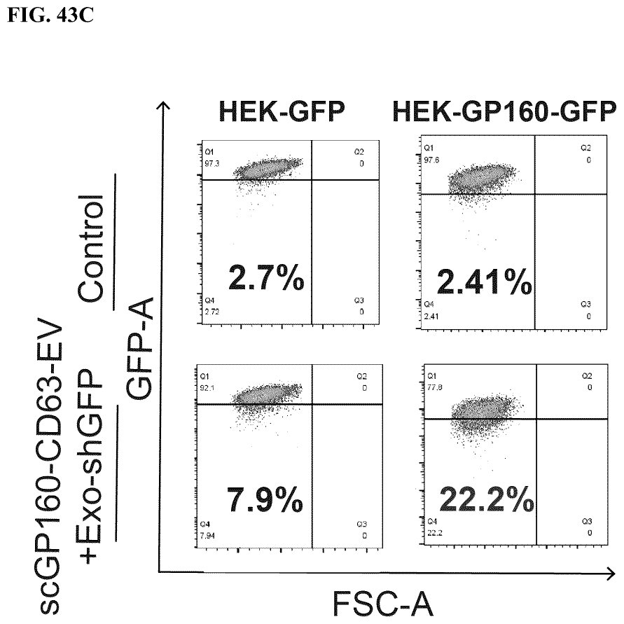

FIG. 4A-41B. Ago2 sh362 variants transfected into a HEK293-GFP cell line. (FIG. 41A) HEK293-HIV-LTR-GFP cells were transfected with the Agosh-362 and its promoter (tRNAlys) or sequence and motif variants (5′A-3′Delta and ExoMotif-2). Knockdown of GFP was assessed at 72 hrs later by flow cytometry. A vector with the U6 promoter (control) or untreated cells (mock) was included as a negative control. The error bars were generated by transfections performed in duplicate. (FIG. 41B) HEK293 cells were triple transfected with a vector expressing the Agosh-362 and its promoter and sequence variant, and a vector expressing a FLAG-tagged Ago2 (Ago2) and a Vesicular stomatitis virus G protein (VSV-G). The EVs were purified using differential ultracentrifugation. The purified EV fractions were added to a HEK293-GFP cell line and knockdown of GFP was assessed at 72 hrs post-addition by flow cytometry. % negative GFP and MFI are presented. The error bars were generated by transfections performed in triplicate. Results show improved potency with Exo-shRNA variants targeting the promoter of HIV is not predicted by Exo-sh362 activity.

FIG. 42. EVs derived from stable Exo-shRNA expressing HEK293 cells can facilitate knockdown in recipient reporter cells. EVs were purified from a stable HEK293 cell line expressing the U6-Agosh-GFPb or tRNAlys-Agosh-GFPb and FLAG-Ago2-IRES-Puromycin (293-U6-AgoshGFPb-FLA G-Ago2 or 293-tRNA lys-AgoshGFPb-FLAG-Ago2). A Iso, EVs were purified from a stable HEK293-FLAG-Ago2-IRES-Puromycin cell line transfected with a U6-Agosh-GFPb or tRNA lys-Agosh-GFPb vector (293-FLAG-Ago2+U6-Agosh-GFPb or 293-FLA G-Ago2+tRNA lys-AgoshGFPb). The purified EV fractions were added to a HEK293-GFP cell line and knockdown of GFP was assessed at 72 hrs post-addition by flow cytometry. MFI is presented. Untreated (mock) cells were included as a negative control. Experiments were performed in triplicate. The results show that stably transfected Ago2 and Ago2+Exo-shRNA cell lines facilitate transfer of EV knockdown.

FIGS. 43A-43C. Surface-modified Exo-shRNA EV can be targeted to a receptor-expressing cell line and facilitate reporter knockdown. (FIG. 43A) Schematic of a CD63 receptor and a CD63 embedded with a single-chain variable fragment (scFv) targeted to GP160 (scGP160-CD63). (FIG. 43B) Schematic of the production setup for generating surface-modified EVs loaded with matured RNAi effectors. HEK293 cells were quadruple transfected with a vector expressing the Dicer-independent shRNA target GFP (Agosh-GFPb), a vector expressing a FLAG-tagged Ago2 (Ago2), scGP160-CD63 targeting vectors, and a mutated Vesicular stomatitis virus G protein (VSV-Gmut). The VSV-Gmut contains a K47Q R354Q mutation and cannot bind the LDL receptor (LDL-R), but can still facilitate fusion and RNAi release if taken up through the targeted GP160 receptor. The EVs were purified using differential ultracentrifugation. (FIG. 43C) A HEK293 cell line with a GFP reporter (HEK-GFP) was generated that stably expressed a membrane-bound GP160 from HIV-1 (HEK-GP160-GFP). The scGP160-CD63 modified EVs loaded with the Exo-shGFP were added to cells and 72 hrs later the level of GFP was assessed. The EVs were added to a control HEK293-GFP without the GP160 receptor. Mock-treated cells were included as a control. The level of GFP versus FSC are presented. The scGP160-CD63 Exo-shGFP EVs resulted in significantly higher knockdown of GFP in the HEK-GP160-GFP compared to the HEK-GFP cells, demonstrating the Exo-shRNA can be receptor targeted through modified EVs.

### DETAILED DESCRIPTION

A s used herein, the singular forms “a”, “an” and “the” are intended to include the plural forms as well, unless the context clearly indicates otherwise.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The abbreviations used herein have their conventional meaning within the chemical and biological arts. The chemical structures and formulae set forth herein are constructed according to the standard rules of chemical valency known in the chemical sciences.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). N on limiting examples, of nucleosides includes, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides, contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. N on-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein may be part of a vector. For example, the nucleic acid provided herein may be part of a viral vector, which may be transduced into a cell. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e, an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety. Because the different proteins in fusion proteins may affect the functionality of other proteins under certain circumstances, peptide linkers may be used between different proteins within the same fusion protein. These peptide linkers may have a flexible structure and separate the proteins within the fusion protein so that each protein in the fusion proteins substantially retains its function. Peptide linkers are known in the art and described, for example, in Chen et al, Adv Drug Deliv Rev, 65(10); 1357-1369 (2013).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. W here there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., AGO2) in other proteins with different numbering systems. For example, by performing a simple sequence alignment with a protein (e.g., AGO2) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three-dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Y et another indication that two nucleic acid sequences are substantially identical is that the same primers can be used to amplify the sequence.

The term “AGO2 protein” or “AGO2” as used herein includes any of the recombinant or naturally-occurring forms of argonaute 2 (AGO2), also referred to as Argonaute RISC catalytic component 2, Eukaryotic translation initiation factor 2C 2, PAZ Piwi domain protein (PPD), protein slicer, or variants or homologs thereof that maintain AGO2 activity (e.g. within at least 50%, 80%, 90%, 95%, 96%, 97%, 98%, 99% or 100% activity compared to AGO2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring AGO2 protein. In embodiments, AGO2 is substantially identical to the protein identified by the UniProt reference number Q9U KV8 or a variant or homolog having substantial identity thereto. In embodiments, AGO2 has at least 90%, 95%, 96%, 97%, 98%, 99% or 100% amino acid sequence identity across the whole sequence or a portion of the sequence (e.g. a 50, 100, 150 or 200 continuous amino acid portion) of SEQ ID NO:156.

The term “KRAS protein” or “KRAS” as used herein includes any of the recombinant or naturally-occurring forms of KRAS, also referred to as GTPase KRas, or variants or homologs thereof that maintain KRAS activity (e.g. within at least 50%, 80%, 90%, 95%, 96%, 97%, 98%, 99% or 100% activity compared to KRAS). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KRA S protein. In embodiments, KRAS is substantially identical to the protein identified by the UniProt reference number P01116 or a variant or homolog having substantial identity thereto. In embodiments, KRAS has at least 90%, 95%, 96%, 97%, 98%, 99% or 100% amino acid sequence identity across the whole sequence or a portion of the sequence (e.g. a 50, 100, 150 or 200 continuous amino acid portion) of SEQ ID NO:217. In embodiments, the KRAS protein is a dominant negative KRAS (S17N) mutant. “Dominant negative KRAS mutant” refers to a KRAS protein including an N residue at a position corresponding to position 17 of SEQ ID NO:217. In embodiments, the KRAS protein has a substitution mutation at any one of positions corresponding to position 12 or 17 of SEQ ID NO:217. In embodiments, the KRAS protein includes a D residue at a position corresponding to position 12 of SEQ ID NO:217. In embodiments, the KRAS protein includes a V residue at a position corresponding to position 12 of SEQ ID NO:217.

The term “connexin 43 protein” or “connexin 43” as used herein includes any of the recombinant or naturally-occurring forms of connexin 43 (Cx43), also referred to as Gap junction alpha-1 protein, Gap junction 43 kDa heart protein or variants or homologs thereof that maintain connexin 43 activity (e.g. within at least 50%, 80%, 90%, 95%, 96%, 97%, 98%, 99% or 100% activity compared to connexin 43). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring connexin 43 protein. In embodiments, connexin 43 is substantially identical to the protein identified by the UniProt reference number P17302 or a variant or homolog having substantial identity thereto.

The term “syncytin-A protein” or “syncytin-A” as used herein includes any of the recombinant or naturally-occurring forms of syncytin-A (SynA), also referred to as Syncytin-2 or variants or homologs thereof that maintain syncytin-A activity (e.g. within at least 50%, 80%, 90%, 95%, 96%, 97%, 98%, 99% or 100% activity compared to syncytin-A). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring syncytin-A protein. In embodiments, syncytin-A is substantially identical to the protein identified by the UniProt reference number P60508 or a variant or homolog having substantial identity thereto. In embodiments, syncytin-A is substantially identical to the protein identified by the UniProt reference number Q5G5D5 or a variant or homolog having substantial identity thereto. In embodiments, syncytin-A is substantially identical to the protein identified by the UniProt reference number Q9UQF0 or a variant or homolog having substantial identity thereto.

The term “myoferlin protein” or “myoferlin” as used herein includes any of the recombinant or naturally-occurring forms of myoferlin, also referred to as Fer-1-like protein 3 or variants or homologs thereof that maintain myoferlin activity (e.g. within at least 50%, 80%, 90%, 95%, 96%, 97%, 98%, 99% or 100% activity compared to myoferlin). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring myoferlin protein. In embodiments, myoferlin is substantially identical to the protein identified by the UniProt reference number or a variant or homolog having substantial identity thereto.

The term “VSV-G protein” or “VSV-G” as used herein includes any of the recombinant or naturally-occurring forms of vesicular stomatitis virus glycoprotein (VSV-G), or variants or homologs thereof that maintain VSV-G activity (e.g. within at least 50%, 80%, 90%, 95%, 96%, 97%, 98%, 99% or 100% activity compared to VSV-G). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring VSV-G protein. In embodiments, VSV-G is substantially identical to the protein identified by the UniProt reference number P04884 or a variant or homolog having substantial identity thereto. In embodiments, VSV-G is substantially identical to the protein identified by the UniProt reference number P03522 or a variant or homolog having substantial identity thereto.

The term “Sindbis virus glycoprotein” as used herein includes any of the recombinant or naturally-occurring forms of Sindbis virus glycoprotein (SINmu), also referred to as Structural polyprotein, p130 or variants or homologs thereof that maintain Sindbis virus glycoprotein activity (e.g. within at least 50%, 80%, 90%, 95%, 96%, 97%, 98%, 99% or 100% activity compared to Sindbis virus glycoprotein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Sindbis virus glycoprotein. In embodiments, Sindbis virus glycoprotein is substantially identical to the protein identified by the UniProt reference number P03316 or a variant or homolog having substantial identity thereto.

The term “baboon retroviral envelope glycoprotein” as used herein includes any of the recombinant or naturally-occurring forms of baboon retroviral envelope glycoprotein (BaEV), also referred to as Envelope glycoprotein, Env polyprotein, or variants or homologs thereof that maintain baboon retroviral envelope glycoprotein activity (e.g. within at least 50%, 80%, 90%, 95%, 96%, 97%, 98%, 99% or 100% activity compared to baboon retroviral envelope glycoprotein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring baboon retroviral envelope glycoprotein. In embodiments, baboon retroviral envelope glycoprotein is substantially identical to the protein identified by the UniProt reference number P10269 or a variant or homolog having substantial identity thereto.

The term “measles virus glycoprotein” as used herein includes any of the recombinant or naturally-occurring forms of measles virus glycoprotein, also referred to as Fusion glycoprotein F0, Hemagglutinin glycoprotein, or variants or homologs thereof that maintain measles virus glycoprotein activity (e.g. within at least 50%, 80%, 90%, 95%, 96%, 97%, 98%, 99% or 100% activity compared to measles virus glycoprotein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring measles virus glycoprotein. In embodiments, measles virus glycoprotein is substantially identical to the protein identified by the UniProt reference number Q786F3 or a variant or homolog having substantial identity thereto. In embodiments, measles virus glycoprotein is substantially identical to the protein identified by the UniProt reference number P08362 or a variant or homolog having substantial identity thereto.

The term “nipah virus envelope glycoprotein” as used herein includes any of the recombinant or naturally-occurring forms of nipah virus envelope glycoprotein, also referred to as Fusion glycoprotein F0, Protein F or variants or homologs thereof that nipah virus envelope glycoprotein activity (e.g. within at least 50%, 80%, 90%, 95%, 96%, 97%, 98%, 99% or 100% activity compared to nipah virus envelope glycoprotein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring nipah virus envelope glycoprotein. In embodiments, nipah virus envelope glycoprotein is substantially identical to the protein identified by the UniProt reference number Q91H63 or a variant or homolog having substantial identity thereto.

The term “CD9 protein” or “CD9” as used herein includes any of the recombinant or naturally-occurring forms of CD9 protein, also referred to as 5H9 antigen, Cell growth-inhibiting gene 2 protein, Tetraspanin-29, or variants or homologs thereof that CD9 protein activity (e.g. within at least 50%, 80%, 90%, 95%, 96%, 97%, 98%, 99% or 100% activity compared to CD9 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD9 protein. In embodiments, CD9 protein is substantially identical to the protein identified by the UniProt reference number P21926 or a variant or homolog having substantial identity thereto.

The term “CD37 protein” or “CD37” as used herein includes any of the recombinant or naturally-occurring forms of CD37 protein, also referred to as Leukocyte antigen CD37, Tetraspanin-26, or variants or homologs thereof that CD37 protein activity (e.g. within at least 50%, 80%, 90%, 95%, 96%, 97%, 98%, 99% or 100% activity compared to CD37 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD37 protein. In embodiments, CD37 protein is substantially identical to the protein identified by the UniProt reference number P11049 or a variant or homolog having substantial identity thereto.

The term “CD53 protein” or “CD53” as used herein includes any of the recombinant or naturally-occurring forms of CD53 protein, also referred to as Leukocyte antigen CD53, Tetraspanin-25, Cell surface glycoprotein CD53, or variants or homologs thereof that CD53 protein activity (e.g. within at least 50%, 80%, 90%, 95%, 96%, 97%, 98%, 99% or 100% activity compared to CD53 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD53 protein. In embodiments, CD53 protein is substantially identical to the protein identified by the UniProt reference number P19397 or a variant or homolog having substantial identity thereto.

The term “CD68 protein” or “CD68” as used herein includes any of the recombinant or naturally-occurring forms of CD68 protein, also referred to as Macrosialin, Gp110, or variants or homologs thereof that CD68 protein activity (e.g. within at least 50%, 80%, 90%, 95%, 96%, 97%, 98%, 99% or 100% activity compared to CD68 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD68 protein. In embodiments, CD68 protein is substantially identical to the protein identified by the UniProt reference number P34810 or a variant or homolog having substantial identity thereto.

The term “CD63” refers to a protein that, in humans, is encoded by the CD63 gene. CD63 is associated with membranes of extracellular vesicles, intracellular vesicles, and exosomes. The term “CD63” as provided herein includes any of the protein naturally occurring forms, homologs or variants that maintain the activity of CD63 (e.g., within at least 50%, 80%, 90%, 95%, 96%, 97%, 98%, 99% or 100% activity compared to the native protein). In aspects, variants or homologs have at least 90%, 95%, 96%, 97%, 98%, 99% or 100% amino acid sequence identity across the whole sequence or a portion of the sequence (e.g. a 10, 20, 25, 30, 40, 50, 100, 150 or 200 continuous amino acid portion) compared to a naturally occurring form. In aspects, the CD63 protein has the amino acid sequence identified as UniProt Reference Number P08962. In aspects, the CD63 protein has the amino acid sequence identified as UniProt Reference Number A0A024RB05.

The term “CD81 protein” or “CD81” as used herein includes any of the recombinant or naturally-occurring forms of CD81 protein, also referred to as Tetraspanin-28, 26 kDa cell surface protein TA PA-1, or variants or homologs thereof that CD81 protein activity (e.g. within at least 50%, 80%, 90%, 95%, 96%, 97%, 98%, 99% or 100% activity compared to CD81 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD81 protein. In embodiments, CD81 protein is substantially identical to the protein identified by the UniProt reference number P60033 or a variant or homolog having substantial identity thereto.

The term “CD82 protein” or “CD82” as used herein includes any of the recombinant or naturally-occurring forms of CD82 protein, also referred to as C33 antigen, Tetraspanin-27, Inducible membrane protein R2, IA4, or variants or homologs thereof that CD82 protein activity (e.g. within at least 50%, 80%, 90%, 95%, 96%, 97%, 98%, 99% or 100% activity compared to CD82 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD82 protein. In embodiments, CD82 protein is substantially identical to the protein identified by the UniProt reference number P27701 or a variant or homolog having substantial identity thereto.

The term “LAMP-1 protein” or “LAMP-1” as used herein includes any of the recombinant or naturally-occurring forms of LAMP-1 protein, also referred to as Lysosome-associated membrane glycoprotein 1, CD107 antigen-like family member A, CD107a, or variants or homologs thereof that LAMP-1 protein activity (e.g. within at least 50%, 80%, 90%, 95%, 96%, 97%, 98%, 99% or 100% activity compared to LAMP-1 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring LAMP-1 protein. In embodiments, LAMP-1 protein is substantially identical to the protein identified by the UniProt reference number P11279 or a variant or homolog having substantial identity thereto.

The term “LAMP-2 protein” or “LAMP-2” as used herein includes any of the recombinant or naturally-occurring forms of LAMP-2 protein, also referred to as Lysosome-associated membrane glycoprotein 2, CD107 antigen-like family member B, CD107b, or variants or homologs thereof that LAMP-2A protein activity (e.g. within at least 50%, 80%, 90%, 95%, 96%, 97%, 98%, 99% or 100% activity compared to LAMP-2A protein). In embodiments, LAMP-2 is associated with membranes of extracellular vesicles, intracellular vesicles, and exosomes. In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring LAMP-2A protein. In embodiments, LAMP-2 protein is substantially identical to the protein identified by the UniProt reference number P13473 or a variant or homolog having substantial identity thereto. In embodiments, the LAMP-2 protein is the LAMP-2A isoform. In embodiments, the LAMP-2 protein is the LAMP-2B isoform. In embodiments, the LAMP-2 protein is the LAMP-2C isoform. In embodiments, the LAMP-2A, LAMP-2B, and LAMP-2C isoforms are described in more detail in Qiao L, Hu J, Qiu X, Wang C, Peng J, Zhang C, Zhang M, Lu H, Chen W. LAMP2A, LAMP2B and LAMP2C: similar structures, divergent roles. Autophagy. 2023 November; 19(11):2837-2852. doi: 10.1080/15548627.2023.2235196. Epub 2023 Jul. 21. PMID: 37469132; PMCID: PMC10549195, which is incorporated herein in its entirety and for all purposes.

The term “lactadherin protein” or “lactadherin” as used herein includes any of the recombinant or naturally-occurring forms of lactadherin protein, also referred to as Breast epithelial antigen BA 46, HMFG, MFGM or variants or homologs thereof that lactadherin protein activity (e.g. within at least 50%, 80%, 90%, 95%, 96%, 97%, 98%, 99% or 100% activity compared to lactadherin protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring lactadherin protein. In embodiments, lactadherin protein is substantially identical to the protein identified by the UniProt reference number Q08431 or a variant or homolog having substantial identity thereto.

The terms “PTGFRN” and “prostaglandin F2 receptor negative regulator” refer to a protein that, in humans, is encoded by the PTGFRN gene. PTGFRN is associated with membranes of extracellular vesicles, intracellular vesicles, and exosomes. The term “PTGFRN” as provided herein includes any of the protein naturally occurring forms, homologs or variants that maintain the activity of PTGFRN (e.g., within at least 50%, 80%, 90%, 95%, 96%, 97%, 98%, 99% or 100% activity compared to the native protein). In aspects, variants or homologs have at least 90%, 95%, 96%, 97%, 98%, 99% or 100% amino acid sequence identity across the whole sequence or a portion of the sequence (e.g. a 10, 20, 25, 30, 40, 50, 100, 150 or 200 continuous amino acid portion) compared to a naturally occurring form. In aspects, the PTGFRN protein has the amino acid sequence identified as UniProt Reference Number Q9P2B2.

The term “CHMP4C protein” or “CHMP4C” as used herein includes any of the recombinant or naturally-occurring forms of Charged Multivesicular Body Protein 4C (AGO2), also referred to as Chromatin-modifying protein 4c, SN F7 homolog associated with Alix 3, or variants or homologs thereof that maintain CHMP4C activity (e.g. within at least 50%, 80%, 90%, 95%, 96%, 97%, 98%, 99% or 100% activity compared to CHMP4C). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CHMP4C protein. In embodiments, CHMP4C is substantially identical to the protein identified by the UniProt reference number Q96CF2 or a variant or homolog having substantial identity thereto.

The term “VPS4B protein” or “VPS4B” as used herein includes any of the recombinant or naturally-occurring forms of Vacuolar Protein Sorting 4 Homolog B (VPS4B), also referred to as Cell migration-inducing gene 1 protein, Suppressor of K(+) transport growth defect 1 (Protein SKD1), or variants or homologs thereof that maintain VPS4B activity (e.g. within at least 50%, 80%, 90%, 95%, 96%, 97%, 98%, 99% or 100% activity compared to VPS4B). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring VPS4B protein. In embodiments, VPS4B is substantially identical to the protein identified by the UniProt reference number 075351 or a variant or homolog having substantial identity thereto.

The term “Akt3 protein” or “Akt3” as used herein includes any of the recombinant or naturally-occurring forms of Akt3 protein, also referred to as RAC-gamma serine/threonine-protein kinase, Protein kinase A kt-3, or variants or homologs thereof that maintain Akt3 protein activity (e.g. within at least 50%, 80%, 90%, 95%, 96%, 97%, 98%, 99% or 100% activity compared to Akt3 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Akt3 protein. In embodiments, Akt3 is substantially identical to the protein identified by the UniProt reference number Q9Y 243 or a variant or homolog having substantial identity thereto.

The term “MAPKAPK2 protein” or “MAPKAPK2” as used herein includes any of the recombinant or naturally-occurring forms of MAPKAPK2 protein, also referred to as MAP kinase-activated protein kinase 2, MAPK-activated protein kinase 2, or variants or homologs thereof that maintain MAPKAPK2 protein activity (e.g. within at least 50%, 80%, 90%, 95%, 96%, 97%, 98%, 99% or 100% activity compared to MAPKAPK2 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MAPKAPK2 protein. In embodiments, MAPKAPK2 is substantially identical to the protein identified by the UniProt reference number P49137 or a variant or homolog having substantial identity thereto.

The term “MEKI protein” or “MEKI” as used herein includes any of the recombinant or naturally-occurring forms of MEKI protein, also referred to as MAP2K1, CFC3, MAPKK1, MKK1, PRKMK1, mitogen-activated protein kinase kinase 1, MEL, or variants or homologs thereof that maintain MEKI protein activity (e.g. within at least 50%, 80%, 90%, 95%, 96%, 97%, 98%, 99% or 100% activity compared to MEKI protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MEKI protein. In embodiments, MEKI is substantially identical to the protein identified by the UniProt reference number Q02750 or a variant or homolog having substantial identity thereto. In embodiments, MEKI is substantially identical to the protein identified by the UniProt reference number P31938 or a variant or homolog having substantial identity thereto.

The term “MEKII protein” or “MEKII” as used herein includes any of the recombinant or naturally-occurring forms of MEKII protein, also referred to as Dual specificity mitogen-activated protein kinase kinase 2, MAP kinase kinase 2, MAPKK2 or variants or homologs thereof that maintain MEKII protein activity (e.g. within at least 50%, 80%, 90%, 95%, 96%, 97%, 98%, 99% or 100% activity compared to MEKII protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MEKII protein. In embodiments, MEKII is substantially identical to the protein identified by the UniProt reference number P36507 or a variant or homolog having substantial identity thereto.

The term “ERK protein” or “ERK” as used herein includes any of the recombinant or naturally-occurring forms of ERK protein, also referred to as Mitogen-activated protein kinase 1, MAP kinase 1, MAPK 1 or variants or homologs thereof that maintain ERK protein activity (e.g. within at least 50%, 80%, 90%, 95%, 96%, 97%, 98%, 99% or 100% activity compared to ERK protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ERK protein. In embodiments, ERK is substantially identical to the protein identified by the UniProt reference number P28482 or a variant or homolog having substantial identity thereto.

The term “EGFR protein” or “EGFR” as used herein includes any of the recombinant or naturally-occurring forms of Epidermal growth factor receptor (EGFR) protein, also referred to as Proto-oncogene c-ErbB-1, Receptor tyrosine-protein kinase erbB-1 or variants or homologs thereof that maintain EGFR protein activity (e.g. within at least 50%, 80%, 90%, 95%, 96%, 97%, 98%, 99% or 100% activity compared to EGFR protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, EGFR is substantially identical to the protein identified by the UniProt reference number P00533 or a variant or homolog having substantial identity thereto.

The term “p38-MAPK protein” or “p38-MAPK” as used herein includes any of the recombinant or naturally-occurring forms of p38-MAPK, also referred to as mitogen-activated protein kinase 11, mitogen-activated protein kinase 12, mitogen-activated protein kinase 13, mitogen-activated protein kinase 14, or variants or homologs thereof that maintain p38-MAPK protein activity (e.g. within at least 50%, 80%, 90%, 95%, 96%, 97%, 98%, 99% or 100% activity compared to p38-MAPK protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring p38-MAPK protein. In embodiments, p38-MAPK is substantially identical to the protein identified by the UniProt reference number Q15759 or a variant or homolog having substantial identity thereto. In embodiments, p38-MAPK is substantially identical to the protein identified by the UniProt reference number P53778 or a variant or homolog having substantial identity thereto. In embodiments, p38-MAPK is substantially identical to the protein identified by the UniProt reference number O15264 or a variant or homolog having substantial identity thereto. In embodiments, p38-MAPK is substantially identical to the protein identified by the UniProt reference number Q16539 or a variant or homolog having substantial identity thereto

The term “PTPN1 protein” or “PTPN1” as used herein includes any of the recombinant or naturally-occurring forms of Tyrosine-protein phosphatase non-receptor type 1 (PTPN1), also referred to as protein-tyrosine phosphatase 1B (PTP-1B), or variants or homologs thereof that maintain PTPN1 protein activity (e.g. within at least 50%, 80%, 90%, 95%, 96%, 97%, 98%, 99% or 100% activity compared to PTPN1 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PTPN1 protein. In embodiments, PTPN1 is substantially identical to the protein identified by the UniProt reference number P18031 or a variant or homolog having substantial identity thereto.

The term “EV-factor ALIX protein” or “EV-factor ALIX” as used herein includes any of the recombinant or naturally-occurring forms of EV-factor ALIX, also referred to as programmed cell death 6-interacting protein, ALIX, PD CD6-interacting protein, ALG-2-interacting protein 1 or variants or homologs thereof that maintain EV-factor ALIX protein activity (e.g. within at least 50%, 80%, 90%, 95%, 96%, 97%, 98%, 99% or 100% activity compared to EV-factor ALIX protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EV-factor ALIX protein. In embodiments, EV-factor ALIX is substantially identical to the protein identified by the UniProt reference number Q8WUM4 or a variant or homolog having substantial identity thereto.

“siRNA”, “small interfering RNA”, or “siRNA nucleic acid” as provided herein refers to a double-stranded or single-stranded ribonucleic acid that has the ability to reduce or inhibit expression of a gene or the activity of a target nucleic acid (e.g., a single-stranded or double-stranded RNA or a single-stranded or double-stranded DNA). Where the siRNA is a double-stranded RNA, the complementary portions of the ribonucleic acid that hybridize to form the double stranded molecule typically have substantial or complete identity. In embodiments, the siRNA is a double-stranded RNA. In embodiments, the siRNA is a single-stranded RNA. In embodiments, an siRNA is a nucleic acid that has substantial or complete identity to a target RNA. In embodiments, the siRNA hybridizes to a target RNA. In embodiments, the siRNA inhibits gene expression by interacting with a complementary target RNA thereby interfering with the endogenous behavior of the complementary target RNA. In embodiments, the target RNAis an mRNA. In embodiments, the siRNA hybridizes to a target promoter sequence. In embodiments, the siRNA inhibits gene expression by hybridizing to a target promoter sequence and decreases or inhibits transcription of a gene operably linked to said target promoter sequence. In embodiments, the siRNA is about 8-50 nucleotides in length (e.g., each complementary sequence of the double stranded siRNA is 8-50 nucleotides in length, and the double stranded siRNA is about 8-50 base pairs in length). The siRNA s provided herein regulate expression of a target gene or activity of a target nucleic by hybridizing to the mRNA of the gene or by hybridizing to the promoter of the target nucleic or the target nucleic acid itself. In embodiments, where the siRNA hybridizes to a promoter of a gene thereby modulating the expression of said gene, the siRNA may be referred to as “antigen RNA” or “agRNA.”

The terms “short hairpin RNA” or “shRNA” refer to an anti-sense ribonucleic acid sequence, which is capable of binding (hybridizing) and inhibiting activity of a target RNA (e.g., mRNA). In embodiments, the shRNA is partially or entirely complementary to a target RNA. An shRNA typically includes a double-stranded RNA structure, also referred to as an RNA duplex or RNA stem loop structure. The RNA stem loop structure of an shRNA includes a stem typically 10-30 base pairs in length and a loop typically 1 to 10 nt in length.

“Dicer-dependent short hairpin RNA” or “dicer-dependent shRNA” refers to a double-stranded RNA (e.g. RNA duplex, RNA stem-loop) that is processed by the Dicer enzyme into an RNA of limited length (e.g. about 25 to 30 nucleotides in length). In embodiments, the processed RNAis an effector of RNA interference. The stem of the double-stranded RNA (e.g. RNA duplex, RNA stem-loop) of a dicer-dependent shRNA nucleic acid is greater than 19 base pairs in length.

“Dicer-independent short hairpin RNA” or “D icer-independent shRNA” refers to a double-stranded RNA (e.g. RNA duplex, RNA stem-loop) that is processed by the Argonaute 2 (AGO2) protein. The stem of the RNA stem-loop of a dicer-independent shRNA is 19 base pairs (bp) or less in length. In embodiments, the stem is 19 bp, 18 bp, 17 bp, 16 bp, 15 bp, 14 bp, 13 bp, 12 bp, 11 bp, or 10 bp in length. The shorter stem length (e.g. 19 bp or less) of a Dicer-independent shRNA nucleic acid causes the shRNA nucleic acid to be bypassed by the Dicer enzyme. For example, in embodiments, the stem of a Dicer-independent RNAis too short to be efficiently bound by Dicer. Thus, a Dicer-independent RNA bypasses processing by Dicer and instead is cleaved by the AGO2 enzyme. In embodiments, AGO2 cleaves the sense strand (e.g. passenger strand, sense strand) of an shRNA and retains the mature targeting strand (e.g. antisense strand) for effecting RNA interference on a target nucleic acid sequence (e.g. target mRNA sequence, target promoter sequence).

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In aspects, the antisense nucleic acid hybridizes to the target nucleic acid in vitro. In aspects, the antisense nucleic acid hybridizes to the target nucleic acid in a cell. In aspects, the antisense nucleic acid hybridizes to the target nucleic acid in an organism. In aspects, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and anomeric sugar-phosphate, backbone-modified nucleotides.

“Hybridize” and “hybridization” refer to the pairing of complementary (including partially complementary) nucleic acid strands. Hybridization and the strength of hybridization (e.g., the strength of the association between nucleic acid strands) is impacted by factors known in the art including the degree of complementarity between the nucleic acid, stringency of the conditions involved affected by such conditions as the concentration of salts, the melting temperature (Tm) of the formed hybrid, the presence of other components, the molarity of the hybridizing strands and the G:C content of the nucleic acid strands. When one nucleic acid is said to “hybridize” to another nucleic acid, it means that there is some complementarity between the two nucleic acids or that the two nucleic acids form a hybrid under high or low stringency conditions.

The term “complement,” as used herein, refers to a nucleotide (e.g., RNA or DNA) or a sequence of nucleotides capable of base pairing with a complementary nucleotide or sequence of nucleotides. A s described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. W here the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids. In embodiments, the vector is a lentivirus vector.

The term “extracellular vesicle” refers to a cell-derived vesicle comprising a membrane that encloses an internal space. Extracellular vesicles comprise all membrane-bound vesicles that have a smaller diameter than the cell from which they are derived. Generally extracellular vesicles range in diameter from 20 nm to 1000 nm and can comprise various macromolecular cargo either within the internal space, displayed on the external surface of the extracellular vesicle, and/or spanning the membrane. The cargo can comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and cultured cells. In embodiments, the extracellular vesicle is an exosome.

In embodiments, the EV has a diameter of 20 nm to 1000 nm. In embodiments, the EV has a diameter of 40 nm to 1000 nm. In embodiments, the EV has a diameter of 60 nm to 1000 nm. In embodiments, the EV has a diameter of 80 nm to 1000 nm. In embodiments, the EV has a diameter of 100 nm to 1000 nm. In embodiments, the EV has a diameter of 120 nm to 1000 nm. In embodiments, the EV has a diameter of 140 nm to 1000 nm. In embodiments, the EV has a diameter of 160 nm to 1000 nm. In embodiments, the EV has a diameter of 180 nm to 1000 nm. In embodiments, the EV has a diameter of 200 nm to 1000 nm. In embodiments, the EV has a diameter of 220 nm to 1000 nm. In embodiments, the EV has a diameter of 240 nm to 1000 nm. In embodiments, the EV has a diameter of 260 nm to 1000 nm. In embodiments, the EV has a diameter of 280 nm to 1000 nm. In embodiments, the EV has a diameter of 300 nm to 1000 nm. In embodiments, the EV has a diameter of 320 nm to 1000 nm. In embodiments, the EV has a diameter of 340 nm to 1000 nm. In embodiments, the EV has a diameter of 360 nm to 1000 nm. In embodiments, the EV has a diameter of 380 nm to 1000 nm. In embodiments, the EV has a diameter of 400 nm to 1000 nm. In embodiments, the EV has a diameter of 420 nm to 1000 nm. In embodiments, the EV has a diameter of 440 nm to 1000 nm. In embodiments, the EV has a diameter of 460 nm to 1000 nm. In embodiments, the EV has a diameter of 480 nm to 1000 nm. In embodiments, the EV has a diameter of 500 nm to 1000 nm. In embodiments, the EV has a diameter of 520 nm to 1000 nm. In embodiments, the EV has a diameter of 540 nm to 1000 nm. In embodiments, the EV has a diameter of 560 nm to 1000 nm. In embodiments, the EV has a diameter of 580 nm to 1000 nm. In embodiments, the EV has a diameter of 600 nm to 1000 nm. In embodiments, the EV has a diameter of 620 nm to 1000 nm. In embodiments, the EV has a diameter of 640 nm to 1000 nm. In embodiments, the EV has a diameter of 660 nm to 1000 nm. In embodiments, the EV has a diameter of 680 nm to 1000 nm. In embodiments, the EV has a diameter of 700 nm to 1000 nm. In embodiments, the EV has a diameter of 120 nm to 1000 nm. In embodiments, the EV has a diameter of 40 nm to 1000 nm. In embodiments, the EV has a diameter of 60 nm to 1000 nm. In embodiments, the EV has a diameter of 80 nm to 1000 nm. In embodiments, the EV has a diameter of 100 nm to 1000 nm. In embodiments, the EV has a diameter of 720 nm to 1000 nm. In embodiments, the EV has a diameter of 740 nm to 1000 nm. In embodiments, the EV has a diameter of 760 nm to 1000 nm. In embodiments, the EV has a diameter of 780 nm to 1000 nm. In embodiments, the EV has a diameter of 800 nm to 1000 nm. In embodiments, the EV has a diameter of 820 nm to 1000 nm. In embodiments, the EV has a diameter of 840 nm to 1000 nm. In embodiments, the EV has a diameter of 860 nm to 1000 nm. In embodiments, the EV has a diameter of 880 nm to 1000 nm. In embodiments, the EV has a diameter of 900 nm to 1000 nm. In embodiments, the EV has a diameter of 920 nm to 1000 nm. In embodiments, the EV has a diameter of 940 nm to 1000 nm. In embodiments, the EV has a diameter of 960 nm to 1000 nm. In embodiments, the EV has a diameter of 980 nm to 1000 nm.

In embodiments, the EV has a diameter of 20 nm to 980 nm. In embodiments, the EV has a diameter of 20 nm to 960 nm. In embodiments, the EV has a diameter of 20 nm to 940 nm. In embodiments, the EV has a diameter of 20 nm to 920 nm. In embodiments, the EV has a diameter of 20 nm to 900 nm. In embodiments, the EV has a diameter of 20 nm to 880 nm. In embodiments, the EV has a diameter of 20 nm to 860 nm. In embodiments, the EV has a diameter of 20 nm to 840 nm. In embodiments, the EV has a diameter of 20 nm to 820 nm. In embodiments, the EV has a diameter of 20 nm to 800 nm. In embodiments, the EV has a diameter of 20 nm to 780 nm. In embodiments, the EV has a diameter of 20 nm to 760 nm. In embodiments, the EV has a diameter of 20 nm to 740 nm. In embodiments, the EV has a diameter of 20 nm to 720 nm. In embodiments, the EV has a diameter of 20 nm to 700 nm. In embodiments, the EV has a diameter of 20 nm to 680 nm. In embodiments, the EV has a diameter of 20 nm to 660 nm. In embodiments, the EV has a diameter of 20 nm to 640 nm. In embodiments, the EV has a diameter of 20 nm to 620 nm. In embodiments, the EV has a diameter of 20 nm to 600 nm. In embodiments, the EV has a diameter of 20 nm to 580 nm. In embodiments, the EV has a diameter of 20 nm to 560 nm. In embodiments, the EV has a diameter of 20 nm to 540 nm. In embodiments, the EV has a diameter of 20 nm to 520 nm. In embodiments, the EV has a diameter of 20 nm to 500 nm. In embodiments, the EV has a diameter of 20 nm to 480 nm. In embodiments, the EV has a diameter of 20 nm to 460 nm. In embodiments, the EV has a diameter of 20 nm to 440 nm. In embodiments, the EV has a diameter of 20 nm to 420 nm. In embodiments, the EV has a diameter of 20 nm to 400 nm. In embodiments, the EV has a diameter of 20 nm to 380 nm. In embodiments, the EV has a diameter of 20 nm to 360 nm. In embodiments, the EV has a diameter of 20 nm to 340 nm. In embodiments, the EV has a diameter of 20 nm to 320 nm. In embodiments, the EV has a diameter of 20 nm to 300 nm. In embodiments, the EV has a diameter of 20 nm to 280 nm. In embodiments, the EV has a diameter of 20 nm to 260 nm. In embodiments, the EV has a diameter of 20 nm to 240 nm. In embodiments, the EV has a diameter of 20 nm to 220 nm. In embodiments, the EV has a diameter of 20 nm to 200 nm. In embodiments, the EV has a diameter of 20 nm to 180 nm. In embodiments, the EV has a diameter of 20 nm to 160 nm. In embodiments, the EV has a diameter of 20 nm to 140 nm. In embodiments, the EV has a diameter of 20 nm to 120 nm. In embodiments, the EV has a diameter of 20 nm to 100 nm. In embodiments, the EV has a diameter of 20 nm to 80 nm. In embodiments, the EV has a diameter of 20 nm to 60 nm. In embodiments, the EV has a diameter of 20 nm to 40 nm. In embodiments, the EV has a diameter of 20 nm, 40 nm, 60 nm, 80 nm, 100 nm, 120 nm, 140 nm, 160 nm, 180 nm, 200 nm, 220 nm, 240 nm, 260 nm, 280 nm, 300 nm, 320 nm, 340 nm, 360 nm, 380 nm, 400 nm, 420 nm, 440 nm, 460 nm, 480 nm, 500 nm, 520 nm, 540 nm, 560 nm, 580 nm, 600 nm, 620 nm, 640 nm, 660 nm, 680 nm, 700 nm, 720 nm, 740 nm, 760 nm, 780 nm, 800 nm, 820 nm, 840 nm, 860 nm, 880 nm, 900 nm, 920 nm, 940 nm, 960 nm, 980 nm, or 1000 nm.

In embodiments, the EV has a diameter of 20 nm to 200 nm. In embodiments, the EV has a diameter of 40 nm to 200 nm. In embodiments, the EV has a diameter of 60 nm to 200 nm. In embodiments, the EV has a diameter of 80 nm to 200 nm. In embodiments, the EV has a diameter of 100 nm to 200 nm. In embodiments, the EV has a diameter of 120 nm to 200 nm. In embodiments, the EV has a diameter of 140 nm to 200 nm. In embodiments, the EV has a diameter of 160 nm to 200 nm. In embodiments, the EV has a diameter of 180 nm to 200 nm.

In embodiments, the EV has a diameter of 20 nm to 180 nm. In embodiments, the EV has a diameter of 20 nm to 180 nm. In embodiments, the EV has a diameter of 20 nm to 160 nm. In embodiments, the EV has a diameter of 20 nm to 140 nm. In embodiments, the EV has a diameter of 20 nm to 120 nm. In embodiments, the EV has a diameter of 20 nm to 100 nm. In embodiments, the EV has a diameter of 20 nm to 80 nm. In embodiments, the EV has a diameter of 20 nm to 60 nm. In embodiments, the EV has a diameter of 20 nm to 40 nm. In embodiments, the EV has a diameter of 20 nm, 40 nm, 60 nm, 80 nm, 100 nm, 120 nm, 140 nm, 160 nm, 180 nm, or 200 nm.

In embodiments, the EV has a diameter of 100 nm to 200 nm. In embodiments, the EV has a diameter of 150 nm to 200 nm. In embodiments, the EV has a diameter of 100 nm to 180 nm. In embodiments, the EV has a diameter of 120 nm to 180 nm. In embodiments, the EV has a diameter of 140 nm to 165 nm.

The term “exosome” refers to a cell-derived small (between 20-300 nm in diameter) vesicle comprising a membrane that encloses an internal space, and which is generated from the cell by direct plasma membrane budding or by fusion of the late endosome with the plasma membrane. The exosome comprises lipid and/or fatty acid and optionally comprises a payload (e.g., a therapeutic agent), a receiver (e.g., a targeting peptide), a polynucleotide (e.g., a nucleic acid, RNA, or DNA), a sugar (e.g., a simple sugar, polysaccharide, or glycan) or other molecules or drugs. The exosome can be derived from a producer cell, and isolated from the producer cell based on its size, density, biochemical parameters, or a combination thereof. An exosome is a species of extracellular vesicle.

The term “isolated”, when applied to an extracellular vesicle denotes that the extracellular vesicle is essentially free of other cellular components of the cell from which it originated. In embodiments, purity and homogeneity are typically determined using analytical chemistry techniques such as polyacrylamide gel electrophoresis or high performance liquid chromatography. An extracellular vesicle that is the predominant species present in a preparation is substantially purified. In embodiments, an extracellular vesicle described herein is an isolated extracellular vesicle. In embodiments, the isolated extracellular vesicle includes one or more cargo molecules encapsulated by the membrane of said extracellular vesicle. For example, in embodiments, the isolated extracellular vesicle includes an siRNA nucleic acid as provided herein including embodiments thereof. In embodiments, the nucleic acids described herein (e.g. shRNA nucleic acid, siRNA nucleic acid) are isolated nucleic acids.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

The term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In aspects inhibition means negatively affecting (e.g. decreasing) the concentration or levels of the protein relative to the concentration or level of the protein in the absence of the inhibitor. In embodiments, inhibition refers to decreasing the expression level of a protein. In embodiments, inhibition refers to decreasing the expression level of a gene. In aspects, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In aspects, inhibition refers to a reduction of activity of a target protein resulting from a direct interaction (e.g. an inhibitor binds to the target protein). In aspects, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. N on-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. In embodiments, a nucleic acid molecule is introduced into a cell using a site-specific gene editing method known in the art, for example use of transcription activator-like effector nuclease (TALEN), zinc finger nuclease (ZFN), CRISPR/Cas technology. Further examples of methods for transducing a nucleic acid into a cell may be found in: Anzalone A V, et al. Programmable deletion, replacement, integration and inversion of large DNA sequences with twin prime editing. Nat Biotechnol. 2022 May; 40(5):731-740. doi: 10.1038/s41587-021-01133-w. Epub 2021 Dec. 9. PMID: 34887556; PMCID: PMC9117393; Wang C, et al. dCas9-based gene editing for cleavage-free genomic knock-in of long sequences. Nat Cell Biol. 2022 February; 24(2):268-278. doi: 10.1038/s41556-021-00836-1. Epub 2022 Feb. 10. PMID: 35145221; PMCID: PMC8843813; Yarnall M T N, et al. Drag-and-drop genome insertion of large sequences without double-strand DNA cleavage using CRISPR-directed integrases. Nat Biotechnol. 2023 April; 41(4):500-512. doi: 10.1038/s41587-022-01527-4. Epub 2022 Nov. 24. PMID: 36424489; PMCID: PMC10257351; Lampe G D, et al. Targeted DNA integration in human cells without double-strand breaks using C R ISPR-associated transposases. Nat Biotechnol. 2024 January; 42(1):87-98. doi: 10.1038/s41587-023-01748-1. Epub 2023 Mar. 29. PMID: 36991112; PMCID: PMC10620015, which are incorporated herein in their entirety and for all purposes. For viral-based methods of transfection any useful viral vector (e.g. adenovirus vector) may be used in the methods described herein. In embodiments, transduction occurs by introduction of a virus or viral vector (e.g. lentivirus vector, adenovirus vector, etc.) into the cell. Examples for viral vectors include, but are not limited to retroviral, adenoviral, lentiviral and adeno-associated viral vectors. In some embodiments, the nucleic acid molecules are introduced into a cell using an adenoviral vector following standard procedures well known in the art. The terms “transfection” or “transduction” also refer to introducing proteins into a cell from the external environment. In embodiments, transduction or transfection of a protein relies on attachment of a peptide or protein capable of crossing the cell membrane to the protein of interest. So, e.g., Ford et al. (2001) Gene Therapy 8:1-4 and Prochiantz (2007) Nat Methods 4:119-20.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The terms “expression level,” “amount,” or “level” of a gene is a detectable level in a biological sample. “Expression” generally refers to the process by which information (e.g., gene-encoded and/or epigenetic) is converted into the structures present and operating in the cell. Therefore, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, miRNA, transfer RNA, ribosomal RNA, non-coding RNA). Expression levels can be measured by methods known to one skilled in the art and also disclosed herein. The expression level or amount of a biomarker (e.g., RNA, miRNA) can be used to diagnose and/or treat a subject with pancreatic cancer.

The term “target gene” refers to any nucleic acid sequence which contains an identified genes or a target region within a gene, including intergenic regions, non-coding regions, untranscribed regions, introns, exons, and transgenes. The target gene (or a target site within the gene) can be a gene derived from a cell, an endogenous gene, a transgene, or exogenous genes such as genes of a pathogen, for example a virus, which is present in the cell after infection thereof. The cell containing the target gene can be derived from or contained in any organism.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. For example, in embodiments, the nucleic acid encoding the AGO2 protein provided herein including embodiments thereof does not originate from the cell which expresses the AGO2. Thus, in embodiments, the nucleic acid encoding the AGO2 protein is a recombinant nucleic acid. In embodiments, the AGO2 protein is a recombinant protein. Similarly, in embodiments, the nucleic acid encoding the shRNA nucleic acid provided herein including embodiments thereof does not originate from the cell which expresses the shRNA. Thus, in embodiments, the shRNA nucleic acid provided herein including embodiments thereof is a recombinant shRNA nucleic acid. In embodiments, the siRNA nucleic acid provided herein including embodiments thereof is a recombinant siRNA nucleic acid. In another example, recombinant cells express genes that are not found within the native (non-recombinant) form of the cell or express native genes that are otherwise abnormally expressed, under expressed or not expressed at all. In embodiments, the cell provided herein including embodiments thereof is a recombinant cell. For example, in embodiments, the stably transfected cell provided herein including embodiments thereof is a recombinant cell. Transgenic cells and plants are those that express a heterologous gene or coding sequence, typically as a result of recombinant methods.

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Thus in embodiments, the nucleic acid encoding the AGO2 protein provided herein is a heterologous nucleic acid. In embodiments, the nucleic acid encoding the shRNA nucleic acid is a heterologous nucleic acid. Similarly, a heterologous protein indicates that the protein including two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous shRNA nucleic acid” as referred to herein is an shRNA nucleic acid that does not originate from the cell or organism it is expressed by. Conversely, the term “endogenous” refers to a molecule or substance that is native to, or originates within, a given cell or organism. In embodiments, the nucleic acid encoding the AGO2 protein is exogenous to the cell. Thus, in embodiments, the AGO2 protein is an exogenous AGO2 protein. In embodiments, the nucleic acid encoding the shRNA nucleic acid is exogenous to the cell. In embodiments, the shRNA is an exogenous shRNA nucleic acid. In embodiments, the siRNA is an exogenous siRNA.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

A “control” or “standard control” refers to a sample, measurement, or value that serves as a reference, usually a known reference, for comparison to a test sample, measurement, or value. For example, a test measurement can be the expression level of a gene in a cell including the EV provided herein including embodiments thereof, compared to the expression level of a gene in a cell lacking the EV provided herein including embodiments thereof. A test measurement can be the expression level of a protein in a cell including an inhibitor (e.g. shRNA nucleic acid, siRNA nucleic acid) relative to the expression level of the protein in a cell lacking the inhibitor. Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

A “stem cell” is a cell characterized by the ability of self-renewal through mitotic cell division and the potential to differentiate into a tissue or an organ. Among mammalian stem cells, embryonic stem cells (ES cells) and somatic stem cells (e.g., HSC) can be distinguished. In embodiments, the stem cell is an embryonic stem cell. Embryonic stem cells are derived from a blastocyst and give rise to embryonic tissues, whereas somatic stem cells reside in adult tissues for the purpose of tissue regeneration and repair. In embodiments, an embryonic stem cell is pluripotent and has the potential to differentiate into any type of cell. Mesenchymal stem cell refers to a multipotent stromal cell. In embodiments, a mesenchymal stem cell has the potential to differentiate into an osteoblast, chondrocyte, myocyte, or adipocyte.

The term “neural stem cell” refers to a neural progenitor cell. In embodiments, a neural stem cell refers to a cell capable of differentiating into a neuron, oligodendrocyte, glia cell, or astrocyte. In embodiments, a neural stem cell is a precursor to neuron, astrocyte, or oligodendrocyte. In embodiments, a neural stem cell is cultured in the presence of one or more growth factors or differentiating agents capable of inducing differentiating the neural progenitor cell to a neuron, oligodendrocyte, glia cell, or astrocyte.

The term “induced pluripotent stem cell” or “iPSC” refers to a pluripotent stem cell generated from a somatic cell. In embodiments, the iPSC may be cultured under conditions wherein the iPSC proliferates and differentiates into a specific type of cell.

The term “epithelial cell” refers to cells that often form the tissue that lines the surfaces of organs and cavities in the body. Epithelial cells may be derived from all three embryonic germ layers including the ectoderm, the mesoderm and the endoderm. For example, in embodiments, epithelial cells form the barrier of organs in the respiratory, reproductive, urinary, circulatory, and gastrointestinal systems. Epithelial cells may be characterized by shape, including squamous, columnar, or cuboidal.

As used herein, the term “T cells” or “T lymphocytes” are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

The term “macrophage” refers to a type of white blood cell that plays a role in the innate immune system. Macrophages are antigen presenting cells, and may present antigens on MHC II molecules on their surface for recognition by a corresponding TCR. Macrophages are capable of up-taking pathogens and secreting cytokines for modulating the immune response.

The terms “natural killer cell” and “NK cell” are used in accordance with their plain ordinary meaning and refer to a type of cytotoxic lymphocyte involved in the innate immune system. The role NK cells play is typically analogous to that of cytotoxic T cells in the vertebrate adaptive immune response. NK cells may provide rapid responses to virus-infected cells, acting at around 3 days after infection, and respond to tumor formation. Typically, immune cells detect major histocompatibility complex (MHC) presented on infected cell surfaces, triggering cytokine release, causing lysis or apoptosis. NK cells typically have the ability to recognize stressed cells in the absence of antibodies and MHC, allowing for a much faster immune reaction.

### Methods for Making Extracellular Vesicles

Provided herein, inter alia, are methods for making an extracellular vesicle (EV) including an siRNA nucleic acid cargo effective for decreasing expression of a target gene. The methods include transducing a cell with a nucleic acid encoding an argonaute 2 (AGO2) protein and a nucleic acid encoding a short hairpin RNA (shRNA) nucleic acid, culturing the cell under conditions conducive for the cell to express the AGO2 protein and the shRNA nucleic acid, thereby forming the extracellular vesicle including the siRNA nucleic acid, and isolating the extracellular vesicle including the siRNA nucleic acid from the cell.

The extracellular vesicle generated by the methods provided herein including embodiments thereof include an siRNA nucleic acid within its internal space (e.g. encapsulated by the extracellular vesicle membrane). In embodiments, the AGO2 protein degrades the non-targeting strand (e.g. passenger strand, sense strand) of the siRNA. Thus, in embodiments, the EV generated by the methods provided herein including embodiments thereof is selectively loaded with the targeting strand of the siRNA (e.g. antisense strand) nucleic acid. Thus, it is contemplated that the siRNA cargo of the EV avoids off-target effects resulting from non-specific binding by a passenger strand (e.g. sense strand) of the siRNA nucleic acid. In an aspect is provided a method for making an extracellular vesicle (EV) including a small interfering RNA (siRNA) nucleic acid, the method including: i) transfecting a cell with a nucleic acid encoding an argonaute 2 (AGO2) protein and a nucleic acid encoding a short hairpin RNA (shRNA) nucleic acid, and ii) culturing the cell under conditions conducive for the cell to express the AGO2 protein and the shRNA nucleic acid, thereby forming the extracellular vesicle including the siRNA nucleic acid. In another aspect is provided a method for making an extracellular vesicle (EV) including a small interfering RNA (siRNA) nucleic acid, the method including: i) transfecting a cell with a nucleic acid encoding an argonaute 2 (AGO2) protein and a nucleic acid encoding a short hairpin RNA (shRNA) nucleic acid, ii) culturing the cell under conditions conducive for the cell to express the AGO2 protein and the shRNA nucleic acid, thereby forming the extracellular vesicle including the siRNA nucleic acid, and iii) isolating the extracellular vesicle including the siRNA nucleic acid from the cell.

In embodiments, the nucleic acid encoding the AGO2 protein and the nucleic acid encoding the shRNA nucleic acid are each independently part of a vector. In embodiments, the nucleic acid encoding the AGO2 protein and the nucleic acid encoding the shRNA nucleic acid are part of the same vector. In embodiments, the nucleic acid encoding the AGO2 protein and the nucleic acid encoding the shRNA nucleic acid are part of different vectors (e.g. a first vector and a second vector). In embodiments, the nucleic acid encoding the AGO2 protein is part of a first viral vector. In embodiments, the nucleic acid encoding the shRNA nucleic acid is part of a second viral vector.

In embodiments, the method provided herein including embodiments thereof include use of a stably transduced cell capable of expressing the shRNA nucleic acid provided herein including embodiments thereof and the AGO2 protein provided herein including embodiments thereof. The term “stably transduced cell” is used in accordance with its plain ordinary meaning in the art and refers to cell transduced with a nucleic acid wherein the nucleic acid is incorporated into genome of the cell. In embodiments, the cell is transduced with a nucleic acid encoding an AGO2 protein. In embodiments, the cell is transduced with a nucleic acid encoding an shRNA nucleic acid. Thus, in embodiments, the stably transduced cell includes a nucleic acid encoding the AGO2 protein integrated into the genome of the cell. In embodiments, the stably transduced cell includes a nucleic acid encoding the shRNA nucleic acid integrated into the genome of the cell. Thus, Thus, in an aspect is provided a method for making an extracellular vesicle (EV) including a small interfering RNA (siRNA) nucleic acid, the method including: culturing a cell stably transduced with a nucleic acid encoding an argonaute 2 (AGO2) protein and a nucleic acid encoding a short hairpin RNA (shRNA) nucleic acid under conditions conducive for the cell to express the AGO2 protein and the shRNA nucleic acid, thereby forming the extracellular vesicle including the siRNA nucleic acid. In another aspect is provided a method for making an extracellular vesicle (EV) including a small interfering RNA (siRNA) nucleic acid, the method including: i) culturing a cell stably transduced with a nucleic acid encoding an argonaute 2 (AGO2) protein and a nucleic acid encoding a short hairpin RNA (shRNA) nucleic acid under conditions conducive for the cell to express the AGO2 protein and the shRNA nucleic acid, thereby forming the extracellular vesicle including the siRNA nucleic acid, and ii) isolating the extracellular vesicle including the siRNA nucleic acid from the cell.

For the methods provided herein, including embodiments thereof, the AGO2 protein is exterior to the membrane of said extracellular vesicle. For example, in embodiments, the AGO2 protein is within the cytoplasmic space of the cell and exterior to the membrane of the extracellular vesicle. In embodiments, the AGO2 protein is within the internal space of said extracellular vesicle (e.g. encapsulated by the membrane of the extracellular vesicle).

For the methods provided herein, in embodiments, the cell is cultured for about 24 hours to about 7 days. In embodiments, the cell is cultured for about 2 days to about 7 days. In embodiments, the cell is cultured for about 3 days to about 7 days. In embodiments, the cell is cultured for about 4 days to about 7 days. In embodiments, the cell is cultured for about 5 days to about 7 days. In embodiments, the cell is cultured for about 6 days to about 7 days.

In embodiments, the cell is cultured for about 24 hours to about 6 days. In embodiments, the cell is cultured for about 24 hours to about 5 days. In embodiments, the cell is cultured for about 24 hours to about 4 days. In embodiments, the cell is cultured for about 24 hours to about 3 days. In embodiments, the cell is cultured for about 24 hours to about 2 days. In embodiments, the cell is cultured for about 24 hours, 1 day, 2 days, 3 days, 4 days, 5 days, 6 days, or 7 days.

In embodiments, the cell is cultured for at least 24 hours prior to isolating the extracellular vesicle. In embodiments, the cell is cultured for at least 36 hours prior to isolating the extracellular vesicle. In embodiments, the cell is cultured for at least 48 hours prior to isolating the extracellular vesicle. In embodiments, the cell is cultured for at least 60 hours prior to isolating the extracellular vesicle. In embodiments, the cell is cultured for at least 72 hours prior to isolating the extracellular vesicle. In embodiments, the cell is cultured for at least 84 hours prior to isolating the extracellular vesicle. In embodiments, the cell is cultured for at least 96 hours prior to isolating the extracellular vesicle. In embodiments, the cell is cultured for at least 108 hours prior to isolating the extracellular vesicle. In embodiments, the cell is cultured no more than about 5 days prior to isolating the extracellular vesicle.

The term “culture” or “cell culture” refers to the maintenance of cells in an artificial, in vitro environment. A “cell culture system” is used herein to refer to culture conditions in which a population of cells may be grown as monolayers or in suspension. “Culture medium” is used herein to refer to a nutrient solution for the culturing, growth, proliferation, or differentiation of cells, for example, neural stem cells (NSC), a mesenchymal stem cells (MSC), induced pluripotent stem cells (iPSC), an embryonic stem cells (ESC), immune cells, or epithelial cells. In embodiments, the cell is cultured in Freestyle 393 medium, CDM 4PERMAb medium, NeuroCult™ Neural Stem Cell Culture Medium, in RoosterNourish™-MSC-XF, StemPro™ MSC SFM XenoFree, StemPro™ MSC SFM, or ReN cell NSC Maintenance Medium. In embodiments, the medium includes serum. In embodiments, the medium is serum-free. For example, in embodiments, the culture includes 1 to 20% fetal bovine serum. In embodiments, the culture includes 5 to 10% fetal bovine serum. In embodiments, the serum is exosome-depleted serum. In embodiments, the culture includes one or more amino acids at a concentration between 0.005 to 5 g/L. In embodiments, the one or more amino acids is L-Arginine, L-Cyestein, L-Cystine, L-Glutamine, L-Histidine, L-Isoleucine, L-Leucine, L-Lysine, L-M ethionine, L-Phenylalanine, L-Threonine, L-Tryptophan, L-Tyrosine, L-Valine, Glycine, L-Alanine, L-Asparagine, L-Aspartic acid, L-Glutamic acid, L-Proline, L-Serine, Phospho-L-tyrosine, S-sulfo-L-cystein, L-Alanyl L-tyrosine, L-alanyl-L-glutamine or a combination thereof. Culture conditions conducive for generating extracellular vesicles are described in more detail in: Jimenez, L., et al. (2023). Culture conditions greatly impact the levels of vesicular and extravesicular Ago2 and RNA in extracellular vesicle preparations. Journal of Extracellular Vesicles, 12, e12366.https://doi.org/10.1002/jev2.12366; and Jeske, R., et al. (2022) Upscaling human mesenchymal stromal cell production in a novel vertical-wheel bioreactor enhances extracellular vesicle secretion and cargo profile. Bioactive Materials. 25, p732-747, https://doi.org/10.1016/j.bioactmat.2022.07.004, which are incorporated herein in their entirely and for all purposes.

For the method provided herein, in embodiments, the method further includes isolating said extracellular vesicle comprising said siRNA nucleic acid from said cell. In embodiments, isolating the extracellular vesicle from the cell includes differential centrifugation, density gradient centrifugation, ultrafiltration, immunoaffinity, ultracentrifugation, size exclusion chromatography, tangential flow filtration (TFF), or asymmetrical flow field-flow fractionation (AF4). In embodiments, isolating the extracellular vesicle from the cell includes differential centrifugation. In embodiments, isolating the extracellular vesicle from the cell includes density gradient centrifugation. In embodiments, isolating the extracellular vesicle from the cell includes ultrafiltration. In embodiments, isolating the extracellular vesicle from the cell includes immunoaffinity. In embodiments, isolating the extracellular vesicle from the cell includes ultracentrifugation. In embodiments, isolating the extracellular vesicle from the cell includes size exclusion chromatography. In embodiments, isolating the extracellular vesicle from the cell includes tangential flow filtration (TFF). In embodiments, isolating the extracellular vesicle from the cell includes asymmetrical flow field-flow fractionation (AF4). In embodiments, the extracellular vesicle is isolated based on an EV marker associated with the EV membrane. For example, in embodiments, the EV may be isolated using an antibody specific for an EV membrane marker. Thus, in embodiments, the EV is identified and separated using an antibody specific for an EV membrane-bound EV surface marker, including tetraspanins; CD9, CD63, CD81, or a combination thereof. In another example, the extracellular vesicle may be isolated based on size and/or density. In another example, the extracellular vesicle may be isolated based on particle surface charge. In another example, the extracellular vesicle may be isolated based on size and/or molecular weight. In embodiments, isolating the extracellular vesicle from the cell includes differential ultracentrifugation. In embodiments, isolating the extracellular vesicle from the cell includes filtration. In embodiments, isolating the extracellular vesicle from the cell includes size exclusion chromatography. Methods for isolating extracellular vesicle are described in Yu Z L., et. al.; Untouched isolation enables targeted functional analysis of tumour-cell-derived extracellular vesicles from tumour tissues. J Extracell Vesicles. 2022 April; 11(4):e12214. doi: 10.1002/jev2.12214. PMID: 35436039; PMCID: PMC9014807; and Zhang, H., Lyden, D. Asymmetric-flow field-flow fractionation technology for exomere and small extracellular vesicle separation and characterization. Nat Protoc 14, 1027-1053 (2019). https://doi.org/10.1038/s41596-019-0126-x, which are incorporated herein in their entirety and for all purposes.

In embodiments, the shRNA nucleic acid provided herein including embodiments thereof is a Dicer-independent shRNA nucleic acid. As described above, a Dicer-independent shRNA nucleic acid refers to an shRNA that bypasses processing by Dicer and instead is cleaved by the AGO2 enzyme. In embodiments, Ago2 cleaves the sense strand (e.g. passenger strand, sense strand) of an shRNA and retains the mature targeting strand (e.g. antisense strand) for effecting RNA interference on a target nucleic acid sequence (e.g. target mRNA sequence, target promoter sequence). The stem of the stem-loop structure of a Dicer-independent shRNA is 19 bp or less in length, and therefore cannot be processed by dicer. In embodiments, the shRNA nucleic acid includes a stem loop including a stem sequence no more than about 19 base pairs in length.

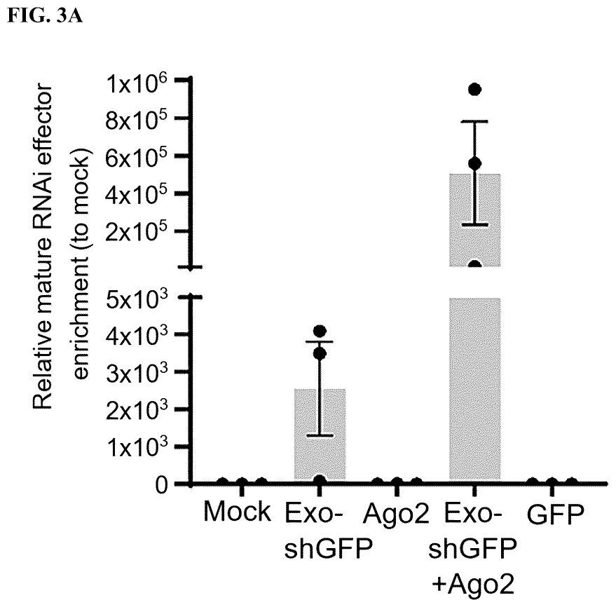

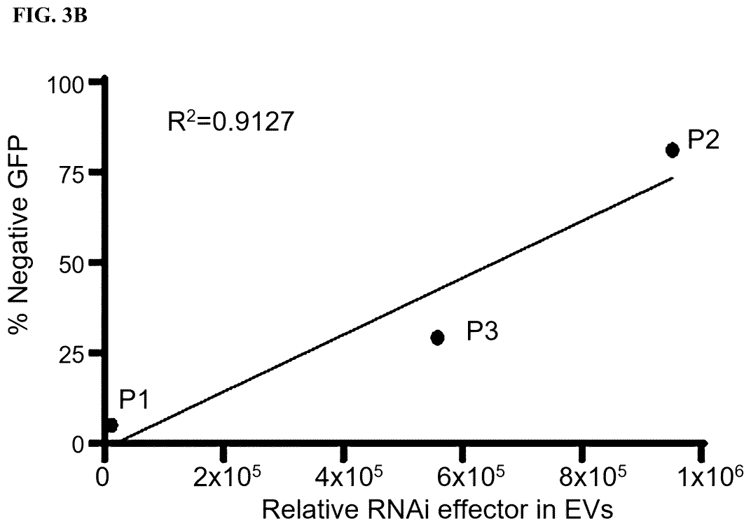

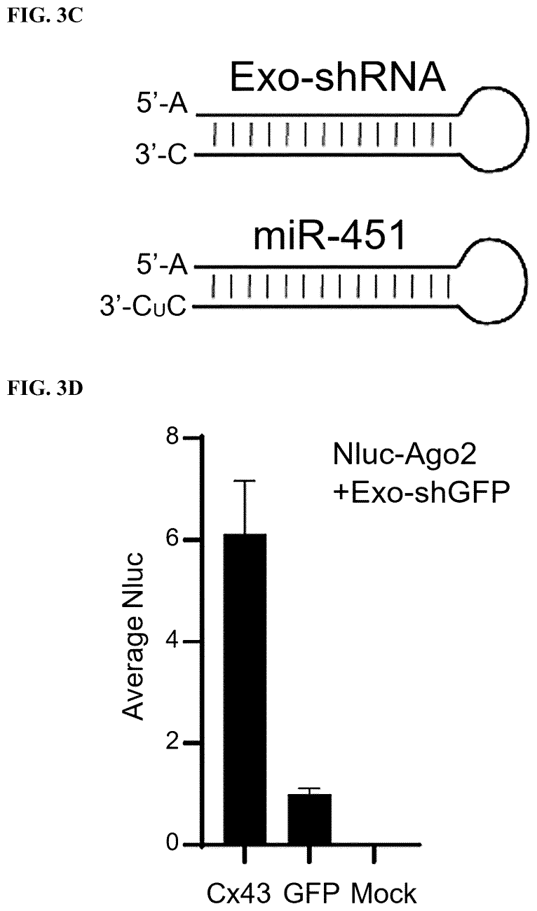

The terms “stem loop” or “RNA stem loop” are interchangeable, and are used in accordance to their ordinary meaning in the art, and refer to a region of an RNA that that includes two nucleotide sequences (e.g. stem sequences) that base pair to form a double-stranded structure (e.g. stem), and a non-base paired structure (e.g. loop) at one end of the double-stranded structure. Thus, the stem sequence refers to one of the nucleotide sequences that base pairs with a second nucleotide sequence to form the double-stranded stem of the stem loop. The stem of the RNA stem loop is typically from about 4 to about 40 base pairs in length. In embodiments, the stem of a Dicer-independent shRNA is 19 base pairs or less in length. In embodiments, the stem of a Dicer-dependent shRNA is 20 base pairs or more in length. The stem may include internal-loops, also known as “bulges”, where the double-stranded RNA separates due to non-base pairing (e.g. unmatched base pair) between nucleotides. As described above, the loop forms a non-based paired structure at one end of the RNA stem loop. Thus, the loop caps one end of the RNA stem loop. Example RNA stem loops are shown, for example, in FIG. 3C.

In embodiments, the stem sequence is about 8 base pairs to about 19 base pairs in length. In embodiments, the stem sequence is about 9 base pairs to about 19 base pairs in length. In embodiments, the stem sequence is about 10 base pairs to about 19 base pairs in length. In embodiments, the stem sequence is about 11 base pairs to about 19 base pairs in length. In embodiments, the stem sequence is about 12 base pairs to about 19 base pairs in length. In embodiments, the stem sequence is about 13 base pairs to about 19 base pairs in length. In embodiments, the stem sequence is about 14 base pairs to about 19 base pairs in length. In embodiments, the stem sequence is about 15 base pairs to about 19 base pairs in length. In embodiments, the stem sequence is about 16 base pairs to about 19 base pairs in length. In embodiments, the stem sequence is about 17 base pairs to about 19 base pairs in length. In embodiments, the stem sequence is about 18 base pairs to about 19 base pairs in length.

In embodiments, the stem sequence is about 8 base pairs to about 18 base pairs in length. In embodiments, the stem sequence is about 8 base pairs to about 17 base pairs in length. In embodiments, the stem sequence is about 8 base pairs to about 16 base pairs in length. In embodiments, the stem sequence is about 8 base pairs to about 15 base pairs in length. In embodiments, the stem sequence is about 8 base pairs to about 14 base pairs in length. In embodiments, the stem sequence is about 8 base pairs to about 13 base pairs in length. In embodiments, the stem sequence is about 8 base pairs to about 12 base pairs in length. In embodiments, the stem sequence is about 8 base pairs to about 11 base pairs in length. In embodiments, the stem sequence is about 8 base pairs to about 10 base pairs in length. In embodiments, the stem sequence is about 8 base pairs to about 9 base pairs in length. In embodiments, the stem sequence is no more than 19 base pairs in length. In embodiments, the stem sequence is less than 19 base pairs in length. In embodiments, the stem sequence is 16 to 18 base pairs in length. In embodiments, the stem sequence is about 8, 9, 10, 11, 12, 13, 14, 15, 16, 17, 18, or 19 base pairs in length. In embodiments, the stem sequence is 8 base pairs in length. In embodiments, the stem sequence is 9 base pairs in length. In embodiments, the stem sequence is 10 base pairs in length. In embodiments, the stem sequence is 11 base pairs in length. In embodiments, the stem sequence is 12 base pairs in length. In embodiments, the stem sequence is 13 base pairs in length. In embodiments, the stem sequence is 14 base pairs in length. In embodiments, the stem sequence is 15 base pairs in length. In embodiments, the stem sequence is 16 base pairs in length. In embodiments, the stem sequence is 17 base pairs in length. In embodiments, the stem sequence is 18 base pairs in length. In embodiments, the stem sequence is 19 base pairs in length. In embodiments, the stem sequence is no greater than 19 base pairs in length.

In embodiments, shRNA nucleic acid includes a stem loop including a loop sequence about 1 nucleotide to about 8 nucleotides in length. In embodiments, the loop sequence is about 2 nucleotides to about 8 nucleotides in length. In embodiments, the loop sequence is about 3 nucleotides to about 8 nucleotides in length. In embodiments, the loop sequence is about 4 nucleotides to about 8 nucleotides in length. In embodiments, the loop sequence is about 5 nucleotides to about 8 nucleotides in length. In embodiments, the loop sequence is about 6 nucleotides to about 8 nucleotides in length. In embodiments, the loop sequence is about 7 nucleotides to about 8 nucleotides in length.

In embodiments, the loop sequence is about 1 nucleotide to about 7 nucleotides in length. In embodiments, the loop sequence is about 1 nucleotide to about 6 nucleotides in length. In embodiments, the loop sequence is about 1 nucleotide to about 5 nucleotides in length. In embodiments, the loop sequence is about 1 nucleotide to about 4 nucleotides in length. In embodiments, the loop sequence is about 1 nucleotide to about 3 nucleotides in length. In embodiments, the loop sequence is about 1 nucleotide to about 2 nucleotides in length. In embodiments, the loop sequence is about 1, 2, 3, 4, 5, 6, 7, or 8 nucleotides in length.

In embodiments, loop sequence is about 3 nucleotides to about 5 nucleotides in length. In embodiments, the loop sequence is 5 nucleotides in length. In embodiments, the loop sequence is 4 nucleotides in length. In embodiments, the loop sequence is 3 nucleotides in length.

For the method provided herein, in embodiments, the shRNA includes an overhang sequence, wherein the overhang sequence increases enrichment of an siRNA nucleic acid in the extracellular vesicle relative to an siRNA nucleic acid derived from the same shRNA nucleic acid lacking the overhang sequence. In embodiments, the shRNA nucleic acid includes an overhang sequence on the 5′ end. In embodiments, the shRNA nucleic acid includes an overhang sequence on the 3′ end. “Overhang sequence” is used in accordance to its plain ordinary meaning and refers to one or more unpaired nucleotides at the end of a double-stranded nucleic acid. An overhang sequence extends beyond the double-stranded sequence. In embodiments, the overhang sequence extends beyond the complementary sequence. In embodiments, the overhang sequence is about 1 nucleotide to about 10 nucleotides in length. In embodiments, the overhang sequence is about 2 nucleotides to about 10 nucleotides in length. In embodiments, the overhang sequence is about 3 nucleotides to about 10 nucleotides in length. In embodiments, the overhang sequence is about 4 nucleotides to about 10 nucleotides in length. In embodiments, the overhang sequence is about 5 nucleotides to about 10 nucleotides in length. In embodiments, the overhang sequence is about 6 nucleotides to about 10 nucleotides in length. In embodiments, the overhang sequence is about 7 nucleotides to about 10 nucleotides in length. In embodiments, the overhang sequence is about 8 nucleotides to about 10 nucleotides in length. In embodiments, the overhang sequence is about 9 nucleotides to about 10 nucleotides in length.

In embodiments, the overhang sequence is about 1 nucleotide to about 9 nucleotides in length. In embodiments, the overhang sequence is about 1 nucleotide to about 8 nucleotides in length. In embodiments, the overhang sequence is about 1 nucleotide to about 7 nucleotides in length. In embodiments, the overhang sequence is about 1 nucleotide to about 6 nucleotides in length. In embodiments, the overhang sequence is about 1 nucleotide to about 5 nucleotides in length. In embodiments, the overhang sequence is about 1 nucleotide to about 4 nucleotides in length. In embodiments, the overhang sequence is about 1 nucleotide to about 3 nucleotides in length. In embodiments, the overhang sequence is about 1 nucleotide to about 3 nucleotides in length.

In embodiments, the overhang sequence includes UC. In embodiments, the overhang sequence is UC. In embodiments, the overhang sequence includes CUC. In embodiments, the overhang sequence is CUC.

In embodiments, the shRNA includes an unmatched base pair, also referred to as a mismatched base pair, wherein the unmatched base pair increases enrichment of an siRNA nucleic acid in the extracellular vesicle relative to an siRNA nucleic acid derived from the same shRNA nucleic acid lacking the unmatched base pair. “Unmatched base pair” and “mismatched base pair” are interchangeable, and are used in accordance to their plain ordinary meaning in the art, and refer to when a nucleotide is paired with a non-complementary base on the opposite strand. Examples of an unmatched base pair include a G/U or A/C pairing. In embodiments the unmatched base pair is an unmatched base pair at the terminal ends (e.g. 5′ and the 3)′ of the shRNA nucleic acid. In embodiments, the unmatched base pair is an adenine at the 5′ end of the shRNA and a cytosine at the 3′ end of the shRNA.

In embodiments, the shRNA nucleic acid includes the sequence of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:71, SEQ ID NO:82, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, or SEQ ID NO:152. In embodiments, the shRNA nucleic acid includes the sequence of SEQ ID NO:42. In embodiments, the shRNA nucleic acid includes the sequence of SEQ ID NO:43. In embodiments, the shRNA nucleic acid includes the sequence of SEQ ID NO:44. In embodiments, the shRNA nucleic acid includes the sequence of SEQ ID NO:45. In embodiments, the shRNA nucleic acid includes the sequence of SEQ ID NO:46. In embodiments, the shRNA nucleic acid includes the sequence of SEQ ID NO:47. In embodiments, the shRNA nucleic acid includes the sequence of SEQ ID NO:48. In embodiments, the shRNA nucleic acid includes the sequence of SEQ ID NO:49. In embodiments, the shRNA nucleic acid includes the sequence of SEQ ID NO:50. In embodiments, the shRNA nucleic acid includes the sequence of SEQ ID NO:51. In embodiments, the shRNA nucleic acid includes the sequence of SEQ ID NO:52. In embodiments, the shRNA nucleic acid includes the sequence of SEQ ID NO:53. In embodiments, the shRNA nucleic acid includes the sequence of SEQ ID NO:54. In embodiments, the shRNA nucleic acid includes the sequence of SEQ ID NO:55. In embodiments, the shRNA nucleic acid includes the sequence of SEQ ID NO:56. In embodiments, the shRNA nucleic acid includes the sequence of SEQ ID NO:57. In embodiments, the shRNA nucleic acid includes the sequence of SEQ ID NO:58. In embodiments, the shRNA nucleic acid includes the sequence of SEQ ID NO:59. In embodiments, the shRNA nucleic acid includes the sequence of SEQ ID NO:60. In embodiments, the shRNA nucleic acid includes the sequence of SEQ ID NO:61. In embodiments, the shRNA nucleic acid includes the sequence of SEQ ID NO:62. In embodiments, the shRNA nucleic acid includes the sequence of SEQ ID NO:63. In embodiments, the shRNA nucleic acid includes the sequence of SEQ ID NO:64. In embodiments, the shRNA nucleic acid includes the sequence of SEQ ID NO:65. In embodiments, the shRNA nucleic acid includes the sequence of SEQ ID NO:66. In embodiments, the shRNA nucleic acid includes the sequence of SEQ ID NO:67. In embodiments, the shRNA nucleic acid includes the sequence of SEQ ID NO:68. In embodiments, the shRNA nucleic acid includes the sequence of SEQ ID NO:69. In embodiments, the shRNA nucleic acid includes the sequence of SEQ ID NO:70. In embodiments, the shRNA nucleic acid includes the sequence of SEQ ID NO:71. In embodiments, the shRNA nucleic acid includes the sequence of SEQ ID NO:72. In embodiments, the shRNA nucleic acid includes the sequence of SEQ ID NO:73. In embodiments, the shRNA nucleic acid includes the sequence of SEQ ID NO:74. In embodiments, the shRNA nucleic acid includes the sequence of SEQ ID NO:75. In embodiments, the shRNA nucleic acid includes the sequence of SEQ ID NO:76. In embodiments, the shRNA nucleic acid includes the sequence of SEQ ID NO:77. In embodiments, the shRNA nucleic acid includes the sequence of SEQ ID NO:78. In embodiments, the shRNA nucleic acid includes the sequence of SEQ ID NO:79. In embodiments, the shRNA nucleic acid includes the sequence of SEQ ID NO:80. In embodiments, the shRNA nucleic acid includes the sequence of SEQ ID NO:71. In embodiments, the shRNA nucleic acid includes the sequence of SEQ ID NO:82. In embodiments, the shRNA nucleic acid includes the sequence of SEQ ID NO:97. In embodiments, the shRNA nucleic acid includes the sequence of SEQ ID NO:98. In embodiments, the shRNA nucleic acid includes the sequence of SEQ ID NO:99. In embodiments, the shRNA nucleic acid includes the sequence of SEQ ID NO:100. In embodiments, the shRNA nucleic acid includes the sequence of SEQ ID NO:101. In embodiments, the shRNA nucleic acid includes the sequence of SEQ ID NO:102. In embodiments, the shRNA nucleic acid includes the sequence of SEQ ID NO:103. In embodiments, the shRNA nucleic acid includes the sequence of SEQ ID NO:104. In embodiments, the shRNA nucleic acid includes the sequence of SEQ ID NO:105. In embodiments, the shRNA nucleic acid includes the sequence of SEQ ID NO:106. In embodiments, the shRNA nucleic acid includes the sequence of SEQ ID NO:107. In embodiments, the shRNA nucleic acid includes the sequence of SEQ ID NO:108. In embodiments, the shRNA nucleic acid includes the sequence of SEQ ID NO:109. In embodiments, the shRNA nucleic acid includes the sequence of SEQ ID NO:110. In embodiments, the shRNA nucleic acid includes the sequence of SEQ ID NO:132. In embodiments, the shRNA nucleic acid includes the sequence of SEQ ID NO:133. In embodiments, the shRNA nucleic acid includes the sequence of SEQ ID NO:134. In embodiments, the shRNA nucleic acid includes the sequence of SEQ ID NO:135. In embodiments, the shRNA nucleic acid includes the sequence of SEQ ID NO:136. In embodiments, the shRNA nucleic acid includes the sequence of SEQ ID NO:137. In embodiments, the shRNA nucleic acid includes the sequence of SEQ ID NO:138. In embodiments, the shRNA nucleic acid includes the sequence of SEQ ID NO:139. In embodiments, the shRNA nucleic acid includes the sequence of SEQ ID NO:140. In embodiments, the shRNA nucleic acid includes the sequence of SEQ ID NO:141. In embodiments, the shRNA nucleic acid includes the sequence of SEQ ID NO:142. In embodiments, the shRNA nucleic acid includes the sequence of SEQ ID NO:143. In embodiments, the shRNA nucleic acid includes the sequence of SEQ ID NO:144. In embodiments, the shRNA nucleic acid includes the sequence of SEQ ID NO:145. In embodiments, the shRNA nucleic acid includes the sequence of SEQ ID NO:146. In embodiments, the shRNA nucleic acid includes the sequence of SEQ ID NO:147. In embodiments, the shRNA nucleic acid includes the sequence of SEQ ID NO:148. In embodiments, the shRNA nucleic acid includes the sequence of SEQ ID NO:149. In embodiments, the shRNA nucleic acid includes the sequence of SEQ ID NO:150. In embodiments, the shRNA nucleic acid includes the sequence of SEQ ID NO:152.

For the method provided herein, in embodiments, the shRNA nucleic acid includes an exosome-specific RNA motif. As used herein, “exosome-specific RNA motif” refers to an RNA sequence (e.g. about 2 to about 20 nucleotides in length) commonly found in the sequences of RNA nucleic acids within the internal spaces of extracellular vesicles. Exosome-specific RNA motifs typically bind to proteins associated with inclusion of RNA sequences into extracellular vesicles. In embodiments, the exosome-specific RNA motif interacts with an endogenous protein associated with extracellular vesicle biogenesis. In embodiments, the exosome-specific RNA motif binds or recruits a protein (e.g. ALIX, SYNCRIP, hnRNPA2B1, etc.) associated with sorting cargo into extracellular vesicles. In embodiments, the exosome-specific RNA motif interacts with an extracellular vesicle membrane-associated protein. An exosome-specific motif within an RNA sequence therefore facilitates loading of the RNA into an extracellular vesicle. In embodiments, presence of an exosome-specific RNA motif in an RNA (e.g. shRNA) increases the level of said RNA or a sequence derived from said RNA (e.g. siRNA) in an extracellular vesicle relative to an RNA lacking said exosome-specific RNA motif. In embodiments, presence of an exosome-specific RNA motif in an RNA increases the percentage of extracellular vesicles in a population of extracellular vesicles including the RNA or a sequence derived from said RNA (e.g. siRNA) relative to an RNA that lacks the exosome-specific RNA motif. In embodiments, the exosome-specific RNA motif is a G-rich or a C-rich RNA sequence. In embodiments, the exosome-specific RNA motif is an RNA sequence with a low occurrence of the nucleotide base A. In embodiments, the exosome-specific RNA motif is derived from a nucleic acid derived from a hepatocyte. In embodiments, the exosome-specific RNA motif is derived from a nucleic acid derived from an astrocyte. In embodiments, the exosome-specific RNA motif is derived from a nucleic acid derived from a neuron.

In embodiments, the exosome specific RNA motif includes an miR-451 motif, an exo-motif, an hExomotif, a polyuridylation motif, an LA motif, an astrocyte/neuron motif, an extended exomotif, or a core exomotif. In embodiments, the exosome specific RNA motif includes an miR-451 motif. In embodiments, the exosome specific RNA motif includes an exo-motif. In embodiments, the exosome specific RNA motif includes an hExomitif. In embodiments, the exosome specific RNA motif includes a polyuridylation motif. In embodiments, the exosome specific RNA motif includes an LA motif. In embodiments, the exosome specific RNA motif includes an astrocyte/neuron motif. In embodiments, the exosome specific RNA motif includes an extended exomotif. In embodiments, the exosome specific RNA motif includes a core exomotif. In embodiments, the exosome specific RNA motif is not an miR-451 motif. In embodiments, the shRNA does not include an miR-451 motif.

In embodiments, the miR-451 motif is the sequence of CUC. In embodiments, the Exo-motif is the sequence of GGAG or CCCU. In embodiments, the Exo-motif is the sequence of GGAG. In embodiments, the Exo-motif is the sequence of CCCU. In embodiments, the hExomotif is the sequence of GGCU. In embodiments the astrocyte motif is the sequence of GUAC or CAGUAG. In embodiments the astrocyte motif is the sequence of GUAC. In embodiments the astrocyte motif is the sequence of CAGUAG. In embodiments the neuron motif is the sequence of GUAC or CAGUAG. In embodiments the neuron motif is the sequence of GUAC. In embodiments the neuron motif is the sequence of CAGUAG. In embodiments, the LA motif is the sequence of UGGA or a PolyU sequence. In embodiments, the LA motif is the sequence of UGGA. In embodiments, the LA motif is a PolyU sequence. In embodiments, the Poly uridylation motif is a PolyU sequence. In embodiments, the core motif is the sequence of UGUGU, UGUGUGU, CCGGAG, CGGGAG, CAGUAG CAUG, GUGC, CCCC, or CGGG. In embodiments, the core motif is the sequence of UGUGU. In embodiments, the core motif is the sequence of UGUGUGU. In embodiments, the core motif is the sequence of CCGGAG. In embodiments, the core motif is the sequence of CGGGAG. In embodiments, the core motif is the sequence of CAGUAG. In embodiments, the core motif is the sequence of CAUG. In embodiments, the core motif is the sequence of GUGC. In embodiments, the core motif is the sequence of CCCC. In embodiments, the core motif is the sequence of CGGG. In embodiments, the extended motif is the sequence of UGUGU, UGUGUGU, CCGGAG, CGGGAG, CAGUAG, CAUG, GUGC, CCCC, or CGGG. In embodiments, the extended motif is the sequence of UGUGU. In embodiments, the extended motif is the sequence of UGUGUGU. In embodiments, the extended motif is the sequence of CCGGAG. In embodiments, the extended motif is the sequence of CGGGAG. In embodiments, the extended motif is the sequence of CAGUAG. In embodiments, the extended motif is the sequence of CAUG. In embodiments, the extended motif is the sequence of GUGC. In embodiments, the extended motif is the sequence of CCCC. In embodiments, the extended motif is the sequence of CGGG. In embodiments, the core motif is the sequence of UGUGU, UGUGUGU, CCGGAG, CGGGAG, CAGUAG CAUG, GUGC, CCCC, or CGGG. In embodiments, the core motif is the sequence of UGUGU. In embodiments, the core motif is the sequence of UGUGUGU. In embodiments, the core motif is the sequence of CCGGAG. In embodiments, the core motif is the sequence of CGGGAG. In embodiments, the core motif is the sequence of CAGUAG. In embodiments, the core motif is the sequence of CAUG. In embodiments, the core motif is the sequence of GUGC. In embodiments, the core motif is the sequence of CCCC. In embodiments, the core motif is the sequence of CGGG.

In embodiments, the exosome-specific RNA motif includes the sequence of CUC, GGAC, CCCU, GGCU, GUAC, CAGUAG, UGGA, polyU, UGUGU, UGUGUGU, CCGGAG, CGGGAG, CAGUAG CAUG, GUGC, CCCC, or CGGG. In embodiments, the exosome-specific RNA motif includes the sequence of CUC. In embodiments, the exosome-specific RNA motif includes the sequence of CUC. In embodiments, the exosome-specific RNA motif includes the sequence of GGAC. In embodiments, the exosome-specific RNA motif includes the sequence of CCCU. In embodiments, the exosome-specific RNA motif includes the sequence of GGCU. In embodiments, the exosome-specific RNA motif includes the sequence of GUAC. In embodiments, the exosome-specific RNA motif includes the sequence of CAGUAG. In embodiments, the exosome-specific RNA motif includes the sequence of UGGA. In embodiments, the exosome-specific RNA motif includes a polyU sequence. In embodiments, the exosome-specific RNA motif includes the sequence of UGUGU. In embodiments, the exosome-specific RNA motif includes the sequence of UGUGUGU. In embodiments, the exosome-specific RNA motif includes the sequence of CCGGAG. In embodiments, the exosome-specific RNA motif includes the sequence of CGGGAG. In embodiments, the exosome-specific RNA motif includes the sequence of CAGUAG. In embodiments, the exosome-specific RNA motif includes the sequence of CAUG. In embodiments, the exosome-specific RNA motif includes the sequence of GUGC. In embodiments, the exosome-specific RNA motif includes the sequence of CCCC. In embodiments, the exosome-specific RNA motif includes the sequence of CGGG. In embodiments, the exosome-specific RNA motif is the sequence of CUC. In embodiments, the exosome-specific RNA motif is the sequence of CUC. In embodiments, the exosome-specific RNA motif is the sequence of GGAC. In embodiments, the exosome-specific RNA motif is the sequence of CCCU. In embodiments, the exosome-specific RNA motif is the sequence of GGCU. In embodiments, the exosome-specific RNA motif is the sequence of GUAC. In embodiments, the exosome-specific RNA motif is the sequence of CAGUAG. In embodiments, the exosome-specific RNA motif is the sequence of UGGA. In embodiments, the exosome-specific RNA motif is a polyU sequence. In embodiments, the exosome-specific RNA motif is the sequence of UGUGU. In embodiments, the exosome-specific RNA motif is the sequence of UGUGUGU. In embodiments, the exosome-specific RNA motif is the sequence of CCGGAG. In embodiments, the exosome-specific RNA motif is the sequence of CGGGAG. In embodiments, the exosome-specific RNA motif is the sequence of CAGUAG. In embodiments, the exosome-specific RNA motif is the sequence of CAUG. In embodiments, the exosome-specific RNA motif is the sequence of GUGC. In embodiments, the exosome-specific RNA motif is the sequence of CCCC. In embodiments, the exosome-specific RNA motif is the sequence of CGGG.

In embodiments, the exosome specific RNA motif is at the 3′ end of the shRNA nucleic acid. In embodiments, the exosome specific RNA motif is an overhang sequence at the 3′ end of the shRNA nucleic acid.

In embodiments, the nucleic acid encoding the shRNA nucleic acid is operably linked to a small RNA promoter. A nucleic acid is “operably linked” when it is placed into a functional relationship with another nucleic acid sequence (e.g. a small RNA promoter sequence). For example, a promoter or enhancer is operably linked to a coding sequence if it affects the transcription of the sequence; or a ribosome binding site is operably linked to a coding sequence if it is positioned so as to facilitate translation. Operably linked means that the nucleotide sequences being linked are typically contiguous. However, as enhancers generally function when separated from the promoter by several kilobases and intronic sequences may be of variable lengths, some polynucleotide elements may be operably linked but not directly flanked and may even function in trans from a different allele or chromosome. In embodiments, the small RNA promoter facilitates shuttling of the shRNA to the cytoplasm. For example, accumulation of the shRNA to the cytoplasm may facilitate packaging of the processed siRNA into an extracellular vesicle.

In embodiments, the small RNA promoter is a U6, mU6, H1, 7SK, U1, T7, SP6, T3, tRNA-Lys3, tRNA-Val, tRNA-Ser, tRNA-gly, tRNA-Gln, tRNA-Pro, tRNA-Leu, or tRNA-Asn promoter. In embodiments, the small RNA promoter is a U6 promoter. In embodiments, the small RNA promoter is a mU6 promoter. In embodiments, the small RNA promoter is a H1 promoter. In embodiments, the small RNA promoter is a 7SK promoter. In embodiments, the small RNA promoter is a U1 promoter. In embodiments, the small RNA promoter is a T7 promoter. In embodiments, the small RNA promoter is a SP6 promoter. In embodiments, the small RNA promoter is a T3 promoter. In embodiments, the small RNA promoter is a tRNA-Lys3 promoter. In embodiments, the small RNA promoter is a tRNA-Val promoter. In embodiments, the small RNA promoter is a tRNA-Ser promoter. In embodiments, the small RNA promoter is a tRNA-gly promoter. In embodiments, the small RNA promoter is a tRNA-Gln promoter. In embodiments, the small RNA promoter is a tRNA-Pro promoter. In embodiments, the small RNA promoter is a tRNA-Lue promoter. In embodiments, the small RNA promoter is a or tRNA-Asn promoter.

In embodiments, the U6 promoter includes the sequence of SEQ ID NO:160. In embodiments, the mU6 promoter includes the sequence of SEQ ID NO:161. In embodiments, the H1 promoter includes the sequence of SEQ ID NO:162. In embodiments, the 7SK promoter includes the sequence of SEQ ID NO:163. In embodiments, the U1 promoter includes the sequence of SEQ ID NO:164. In embodiments, the T7 promoter includes the sequence of SEQ ID NO:165. In embodiments, the SP6 promoter includes the sequence of SEQ ID NO:166. In embodiments, the T3 promoter includes the sequence of SEQ ID NO:167. In embodiments, the tRNA lys3 promoter includes the sequence of SEQ ID NO:168. In embodiments, the tRNA val promoter includes the sequence of SEQ ID NO:169. In embodiments, the tRNA ser promoter includes the sequence of SEQ ID NO:170. In embodiments, the tRNA gly promoter includes the sequence of SEQ ID NO:171. In embodiments, the tRNA gln promoter includes the sequence of SEQ ID NO:172. In embodiments, the tRNA pro promoter includes the sequence of SEQ ID NO:173. In embodiments, the tRNA leu promoter includes the sequence of SEQ ID NO:174. In embodiments, the tRNA asn promoter includes the sequence of SEQ ID NO:175. In embodiments, the U6 promoter is the sequence of SEQ ID NO:160. In embodiments, the mU6 promoter is the sequence of SEQ ID NO:161. In embodiments, the H1 promoter is the sequence of SEQ ID NO:162. In embodiments, the 7SK promoter is the sequence of SEQ ID NO:163. In embodiments, the U1 promoter is the sequence of SEQ ID NO:164. In embodiments, the T7 promoter is the sequence of SEQ ID NO:165. In embodiments, the SP6 promoter is the sequence of SEQ ID NO:166. In embodiments, the T3 promoter is the sequence of SEQ ID NO:167. In embodiments, the tRNA lys3 promoter is the sequence of SEQ ID NO:168. In embodiments, the tRNA val promoter is the sequence of SEQ ID NO:169. In embodiments, the tRNA ser promoter is the sequence of SEQ ID NO:170. In embodiments, the tRNA gly promoter is the sequence of SEQ ID NO:171. In embodiments, the tRNA gln promoter is the sequence of SEQ ID NO:172. In embodiments, the tRNA pro promoter is the sequence of SEQ ID NO:173. In embodiments, the tRNA leu promoter is the sequence of SEQ ID NO:174. In embodiments, the tRNA asn promoter is the sequence of SEQ ID NO:175.

For the nucleic acid encoding the shRNA nucleic acid provided herein, in embodiments, transcription is initiated at an alternative initiator nucleotide. For example, transcription of the shRNA nucleic acid may be initiated at a different nucleotide than what is typical for the promoter used. “Initiator nucleotide” is used in accordance to its plain ordinary meaning in the art and refers to a specific base where RNA polymerase begins to transcribe a sequence (e.g. a gene, a shRNA encoding nucleic acid). The initiator nucleotide may be referred to as the transcription start site or TSS. In embodiments, the alternative initiator nucleotide is an A or a G. Thus, for the nucleic acid encoding the shRNA nucleic acid, in embodiments, transcription is initiated from an A or a G. In embodiments, transcription is initiated from an A. In embodiments, transcription is initiated from a G.

For the method provided herein, in embodiments, the siRNA nucleic acid is a single-stranded RNA (ssRNA) or a double-stranded RNA (dsRNA). In embodiments, the siRNA is a single-stranded RNA. In embodiments, the single-stranded siRNA nucleic acid the antisense (AS) strand of the siRNA. For example, in embodiments, the AGO2 may cleave and/or degrade the sense strand (e.g. passenger strand) of the shRNA, thereby generating an single-stranded siRNA including the antisense strand of the siRNA capable of hybridizing to a target sequence (e.g. mRNA, promoter sequence, etc.). Thus, in embodiments, the siRNA nucleic acid is a ssRNA.

In embodiments, the siRNA nucleic acid is about 10 nucleotides to about 30 nucleotides in length. In embodiments, the siRNA nucleic acid is about 11 nucleotides to about 30 nucleotides in length. In embodiments, the siRNA nucleic acid is about 12 nucleotides to about 30 nucleotides in length. In embodiments, the siRNA nucleic acid is about 13 nucleotides to about 30 nucleotides in length. In embodiments, the siRNA nucleic acid is about 14 nucleotides to about 30 nucleotides in length. In embodiments, the siRNA nucleic acid is about 15 nucleotides to about 30 nucleotides in length. In embodiments, the siRNA nucleic acid is about 16 nucleotides to about 30 nucleotides in length. In embodiments, the siRNA nucleic acid is about 17 nucleotides to about 30 nucleotides in length. In embodiments, the siRNA nucleic acid is about 18 nucleotides to about 30 nucleotides in length. In embodiments, the siRNA nucleic acid is about 19 nucleotides to about 30 nucleotides in length. In embodiments, the siRNA nucleic acid is about 20 nucleotides to about 30 nucleotides in length. In embodiments, the siRNA nucleic acid is about 21 nucleotides to about 30 nucleotides in length. In embodiments, the siRNA nucleic acid is about 22 nucleotides to about 30 nucleotides in length. In embodiments, the siRNA nucleic acid is about 23 nucleotides to about 30 nucleotides in length. In embodiments, the siRNA nucleic acid is about 24 nucleotides to about 30 nucleotides in length. In embodiments, the siRNA nucleic acid is about 25 nucleotides to about 30 nucleotides in length. In embodiments, the siRNA nucleic acid is about 26 nucleotides to about 30 nucleotides in length. In embodiments, the siRNA nucleic acid is about 27 nucleotides to about 30 nucleotides in length. In embodiments, the siRNA nucleic acid is about 28 nucleotides to about 30 nucleotides in length. In embodiments, the siRNA nucleic acid is about 29 nucleotides to about 30 nucleotides in length.

In embodiments, the siRNA nucleic acid is about 10 nucleotides to about 29 nucleotides in length. In embodiments, the siRNA nucleic acid is about 10 nucleotides to about 28 nucleotides in length. In embodiments, the siRNA nucleic acid is about 10 nucleotides to about 27 nucleotides in length. In embodiments, the siRNA nucleic acid is about 10 nucleotides to about 26 nucleotides in length. In embodiments, the siRNA nucleic acid is about 10 nucleotides to about 25 nucleotides in length. In embodiments, the siRNA nucleic acid is about 10 nucleotides to about 24 nucleotides in length. In embodiments, the siRNA nucleic acid is about 10 nucleotides to about 23 nucleotides in length. In embodiments, the siRNA nucleic acid is about 10 nucleotides to about 22 nucleotides in length. In embodiments, the siRNA nucleic acid is about 10 nucleotides to about 21 nucleotides in length. In embodiments, the siRNA nucleic acid is about 10 nucleotides to about 20 nucleotides in length. In embodiments, the siRNA nucleic acid is about 10 nucleotides to about 19 nucleotides in length. In embodiments, the siRNA nucleic acid is about 10 nucleotides to about 18 nucleotides in length. In embodiments, the siRNA nucleic acid is about 10 nucleotides to about 17 nucleotides in length. In embodiments, the siRNA nucleic acid is about 10 nucleotides to about 16 nucleotides in length. In embodiments, the siRNA nucleic acid is about 10 nucleotides to about 15 nucleotides in length. In embodiments, the siRNA nucleic acid is about 10 nucleotides to about 14 nucleotides in length. In embodiments, the siRNA nucleic acid is about 10 nucleotides to about 13 nucleotides in length. In embodiments, the siRNA nucleic acid is about 10 nucleotides to about 12 nucleotides in length. In embodiments, the siRNA nucleic acid is about 10 nucleotides to about 11 nucleotides in length. In embodiments, the siRNA nucleic acid is about 10, 11, 12, 13, 14, 15, 16, 17, 18, 19, 20, 21, 22, 23, 24, 25, 26, 27, 28, 29, or 30 nucleotides in length.

In embodiments, the siRNA nucleic acid is about 15 nucleotides to about 25 nucleotides in length. In embodiments, the siRNA nucleic acid is about 16 nucleotides to about 25 nucleotides in length. In embodiments, the siRNA nucleic acid is about 17 nucleotides to about 25 nucleotides in length. In embodiments, the siRNA nucleic acid is about 18 nucleotides to about 25 nucleotides in length. In embodiments, the siRNA nucleic acid is about 19 nucleotides to about 25 nucleotides in length. In embodiments, the siRNA nucleic acid is about 20 nucleotides to about 25 nucleotides in length. In embodiments, the siRNA nucleic acid is about 21 nucleotides to about 25 nucleotides in length. In embodiments, the siRNA nucleic acid is about 22 nucleotides to about 25 nucleotides in length. In embodiments, the siRNA nucleic acid is about 23 nucleotides to about 25 nucleotides in length. In embodiments, the siRNA nucleic acid is about 24 nucleotides to about 25 nucleotides in length.

In embodiments, the siRNA nucleic acid is about 15 nucleotides to about 24 nucleotides in length. In embodiments, the siRNA nucleic acid is about 15 nucleotides to about 23 nucleotides in length. In embodiments, the siRNA nucleic acid is about 15 nucleotides to about 22 nucleotides in length. In embodiments, the siRNA nucleic acid is about 15 nucleotides to about 21 nucleotides in length. In embodiments, the siRNA nucleic acid is about 15 nucleotides to about 20 nucleotides in length. In embodiments, the siRNA nucleic acid is about 15 nucleotides to about 19 nucleotides in length. In embodiments, the siRNA nucleic acid is about 15 nucleotides to about 18 nucleotides in length. In embodiments, the siRNA nucleic acid is about 15 nucleotides to about 17 nucleotides in length. In embodiments, the siRNA nucleic acid is about 15 nucleotides to about 16 nucleotides in length. In embodiments, said siRNA is about 18 nucleotides to about 20 nt in length. In embodiments, the siRNA nucleic acid is about 10, 11, 12, 13, 14, 15, 16, 17, 18, 19, 20, 21, 22, 23, 24, 25, 26, 27, 28, 29, or 30 nucleotides in length. In embodiments, the siRNA nucleic acid is no more than about 10, 11, 12, 13, 14, 15, 16, 17, 18, 19, 20, 21, 22, 23, 24, 25, 26, 27, 28, 29, or 30 nucleotides in length.

For the Dicer-independent shRNA nucleic acid provided herein, in embodiments, Dicer cleaves the passenger (e.g. sense, non-targeting) strand of the shRNA nucleic acid. In embodiments, the remaining anti-sense (e.g. targeting) strand of the siRNA includes a single-stranded RNA including a stem sequence and the loop sequence of the shRNA nucleic acid. Thus, in embodiments, the siRNA nucleic acid includes the loop sequence of the shRNA stem loop. In embodiments, the loop is at least partially complementary to a target nucleic acid sequence.

For the siRNA provided herein, in embodiments, the siRNA is capable of hybridizing to a target nucleic acid sequence. Thus, in embodiments, said siRNA nucleic acid is at least partially complementary to a target nucleic acid sequence. In embodiments, target nucleic acid sequence encodes a disease target. In embodiments, the disease is a neurodegenerative disease. For example, in embodiments, the nucleic acid encodes an isoform of the major prion protein. In embodiments, the nucleic acid encodes tau protein. In embodiments, the nucleic acid encodes transthyretin (TTR) protein. In embodiments, the target nucleic acid sequence encodes a cancer protein. “Cancer protein” as used herein refers to a protein expressed in higher levels in a cancer cell relative to a healthy cell. In embodiments, the target nucleic acid encodes a viral protein. In embodiments, the viral protein is an HIV protein. In embodiments, the target nucleic acid includes a promoter sequence. In embodiments, the promoter sequence is an HIV promoter sequence. In embodiments, the promoter sequence includes an HIV long terminal repeat (LTR) sequence.

For the method provided herein, in embodiments, the AGO2 protein includes one or more mutations that increase colocalization of AGO2 protein to an extracellular vesicle relative to an AGO2 protein lacking the mutation. For example, in embodiments, the mutation may increase colocalization of AGO2 protein to the site of extracellular vesicle biogenesis (e.g. multivesicular body) relative to an AGO2 protein lacking the mutation. In embodiments, the mutation increases the level of AGO2 protein within an extracellular vesicle relative to an AGO2 protein lacking the mutation.

In embodiments, the AGO2 protein provided herein including embodiments thereof may include one or more modifications wherein one or more one phosphorylation sites is modified or deleted. In embodiments, the one or more modifications includes removal of phosphorylation site. In embodiments, the one or more mutations is a substitution of a deletion of a residue capable of being phosphorylated. For example, in embodiments, the one or more mutations may be at a phosphorylation site, wherein phosphorylation of the residue is associated with regulating AGO2 sorting into multivesicular bodies. In embodiments, the one or more mutations increases colocalization of AGO2 protein to an extracellular vesicle relative to an AGO2 protein lacking the mutation. In embodiments, the mutation increases colocalization of AGO2 protein to the site of extracellular vesicle biogenesis (e.g. multivesicular body) relative to an AGO2 protein lacking the mutation. In embodiments, the mutation increases the level of AGO2 protein within an extracellular vesicle relative to an AGO2 protein lacking the mutation. In embodiments, the one or more mutations increases signaling associated with Ago2 trafficking into an extracellular vesicle. In embodiments, the one or more mutations may be at a residue capable of being phosphorylated by any one or Akt3, MAPKAPK2 (MK2), MEKI, MEKII, ERK, EGFR, p38-MAPK, or a combination thereof. In embodiments, the AGO2 protein includes an S387A mutation at a position corresponding to position 387 of SEQ ID NO:159.

For the method provided herein, in embodiments, the cell expresses a fusogen protein. In embodiments, the cell includes a fusogen protein. The term “fusogen” or “fusogen protein” refers to a protein that facilitates merging of two separate membranes (e.g. membrane fusion) into one membrane. For example, a fusogen may facilitate fusion of a vesicle and a target organelle within a cell. In another example, a fusogen may facilitate the fusion of the lipid bilayer membrane between two different cells. In another example, a fusogen may facilitate fusion of the membrane of an enveloped virus or virus-like structure to a host cell membrane, thereby allowing entry of the virus into the host cell. In another example, a fusogen may facilitate fusion of an extracellular vesicle membrane with the membrane of a target cell, thereby allowing entry of extracellular vesicle cargo (e.g. siRNA) into the target cell.

Thus, for the method provided herein, in embodiments, step i) further includes transfecting the cell with a nucleic acid encoding a fusogen protein. In embodiments, step ii) includes culturing the cell under conditions to express the fusogen protein. In embodiments, the cell expresses the fusogen protein. In embodiments, the fusogen protein is associated with an extracellular vesicle membrane. In embodiments, the fusogen protein includes a transmembrane portion embedded in the extracellular vesicle membrane. In embodiments, the fusogen protein is associated with the exterior of the extracellular vesicle membrane.

In embodiments, the fusogen protein is gap junction protein, connexin 43, syncytin-A (SynA), myoferlin, vesicular stomatitis virus G (VSV-G), Sindbis virus glycoprotein (SINmu), baboon retroviral envelope glycoprotein (BaEV), measles virus glycoprotein, nipah virus envelope glycoprotein, GALA peptide, EALA peptide, or KALA peptide. In embodiments, the fusogen protein is connexin 43. In embodiments, the fusogen protein is syncytin-A (SynA). In embodiments, the fusogen protein is myoferlin. In embodiments, the fusogen protein is vesicular stomatitis virus G (VSV-G). In embodiments, the fusogen protein is Sindbis virus glycoprotein (SINmu). In embodiments, the fusogen protein is baboon retroviral envelope glycoprotein (BaEV). In embodiments, the fusogen protein is measles virus glycoprotein. In embodiments, the fusogen protein is nipah virus envelope glycoprotein. In embodiments, the fusogen protein is GALA peptide. In embodiments, the fusogen protein is EALA peptide. In embodiments, the fusogen protein is KALA peptide.

In embodiments, the GALA peptide includes the sequence of SEQ ID NO:157. In embodiments, the GALA peptide is the sequence of SEQ ID NO:157. In embodiments, the EALA peptide includes the sequence of SEQ ID NO:158. In embodiments, the EALA peptide is the sequence of SEQ ID NO:158. In embodiments, the KALA peptide includes the sequence of SEQ ID NO:159. In embodiments, the KALA peptide is the sequence of SEQ ID NO:159.

For the method provided herein, in embodiments, the method includes transfecting a cell with a nucleic acid encoding a protein that can be used to target the extracellular vesicle to a particular cell, organ or tissue. Thus, in embodiments, step i) further includes transfecting said cell with a nucleic acid encoding a fusion protein including: a) an extracellular vesicle membrane-associated protein, and b) a targeting protein or a detectable moiety. A n “extracellular vesicle membrane-associated protein” refers to a protein on the membrane of an extracellular vesicle, such as a transmembrane protein, an integral protein, or a peripheral protein. Extracellular vescile membrane-associated protein include various CD proteins, transporters, integrins, lectins and cadherins. Exemplary membrane-associated proteins include PDGFR, CD9, CD37, CD53, CD63, CD68, CD81, CD82, LAMP-1, LAMP-2A, LAMP-2B, LAMP-2C, lactadherin, PTGFRN, BSG, IGSF3, IGSF8, ITGB1, ITGA4, SLC3A2, IGSF2, and ATP transporter proteins (ATP1A1, ATP1A2, ATP1A3, ATP1A4, ATP1B3, ATP2B1, ATP2B2, ATP2B3, ATP2B4). In embodiments, the extracellular vesicle membrane-associated protein is PDGFR, CD9, CD37, CD53, CD63, CD68, CD81, CD82, LAMP-1, LAMP-2A, LAMP-2B, LAMP-2C, lactadherin, or PTGFRN. In embodiments, the extracellular vesicle membrane-associated protein is PDGFR. In embodiments, the extracellular vesicle membrane-associated protein is CD9. In embodiments, the extracellular vesicle membrane-associated protein is CD37. In embodiments, the extracellular vesicle membrane-associated protein is CD53. In embodiments, the extracellular vesicle membrane-associated protein is CD63. In embodiments, the extracellular vesicle membrane-associated protein is CD68. In embodiments, the extracellular vesicle membrane-associated protein is CD81. In embodiments, the extracellular vesicle membrane-associated protein is CD82. In embodiments, the extracellular vesicle membrane-associated protein is LAMP-1. In embodiments, the extracellular vesicle membrane-associated protein is LAMP-2A. In embodiments, the extracellular vesicle membrane-associated protein is LAMP-2B. In embodiments, the extracellular vesicle membrane-associated protein is LAMP-2C. In embodiments, the extracellular vesicle membrane-associated protein is lactadherin. In embodiments, the extracellular vesicle membrane-associated protein is PTGFRN. A “target protein” refers to a protein that can be used to target the exosome to a specific organ, tissue, cell, virus, protein, or bacteria for a treatment using the exosomes described herein. In aspects, the target protein binds to or is capable of binding to a cell, protein, virus, or bacteria of interest. In embodiments, the targeting protein is a single chain variable fragment. Fusion proteins including an extracellular membrane associated protein and a targeting protein or detectable moiety are described in more detail in US 2023/0149319, which is incorporated herein in its entirety and for all purposes.

In embodiments, the detectable moiety is a fluorescent protein. In embodiments, the detectable moiety is green fluorescent protein (GFP) or luciferase.

In embodiments, decreasing the level of Charged Multivesicular Body Protein 4C (CHMP4C), Vacuolar Protein Sorting 4 Homolog B (VPS4B), or a combination thereof in a cell increases the level of EV in the cell. For example, in embodiments, decreasing or inhibiting expression of CHMP4C in a cell increases production of EV in the cell. In embodiments, decreasing or inhibiting expression of VPS4B in a cell increases production of EV in the cell. Thus, for the method provided herein, in embodiments, step i) further includes transfecting a cell with a nucleic acid encoding a nucleic acid encoding a Charged Multivesicular Body Protein 4C (CHMP4C) inhibitor, a nucleic acid encoding a Vacuolar Protein Sorting 4 Homolog B (VPS4B) inhibitor, or a combination thereof. In embodiments, step ii) further includes culturing the cell under conditions conducive for expressing the Charged Multivesicular Body Protein 4C (CHMP4C) inhibitor, the Vacuolar Protein Sorting 4 Homolog B (VPS4B) inhibitor, or a combination thereof. Thus, in embodiments, the cell includes a Charged Multivesicular Body Protein 4C (CHMP4C) inhibitor, a Vacuolar Protein Sorting 4 Homolog B (VPS4B) inhibitor, or a combination thereof. In embodiments, the cell includes a Charged Multivesicular Body Protein 4C (CHMP4C) inhibitor. In embodiments, the cell includes a Vacuolar Protein Sorting 4 Homolog B (VPS4B) inhibitor. In embodiments, the inhibitor is an antisense oligonucleotide (ASO), shRNA, or siRNA. For example, in embodiments, the ASO hybridizes to a CHMP4C gene or a VPS4B gene. In embodiments, the ASO hybridizes to a nucleic acid encoding CHMP4C or VPS4B. In embodiments, the shRNA or siRNA hybridizes to an CHMP4C RNA or a VPS4B RNA. In embodiments, the inhibitor hybrids to a CHMP4C promoter or a VPS4B promoter. In embodiments, the VPS4B inhibitor includes the sequence of SEQ ID NO:131. In embodiments, the VPS4B inhibitor is the sequence of SEQ ID NO:131. In embodiments, the CHMP4C inhibitor includes the sequence of SEQ ID NO:132. In embodiments, the CHMP4C inhibitor is the sequence of SEQ ID NO:132.

In embodiments, the cell does not substantially express CHMP4C, VPS4B, or a combination thereof. When a cell does not “substantially express” a gene, one or more of a transcription or translation product of that gene is undetectable using convention methods for detecting gene expression. For example, a protein is not substantially expressed, wherein the protein is undetectable using conventional methods and compositions well known in the art to detect protein or quantify the protein. (e.g. Western Blot, ELISA, SDS PAGE gel, etc.).

In embodiments, the cell expresses lower levels of CHMP4C or VPS4B relative to a cell that that lacks the nucleic acid inhibitor targeting CHMP4C or VPS4B. In embodiments, the cell expresses lower levels of Akt3, MAPKAPK2 (MK2), MEKI, MEKII, ERK, EGFR, or p38-MAPK relative to a cell that lacks the nucleic acid inhibitor targeting Akt3, MAPKAPK2 (MK2), MEKI, MEKII, ERK, EGFR, or p38-MAPK.

In embodiments, the cell includes a nucleic acid encoding an Ago2 cellular retention factor inhibitor. “Ago2 cellular retention factor” refers to a compound (e.g. protein) associated with inhibiting or decreasing trafficking or localization of Ago2 protein into extracellular vesicles and/or cellular organelles associated with extracellular vesicle biogenesis, for example, multivesicular bodies (MVBs) or late endosomes. Thus, in embodiments, an Ago2 cellular retention factor decreases sorting or packaging of Ago2 protein into the intraluminal vesicles (I LVs) within MVBs, and therefore decreases Ago2 secretion from the cell via exosomes. In embodiments, an Ago2 cellular retention factor increases cytoplasmic sequestration of Ago2, thereby decreasing Ago2 binding to proteins associated with EV-sorting. In embodiments, an Ago2 cellular retention factor inhibits protein-protein interactions necessary for Ago2 protein recruitment to MVBs, for example, protein interactions with ESCRT components, including GW182 and ALIX. In embodiments, an Ago2 cellular retention factor modulates post-translational modifications (e.g., phosphorylation, ubiquitination) associated with Ago2 trafficking to cellular organelles associated with extracellular vesicle biogenesis. In embodiments, an Ago2 cellular retention factor modifies membrane lipid interactions or binding motifs that induce Ago2 association with MVB membranes. Thus, in embodiments, an Ago2 cellular retention factor decreases trafficking of Ago2 protein into multivesicular bodies, the site of extracellular vesicle biogenesis. In embodiments, an Ago2 cellular retention factor decreases the level of Ago2 protein in an extracellular vesicle. Thus, in embodiments, step i) further includes transfecting a cell with a nucleic acid encoding an Ago2 cellular retention factor inhibitor. In embodiments, step ii) further includes culturing the cell under conditions conducive for expressing the Ago2 cellular retention factor inhibitor. In embodiments, the Ago2 cellular retention factor is Akt3, MAPKAPK2 (MK2), MEKI, MEKII, ERK, EGFR, p38-MAPK, or a combination thereof. In embodiments, the Ago2 cellular retention factor is Akt3. In embodiments, the Ago2 cellular retention factor is MAPKAPK2 (MK2). In embodiments, the Ago2 cellular retention factor is MEKI. In embodiments, the Ago2 cellular retention factor is MEKII. In embodiments, the Ago2 cellular retention factor is ERK. In embodiments, the Ago2 cellular retention factor is EGFR. In embodiments, the Ago2 cellular retention factor is p38-MAPK. In embodiments, the inhibitor is an antisense oligonucleotide (ASO), shRNA, or siRNA. In embodiments, the inhibitor is a nucleic acid capable of hybridizing to a Ago2 cellular retention factor gene, a nucleic acid encoding a Ago2 cellular retention factor, or a Ago2 cellular retention factor promoter. In embodiments, the cell does not substantially express Akt3, MAPKAPK2 (MK2), MEKI, MEKII, ERK, EGFR, or p38-MAPK, or a combination thereof. In embodiments, the cell does not substantially express Akt3. In embodiments, the cell does not substantially express MAPKAPK2 (MK2). In embodiments, the cell does not substantially express MEKI. In embodiments, the cell does not substantially express MEKII. In embodiments, the cell does not substantially express ERK. In embodiments, the cell does not substantially express EGFR. In embodiments, the cell does not substantially express p38-MAPK. In embodiments, the cell expresses lower levels of Akt3, MAPKAPK2 (MK2), MEKI, MEKII, ERK, EGFR, or p38-MAPK relative to a cell that lacking the Akt3, MAPKAPK2 (MK2), MEKI, MEKII, ERK, EGFR, or p38-MAPK inhibitor.

In embodiments, the cell is a clonal knockout that does not express an Ago2 cellular retention factor provided herein including embodiments thereof. “Clonal knockout” refers to a cell from a cell line derived from a single cell which has been genetically modified to lack a specific gene or lack a functional version of the specific gene (e.g. an Ago2 cellular retention factor gene). Thus, in embodiments, the clonal knockout lacks a Akt3, MAPKAPK2 (MK2), MEKI, MEKII, ERK, EGFR, or p38-MAPK gene. In embodiments, the clonal knockout lacks a functional Akt3, MAPKAPK2 (MK2), MEKI, MEKII, ERK, EGFR, or p38-MAPK gene.

In embodiments, the cell is a clonal knockout that does not express CHMP4C, VPS4B or a combination thereof. In embodiments, the cell is a clonal knockout that does not express CHMP4C. In embodiments, the cell is a clonal knockout that does not express VPS4B.

In embodiments, step i) further includes transfecting a cell with a nucleic acid encoding a Cavelolin1 (CAV1) inhibitor. In embodiments, step ii) further includes culturing the cell under conditions conducive for expressing the Cavelolin1 (CAV1) inhibitor. In embodiments, the cell is a clonal knockout that does not express CAV1.

For the method provided herein, in embodiments, the cell includes a nucleic acid encoding an Ago2 sorting factor. “Ago2 sorting factor” refers to a protein associated with increasing trafficking or localization of Ago2 protein into extracellular vesicles and/or cellular organelles associated with extracellular vesicle biogenesis, for example, multivesicular bodies (MVBs) or late endosomes. In embodiments, an Ago sorting factor increases sorting or packaging of Ago2 protein into the intraluminal vesicles (ILVs) within MVBs, and therefore increases Ago2 secretion from the cell via exosomes. In embodiments, an Ago2 sorting factor increases or upregulates signaling necessary for Ago2 protein recruitment to MVBs. In embodiments, the Ago2 sorting factor increases localization of Ago2 protein into a multivesicular body. Thus, in embodiments, the cell expresses the Ago2 sorting factor. In embodiments, the Ago2 sorting factor is PTPN1, a dominant negative KRAS (S17N) mutant, Cx43, EV-factor ALIX, or a combination thereof. In embodiments, the Ago2 sorting factor is PTPN1. In embodiments, the Ago2 sorting factor is a dominant negative KRAS (S17N) mutant. In embodiments, the Ago2 sorting factor is Cx43. In embodiments, the Ago2 sorting factor is EV-factor ALIX.

For the method provided herein, in embodiments, the cell is a neural stem cell (NSC), a mesenchymal stem cell (MSC), an induced pluripotent stem cell (iPSC), an embryonic stem cell (ESC), an immune cell, or an epithelial cell. In embodiments, the cell is a neural stem cell (NSC). In embodiments, the cell is a mesenchymal stem cell (MSC). In embodiments, the cell is an induced pluripotent stem cell (iPSC). In embodiments, the cell is an embryonic stem cell (ESC). In embodiments, the cell is an immune cell. In embodiments, the cell is an epithelial cell. In embodiments, the cell is a HEK293 cell.

### Cells

The extracellular vesicle provided herein including embodiments therof may be produced in a cell using a method provided herein including embodiments thereof. For example, the extracellular vesicle may be produced in a cell by i) transfecting the cell with a nucleic acid encoding an argonaute 2 (AGO2) protein and a nucleic acid encoding a short hairpin RNA (shRNA) nucleic acid, ii) culturing the cell under conditions conducive for the cell to express the AGO2 protein and the shRNA nucleic acid, thereby forming a cell including the extracellular vesicle including the siRNA nucleic acid. Thus, in an aspect is provided a cell including an argonaute 2 (AGO2) protein and an extracellular vesicle including a small interfering (siRNA) nucleic acid.

As described throughout the specification, in embodiments, the extracellular vesicle provided herein including embodiments thereof may be produced in a stably transduced cell capable of expressing the shRNA nucleic acid provided herein including embodiments thereof and the AGO2 protein provided herein including embodiments thereof. Thus, in embodiments, the cell is stably transduced with a nucleic acid encoding an argonaute 2 (AGO2) protein and a nucleic acid encoding a short hairpin RNA (shRNA).

In embodiments, said siRNA nucleic acid is derived from a short hairpin RNA (shRNA) including the sequence of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:71, SEQ ID NO:82, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, or SEQ ID NO:152.

In embodiments, the siRNA nucleic acid is a single-stranded RNA (ssRNA) or a double-stranded RNA (dsRNA). In embodiments, the siRNA nucleic acid is a single-stranded RNA (ssRNA). In embodiments, the single-stranded siRNA is a mature targeting strand (e.g. antisense strand) derived from an shRNA provided herein including embodiments thereof.

In embodiments, the siRNA nucleic acid is about 10 nucleotides to about 30 nucleotides in length. In embodiments, the siRNA nucleic acid is at least partially complementary to a target nucleic acid sequence. In embodiments, the target nucleic acid sequence encodes a cancer protein. In embodiments, the said target nucleic acid sequence encodes a viral protein. In embodiments, the target nucleic acid sequence includes a promoter sequence.

In embodiments, the AGO2 protein includes an S387A mutation at a position corresponding to position 387 of SEQ ID NO:159.

In embodiments, the extracellular vesicle further includes a fusogen protein associated with the extracellular membrane of said extracellular vesicle. In embodiments, the fusogen protein is connexin 43, syncytin-A (SynA), myoferlin, vesicular stomatitis virus G (VSV-G), Sindbis virus glycoprotein (SINmu), baboon retroviral envelope glycoprotein (BaEV), measles virus glycoprotein, nipah virus envelope glycoprotein, GALA peptide, EALA peptide, or KALA peptide. In embodiments, the fusogen protein is connexin 43 or VSV-G.

In embodiments, the AGO2 protein is exterior to the membrane of said extracellular vesicle. In embodiments, the AGO2 protein is within the internal space of said extracellular vesicle. For example, the AGO2 protein may be encapsulated within the membrane of the extracellular vesicle.

In embodiments, the extracellular vesicle includes a fusion protein including: a) an extracellular membrane-associated protein and b) a target protein or a detectable moiety. In embodiments, the extracellular vesicle membrane-associated protein is PDGFR, CD9, CD37, CD53, CD63, CD68, CD81, CD82, LAMP-1, LAMP-2A, LAMP-2B, LAMP-2C, lactadherin, or PTGFRN. In embodiments, the extracellular vesicle membrane-associated protein is CD63.

In embodiments, the cell includes a plurality of the extracellular vesicle provided herein including embodiments thereof. In embodiments, the cell includes about 10³ to about 10¹⁵ extracellular vesicles. In embodiments, the cell includes about 10⁴ to about 10¹⁵ extracellular vesicles. In embodiments, the cell includes about 10⁵ to about 10¹⁵ extracellular vesicles. In embodiments, the cell includes about 10⁶ to about 10¹⁵ extracellular vesicles. In embodiments, the cell includes about 10⁷ to about 10¹⁵ extracellular vesicles. In embodiments, the cell includes about 10⁸ to about 10¹⁵ extracellular vesicles. In embodiments, the cell includes about 10⁹ to about 10¹⁵ extracellular vesicles. In embodiments, the cell includes about 10¹⁰ to about 10¹⁵ extracellular vesicles. In embodiments, the cell includes about 10¹¹ to about 10¹⁵ extracellular vesicles. In embodiments, the cell includes about 10¹² to about 10¹⁵ extracellular vesicles. In embodiments, the cell includes about 10¹³ to about 10¹⁵ extracellular vesicles. In embodiments, the cell includes about 10¹⁴ to about 10¹⁵ extracellular vesicles.

In embodiments, the cell includes about 10³ to about 10¹⁴ extracellular vesicles. In embodiments, the cell includes about 10³ to about 10¹³ extracellular vesicles. In embodiments, the cell includes about 10³ to about 10¹² extracellular vesicles. In embodiments, the cell includes about 10³ to about 10¹¹ extracellular vesicles. In embodiments, the cell includes about 10³ to about 10¹⁰ extracellular vesicles. In embodiments, the cell includes about 10³ to about 10⁹ extracellular vesicles. In embodiments, the cell includes about 10³ to about 10⁸ extracellular vesicles. In embodiments, the cell includes about 10³ to about 10⁷ extracellular vesicles. In embodiments, the cell includes about 10³ to about 10⁶ extracellular vesicles. In embodiments, the cell includes about 10³ to about 10⁵ extracellular vesicles. In embodiments, the cell includes about 10³ to about 10⁴ extracellular vesicles. In embodiments, the cell includes about 10³, 10⁴, 10⁵, 10⁶, 10⁷, 10⁸, 10⁹, 10¹⁰, 10¹¹, 10¹², 10¹³, 10¹⁴, or 10¹⁵ extracellular vesicles.

In embodiments, the cell includes about 100 to about 100,000 extracellular vesicles. In embodiments, the cell includes about 100 to about 10,000 extracellular vesicles. In embodiments, the cell includes about 100 to about 5,000 extracellular vesicles. In embodiments, the cell includes about 100 to about 1,000 extracellular vesicles. In embodiments, the cell includes about 100 to about 500 extracellular vesicles. In embodiments, the cell includes about 50 to about 100 extracellular vesicles. In embodiments, the cell includes about 10 to about 100 extracellular vesicles. In embodiments, the cell includes about 10,000 extracellular vesicles. In embodiments, the cell includes no more than about 10,000 extracellular vesicles. In embodiments, the cell includes about 50,000 extracellular vesicles. In embodiments, the cell includes no more than about 50,000 extracellular vesicles.

In embodiments, the extracellular vesicle includes an siRNA nucleic acid provided herein including embodiments thereof. In embodiments, each extracellular vesicle includes one siRNA nucleic acid provided herein including embodiments thereof. In embodiments, each extracellular vesicle includes a plurality of the siRNA nucleic acid provided herein including embodiments thereof.

In embodiments, the extracellular vesicle includes about 1 to about 20,000 siRNA nucleic acid molecules. In embodiments, the extracellular vesicle includes about 1000 to about 20,000 siRNA nucleic acid molecules. In embodiments, the extracellular vesicle includes about 2000 to about 20,000 siRNA nucleic acid molecules. In embodiments, the extracellular vesicle includes about 3000 to about 20,000 siRNA nucleic acid molecules. In embodiments, the extracellular vesicle includes about 4000 to about 20,000 siRNA nucleic acid molecules. In embodiments, the extracellular vesicle includes about 5000 to about 20,000 siRNA nucleic acid molecules. In embodiments, the extracellular vesicle includes about 6000 to about 20,000 siRNA nucleic acid molecules. In embodiments, the extracellular vesicle includes about 7000 to about 20,000 siRNA nucleic acid molecules. In embodiments, the extracellular vesicle includes about 8000 to about 20,000 siRNA nucleic acid molecules. In embodiments, the extracellular vesicle includes about 9000 to about 20,000 siRNA nucleic acid molecules. In embodiments, the extracellular vesicle includes about 10,000 to about 20,000 siRNA nucleic acid molecules. In embodiments, the extracellular vesicle includes about 11,000 to about 20,000 siRNA nucleic acid molecules. In embodiments, the extracellular vesicle includes about 12,000 to about 20,000 siRNA nucleic acid molecules. In embodiments, the extracellular vesicle includes about 13,000 to about 20,000 siRNA nucleic acid molecules. In embodiments, the extracellular vesicle includes about 14,000 to about 20,000 siRNA nucleic acid molecules. In embodiments, the extracellular vesicle includes about 15,000 to about 20,000 siRNA nucleic acid molecules. In embodiments, the extracellular vesicle includes about 16,000 to about 20,000 siRNA nucleic acid molecules. In embodiments, the extracellular vesicle includes about 17,000 to about 20,000 siRNA nucleic acid molecules. In embodiments, the extracellular vesicle includes about 18,000 to about 20,000 siRNA nucleic acid molecules. In embodiments, the extracellular vesicle includes about 19,000 to about 20,000 siRNA nucleic acid molecules.

In embodiments, the extracellular vesicle includes about 1 to about 19,000 siRNA nucleic acid molecules. In embodiments, the extracellular vesicle includes about 1 to about 18,000 siRNA nucleic acid molecules. In embodiments, the extracellular vesicle includes about 1 to about 17,000 siRNA nucleic acid molecules. In embodiments, the extracellular vesicle includes about 1 to about 16,000 siRNA nucleic acid molecules. In embodiments, the extracellular vesicle includes about 1 to about 15,000 siRNA nucleic acid molecules. In embodiments, the extracellular vesicle includes about 1 to about 14,000 siRNA nucleic acid molecules. In embodiments, the extracellular vesicle includes about 1 to about 13,000 siRNA nucleic acid molecules. In embodiments, the extracellular vesicle includes about 1 to about 12,000 siRNA nucleic acid molecules. In embodiments, the extracellular vesicle includes about 1 to about 11,000 siRNA nucleic acid molecules. In embodiments, the extracellular vesicle includes about 1 to about 10,000 siRNA nucleic acid molecules. In embodiments, the extracellular vesicle includes about 1 to about 9,000 siRNA nucleic acid molecules. In embodiments, the extracellular vesicle includes about 1 to about 8,000 siRNA nucleic acid molecules. In embodiments, the extracellular vesicle includes about 1 to about 7,000 siRNA nucleic acid molecules. In embodiments, the extracellular vesicle includes about 1 to about 6,000 siRNA nucleic acid molecules. In embodiments, the extracellular vesicle includes about 1 to about 5,000 siRNA nucleic acid molecules. In embodiments, the extracellular vesicle includes about 1 to about 4,000 siRNA nucleic acid molecules. In embodiments, the extracellular vesicle includes about 1 to about 3,000 siRNA nucleic acid molecules. In embodiments, the extracellular vesicle includes about 1 to about 2,000 siRNA nucleic acid molecules. In embodiments, the extracellular vesicle includes about 1 to about 1,000 siRNA nucleic acid molecules. In embodiments, the extracellular vesicle includes about 1, 1000, 2000, 3000, 4000, 5000, 6000, 7000, 8000, 9000, 10000, 11000, 12000, 13000, 14000, 15000, 16000, 17000, 18000, 19000, or 20000 siRNA nucleic acid molecules.

In embodiments, the extracellular vesicle includes about 100 to about 5000 siRNA nucleic acid molecules. In embodiments, the extracellular vesicle includes about 500 to about 5000 siRNA nucleic acid molecules. In embodiments, the extracellular vesicle includes about 1000 to about 5000 siRNA nucleic acid molecules. In embodiments, the extracellular vesicle includes about 1500 to about 5000 siRNA nucleic acid molecules. In embodiments, the extracellular vesicle includes about 2000 to about 5000 siRNA nucleic acid molecules. In embodiments, the extracellular vesicle includes about 2500 to about 5000 siRNA nucleic acid molecules. In embodiments, the extracellular vesicle includes about 3000 to about 5000 siRNA nucleic acid molecules. In embodiments, the extracellular vesicle includes about 3500 to about 5000 siRNA nucleic acid molecules. In embodiments, the extracellular vesicle includes about 4000 to about 5000 siRNA nucleic acid molecules. In embodiments, the extracellular vesicle includes about 4500 to about 5000 siRNA nucleic acid molecules.

In embodiments, the extracellular vesicle includes about 100 to about 4500 siRNA nucleic acid molecules. In embodiments, the extracellular vesicle includes about 100 to about 4000 siRNA nucleic acid molecules. In embodiments, the extracellular vesicle includes about 100 to about 3500 siRNA nucleic acid molecules. In embodiments, the extracellular vesicle includes about 100 to about 3000 siRNA nucleic acid molecules. In embodiments, the extracellular vesicle includes about 100 to about 2500 siRNA nucleic acid molecules. In embodiments, the extracellular vesicle includes about 100 to about 2000 siRNA nucleic acid molecules. In embodiments, the extracellular vesicle includes about 100 to about 1500 siRNA nucleic acid molecules. In embodiments, the extracellular vesicle includes about 100 to about 1000 siRNA nucleic acid molecules. In embodiments, the extracellular vesicle includes about 100 to about 500 siRNA nucleic acid molecules. In embodiments, the extracellular vesicle includes about 100, 500, 1000, 1500, 2000, 2500, 3000, 3500, 4000, 4500 or 5000 siRNA nucleic acid molecules.

In embodiments, the extracellular vesicle includes about 1 to about 200 siRNA nucleic acid molecules. In embodiments, the extracellular vesicle includes about 1 to about 180 siRNA nucleic acid molecules. In embodiments, the extracellular vesicle includes about 1 to about 160 siRNA nucleic acid molecules. In embodiments, the extracellular vesicle includes about 1 to about 140 siRNA nucleic acid molecules. In embodiments, the extracellular vesicle includes about 1 to about 120 siRNA nucleic acid molecules. In embodiments, the extracellular vesicle includes about 1 to about 100 siRNA nucleic acid molecules. In embodiments, the extracellular vesicle includes about 1 to about 80 siRNA nucleic acid molecules. In embodiments, the extracellular vesicle includes about 1 to about 60 siRNA nucleic acid molecules. In embodiments, the extracellular vesicle includes about 1 to about 40 siRNA nucleic acid molecules. In embodiments, the extracellular vesicle includes about 1 to about 20 siRNA nucleic acid molecules.

In embodiments, the extracellular vesicle includes about 1 to about 180 siRNA nucleic acid molecules. In embodiments, the extracellular vesicle includes about 1 to about 160 siRNA nucleic acid molecules. In embodiments, the extracellular vesicle includes about 1 to about 140 siRNA nucleic acid molecules. In embodiments, the extracellular vesicle includes about 1 to about 120 siRNA nucleic acid molecules. In embodiments, the extracellular vesicle includes about 1 to about 100 siRNA nucleic acid molecules. In embodiments, the extracellular vesicle includes about 1 to about 80 siRNA nucleic acid molecules. In embodiments, the extracellular vesicle includes about 1 to about 60 siRNA nucleic acid molecules. In embodiments, the extracellular vesicle includes about 1 to about 40 siRNA nucleic acid molecules. In embodiments, the extracellular vesicle includes about 1 to about 20 siRNA nucleic acid molecules. In embodiments, the extracellular vesicle includes about 1, 20, 40, 60, 80, 100, 120, 140, 160, 180, or 200 siRNA nucleic acid molecules.

In embodiments, the cell does not substantially express CHMP4C, VPS4B, or a combination thereof. In embodiments, the cell does not substantially express Akt3, MAPKAPK2 (MK2), MEKI, MEKII, ERK, EGFR, or p38-MAPK, or a combination thereof. In embodiments, the cell does not substantially express Caveolin 1 (CAV1).

In embodiments, the cell is a neural stem cell (NSC), a mesenchymal stem cell (MSC), an induced pluripotent stem cell (iPSC), an embryonic stem cell (ESC), an immune cell, or an epithelial cell. In embodiments, immune cell is a T cell, NK cell, or macrophage.

Provided herein, inter alia, are cell compositions including a cell stably transfected with a nucleic acid encoding an argonaute 2 (AGO2) protein and a nucleic acid encoding a short hairpin RNA (shRNA) nucleic acid, wherein the cell may be used to generate the extracellular vesicle including the siRNA nucleic acid provided herein including embodiments thereof. In an aspect is provided a cell stably transfected with a nucleic acid encoding an argonaute 2 (AGO2) protein and a nucleic acid encoding a short hairpin RNA (shRNA) nucleic acid. As described above, a “stably transfected cell” is used in accordance with its plain ordinary meaning in the art and refers to cell transfected with a nucleic acid wherein the nucleic acid is incorporated into genome of the cell. Thus, in embodiments, the nucleic acid encoding the AGO2 protein is integrated into the genome of the cell. In embodiments, the nucleic acid encoding the shRNA is integrated into the genome of the cell.

In embodiments, the shRNA nucleic acid is a dicer-independent shRNA nucleic acid. In embodiments, the shRNA nucleic acid includes a stem loop including a stem sequence no more than about 19 base pairs in length. In embodiments, the stem loop includes a loop sequence about 1 nucleotide to about 8 nucleotides in length. In embodiments, the loop sequence is about 3 nucleotides to about 5 nucleotides in length. In embodiments, the loop sequence is 4 nucleotides in length. In embodiments, the shRNA nucleic acid includes an unmatched base pair. In embodiments, the shRNA nucleic acid includes an overhang sequence on the 3′ end.

In embodiments, the overhang sequence includes an exosome-specific RNA motif. In embodiments, the exosome-specific RNA motif is about 2 nucleotides to about 20 nucleotides in length. In embodiments, the exosome-specific RNA motif is about 3 nucleotides to about 8 nucleotides in length. In embodiments, the exosome specific RNA motif is an miR-451 motif, an exo-motif, an hExomotif, a polyuridylation motif, an LA motif, an astrocyte/neuron motif, or an extended exomotif.

In embodiments, the nucleic acid encoding the shRNA nucleic acid is operably linked to a small RNA promoter. “Small RNA promoter” refers to a region of DNA (e.g. promoter) that regulates transcription of small RNA (e.g. miRNA, shRNA, siRNA, and guide RNA). Typically, a small RNAis an RNA molecule shorter than 200 nucleotides in length. In embodiments, a small RNA promoter binds and/or recruits transcription factors and RNA polymerase associated with expression of small RNA. In embodiments, the small RNA promoter is a U6, mU6, H1, 7SK, U1, T7, SP6, T3, tRNA-Lys3, tRNA-Val, tRNA-Ser, tRNA-gly, tRNA-Gln, tRNA-Pro, tRNA-Leu, or tRNA-Asn promoter.

In embodiments, the AGO2 protein includes an S387A mutation at a position corresponding to position 387 of SEQ ID NO:159

In embodiments, the cell includes a nucleic acid encoding a fusogen protein. In embodiment, the cell is stably transfected with the nucleic acid encoding the fusogen protein. In embodiments, the fusogen protein is gap junction protein, connexin 43, syncytin-A (SynA), myoferlin, vesicular stomatitis virus G (VSV-G), Sindbis virus glycoprotein (SINmu), baboon retroviral envelope glycoprotein (BaEV), measles virus glycoprotein, nipah virus envelope glycoprotein, GALA peptide, EALA peptide, or KALA peptide. In embodiments, the fusogen protein is connexin 43 or VSV-G.

In embodiments, the cell includes a nucleic acid encoding a fusion protein including: a) an extracellular vesicle membrane-associated protein, and b) a targeting protein or a detectable moiety. In embodiments, the cell is stably transfected with the nucleic acid encoding the fusion protein. In embodiments, the extracellular vesicle membrane-associated protein is CD9, CD37, CD53, CD63, CD68, CD81, CD82, LAMP-1, LAMP-2A, LAMP-2B, LAMP-2C, lactadherin, or PTGFRN. In embodiments, the extracellular vesicle membrane-associated protein is CD63. In embodiments, the targeting protein is a single chain variable fragment. In embodiments, the detectable moiety is a fluorescent protein.

In embodiments, the cell includes a nucleic acid encoding a Charged Multivesicular Body Protein 4C (CHMP4C) inhibitor, a nucleic acid encoding a Vacuolar Protein Sorting 4 Homolog B (VPS4B) inhibitor, or a combination thereof. In embodiment, the cell is stably transfected with the nucleic acid encoding the Charged Multivesicular Body Protein 4C (CHMP4C) inhibitor, the nucleic acid encoding the Vacuolar Protein Sorting 4 Homolog B (VPS4B) inhibitor, or a combination thereof. In embodiments, the cell is a clonal knockout that does not include a CHMP4C gene or a VPS4B gene. In embodiments, the cell is a clonal knockout that does not include a functional CHMP4C gene or a functional VPS4B gene.

In embodiments, the cell includes a nucleic acid encoding an Ago2 cellular retention factor inhibitor. In embodiment, the cell is stably transfected with the nucleic acid encoding the Ago2 cellular renttion factor inhibitor. In embodiments, the Ago2 cellular retention factor is Akt3, MAPKAPK2 (MK2), MEKI, MEKII, ERK, EGFR, p38-MAPK, or a combination thereof. In embodiments, the cell is a clonal knockout that does not include a Akt3, MAPKAPK2 (MK2), MEKI, MEKII, ERK, EGFR, or p38-MAPK gene. In embodiments, the cell is a clonal knockout that does not include a functional Akt3, MAPKAPK2 (MK2), MEKI, MEKII, ERK, EGFR, or p38-MAPK gene.

In embodiments, the cell includes a nucleic acid encoding an Ago2 sorting factor. In embodiment, the cell is stably transfected with the nucleic acid encoding the Ago2 sorting factor. In embodiments, the Ago2 sorting factor is PTPN1, a dominate negative KRAS (S17N) mutant, Cx43, EV-factor ALIX, or a combination thereof.

In embodiments, the cell is a neural stem cell (NSC), a mesenchymal stem cell (MSC), an induced pluripotent stem cell (iPSC), an embryonic stem cell (ESC), an immune cell, or an epithelial cell. In embodiments, the immune cell is a T cell, NK cell, or macrophage. In embodiments, the cell is a HEK293 cell.

It is understood that the examples and embodiments described herein are for illustrative purposes only and that various modifications or changes in light thereof will be suggested to persons skilled in the art and are to be included within the spirit and purview of this application and scope of the appended claims. A II publications, patents, and patent applications cited herein are hereby incorporated by reference in their entirety for all purposes.

### EXAMPLES

### Example 1: Introduction to Exemplary Studies

Extracellular vesicles (EVs) are emerging as important vehicles in biotherapeutic applications. EVs are small vesicles derived from the cell membrane that carry various DNA, RNA, and protein payloads to elicit effects in recipient cells. Two main EV groups have been extensively explored for these applications: small EVs derived from multivesicular bodies (MVBs), containing the exosome fraction, and membrane-derived macrovesicles. Importantly, both are engineerable EV platforms that can selectively load and deliver cargo as a next-generation delivery system for bioactive macromolecules (1). Delivery remains a significant hurdle in various bioscience fields, like gene therapy, and several viral and non-viral systems are available. However, viral systems are immunogenic, and when transient effects are needed, the long-term transgene expression from viral vectors is unfavorable in these applications. Although non-viral nanoparticles circumvent these issues, synthetic nanoparticles present challenging hurdles such as the difficulty in targeting organs other than the liver and immunogenicity to some lipid components (2). EVs may be selected to enrich in a wide-range of tissues and are able to cross the blood brain barrier (BBB) (3) and are largely impermeable to other systemically administered effectors, making EVs impactful as a biotechnology vehicle. Therefore, EVs are a non-immunogenic, biocompatible, non-toxic delivery vehicle for the delivery of programmable cargo (4).

RNA interference (RNAi) is an established research tool and recently entered the clinic for therapeutic applications. However, here too, delivery remains a barrier to the wider adoption of RNAi-based therapeutics and its research applications in vivo. Various methodologies to selectivity load RNAi into EVs have been explored, but often have low programmability to new targets, the reliance on cumbersome loading procedures, or questionable efficacy when delivering functionally active cargo. To this end, we have identified an innovative non-canonical dicer-independent short hairpin RNA (shRNA), termed the Exo-shRNA, that is highly enriched in the small EV fraction, and because of its simple architecture, it is readily programmable to new targets of interest, representing a broadly impactful technology for the delivery of RNAi using a native vesicle system. Furthermore, due to the Ago2-mediated maturation of the Exo-shRNA that cleaves the ‘passenger’ strand, that is often associated with off-target toxicity, allows for selective ‘target’ strand incorporation into EVs. Furthermore, various optimization steps are required to make EVs a bona fide technology, and this work will include identifying factors to refine this novel RNAi enrichment technology through RNA and protein factors as well as improving EV production and cargo incorporation. This system will have a wide range of applications and by leveraging its simple knock-down readout, it could be used as a functional cargo release assay from EVs, a complementary methodology to current research approaches. Provided herein are methods for highly and selectively enrich RNAi effectors into EVs for in vitro and in vivo applications. Demonstrated herein is a non-canonical shRNA system which can be readily programmed with RNAi effectors for incorporation into a highly optimized EV delivery system to elicit targeted knockdown in cell lines and murine organs.

Experiments described below include:
Characterization of the Exo-shRNA EV system in vitro. A detailed characterization of the Exo-shRNA system programmed to a range of relevant targets, the subpopulations containing the Exo-shRNAs, and their RNA and protein content.
Optimization of the Exo-shRNA EV system. Optimization of the Exo-shRNA system to further enhance RNAi enrichment and potency through the manipulation of RNA expression, shRNA scaffold, Ago2 EV sorting factors, and host factors in EV biogenesis.
Utility of the Exo-shRNA EV system in vitro and in vivo. Assessment of the tropism and safety of the Exo-shRNA EVs to deliver functional RNAi effectors in vitro and in vivo for biotechnological applications.

The studies described herein provide a readily programmable platform for the delivery of RNAi effectors using biocompatible EV delivery as a broadly impactful, next-generation approach for therapeutic and research applications.

### Example 2: Extracellular Vesicles Including siRNA

EVs represent an untapped source for RNAi effector delivery as they are non-immunogenic and non-toxic (4), which could be used as a novel approach to deliver RNAi-based effectors for biomedicine. Various methodologies to selectively load RNAi into EVs have been explored such as EV-enriched microRNA scaffolds (11), exosome-specific RNA motifs (12), artificial loading through aptamer-protein interactions fused to EV markers (13), or post-production loading of RNAi using methods such a transfection, electroporation, or sonication (14). Each has limitations including low programmability to new targets (miRNA scaffolds), fusion of RNAi effectors to EV markers preventing the release of the RNAi effectors in recipient cells, the enrichment of cargo into the EVs using artificial fusion proteins often derived from bacteria or viruses that negate the non-immunogenic EV properties (15), or cumbersome and harsh loading procedures of the EVs post-purification that can alter the EV state. In some cases, there is questionable transfer of functionally active RNAi cargo. Lastly, as RNAi is triggered by dsRNA, RNAi can elicit activity through both the ‘target’ strand and ‘passenger’ strand with toxicity often attributed to non-specific effects through passenger strand off-targeting (16). A Iso, delivering ribonucleoproteins (RNPs) within the EVs would be beneficial for transient, druggable applications. Overall, a methodology that collectively addresses these problems would be impactful for developing EV-delivered RNAi technology. With this in mind, non-canonical dicer-independent shRNAs are processed directly by human Argonaute 2 (Ago2) through its unique ‘slicer’ activity by cleaving the ‘passenger’ strand, and directly loading the mature targeting strand into Ago2 (17). Furthermore, unlike miRNA scaffolds that require significant target optimization due to multiple factors involved in their maturation (18), the simple architecture of dicer-independent shRNAs allows for the easy design to new targets. Of significance, Ago2 associates with multivesicular bodies (MVBs), the site of exosome biogenesis (19), and Ago2 is detected within small EVs with a possible mechanism for active sorting into vesicles (20, 21). Therefore, for this proposed project, we characterized a novel non-canonical dicer-independent shRNA system to generate a highly programmable approach for the enrichment and delivery of RNAi using EVs, termed the ‘Exo-shRNA’ system. There are several features that can improve the Exo-shRNA's enrichment in EVs and achieved through: 1) RNA features and motifs that have been implicated in biasing RNA effectors into EVs (12, 22, 23), 2) host factors that are known to post-translationally modify Ago2 and affect intracellular trafficking and possible sorting into EVs (20, 24), and 3) factors that enhance EV production and, importantly, the concurrent enrichment of artificial cargo. The detailed exploration of the Exo-shRNA system's RNA architecture, Ago2-associated EV sorting factors, and endogenous proteins in EV production will be explored to generate a fully realized RNAi-deliverable EV prototype. Ultimately, the goal of characterizing and optimizing the Exo-shRNA system would be for its broader utility in biotechnological applications. EV tropism is an active area of research, and current research suggests that EVs of similar tissue types are receptive to EV cross-communication (25). The tissue specific uptake of EVs has been implicated in augmenting inflammation, viral infection, tumor immune evasion and metastasis (6). However, the ‘tropism code’ of EVs is likely far more complex and is still being elucidated; and the membrane surface of EVs is an array of ligands that could interact with receptors on a wide range of tissues. The means to identify situations where EVs not only bind to target cells but release their functional cargo into the receptive tissues are needed. Furthermore, identifying non-immunogenic proteins that can enhance cargo release (fusogens) are greatly needed along with methods to easily screen novel fusogenic proteins. Simple methods to comprehensively analyze innate and engineered EV tropism are lacking and a readily programable RNAi EV system with a functional knockdown read-out would, 1) help refine tissues amenable for downstream applications, and 2) provide a functional read-out for tropism studies, broadly impacting EV research.

Experiments described herein focus on investigating a next-generation EV platform for the delivery of RNAi. This system will be a significant advancement over the current systems as, currently, miRNA scaffolds suffer from low programmability due to variable processing by the upstream enzymes (18) and tethering factors to EV makers or motifs could prevent release in the target cell line reducing functionality. Prepacking of EVs also can be prepared in bulk for scalable productions. Importantly, none of the aforementioned approaches can control for strand-selectivity. Furthermore, several Argonautes (Ago1, 3, and 4) can bind RNAi effectors, but as Ago2 is required to process Exo-shRNAs, this substrate specific maturation process ensures a defined functional cargo: a favorable feature of a refined biotechnology. This project will refine a novel readily programmable, strand-selective, EV-enriching shRNA architecture that does not require any difficult-to-program miRNA scaffolds, artificial motifs, or tethering to EV markers as a novel EV-based delivery system. Furthermore, due to the simple knockdown read-out, this technology could be applied to basic research such as the tropism of EVs or developed for RNA i-based therapeutics. The Exo-shRNA system could be combined with other EV features, such as the anti-inflammatory properties of stem cell EVs (26) or artificial surface ligands (27), to augment the functional RNAi payloads, broadening their therapeutic effectiveness. Finally, a detailed exploration of Ago2 in EVs could provide insights into RNAi and EVs as well as host factors that enhance EV production and cargo loading would be broadly applicable to EV biotechnology.

### Development of HIV and Myeloid Targeted EVs

Described below is characterization of the Exo-shRNA system programmed to a range of relevant targets, the subpopulations containing the Exo-shRNAs, and their RNA and protein content.

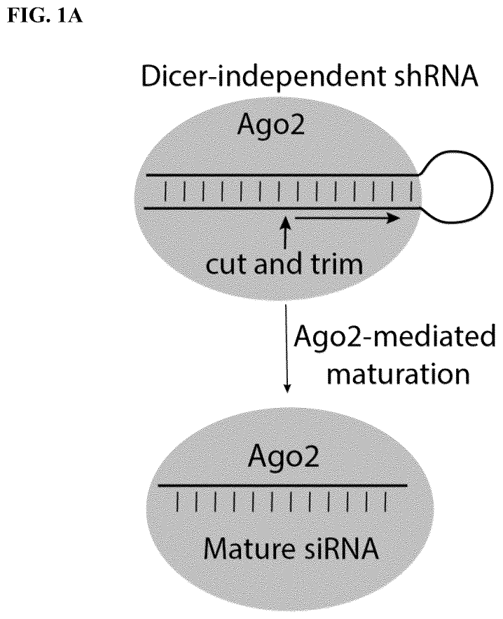

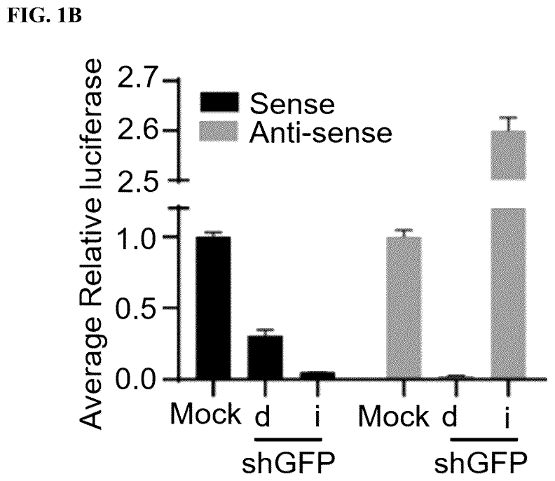

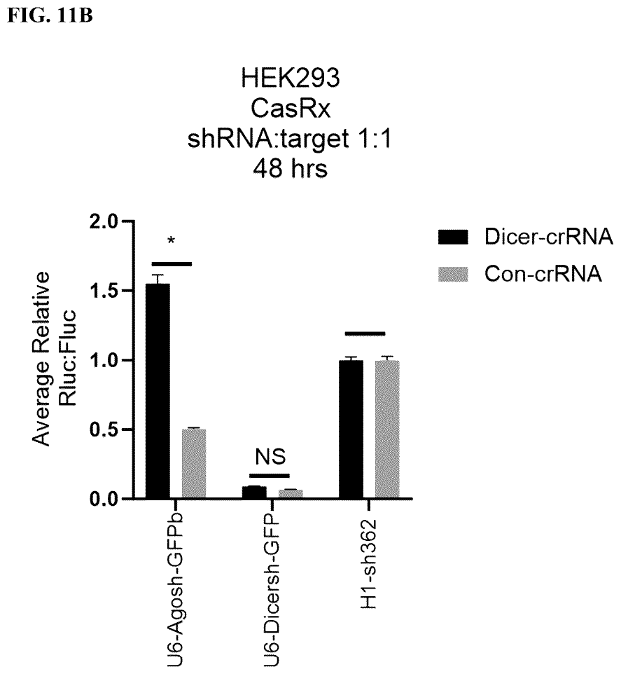

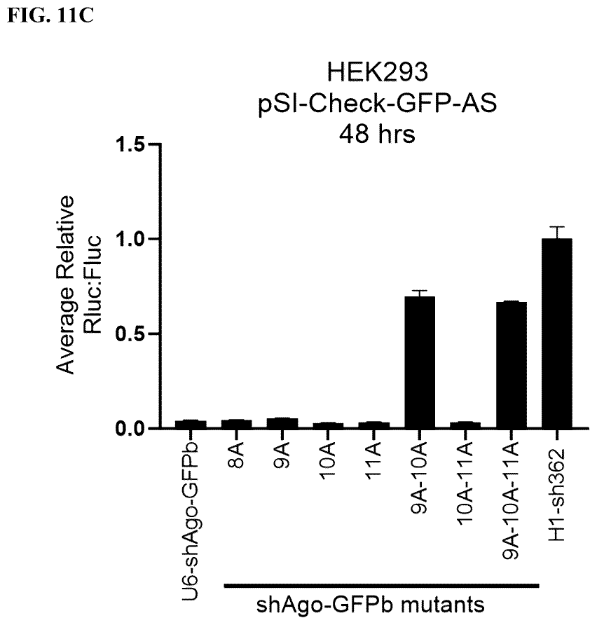

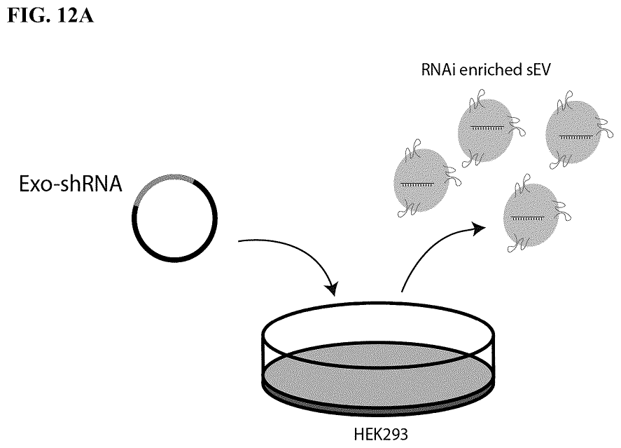

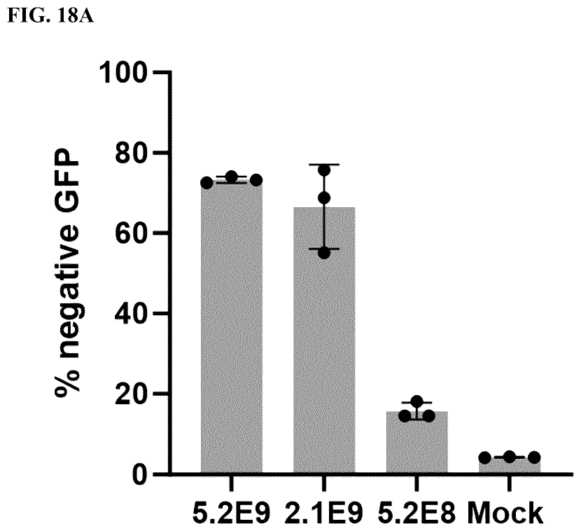

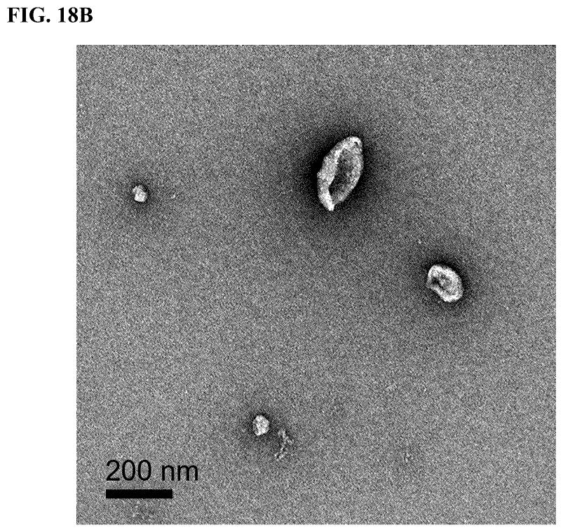

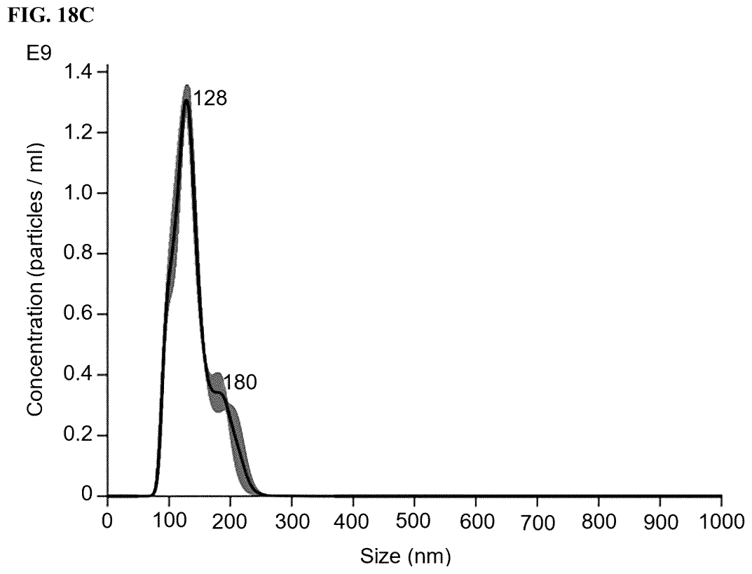

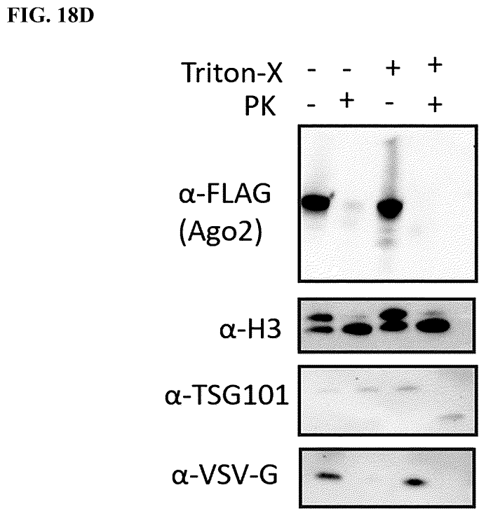

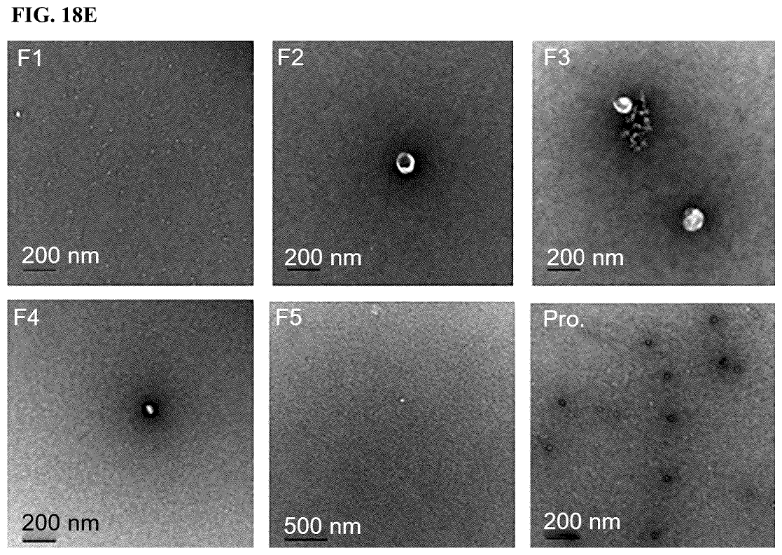

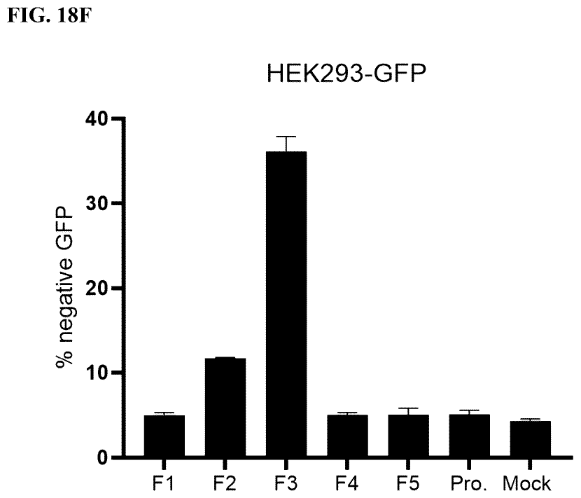

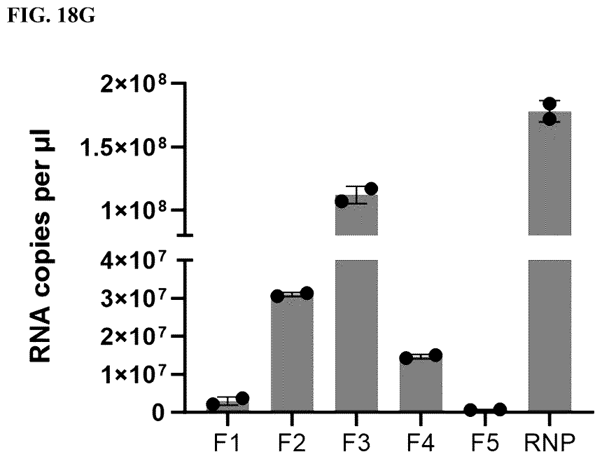

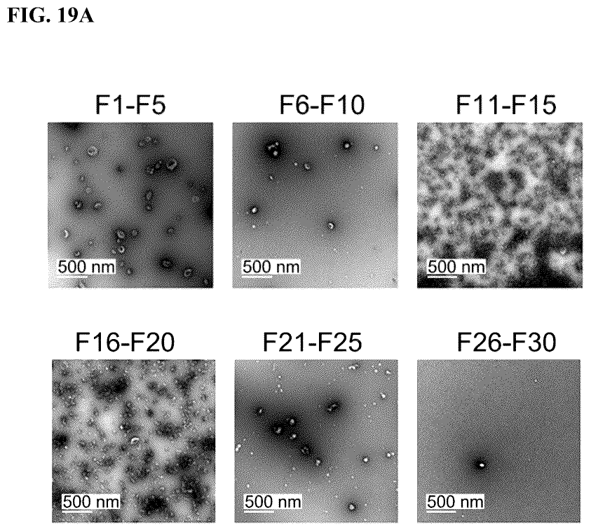

Flexible, scalable, and simple means to preload EVs with RNAi effectors for biomedical and research applications are currently lacking. To address this unmet need, we have identified a programmable means to highly enrich strand-selective RNAi effectors into the small EV fraction (exosomes) (FIG. 1A). We have designed a non-canonical dicer-independent shRNA targeted to GFP (Exo-shGFP) with a short stem sequence (<19 nt in length) that is processed directly by Ago2 (28). We verified Ago2-dependence of the Exo-shGFP through, 1) mutation of Ago2 cleavage sites between the 9th and 10th nucleotides in the non-target strand which abolished target knockdown (FIG. 11C), 2) a reduction in dicer mRNA levels had no effect on Exo-shGFP silencing, unlike its dicer-dependent counterpart (stem >21 nt) (FIGS. 1A and 1B), and 3) the Exo-shGFP demonstrated strand selectivity through knockdown of a sense target only in reporter assays (FIG. 18).

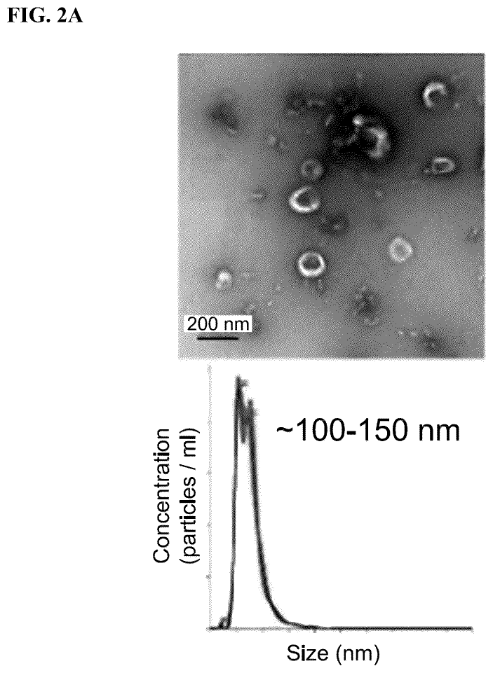

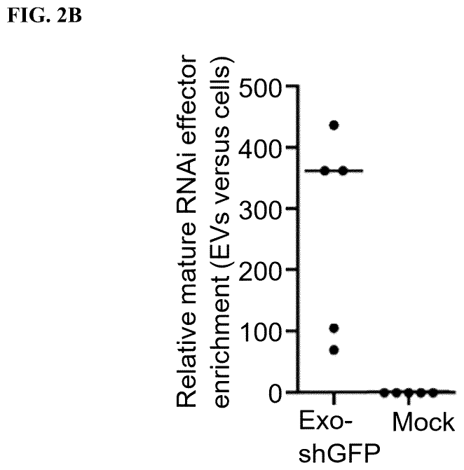

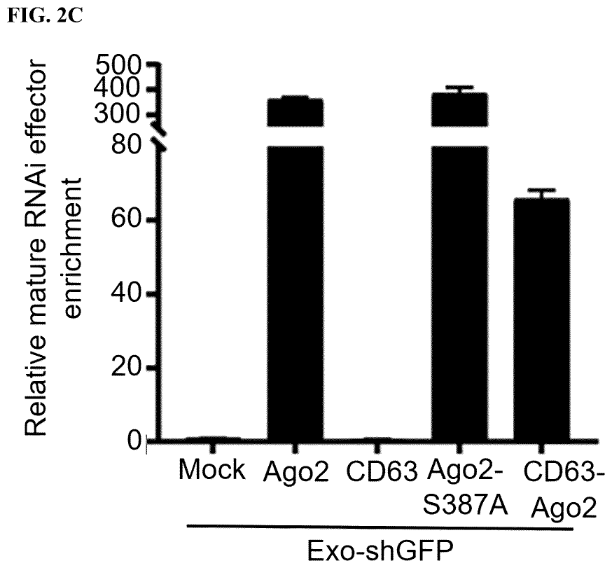

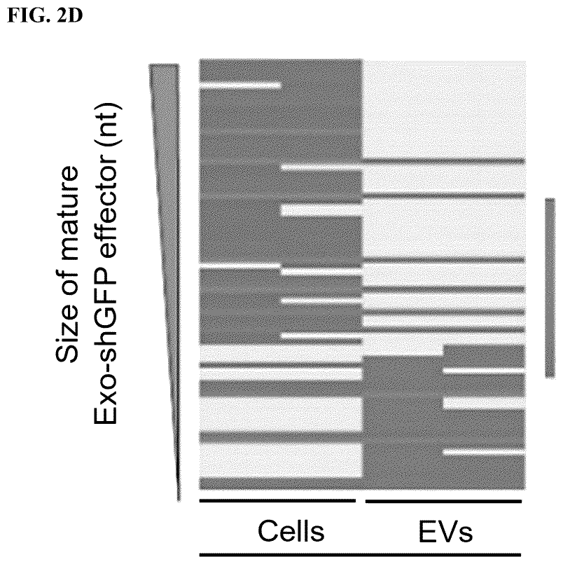

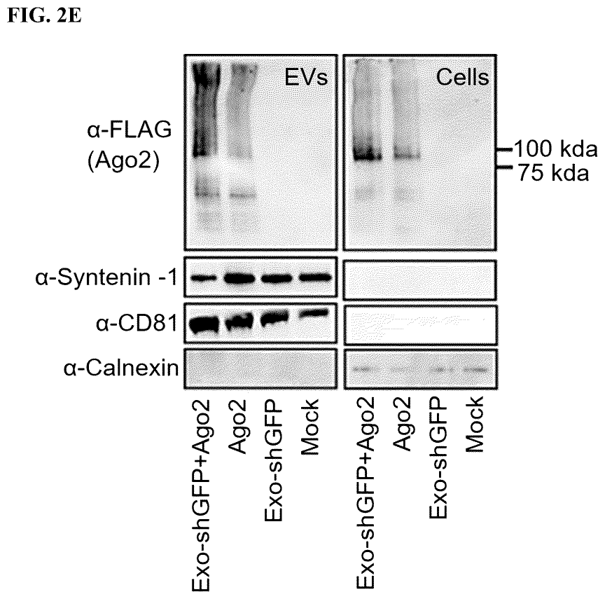

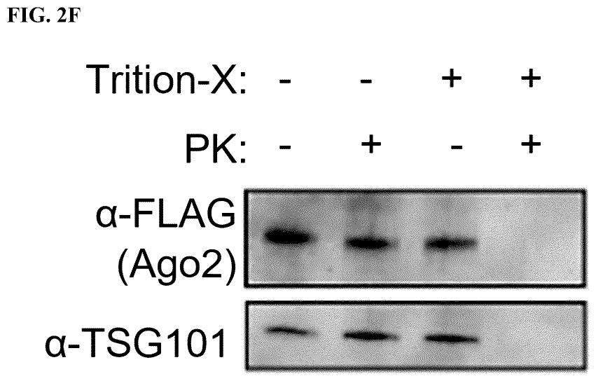

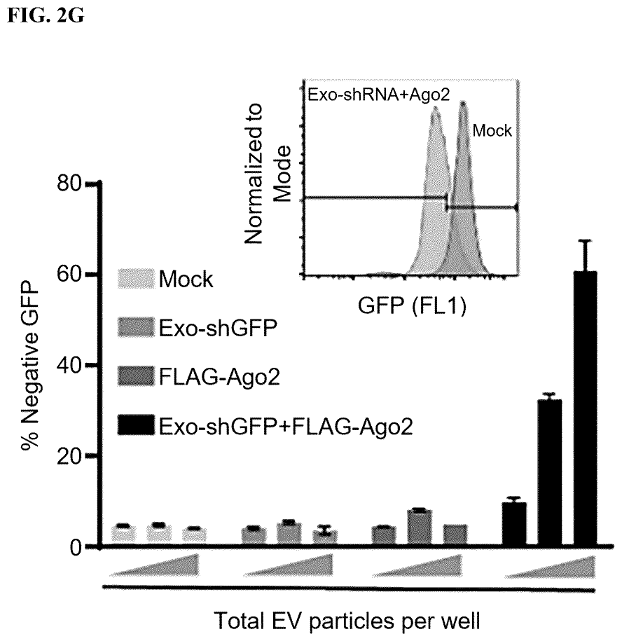

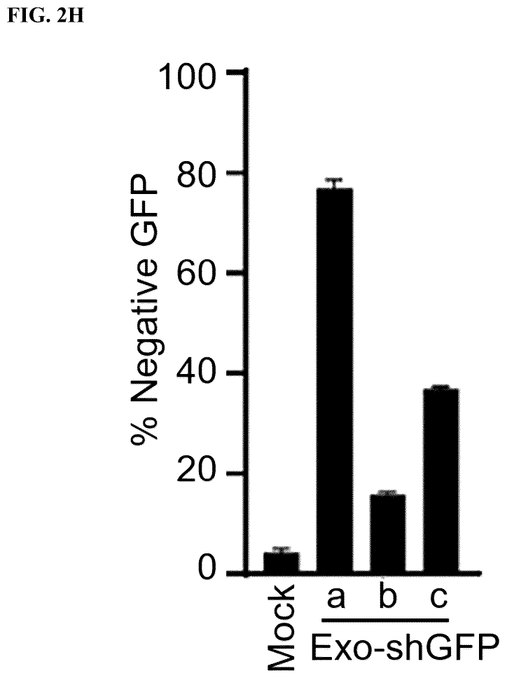

The small EV fraction was purified from HEK293 cells transfected with the Exo-shGFP using established differential ultracentrifugation and filtration methods with an additional wash step, and the cup-shaped EVs morphology and size of ˜100-150 nm was confirmed by transmission electron microscopy (TEM) and nano-tracking analysis (NTA) (FIG. 2A), and a 60-400-fold enrichment of the mature RNAi effector was detected compared to cell lysates by RT-qPCR (FIG. 2B). To further enhance enrichment, EVs were harvested from cells transfected with the Exo-shGFP and vectors overexpressing FLAG-tagged Ago2 (FLAG-Ago2), an ‘EV-sorted’ phosphorylation mutant Ago2-S387A (20), and an Ago2 fused to the EV-marker CD63. Ago2 overexpression further enriched the mature effector by an additional ˜350-fold compared to an Exo-shGFP only (FIG. 2C). Interestingly, there was a distinct enrichment of RNAi effectors with smaller sequences in the EVs (<20 nt) versus cells (>20 nt), suggesting an EV-specific maturation process (FIG. 2D). The presence of FLAG-Ago2 in EVs was confirmed by immunoblotting along with EV-specific markers (FIG. 2E). Critically, a protease protection assay showed that the FLAG-Ago is within the luminal compartment of vesicles and only susceptible to degradation in the presence of a membrane-disrupting detergent (FIG. 2F). To determine if the enriched RNAi-loaded EVs were functional, the Exo-shGFP+Ago2 EVs were added to the media of a recipient HEK293 cell line expressing GFP and resulted in a dose responsive knockdown (FIG. 2G). Clearly, these data demonstrate the transfer of catalytically functional cargo, which require both the Exo-shGFP and FLAG-Ago2 components. Importantly, unlike artificial miRNA scaffolds (18), Exo-shRNAs are compact architectures that don't require any difficult-to-program scaffolds, potentially making the system readily programmable with new shRNA effectors. To demonstrate this point, we assessed if additional dicer-independent shRNAs targeted to GFP were able to facilitate knockdown and two Exo-shGFPs (band c) resulted in knockdown when transferred by EVs (FIG. 2H). Collectively, these data strongly support the basis for further development of a highly programmable strand-selective EV system for the delivery of RNAi, as a potential viable prototype technology.

### Validation of the Exo-shRNA System on Expanded Targets and Cell Sources

Encouraged by the results, the Exo-shRNA's system will be assessed in more detail with relevant endogenous targets associated with cancer, neurological disorders, and viral infections. These targets are not to limit the technology to specific diseases but rather represent proof-of-concept targets for the broad utility of the Exo-shRNAs. We will design Exo-shRNAs to, a) the CCR5 and PTEN genes implicated in the exacerbation of stroke, b) the BACE1 gene implicated in Alzheimer's disease, and c) TW IST and PD-L1 associated with cancer chemoresistance and immune suppression, respectively. Lastly, these targets all represent post-transcriptional gene silencing (PTGS) for downregulation of mRNA, but RNAi can be used to epigenetic silence promoters through transcriptional gene silencing (TGS). These ‘block and lock’ strategies have been proposed to push human immunodeficiency virus (HIV) into ‘deep latency’ as a ‘functional cure’ and Exo-shRNAs towards the promoter of HIV will be developed. These Exo-shRNAs will be generated from HEK293 cells, the media replaced post-transfection with chemically defined media and purified at 96 hrs post-transfection. EV size, morphology, and immunoblotting for EV markers will confirm the small EV purity in line with MISEV2014 guidelines (29). RNA will be extracted using the Maxwell® RSC miRNA Plasma or Serum kit (Promega, WI, USA) and mature RNAi effectors will be determined using the Mir-X™ miRNA First-Strand Synthesis Kit (Takara Bio USA). Absolute amounts of EVs (determined by NTA) will be dosed on the cells and knockdown of each respective Exo-shRNA's target in recipient cell lines will be assessed. The TZM-bl reporter cell line are cervical cancer cells with a HIV promoter driving a luciferase reporter and express CCR5, PTE N, TWIST, and PD-L1, and will be used as a model cell line for these Exo-shRNAs. The Neuro2A cell line will be used as an in vitro model for the BACE1 targeting Exo-shRNAs. After the addition of the EVs, knockdown will be assessed with RT-qPCR and immunoblotting for the cellular targets. To assess the Exo-shHIV, inhibition of the HIV promoter will be assessed by a luciferase reporter assay (Promega, USA). HEK293-derived EVs represent highly engineerable EV s with clinical translatability but EVs from other cell source also hold therapeutic potential (7, 27). Neural stem cells (NSC) are immunomodulatory and have significant biomedical potential (30), and augmenting these EVs with an Exo-shRNA system could enhance NSC EVs research and therapeutic applications. The Exo-shRNAs above will be generated with NSCs EVs and the downstream assays performed as described as proof-of-concept for the Exo-shRNA utility from another tissue source. Collectively, these assays will define the Exo-shRNA as a highly adaptable system to different shRNA targets and the system can be imported to alternative cell sources.

High-Resolution Characterization of the Exo-shGFP within SEC EV Subpopulations

Any new biotechnology would require a detailed product characterization. However, small EVs are a heterogeneous population with various subpopulations enriched for different surface and luminal factors. A detailed analysis of Ago2 enrichment in EVs and in the context of this technology with an overexpressed Ago2 is lacking. To this end, we will use size exclusion chromatology (SEC) methods using the qEV isolation system (Izon Science, USA) to determine the subpopulations enriched for the Exo-shRNA components (Exo-shGFP and FLAG-Ago2). EVs will be purified on the 70 nM qEV column size and 18 fractions of 0.5 ml will be collected, allowing for high resolution separation of subpopulations. The EV size and morphology will be characterized by TEM and NTA. Subsequently, the different fractions will be assessed by RT-qPCR for the mature Exo-shGFP RNAi effectors. Furthermore, immunoblotting for FLAG-Ago2 will be performed on each fraction along with various EV markers (CD63, CD81, CD9) to assess if there is a specific EV marker present with FLAG-Ago2 enrichment. Furthermore, understanding the number of RNP complexes per EVs will be important to generate and refine the Exo-shRNA prototype. Using a known amount of EVs, FLAG-Ago2 per EV in enriched fractions will be determined through two assays, 1) purified small RNA will be subjected to RT-droplet-digital PCR (RT-ddPCR) analysis as described (31), and 2) an ELISA to determine FLAG-Ago2 proteins per EV, compared to a RNA oligomer (synthesized by IDT, USA) and recombinant Ago2 standards, respectively (Sinobiological, China). Finally, the fractions most enriched for Exo-shRNA components will be dosed on target reporter cells to elucidate the potency of the system and further refine the product parameters for effective knockdown by the Exo-shRNA system.

As the EVs are cell-derived products, a detailed understanding of the cargo within the EVs containing the Exo-shRNA system will be assessed. This cargo includes the nature of the processed RNAi and protein components. Any ‘cellular containments’ should be assessed and if overexpression of the Exo-shGFP+FLAG-Ago2 bias the EV proteins towards the artificial RNAi effectors. To assess the species of RNA s, total small RNA s will be extracted as described from the pooled qEV fractions enriched for Exo-shGFP+FLAG-Ago2, and then subjected to total small RNA-seq (City of Hope Integrated Genomics Core (IGC)). EVs will be collected from untreated and FLAG-Ago2 only transfected cells to compare any biasing of RNA content in the Exo-shRNA EVs. Second, to assess the RNA species loaded into FLAG-Ago2, the Ago will be pulled down and purified using an anti-FLAG antibody and the RNA extracted from Ago2 RNP complexes will be subjected to small RNA-seq as described elsewhere (32). The bioinformatic analysis will be performed by the IGC. To elucidate the protein factors associated with the Exo-shRNA complex in EVs, the EVs will be subjected to liquid chromatography mass spectrometry (LC-M S) analysis to determine protein content (Pirrotte Lab). On average, 108-109 EVs will be lysed using an isotonic buffer containing 2% deoxycholate, followed by centrifugation at 16,000 RCF. The clarified lysate will be digested using an adaptation of our previously published protocol (34). Equal amounts of protein will be reduced by DTT (10 mM) and alkylated by iodoacetamide (10 mM), followed by overnight digestion with trypsin. Digested peptides will be analyzed on a Thermo Ultimate 3000 nanoLC coupled to a Thermo Orbitrap Fusion Eclipse, operated in data independent mode. Using this approach, we have shown excellent reproducibility across replicates in EVs isolated from cell cultures (34) and biofluids (35). Proteins will be identified using the M ascot search engine v3.2 (MatrixScience). Proteins will be annotated using Gene Ontology (36-38) and ranked based on abundance and tissue specificity by correlating marker expression with ProteinAtlas evidence. Data will be annotated for canonical EV markers (tetraspanins; CD9, CD63, CD81). Rigor of the proteomics research: Experiments will be conducted in triplicate biological replicates. Analytical variability will be monitored by bracketing samples with commercial E. coli digest and spiked-in synthetic heavy peptide mixture in each sample. Protein and peptide identifications will be filtered to 1% FDR using a reverse-decoy database search strategy. Only unambiguously identified, high confidence peptides will be retained for further data analysis. Data will be expressed as the mean of the top 3 most abundant precursor ions for each identified protein. Furthermore, the Ago2 interactome will be pulled down from exo-shGFP EVs using M2 anti-FLAG and subjected to affinity-precipitation mass spectrometry (AP-MS) following AP-MS methods previously published by the Pirrotte Lab (39-42). Triplicate M2 anti-FLAG (M2, Sigma, USA) pulldowns of Ago2 interactomes of exo-shGFP EVs will enable statistical scoring of interactions above non-specific background measured in mock transfected cells using SA IN T express (43). High confidence interactors to Ago2 (AvgP>0.7) will be prioritized for downstream analysis and interpretation. Furthermore, the RNA-seq, proteomics and interactome analyses will be repeated on the ‘optimized’ Exo-shRNA system to assess any differences in protein content and binding partner interactions compared to the current Exo-shRNA system. Overall, these data will provide important detailed insight into defining the Exo-shRNA system RNA and protein cargo for prototype development.

Thus, provided herein are studies demonstrating a highly programmable Exo-shRNA system with several functionally relevant Exo-shRNAs enriched in EVs from two different technologically relevant cell sources (HEK293 and NSCs). Furthermore, we expect to transfer functional cargo to recipient cells, eliciting knockdown of their targets. As Ago2 is known to co-localized with CD63 (20), we expect Ago2 to enrich in these fractions, but overexpressed Ago2 may also enrich in other EV subpopulations as a unique observation. We also expect that the RNA and proteins in the EVs will be biased to the programmed RNAi effector with a reduction in ‘containments.’ Overall, these observations should define the product characteristics of the programmable RNAi-deliverable EV system.

### Optimization of the Exo-shRNA EV System

Optimization of the Exo-shRNA system to further enhance RNAi enrichment and potency through the manipulation of the RNA expression, shRNA scaffold, Ago2 EV sorting factors, and host factors in EV biogenesis.

The broader utility of an RNAi-deliverable EV-based system would be contingent on establishing the parameters for optimal RNAi effector enrichment and scalability. As shown, overexpression of Ago2 increased enrichment of mature RNAi effectors in EVs, which was required for functional knockdown in target cells and not observed with the Exo-shRNA only (FIGS. 2G and 3A). Furthermore, variation is RNAi enrichment within EVs across three productions correlated strongly with knockdown efficacy (FIG. 3B), revealing that the effectiveness of the Exo-shRNA system is linked to RNAi enrichment. The Exo-shRNA enrichment in EVs could be enhanced through several features: 1) exploration of the sequence features of the RNA scaffold, 2) localization of the shRNAs to the cytoplasm, the site of MVB formation, for maximal EV-enrichment, 3) the manipulation of cellular factors involved in Ago2 sorting into EVs, and 4) rational and unbiased identification of factors affecting EV production and cargo loading. First, the Exo-shRNA scaffold was extrapolated from previous work on dicer-independent shRNAs for potent target knockdown (45), which includes an A:C mismatched base overhang at the 5′ and 3′ ends, respectively. Notably, miR-451 is enriched in EVs (11) and upstream processing of the primary miR-451 results in a pre-miRNA structure with 5′ and 3′ nucleotide extensions, which includes the A-C mismatch along with an additional UC-3′ extension (FIG. 3C). To determine if including additional ‘miR-451’ 3′ extensions onto the Exo-shGFP architecture could affect enrichment, we harvested EVs from HEK293 cells transfected with 3′ sequence variants and observed a stepwise enrichment with an extension towards the complete miR-451 3′ sequence.

Of note, this effect was not observed with blunt (A:U) or truncated 3′ variants (A:Δ), suggesting the 3′ nucleotide composition could affect Exo-shRNA EV enrichment. Alternatively, EV-enriching motifs or tethering of factors to EV markers have been used to enhanced RNA enrichment (12, 13), but covalent linkage could impact RNAi effectiveness through retention of the motif in recipient cells. A unique feature of Ago2-mediated shRNA maturation is the cleavage of the non-target strand (FIG. 1A), which would remove any 3′ extensions. This processing step could be exploited to attach EV-enriching RNA motifs present in producer cells but cleaved off and absent in the recipient cells. Second, the strong small nuclear U6 Pol III promoter was used to express the Exo-shGFP; however, these promoters result in nuclear retention of small RNAs (46). Alternative promoters have been used to improve RNA export and packaging into membrane-budded virus-like particles (47), which may further improve EV loading with RNAi effectors. Third, cellular factors involved in Ago2 phosphorylation affect cellular localization (24, 48) and are implicated in the sorting of Ago2 into EVs (20). To demonstrate this as a viable optimization parameter for Exo-shRNAs, we overexpressed Connexin 43 (Cx43), known to sort highly structured RNAs into EVs (i.e. Ago2-mediated shRNAs), with a Nluc fused Ago2 (Nluc-Ago2) and the Exo-shGFP and observed a 6-fold increase in Nluc signal in supernatant compared to a control protein (GFP) (FIG. 3D). Lastly, exploring factors that increase EV production without compromising cargo enrichment would be beneficial for the scalability of EV technology. EV ‘boosters’ have been developed (15), and although they do increase EV yields, in our hands, they resulted in a lower ratio of cargo packaging (data not shown). To address this cargo ‘dilution’ effect, we have shown that the knockdown of Charged Multivesicular Body Protein 4C (CHMP4C) and Vacuolar Protein Sorting 4 Homolog B (VPS4B) (FIG. 4A) increased the total EV yield (FIG. 4B) (49). Importantly, a nanoluciferase (Nluc) fused to CD63 (CD63-Nluc) resulted in an increased packaging efficiency when inhibiting these factors (FIG. 4C).

### Optimization of Exo-shRNA RNA Scaffold and Promoter

To investigate the optimization of the RNA scaffold, a series of Exo-shGFPs with 3′ extensions will be explored. The extensions to be explored are: 1) the miR-451 extension (11), 2) a hnRNPA2/B1-associated ‘Exo-motif’ (12), 3) the SYNCRIP-associated hExomotif (51), 4) polyuridylation of the Exo-shRNA (23), 5) the LA protein-associated motif (52), 6) astrocyte and neuron-associated motifs (53), and 7) the ‘extended’ Exo-motifs (54). The Exo-shGFP 3′ sequences are summarized in Table 4.

To potentially enhance cytoplasmic accumulation of the Exo-shRNAs for EV packaging, the Exo-shGFP with be cloned downstream of the Pol II U1 promoter (55) and the tRNA lys promoter (47), and compared to the U6 Pol III promoter. For the motif-containing or promoter expressed shRNAs, the EVs will be assessed by RT-qPCR for the mature RNAi effector levels and knockdown efficiency assessed as described in Rationale.

### Optimization of Ago2 Sorting Factors for the Exo-shRNA System

To explore potential EV trafficking factors of Ago2 and their effect on the Exo-shRNA system, clonal knock-out cell lines of HEK293 cells will be generated for factors implicated in the cellular retention of Ago2. These factors include: Caveolin 1 (CAV1), Akt3 (24), MAPKAPK2 (MK2)(24), MEKI and 11 (20), ERK (20), EGFR (56) and p38-MAPK (57). Reciprocally, the overexpression of factors potentially involved in Ago2 sorting into EVs will be explored through the overexpression of PTPN1 (58), a dominate negative KRAS (S17N) mutant (59), Cx43 (60), and the EV-factor ALIX (21). FLAG-Ago2+Exo-shGFP will be transfected into the knockout HEK293 cell lines or WT HEK293 cells with the described overexpression vectors. The EVs will be harvested, and assays performed to verify enrichment and functional knockdown as described herein. Furthermore, immunoblotting will be performed for FLAG-Ago2 to assess the protein levels. Finally, the potential optimal shRNA factors identified. will be combined with the optimal sorting factors, and the absolute levels of mature RNAi effector and Ago2 levels will be determined to quantify functional RNAi effectors per EV as described herein.

Described herein is the identification of a highly optimized RNA structure combined with the optimal sorting factors to generate a refined Exo-shRNA system from practical applications. These data may not only improve the technology as a broadly applicable potent RNAi-deliverable EV platform, but also a means to validate known EV-enriching RNA motifs and factors involved in Ago2 sorting in functionally relevant assays. Our data shows some factors will improve enrichment (FIG. 4C). Further, we will isolate Exo-shRNA containing EVs. EV fractions containing Ago2 are associated with the surface EV-marker CD63 (20), and methods to isolate CD63-expressing EVs could be performed as a mechanical optimization step.

Furthermore, we anticipate that the KG of host factors will enrich the Exo-shRNA system as well as increase EV production, including a CRISPRi screen to reveal novel epigenetic factors critical for EV biogenesis and cargo packaging. These observations will be insightful for EV biology as well as impacting EV-based applications to enhance production. Of note, a CRISPRi screen will be complemented by a targeted approach. with strong preliminary data (FIGS. 3A-3D) to identify protein factors that could improve the Exo-shRNA system.

### Utility of the Exo-shRNA EV System In Vitro and In Vivo

Assessment of the tropism and safety of the Exo-shRNA EVs to deliver functional RNAi effectors in vitro and in vivo for biotechnological applications.

Although EVs potentially have various surface factors that allow for receptor binding that affect EV uptake, depending on the mechanism of entry, additional elements may be required for escape from the endosomal compartment, otherwise trafficked for lysosomal degradation. The plethora of interactions that could dictate the EV-cell interaction needs further study. Tissue tropism of EVs is poorly understood, especially with regards to cargo release, and remains an important hurdle for the cognizant application of EV-based technologies. Although, natural cargo release of non-coding RNA remains controversial (64), we have demonstrated the transfer of zinc-finger protein transcription regulators delivered by EVs in vitro and in vivo using a non-immunogenic Cx43 (44, 65).

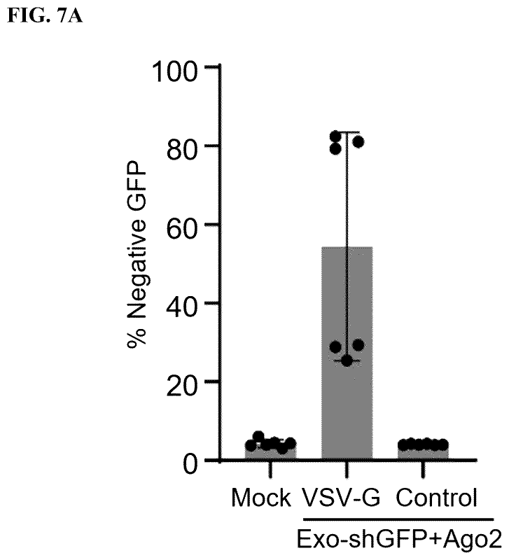

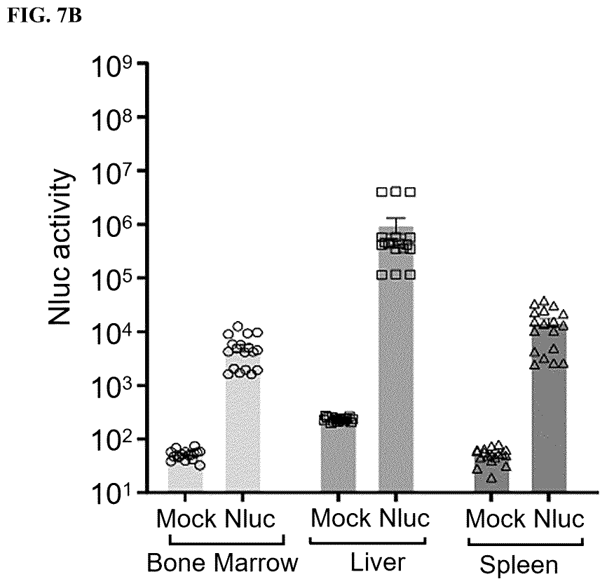

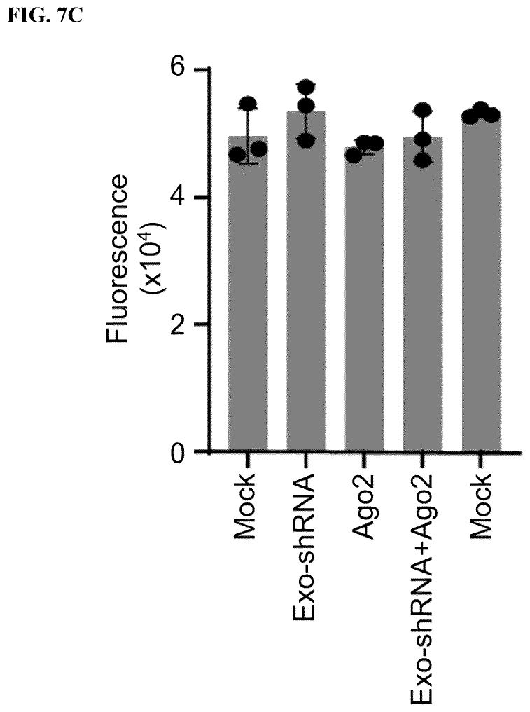

Some miRNA-based EV systems have been used by others but are limited by poor programmability. Cre recombinase delivered with nanoparticles into transgenic mice can measure cargo release through reporter activation (66). However, this system requires a LoxP reporter and does not readily import into various cell lines and mouse models. Nluc fused to known EV factors is another means to assess EV biodistribution, and we have used this system to show that: 1) EVs engineered with ligands can bind and enrich on cell lines expressing the target receptor (67) and 2) implanted CD63-Nluc cells (FIG. 7B) or purified EVs administered systemically (65) resulted in increased Nluc signal in various organs. However, the Nluc system only determines EV accumulation and not cargo release. Ultimately, these data and previous studies demonstrate our ability to explore biodistribution of EVs but highlight the need for new tools to explore EV cargo release. The Exo-shRNA system represents a readily programmable means to load cargo into EVs with a functional RNAi read-out which can benefit hard problems EV research faces, such as tropism and novel fusogen discovery. Apart from highlighting the utility of the Exo-shRNA system, these studies will also further refine its downstream applications.

### Investigation of EV Tropism Using the Exo-shRNA System In Vivo.

To assess the delivery of the Exo-shGFP EVs to different organs in vivo, the Ago2+shGFP or control EVs will be generated for HEK293 cells as described and injected daily over 6 days (total six injections) with a dose of 1×10¹⁰ EVs, which previously elicited effects in vivo when delivering a transcriptional regulator (65). The EVs will be injected into a transgenic mice FVB-Tg(CAG-luc-GFP)L2G85Chco/J as this mouse strain ubiquitously expresses a fusion GFP-luciferase with broad tissue bioluminescence and fluorescence (Jackson laboratories, #008450). The Exo-shGFP would affect both GFP and luciferase. The EVs will be injected intravenously (retroorbital) and intraperitoneal (IP) to assess if the administration route could affect the tissue distribution and knockdown. At 48 hrs after the final EV dose, luciferin will be administrated IP, the mouse euthanized, and a full necropsy performed as described (65). The lung, liver, brain, spleen, intestines, lymph nodes, and kidneys will be removed and the levels of luciferase in organs measured using a LagoX imager (Spectral instruments imaging, USA). The tissue will then be homogenized and Nano-Glo® Luciferase substrate (Promega, USA) added to determine luciferase activity detected on the GloMax Discover Microplate Reader System (Promega, USA). Bone marrow will be extracted and subjected to a luciferase assay to assess distribution of the Exo-shRNA EVs to the hematopoietic compartment. Furthermore, lung, liver, brain, spleen, and bone marrow will be analyzed by immunohistochemistry (IHC) for the presence of FLAG-Ago2 and overlaid with a GFP signal to provide a detailed assessment of Ago2 delivery and reporter knockdown. To assess the distribution in immune cells, mice will be bled by retroorbital bleeding to collect blood prior to euthanasia as well as single cell suspensions obtained from bone marrow and spleen. The levels of GFP will be measured in circulating and splenic/hematopoietic-derived immune cells by flow cytometry using a multiple flow detection panel for assessing lymphoid (CD3, CD4, CD8) and myeloid populations (F4/80 and CD11b). Collectively, these assays will provide a valuable insight into the release of cargo in tissues using the Exo-shRNA system and further refine its potential future applications.

### Investigation of Tropism and Non-Immunogenic Fusogens Using the Exo-shRNA System In Vivo

To complement the in vivo assays, in vitro cellular tropisms of Exo-shGFP EVs will be tested in a range cell lines. Importantly, the Exo-shRNA EVs will be produced with endogenous non-immunogenic fusogens to preserve the native nature of the EVs. These fusogens will be the gap-junction protein, Cx43 used in our previous studies (15), an endogenous retrovirus fusion syncytin identified by others (SynA) (68), or a novel calcium-mediated fusion protein Myoferlin (69). The following cells lines will be selected for stable GFP-luciferase reporter expression and tested for knockdown with the Exo-shGFP EVs: liver (HepG2), lung (Calu-3), brain (SH-SY 5Y), lymphoid (Jurkat T-cell and Raji B-cell), and myeloid cells (RAW 264.7). EVs with a VSV-G will be included as a positive control. A total amount of 1×10⁶, 1×10⁷, and 1×10⁸ EVs will be added to the cell lines in a high-throughput 96-well plate format and the levels of luciferase assessed using a Bright-Glo assay (Promega, WI, USA). Overall, these assays will define the cell type specificity of the unmodified and non-immunogenic fusion-modified Exo-shRNA EV to establish a rapid screening tool for assessing functional cargo release in vitro. To assess the effects of non-immunogenic fusogens in vivo, the Exo-shGFP+Ago2 EVs will be generated for HEK293 cells as described with Cx43, SynA, or Myoferlin. Mock control EVs without fusogen or VSV-G EVs will be included for comparison.\

### The Safety Profile of Exo-shRNA In Vitro and In Vivo

Validation of the potential non-specific effects of a novel technology is required for its application. To determine if the Exo-shRNA EVs had any gross effects on proliferation of cultured cell line, HEK293 cells were treated with the EVs and at 72 hrs post-addition, proliferation was assessed by an AlamarBlue proliferation assay and, encouragingly, showed no substantial effect on cell growth compared to mock cells (untreated) (FIG. 7C). Exo-shRNA EV-treated cells will be subjected to RNA-seq to determine the effects on global RNA levels in the recipient cells. EVs with the FLAG-Ago2 only or mock EVs will be included to determine effects specific to the Exo-shRNA system. GO term and KEGG pathway analysis will be performed on the RNA-seq data to define any affected cellular pathways as previously performed (70). To assess safety in vivo, BA L B/c mice will be injected intravenously (retroorbital) with the empty, FLAG-Ago2, and FLAG-Ago2+Exo-shGFP EVs. The mice will be dosed with a described dose of 1×10¹⁰ EVs and a higher dose of 1×10¹¹ EVs and at 24 hrs, 48 hrs, and 72 hrs. After the final administration, mice will be bled and serum collected for the following assays: 1) a standard liver functionality test detecting serum ALT and AST, and 2) Cytokine & Chemokine 36-Plex Mouse ProcartaPlex™ Panel 1A (Thermo Fisher Scientific, USA) to determine any inflammation (Analytical Pharmacology Core Facility (A PCF): fee for service). The general well-being of the mice will be observed including weight and clinical scores for adverse effects. Finally, three mice from each group will be euthanized at 48 hrs after the final dose and lung, liver and spleen will be removed and analysed using H & E staining to assess pathology markers (Pathology: Molecular Pathology core). These data will provide insight into any adverse effects from the Exo-shRNA EVs and further refine it as a potential biomedical platform.

A parallel goal of this study is to explore non-immunogenic EV delivery systems to leverage the native nature of EV technology, especially for biomedical applications, and we expect to see a modified knockdown profile with our non-immunogenic fusogen panel. Exploring these fusogens would not only allow for the validation of them for in vitro applications, an active area of research for EV engineering, but further demonstrate the utility of this system as a rapid screening tool. Concerning the in vivo assays, we expect the Exo-shRNA loaded EVs to elicit knockdown in organs that have previously been susceptible when delivering a transcriptional regulator using HEK293 EVs (65). These current studies will likely not require a fusogen to observed tissue knockdown in vivo; however, knockdown may be enhanced, or new tissue knockdown profiles observed with alternative fusogens. Importantly, the in vitro cell line assays may inform which organs are more receptive to uptake by a particular non-immunogenic fusogen. Another potential problem is that within the context of the described transgenic mice FV B-Tg(CA G-luc-GFP)L 2G85Chco/J model, the stability of the GFP expressed protein may prove problematic. This model contains an enhanced GFP that has a stable half-life, which may reduce the signal-to-noise ratio needed to observe significant knockdown. Gain-of-function signals are more readily observed than knockdown signal. A Nluc-Ago2 could be used in BALB/c mice to assess signal accumulation in various tissues, and then RNA could be extracted from tissues positive for Nluc signal from the transgenic mice and subjected to more sensitive assays for RNA knockdown. Finally, HEK293s EVs are well tolerated in vivo (4) with a low likelihood that the EVs will cause unwanted effects.

Described herein is development of a next-generation, optimized EV technology to deliver highly enriched and readily programmed RNAi for biomedical and biotechnological applications. Furthermore, the study will have several secondary outcomes: 1) the elucidation of RNA and protein factors that may inform endogenous control of RNAi into EVs, specifically related to dicer-independent shRNAs, 2) factors associated with EV biogenesis and cargo loading which could be applied to other EV payloads, and 3) the validation of various applications of the Exo-shRNA system in elucidating EV tropism and novel fusogens in vitro and in vivo, which would inform future biomedical applications. Overall, the optimized Exo-shRNA system will represent a generalizable, next-generation biocompatible delivery platform for gene knockdown applications.

### Example 3: Materials and Methods

### Vectors:

The shRNA vectors were generated through Genescript (NJ, USA) and inserted into a PUC57 backbone. The sequences contain the small RNA promoter, shRNA sequence and transcription terminator. If needed, the shRNAs vector were verified by sanger sequencing.

The pSI-GFP-S [pSI-GFP sense (+) and pSI-GFP sense (−) and AS (pSI-GFP anti-sense (+) and pSI-GFP anti-sense (−)], or the pSI-362-S [(pSI-362 sense (+) and pSI-362 sense (−) and AS (pSI-362 anti-sense (+) and pSI-362 anti-sense (−)] vectors were generated using standard oligomer cloning procedures by inserting annealed complementary oligomers into a XhoI and NotI digested pSI-Check2.1 vector. To generate the pSI-HPV16-E6/E7 target vector, the E6/E7 target site was synthesized as a DNA gblock™ by integrated DNA technologies (and inserted into a XhoI and NotI digested pSI-Check2.1 vector by Gibson assembly using the NEBuilder® HiFi DNA assembly M aster mix as instructed (NEB, MA, USA). The pSI-Check target vectors were verified by sanger sequencing.

The Cas13d (CasRx) was obtained from Addgene (EF1a-dCasRx-2A-GFP, pxR002, #109050; Addgene, MA, USA). The Dicer-cRNA was generated by PCR amplification of the crRNA sequence off a plasmid template with a U6 Pol III promoter with the U6-F and Cas13d (CasRx) Dicer-crRNA-R using KAPA2G Robust HotStart Ready mix according to manufacturer instructions (Sigma, St. Louis, MO) in a T100 thermocycle (Bio-Rad, CA, USA). The amplicon was inserted into a pCR2.1 TA cloning vector (Cat No. K202020, TA Cloning™ Kit, with pCR™2.1 Vector; Thermo Fisher Scientific, MA, USA). The crRNA vectors were verified by sanger sequencing. The empty CasRx crRNA vector was obtained from Addgene (pXR003: Plasmid #109053; Addgene, MA, USA).

To generate the overexpression vectors, the PTPN1, HRAS, HRAS-S17N, HRAS-G12D, HRAS-G12V, KRAS, KRAS-S17N, KRAS-G12D, NRAS, hnRNPA2B1, SYNC RIP, LA protein, ALYREF, Fus, MEX3C-1, CD63, CD63-U1aRBP were synthesized as a DNA gBlock™ by integrated DNA technologies (IDT, IA, USA) and inserted into a XhoI and NotI digested pCI-Neo mammalian expression vector through Gibson assembly using the NEBuilder® HiFi DNA assembly Master mix as instructed (NEB, MA, USA). The overexpression vectors were verified by sanger sequencing.

The FLAG-Ago2, ALIX, PACT, TRBP, HSP90, as well as EGFR and AKT3 WT and mutant vectors were obtained from Addgene (Addgene, MA, USA). Connexin43 S368A (pDB68) was generated previously and kindly provide to us as a gift from Dr Martin Fussenegger (1). The VSV-G and pcDNA-GFP vector have been previously validated in-house. The pRL was obtained from Promega (Promega, WI, USA).

To generate the Nluc-Ago2 vector, the Nluc fragment was synthesized as a DNA gBlock™ by integrated DNA technologies (IDT, IA, USA) and inserted into a HindIII and BamHI digested FLAG-Ago2 vector using Gibson assembly as described.

### Cell Lines and Culture:

The HEK293, HEK293-GFP, Huh7 cells were cultured in Dulbecco's modified Eagle's medium (DMEM) supplemented with 10% fetal bovine serum (FBS, Thermo Fisher Scientific, MA, USA). The HEK293-GFP cells that stably express a consistent level of GFP (also referred to as pM o-C6 cells) were generated as previously described (2). The A549 cells (ATCC: CCL-185) were cultured in F-12K Medium (Thermo Fisher Scientific, MA, USA) supplemented with 10% fetal bovine serum. The HepG2 (ATCC: HB-8065), SiHa (ATCC: HTB-35), SCC154 (ATCC: CRL-3241), U-87 (ATCC: HTB-14) were cultured in Eagle's Minimum Essential Medium (EMEM) (Thermo Fisher Scientific, MA, USA) supplemented with 10% fetal bovine serum. All cell lines were cultured at 37° C. and 5% CO₂.

To generate the stable cell lines, the cells were transduced with a lentiviral vector packaged with a LV-E1alpha-GFP-Fluc-IRES-Puro reporter. The lentiviral vector was generated by Vectorbuilder (Supplement Document; VectorBuilder Inc, IL, USA). The pseudotyped lentiviral vector with Vesicular stomatitis virus G protein (VSV-G) was generated and titered at the Gene Editing and Viral Vector Core at City Hope using standard procedures. The U87, Huh7, HepG2, SiHa, SCC154, A549 cell lines were transduced with a MOI of 0.2, and at 72 hrs post-transduction, completed media was replaced and supplemented with 1.5 μg/ml puromycin. A consistent population expressing high levels of GFP positive cells were confirmed by flow cytometry using single cells counted to 10000 events on a BD Accuri™ C6 or NovoCyte Quanteon Flow Cytometer Systems and analysed using the Flowjo vX 5.0 software.

### EV Generation and Purification:

To generate the EVs with the enriched RNAi effectors, HEK293 cells were seeded at 3.25×106 cells in a 150 mm plate, and 72 hrs later, the media was replaced on the same day with 15 ml of complete media and after at least 2 hrs, the cells were transfected using PEIMAX® (Cat No. 24765, Polysciences, Inc., PA, USA) at a 1:5 ratio of PEIMAX:DNA. A total of 40 μg of DNA was transfected: 18 μg of FLAG-Ago2, 18 μg of shRNA, 4 μg of VSV-G. W here applicable, PUC19 was substituted as an empty vector control. A plate transfect with 40 μg of GFP was included as a transfection efficiency control. After 24 hrs, the media was removed, washed with 15 ml of PBS, and then 45 ml of FreeStyle™ 293 Expression Medium was added to the plate (Thermo Fisher Scientific, MA, USA).

For purification through differential ultracentrifugation (D-UC), the supernatant was collected at 72 hrs. The viability of the cells was determined at the time of EV s collection, which was greater than 90%. The supernatant was centrifuged at 450×g for 10 min, and then the supernatant was transferred to a new 50 ml conical tube and centrifuged at 10000×g for 30 min. The supernatant was passed through a 0.45 μm filter (MilliporeSigma, MA, USA) and the filtered supernatant was ultracentrifuged at 100000×g for 120 min at 4° C. using a SW32Ti rotor in a Beckman XL-900 ultracentrifuge. The supernatant was discarded and small EVs were resuspended in PBS. The ultracentrifugation step was repeated with a 30 ml PBS wash and the pellet resuspend in 100-200 μl of PBS.

To purify the EVs by Size-exclusion chromatography, the qEV system was used (IZON Science LTD ©, WA, USA). The EVs were prepared by D-UC as described and then applied to the qEV 10/35 nm column system as per the manufacturer's instructions (IZON Science LTD ©, WA, USA). Fractions were collection in 4 ml amounts and either pooled or individual fractions. The EV fractions were subjected to another round of ultracentrifugation as described and the pellet resuspend in 100-200 μl of PBS.

For the iodixanol density gradient purification, the flotation protocol was used as previously described with some modifications (3). Briefly, the D-UC EVs were made up to 3 ml in PBS and mixed with 9 ml of 60% iodixanol solution to a total of total 12 ml (Cat. No. D1556, OptiPrep, Sigma, MO, USA). The 45% iodixanol/EV mixture was added to the bottom of a 38.5 mL Ultra-Clear tube (Cat. No. C14292, Beckman Coulter, CA, USA), and successive layers of 30% (9 mL), 23% (6 mL), and 18% (6 mL) were added carefully on top. The gradient was ultracentrifuged in a swinging-bucket SW32 Ti rotor for 16 hrs at 100000×g at 4° C. Fractions of 1 ml were either pooled or individual fractions subjected to another ultracentrifugation step as described above and the pellet resuspend in 100-200 μl of PBS. Generally, the D-UC, SEC, or DG purified EVs were stored at 4° C. and used fresh, but if appropriate were stored at −80° C. until ready to be used.

### Negative Staining Electron Microscopy:

EVs were absorbed to glow-discharged, carbon-coated 200 mesh Formvar grids. Samples were prepared by conventional negative staining in 1% (w/v) uranyl acetate. Electron microscopy images were taken on an FEI Tecnai 12 transmission electron microscope equipped with a Gatan OneView CMOS camera.

### Nanoparticle Tracking Analysis (NTA):

The EVs were assessed and quantified using NTA on a NanoSight NS300 Instrument (Malvern Panalytical Inc., MA, USA). The NTA samples were run at a 1:1000 dilution. A blue 488 nm laser was used to detect the extracellular vesicles, with a slide shutter level set to 1259 and the slider gain set to 366, and the syringe pump speed set to 30 using a flow-cell top plate module. A threshold setting of 5 was used to determine particle count.

### EV-Mediated RNAi Knockdown of Reporter Cell Lines:

The stable GFP expressing cell line were seeded 1×104 cells per well in a 96-well plate, and either 10 μl of purified EVs or the described particle amount was added to the cells. At 72 hrs post-transfection, the levels of GFP were assessed using flow cytometry. Single cells events were counted to 10000 on a BD Accuri™ C6 or NovoCyte Quanteon Flow Cytometer Systems and analysed using the FlowJo vX 5.0 software.

### Transfections and Luciferase Assays:

For the pSI-Check reporter assays, HEK293 cells were seeded at 4×104 cells per well in a 48-well plate, and 24 hrs later were transfected using Lipofectamine 3000® (Thermo fisher scientific, MA, USA) with 50 ng of pSI-C heck vector and 400 ng of the shRNA vector (1:8) with 50 ng of pcDNA-GFP. At 48-72 hrs post-transfection, the levels of Rluc and Fluc were assessed using a Dual-Luciferase® Reporter Assay and activity detected on the Glomax® Explorer system (Promega, WI, USA). When dosing the shRNA versus pSI-Check target vectors, the shRNA vector was reduced to 200 ng (1:4), 100 ng (1:2), 50 ng (1:1), or 25 ng (1:0.5). To make up to 500 ng total DNA, the remaining amount of DNA was transfected with a stuffer vector (PUC19 or U6 vector that does not express a shRNA). The luciferase assays were performed as described.

To assess the effects of the CasRx and Dicer-crRNA on Dicer mRNA levels, HEK293 cells were seeded at 4×104 cells per well in a 48-well plate, and 24 hrs later were transfected using Lipofectamine 3000® (Thermo fisher scientific, MA, USA) with 250 ng of the CasRx and 250 ng of the Dicer-crRNA vectors (or the pXR003 control-crRNA vector). At 72 hrs post-transfection, the RNA was extracted, and RT-qPCR performed as described below.

For the pSI-Check reporter assays with the CasRx and Dicer-crRNA, HEK293 cells were seeded at 4×104 cells per well in a 48-well plate, and 24 hrs later were transfected using Lipofectamine 3000® (Thermo fisher scientific, MA, USA) with 250 ng of the CasRx, 250 ng of the Dicer-crRNA (or control-crRNA), 50 ng of the pSI-Check vector, 50 ng of the shRNA vector, and 50 ng of pcDNA-GFP. At 48 hrs post-transfection, the levels of Rluc and Fluc were assessed using a Dual-Luciferase® Reporter Assay and activity detected on the Glomax® Explorer system (Promega, WI, USA). The luciferase assays were performed as described.

For the HEK293-GFP (pM O-C6) assays, the cells were seeded and transfected as described with 500 ng of the shRNA vectors. At 72 hrs post-transfection, the levels of GFP were assessed y=using flow cytometry. Single cells events were counted to 10000 on a BD Accuri™ C6 or NovoCyte Quanteon Flow Cytometer Systems and analysed using the FlowJo vX5.0 software.

Extracellular Ago2 Nanoluciferase assay:

HEK293 cells were seeded at 1.2×105 cells per well in a 24-well plate, and 24 hrs later were transfected using Lipofectamine 3000@(Thermo fisher scientific, MA, USA) with 400 ng of the Nluc-Ago2 vector and 400 ng of the shRNA vector with 50 ng of pcDNA-GFP. After 24 hrs, the media was removed and replaced with 1 ml of FreeStyle™ 293 Expression Medium. After 48 hrs post-media change, the supernatant was collected in a 1.5 ml Eppendorf, and centrifuged at 450×g for 10 min, and then 500 μl of supernatant was transferred to a new 1.5 ml Eppendorf and centrifuged at 10000×g for 30 min. Two-hundred and fifty microliters of supernatant was then passed through a 0.2 μm filter (Cat. No. SLLG025SS, Millex-LG, Sigma) and 50 μl of the filtered supernatant was added to a white opaque luminometer plate. The nluc levels were assessed by adding 50 μl of NanoGlo reagent to the supernatant from the Nano-Glo™ Luciferase Assay System and activity detected on the Glomax® Explorer system (Promega, WI, USA).

### Protease and RNAse Protection Assays:

In the protease protection assays, 10 μl of purified EVs were mixed with or without Proteinase K (5 μg/ml; Cat. No. P8107S, NEB, MA, USA) in the absences or presences of Triton-X 100 (0.1%) in a total volume of 20 μl. PBS was used to make up the volumes when PK or Triton-X 100 were not added. The mixture was incubated on ice for 15 min and then Phenylmethylsulfonyl fluoride was added (5 mM; PMSF) to inactive PK and incubated on ice for a further 5 min. An additional 30 μl of lyse buffer that contained 1×RIPA (Cat. No. J 62524-A E, Thermo fisher scientific, MA, USA), 1× Laemmli Sample Buffer (Cat. No. #1610747, Bio-Rad, CA, USA), and b-mercaptoethanol was added. The 50 μl sample was boiled on a preheated T100 thermocycle at 100° C. for 10 min (Bio-Rad, CA, USA). The protein sample (10-20 μl) was loaded on an acrylamide gel and subjected to western blot analysis as described below.

For the RNA se protection assay, the proteinase K reaction was performed as described, and then RNAse A (5 μg/ml; Cat. No. T3018L, NEB, MA, USA) was added and the reaction incubated at 37° C. for 15 min. Then, 200 U of RiboLock RNase Inhibitor (Cat. No. E00382, Thermo fisher scientific, MA, USA) was added and incubated at RT for 5 min. RNA was extracted using the Maxwell® RSC miRNA from Plasma and Serum Kits as per manufacturer instructions (Cat No. A51680; Promega, WI, USA).

### Western Blots:

The EVs or cells were lysed in M-PER™ Mammalian Protein Extraction Reagent supplemented with Halt™ Protease Inhibitor Cocktail and the protein concentration determined by Pierce™ BCA Protein Assay Kit according to manufacturer protocols (Thermo Fisher Scientific, MA, USA). The samples were mixed with 1× Laemmli Sample Buffer (Cat. No. #1610747, Bio-Rad, CA, USA) and b-mercaptoethanol, and boiled on a T100 thermocycle at 95° C. for 5 min (Bio-Rad, CA, USA). Equal amounts of protein from each sample were loaded onto a 4-20% Mini-PROTEAN TGX Precast Protein Gels (Bio-Rad, CA, USA) and transferred using the Trans-Blot® Turbo™ Transfer System with Trans-Blot® Turbo™ Mini Nitrocellulose Transfer Packs (Bio-Rad, CA, USA). The membrane was blocked with 3% BSA TBS-T and subsequently probed with the following antibodies: α-FLAG mouse mAb Anti-Flag® M2 (Cat. No. F1804; Milliporesigma, CA, USA), α-alpha tubulin rabbit polyclonal (Cat. No. 4074; Abcam, Cambridge, United Kingdom), α-CD63 mouse anti body (Cat. No. sc-5275, Santa Cruz, CA, USA), α-CD81 mouse antibody (Cat. No. sc-7637, Santa Cruz, CA, USA), α-TSG101 mouse antibody (Cat. No. ab30871, Abcam, Cambridge, UK), α-ALIX mouse antibody (Cat. No. 21715, C ell Signaling, M A, USA), α-Calnexin rabbit antibody (Cat. No. ab92573, Abcam, Cambridge, UK), and α-Syntenin rabbit antibody (Cat. No. ab133267, Abcam, Cambridge, UK), α-Histone H3 (D1H2) XP® rabbit antibody (Cat. No. 4499, Cell Signaling, MA, USA), α-VSV-G rabbit antibody (Cat. No. ab183497, Abcam, Cambridge, UK) or secondary antibodies used were the HRP-conjugated α-Mouse IgG goat antibody (Cat. No. 1705047; Bio-Rad, CA, USA) or Immun-Star™ Goat Anti-Rabbit (GAR)-HRP Conjugate (Cat. No. 170546; Bio-Rad, CA, USA). The membrane was exposed using a Pierce™ SuperSignal™ West Pico PLUS Chemiluminescent Substrate (Thermo Fisher Scientific, MA, USA) and the signal detected on the Bio-Rad Chemidoc™ Touch Gel-Imaging System and analyzed on Bio-rad Image Lab™ Software V6.0.0. The membranes were thoroughly washed and stripped with Restore™ PLUS Western Blot Stripping Buffer (Thermo Fisher Scientific, MA, USA), if necessary, between repeat probing.

### RNA Extraction and RT-qPCR Assay:

For the detection of matured RNAi effectors in the EVs, the total small RNA was extracted from 50 μl of purified EVs using the Maxwell® RSC miRNA from Plasma and Serum Kits as per manufacturer instructions (Cat No. A S1680; Promega, WI, USA). For cell extracts, the total small RNA was extracted from 1×106 cells using the Maxwell® RSC miRNA Tissue Kit (Cat No. A51460; Promega, WI, USA). A total of 4 μg of cellular RNA or 40 μl of EV RNA were reverse transcribed using the Mir-X™ miRNA First-Strand Synthesis Kit according to the manufacturer instructions (Takara Bio, CA, USA). The RT product was diluted 1 in a 100 in molecule grade water before qPCR detection. Two microliters of RT-template were mixed with the shGFP qPCR F and the mRQ 3′ primers in the TB Green® Fast qPCR Master Mix as per manufacturer instructions (Takara Bio, CA, USA). The U6 forward and reverse primers in the kit were used to detected U6 snRNA as a background control. The reaction was amplified in a LightCycler® 96 (Roche, Basel, Switzerland) with the following conditions: initial denaturation: 95° C. for 10 sec (1 cycle), and then denaturation 95° C. for 5 s, annealing/extension at 60° C. for 20 sec (40 cycles). The data was analyzed with the LightCycler® 96 software (V1.1.0.1320). The primers used to the detect are described. For the primer extension assays, shGFP qPCR F 18 nt, 19 nt, 20 nt, 21 nt, 22 nt primers were used.

For absolute quantification, a GFP siRNA 18 nt in length was synthesized by integrated DNA technologies (siGFP RNA 18 nt; IDT, IA, USA). A total of 3 ng of siGFP RNA, which equals ˜2.01×1011 copies of RNA per μl. The siGFP RNA 18 nt product was serially diluted from 2.01×1011-2.01×100 RNA copies per μl and then 1 μl reverse transcribed in the RT reaction as described above. The qPCR reaction was performed as described to generate a standard curve and calculate the absolute copy numbers of siRNA in each EV sample. To calculate the absolute copy number of siGFP per ul was divided by the volume of EV sample input into the RNA extraction. This value could then be normalized to the number of particles per μl (as determined by NTA) to determine the copies per EV particle.

To detect the Dicer mRNA, RNA was extracted from the HEK293 at 72 hrs post-transfection using the Promega Maxwell™ RSC simplyRNA Tissue Kit (Promega, WI, USA). One-microgram of HEK293 RNA was reverse transcribed using the QuantiTect® Reverse Transcription Kit (Qiagen, Hilden, Germany), and 4 μl of RT-template was amplified with the hDICER1 qPCR F and R primers in a LightCycler® 96 (Roche, Basel, Switzerland) using the KA PA Sybr® Fast qPCR Master Mix (Sigma-aldrich, MO, USA) with the following conditions: initial denaturation: 95° C. for 3 min, denaturation 95° C. for 5 s, annealing/extension at 60° C. for 20 sec. The data was analysed with the LightCycler® 96 software (V1.1.0.1320). GA PD H was detected as a background control.

For analysis of RT products from EVs and cells, forward and reverse reads are first merged using PEAR v0.9.6; improperly merged pairs are discarded. Merged reads are screened for adapter content using cutadapt v3.4 with two passes. The first pass removes reads lacking a polyT run of at least 10 bp, preceded by up to 150 nucleotides on the 5′ end. The second pass requires that the adapter sequence GCTGACCCTGAAGTTCAT (SEQ ID NO:216) is present on the 3′ end, with the default number of mismatches allowed (cDNA bp: 1-9: 0; 10-18: 1). The number of unique “inserts” between the two motifs is counted and the proportion calculated using the total number of polyT-containing reads as the denominator. cDNA sequences are reverse complemented to give the original reported siRNA sequence. Overrepresented siRNAs are those that account for more than 0.1% of reads in a library.

### Statistical Analysis

Graphing and statistical analyses were performed using GraphPad Prism version 8 (V8.1.2).

### EXAMPLE 1 AND EXAMPLE 2 REFERENCES

1. Herrmann I K, Wood M J A, Fuhrmann G. Extracellular vesicles as a next-generation drug delivery platform. Nature nanotechnology. 2021; 16(7):748-59. doi: 10.1038/s41565-021-00931-2.
2. Pastore C. Gene delivery beyond the liver. Nature nanotechnology. 2022; 17(12):1239-. doi: 10.1038/s41565-022-01292-0.
3. Banks W A, Sharma P, Bullock K M, Hansen K M, Ludwig N, Whiteside T L. Transport of Extracellular Vesicles across the Blood-Brain Barrier: Brain Pharmacokinetics and Effects of Inflammation. Int J Mol Sci. 2020; 21(12). Epub 2020/06/25. doi: 10.3390/ijms21124407.
4. Zhu X, Badawi M, Pomeroy S, Sutaria D S, Xie Z, Baek A, Jiang J, Elgamal O A, Mo X, Perle K L, Chalmers J, Schmittgen T D, Phelps M A. Comprehensive toxicity and immunogenicity studies reveal minimal effects in mice following sustained dosing of extracellular vesicles derived from HEK293T cells. J Extracell Vesicles. 2017; 6(1):1324730-. doi: 10.1080/20013078.2017.1324730. PubMed PMID: 28717420.
5. Mashouri L, Yousefi H, Aref A R, Ahadi Am, Molaei F, Alahari S K. Exosomes: composition, biogenesis, and mechanisms in cancer metastasis and drug resistance. Molecular Cancer. 2019; 18(1):75. doi: 10.1186/s12943-019-0991-5.
6. Isola L A, Chen S. Exosomes: The Messengers of Health and Disease. Current Neuropharmacology. 2017; 15(1):157-65. doi: http://dx.doi.org/10.2174/1570159X 14666160825160421.
7. Rezaie J, Feghhi M, Etemadi T. A review on exosomes application in clinical trials: perspective, questions, and challenges. Cell Communication and Signaling. 2022; 20(1):145. doi: 10.1186/s12964-022-00959-4.
8. Fu S, Wang Y, Xia X, Zheng J C. Exosome engineering: Current progress in cargo loading and targeted delivery. Nano Impact. 2020; 20:100261. doi: https://doi.org/10.1016/j.impact.2020.100261.
9. Hou X, Zaks T, Langer R, Dong Y. Lipid nanoparticles form RNA delivery. Nature Reviews Materials. 2021; 6(12):1078-94. doi: 10.1038/s41578-021-00358-0.
10. Hu B, Zhong L, Weng Y, Peng L, Huang Y, Zhao Y, Liang X-J. Therapeutic siRNA: state of the art. Signal Transduction and Targeted Therapy. 2020; 5(1):101. doi: 10.1038/s41392-020-0207-x.
11. Reshke R, Taylor J A, Savard A, Guo H, Rhym L H, Kowalski P S, Trung M T, Campbell C, Little W, Anderson D G, Gibbings D. Reduction of the therapeutic dose of silencing RNA by packaging it in extracellular vesicles via a pre-microRNA backbone. Nature Biomedical Engineering. 2020; 4(1):52-68. doi: 10.1038/s41551-019-0502-4.
12. Villarroya-Beltri C, Gutiérrez-Vázquez C, Sánchez-Cabo F, Perez-Hernández D, Vázquez J, Martin-Cofreces N, Martinez-Herrera D J, Pascual-Montano A, Mittelbrunn M, Sánchez-Madrid F. Sumoylated hnRNPA2B1 controls the sorting of miRNAs into exosomes through binding to specific motifs. Nature Communications. 2013; 4(1):2980. doi: 10.1038/ncomms3980.
13. Hung M E, Leonard J N. A platform for actively loading cargo RNA to elucidate limiting steps in EV-mediated delivery. J Extracell Vesicles. 2016; 5:31027-. doi: 10.3402/jev.v5.31027. PubMed PMID: 27189348.
14. Alvarez-Erviti L, Seow Y, Yin H, Betts C, Lakhal S, Wood M J A. Delivery of siRNA to the mouse brain by systemic injection of targeted exosomes. Nature Biotechnology. 2011; 29(4):341-5. doi: 10.1038/nbt.1807.
15. Kojima R, Bojar D, Rizzi G, Hamri G C-E, El-Baba M D, Saxena P, Auslander S, Tan K R, Fussenegger M. Designer exosomes produced by implanted cells intracerebrally deliver therapeutic cargo for Parkinson's disease treatment. Nature Communications. 2018; 9(1):1305.
16. Bartoszewski R, Sikorski A F. Editorial focus: understanding off-target effects as the key to successful RNAi therapy. Cellular & Molecular Biology Letters. 2019; 24(1):69. doi: 10.1186/s11658-019-0196-3.
17. Herrera-Carrillo E, Berkhout B. Dicer-independent processing of small RNA duplexes: mechanistic insights and applications. Nucleic Acids Research. 2017; 45(18):10369-79. doi: 10.1093/nar/gkx779% J Nucleic Acids Research.
18. Fellmann C, Hoffmann T, Sridhar V, Hopfgartner B, Muhar M, Roth M, Lai Dan Y, Barbosa Ines A M, Kwon Jung S, Guan Y, Sinha N, Zuber J. An Optimized microRNA Backbone for Effective Single-Copy RNAi. Cell Reports. 2013; 5(6):1704-13. doi: https://doi.org/10.1016/j.celrep.2013.11.020.
19. Gibbings D J, Ciaudo C, Erhardt M, Voinnet O. Multivesicular bodies associate with components of miRNA effector complexes and modulate miRNA activity. Nature Cell Biology. 2009; 11(9):1143-9. doi: 10.1038/ncb1929.
20. McKenzie A J, Hoshino D, Hong N H, Cha D J, Franklin J L, Coffey R J, Patton J G, Weaver A M. KRAS-MEK Signaling Controls Ago2 Sorting into Exosomes. Cell Reports. 2016; 15(5):978-87. doi: https://doi.org/10.1016/j.celrep.2016.03.085.
21. Iavello A, Frech V S L, Gai C, Deregibus M C, Quesenberry P J, Camussi G. Role of Alix in miRNA packaging during extracellular vesicle biogenesis. Int J Mol M ed. 2016; 37(4):958-66. doi: 10.3892/ijmm.2016.2488.
22. Squadrito Mario L, Baer C, Burdet F, Maderna C, Gilfillan Gregor D, Lyle R, Ibberson M, De Palma M. Endogenous RNAs Modulate MicroRNA Sorting to Exosomes and Transfer to Acceptor Cells. Cell Reports. 2014; 8(5):1432-46. doi: https://doi.org/10.1016/j.celrep.2014.07.035.
23. Koppers-Lalic D, Hackenberg M, Bijnsdorp Irene V, van Eijndhoven Monique A J, Sadek P, Sie D, Zini N, Middeldorp Jaap M, Ylstra B, de Menezes Renee X, Würdinger T, Meijer Gerrit A, Pegtel D M. Nontemplated Nucleotide Additions Distinguish the Small RNA Composition in Cells from Exosomes. Cell Reports. 2014; 8(6):1649-58. doi: https://doi.org/10.1016/j.celrep.2014.08.027.
24. Horman Shane R, Janas Maja M, Litterst C, Wang B, MacRae Ian J, Sever Mary J, Morrissey David V, Graves P, Luo B, Umesalma S, Qi Hank H, Miraglia Loren J, Novina Carl D, Orth Anthony P. Akt-Mediated Phosphorylation of Argonaute 2 Downregulates Cleavage and Upregulates Translational Repression of MicroRNA Targets. Molecular Cell. 2013; 50(3):356-67. doi: https://doi.org/10.1016/j.molcel.2013.03.015.
25. Wiklander O P B, Nordin J Z, O'Loughlin A, Gustafsson Y, Corso G, Mäger I, Vader P, Lee Y, Sork H, Seow Y, Heldring N, Alvarez-Erviti L, Smith C E, Le Blanc K, Macchiarini P, Jungebluth P, Wood M J A, Andaloussi S E. Extracellular vesicle in vivo biodistribution is determined by cell source, route of administration and targeting 2015; 4(1):26316. doi: https://doi.org/10.3402/jev.v4.26316.
26. Shen Z, Huang W, Liu J, Tian J, Wang S, Rui K. Effects of Mesenchymal Stem Cell-Derived Exosomes on Autoimmune Diseases 2021; 12. doi: 10.3389/fimmu.2021.749192.
27. Dooley K, McConnell R E, Xu K, Lewis N D, Haupt S, Youniss M R, Martin S, Sia C L, McCoy C, Moniz R J, Burenkova O, Sanchez-Salazar J, Jang S C, Choi B, Harrison R A, Houde D, Burzyn D, Leng C, Kirwin K, Ross N L, Finn J D, Gaidukov L, Economides K D, Estes S, Thornton J E, Kulman J D, Sathyanarayanan S, Williams D E. A versatile platform for generating engineered extracellular vesicles with defined therapeutic properties. Molecular Therapy. 2021; 29(5):1729-43. doi: 10.1016/j.ymthe.2021.01.020.
28. Liu Y P, Schopman N C T, Berkhout B. Dicer-independent processing of short hairpin RNAs. Nucleic Acids Research. 2013; 41(6):3723-33. doi: 10.1093/nar/gkt036% J Nucleic Acids Research.
29. Théry C, Witwer K W, Aikawa E, Alcaraz M J, Anderson J D, Andriantsitohaina R, Antoniou A, Arab T, Archer F, Atkin-Smith G K, Ayre D C, Bach J-M, Bachurski D, Baharvand H, Balaj L, Baldacchino S, Bauer N N, Baxter A A, Bebawy M, Beckham C, Bedina Zavec A, Benmoussa A, Berardi A C, Bergese P, Bielska E, Blenkiron C, Bobis-Wozowicz S, Boilard E, Boireau W, Bongiovanni A, Borras F E, Bosch S, Boulanger C M, Breakefield X, Breglio A M, Brennan M A, Brigstock D R, Brisson A, Broekman M L, Bromberg J F, Bryl-Górecka P, Buch S, Buck A H, Burger D, Busatto S, Buschmann D, Bussolati B, Buzás E I, Byrd J B, Camussi G, Carter D R, Caruso S, Chamley L W, Chang Y-T, Chen C, Chen S, Cheng L, Chin A R, Clayton A, Clerici S P, Cocks A, Cocucci E, Coffey R J, Cordeiro-da-Silva A, Couch Y, Coumans F A, Coyle B, Crescitelli R, Criado M F, D'Souza-Schorey C, Das S, Datta Chaudhuri A, de Candia P, De Santana Junior E F, De Wever O, del Portillo H A, Demaret T, Deville S, Devitt A, Dhondt B, Di Vizio D, Dieterich L C, Dolo V, Dominguez Rubio A P, Dominici M, Dourado M R, Driedonks T A, Duarte F V, Duncan H M, Eichenberger R M, Ekström K, EL Andaloussi S, Elie-Caille C, Erdbrügger U, Falcón-Pérez J M, Fatima F, Fish J E, Flores-Bellver M, Försönits A, Frelet-Barrand A, Fricke F, Fuhrmann G, Gabrielsson S, Gámez-Valero A, Gardiner C, Gärtner K, Gaudin R, Gho Y S, Giebel B, GilbertC, Gimona M, Giusti I, Goberdhan D C, Görgens A, Gorski S M, Greening D W, Gross J C, Gualerzi A, Gupta G N, Gustafson D, Handberg A, Haraszti R A, Harrison P, Hegyesi H, Hendrix A, Hill A F, Hochberg F H, Hoffmann K F, Holder B, Holthofer H, Hosseinkhani B, Hu G, Huang Y, Huber V, Hunt S, Ibrahim A G-E, Ikezu T, Inal J M, Isin M, Ivanova A, Jackson H K, Jacobsen S, Jay S M, Jayachandran M, Jenster G, Jiang L, Johnson S M, Jones J C, Jong A, Jovanovic-Talisman T, Jung S, Kalluri R, Kano S-i, Kaur S, Kawamura Y, Keller E T, Khamari D, Khomyakova E, Khvorova A, Kierulf P, Kim K P, Kislinger T, Klingeborn M, Klinke II D J, Kornek M, Kosanovid M M, Kovics A F, Kramer-Albers E-M, Krasemann S, Krause M, Kurochkin I V, Kusuma G D, Kuypers S, Laitinen S, Langevin S M, Languino L R, Lannigan J, Lässer C, Laurent L C, Lavieu G, Lázaro-Ibáñez E, Le Lay S, Lee M-S, Lee Y X F, Lemos D S, Lenassi M, Leszczynska A, Li I T, Liao K, Libregts S F, Ligeti E, Lim R, Lim S K, Line A, Linnemannstons K, Llorente A, Lombard C A, Lorenowicz M J, Lörincz A M, Lötvall J, Lovett J, Lowry M C, Loyer X, Lu Q, Lukomska B, Lunavat T R, Maas S L, Malhi H, Marcilla A, Mariani J, Mariscal J, Martens-Uzunova E S, Martin-Jaular L, Martinez M C, Martins V R, Mathieu M, Mathivanan S, Maugeri M, McGinnis L K, McVey M J, Meckes Jr D G, Meehan K L, Mertens I, Minciacchi V R, Möller A, Møller Jørgensen M, Morales-Kastresana A, Morhayim J, Mullier F, Muraca M, Musante L, Mussack V, Muth D C, Myburgh K H, Najrana T, Nawaz M, Nazarenko I, Nejsum P, Neri C, Neri T, Nieuwland R, Nimrichter L, Nolan J P, Nolte-'t Hoen E N, Noren Hooten N, O'Driscoll L, O'Grady T, O'Loghlen A, Ochiya T, Olivier M, Ortiz A, Ortiz L A, Osteikoetxea X, Øtergaard O, Ostrowski M, Park J, Pegtel D M, Peinado H, Perut F, Pfaffl M W, Phinney D G, Pieters B C, Pink R C, Pisetsky D S, Pogge von Strandmann E, Polakovicova I, Poon I K, Powell B H, Prada I, Pulliam L, Quesenberry P, Radeghieri A, Raffai R L, Raimondo S, Rak J, Ramirez M I, Raposo G, Rayyan M S, Regev-Rudzki N, Ricklefs F L, Robbins P D, Roberts D D, Rodrigues S C, Rohde E, Rome S, Rouschop K M, Rughetti A, Russell A E, Saá P, Sahoo S, Salas-Huenuleo E, Sánchez C, Saugstad J A, Saul M J, Schiffelers R M, Schneider R, Schoyen T H, Scott A, Shahaj E, Sharma S, Shatnyeva O, Shekari F, Shelke G V, Shetty A K, Shiba K, Siljander P R-M, Silva A M, Skowronek A, Snyder II O L, Soares R P, Sódar BW, Soekmadji C, Sotillo J, Stahl P D, Stoorvogel W, Stott S L, Strasser E F, Swift S, Tahara H, Tewari M, Timms K, Tiwari S, Tixeira R, Tkach M, Toh W S, Tomasini R, Torrecilhas A C, Tosar J P, Toxavidis V, Urbanelli L, Vader P, van Balkom B W, van der Grein S G, Van Deun J, van Herwijnen M J, Van Keuren-Jensen K, van Niel G, van Royen M E, van Wijnen A J, Vasconcelos M H, Vechetti Jr I J, Veit T D, Vella L J, Velot É, Verweij F J, Vestad B, Vinas J L, Visnovitz T, Vukman K V, Wahlgren J, Watson D C, Wauben M H, Weaver A, Webber J P, Weber V, Wehman A M, Weiss D J, Welsh J A, Wendt S, Wheelock A M, Wiener Z, Witte L, Wolfram J, Xagorari A, Xander P, Xu J, Yan X, Yáñez-Mó M, Yin H, Yuana Y, Zappulli V, Zarubova J, Zekas V, Zhang J-y, Zhao Z, Zheng L, Zheutlin A R, Zickler A M, Zimmermann P, Zivkovic A M, Zocco D, Zuba-Surma E K. Minimal information for studies of extracellular vesicles 2018 (MISEV2018): a position statement of the International Society for Extracellular Vesicles and update of the MISEV2014 guidelines2018; 7(1):1535750. doi: https://doi.org/10.1080/20013078.2018.1535750.
30. Lee E J, Choi Y, Lee H J, Hwang D W, Lee D S. Human neural stem cell-derived extracellular vesicles protect against Parkinson's disease pathologies. Journal of Nanobiotechnology. 2022; 20(1):198. doi: 10.1186/s12951-022-01356-2.
31. Kuhlmann K, Cieselski M, Schumann J. Relative versus absolute RNA quantification: a comparative analysis based on the example of endothelial expression of vasoactive receptors. Biological Procedures Online. 2021; 23(1):6. doi: 10.1186/s12575-021-00144-w.
32. Geekiyanage H, Rayatpisheh S, Wohlschlegel J A, Brown R, Ambros V. Extracellular microRNAs in human circulation are associated with miRISC complexes tha tare accessible to anti-AGO2 antibody and can bind target mimic oligonucleotides 2020; 117(39):24213-23. doi: doi:10.1073/pnas.2008323117.
33. MacRae I J, Ma E, Zhou M, Robinson C V, Doudna J A. <i>In vitro</i> reconstitution of the human RISC-loading complex 2008; 105(2):512-7. doi: doi:10.1073/pnas.0710869105.
34. Lennon K M, Wakefield D L, Maddox A L, Brehove M S, Willner A N, Garcia-Mansfield K, Meechoovet B, Reiman R, Hutchins E, Miller M M, Goel A, Pirrotte P, Van Keuren-Jensen K, Jovanovic-Talisman T. Single molecule characterization of individual extracellular vesicles from pancreatic cancer 2019; 8(1):1685634. doi: https://doi.org/10.1080/20013078.2019.1685634.
35. Bansal S, McGilvrey M, Garcia-Mansfield K, Sharma R, Bremner R M, Smith M A, Hachem R, Pirrotte P, Mohanakumar T. Global Proteomics Analysis of Circulating Extracellular Vesicles Isolated from Lung Transplant Recipients. ACS Omega. 2020; 5(24):14360-9. doi: 10.1021/acsomega.0c00859.
36. Ashburner M, Ball C A, Blake J A, Botstein D, Butler H, Cherry J M, Davis A P, Dolinski K, Dwight S S, Eppig J T, Harris M A, Hill D P, Issel-Tarver L, Kasarskis A, Lewis S, Matese J C, Richardson J E, Ringwald M, Rubin G M, Sherlock G. Gene Ontology: tool for the unification of biology. Nature Genetics. 2000; 25(1):25-9. doi: 10.1038/75556.
37. Consortium TGO. The Gene Ontology resource: enriching a GOld mine. Nucleic Acids Research. 2020; 49(D1):D325-D34. doi: 10.1093/nar/gkaa1113% J Nucleic Acids Research.
38. Glisovic-Aplenc T, Gill S, Spruce L A, Smith I R, Fazelinia H, Shestova O, Ding H, Tasian S K, Aplenc R, Seeholzer S H. Improved surfaceome coverage with a label-free nonaffinity-purified workflow. Proteomics. 2017; 17(7). Epub 2017/01/25. doi: 10.1002/pmic.201600344. PubM ed PMID: 28116781; PMCID: PMC5518111.
39. Vu L, Ghosh A, Tran C, Tebung W A, Sidibe H, Garcia-Mansfield K, David-Dirgo V, Sharma R, Pirrotte P, Bowser R, Vande Velde C. Defining the Caprin-1 Interactome in Unstressed and Stressed Conditions. Journal of Proteome Research. 2021; 20(6):3165-78. doi: 10.1021/acs.jproteome.lc00016.
40. Tanwar V S, Reddy M A, Das S, Samara V A, Abdollahi M, Dey S, Malek V, Ganguly R, Stapleton K, Lanting L, Pirrotte P, Natarajan R. Palmitic Acid& #x2013; Induced Long Noncoding RNA <i>PARAIL</i> Regulates Inflammation via Interaction With RNA-Binding Protein ELAVL1 (ELAV Like RNA-Binding Protein 1) in Monocytes and Macrophages; 0(0). doi: doi:10.1161/ATV BAHA.122.318536.
41. Boehringer A, Garcia-Mansfield K, Singh G, Bakkar N, Pirrotte P, Bowser R. ALS Associated Mutations in Matrin 3 Alter Protein-Protein Interactions and Impede mRNA Nuclear Export. Scientific Reports. 2017; 7(1):14529. doi: 10.1038/s41598-017-14924-6.
42. Li Y, Collins M, An J, Geiser R, Tegeler T, Tsantilas K, Garcia K, Pirrotte P, Bowser R. Immunoprecipitation and mass spectrometry defines an extensive RBM 45 protein-protein interaction network. Brain Research. 2016; 1647:79-93. doi: https://doi.org/10.1016/j.brainres.2016.02.047.
43. Teo G, Liu G, Zhang J, Nesvizhskii A I, Gingras A-C, Choi H. SAINT express: Improvements and additional features in Significance Analysis of INTeractome software. Journal of Proteomics. 2014; 100:37-43. doi: https://doi.org/10.1016/j.jprot.2013.10.023.
44. Villamizar O, Waters S A, Scott T, Grepo N, Jaffe A, Morris K V. Mesenchymal Stem Cell exosome delivered Zinc Finger Protein activation of cystic fibrosis transmembrane conductance regulator. J Extracell Vesicle. 2021; 10(3):e12053. doi: https://doi.org/10.1002/jev2.12053.
45. Kaadt E, Alsing S, Cecchi C R, Damgaard C K, Corydon T J, Aagaard L. Efficient Knockdown and Lack of Passenger Strand Activity by Dicer-Independent shRNAs Expressed from Pol II-Driven MicroRNA Scaffolds. Molecular Therapy—Nucleic Acids. 2019; 14:318-28. doi: https://doi.org/10.1016/j.omtn.2018.11.013.
46. Boelens W C, Palacios I, Mattaj I W. Nuclear retention of RNA as a mechanism for localization. Rna. 1995; 1(3):273-83. Epub 1995/05/01. PubM ed PMID: 7489499; PMCID: PMC1369080.
47. Gee P, Lung M S Y, Okuzaki Y, Sasakawa N, Iguchi T, Makita Y, Hozumi H, Miura Y, Yang L F, Iwasaki M, Wang X H, Waller M A, Shirai N, Abe Y O, Fujita Y, Watanabe K, Kagita A, Iwabuchi K A, Yasuda M, Xu H, Noda T, Komano J, Sakurai H, Inukai N, Hotta A. Extracellular nanovesicles for packaging of CRISPR-Cas9 protein and sgRNA to induce therapeutic exon skipping. Nature Communications. 2020; 11(1):1334. doi: 10.1038/s41467-020-14957-y.
48. Rüdel S, Wang Y, Lenobel R, Körner R, Hsiao H-H, Urlaub H, Patel D, Meister G. Phosphorylation of human Argonaute proteins affects small RNA binding. Nucleic Acids Research. 2010; 39(6):2330-43. doi: 10.1093/nar/gkq1032% J Nucleic Acids Research.
49. Colombo M, Moita C, van Niel G, Kowal J, Vigneron J, Benaroch P, Manel N, Moita L F, Thery C, Raposo G. Analysis of ESCRT functions in exosome biogenesis, composition and secretion highlights the heterogeneity of extracellular vesicles. Journal of Cell Science. 2013; 126(24):5553-65. doi: 10.1242/jcs.128868% J Journal of Cell Science.
50. Xu X, Chan A K N, Li M, Liu Q, Mattson N, Pangeni Pokharel S, Chang W-H, Yuan Y-C, Wang J, Moore R E, Pirrotte P, Wu J, Su R, Müschen M, Rosen S T, Chen J, Y ang L, Chen C-W. ACTR5 controls CDKN2A and tumor progression in an IN080-independent manner 2022; 8(51):eadc8911. doi: doi:10.1126/sciadv.adc8911.
51. Santangelo L, Giurato G, Cicchini C, Montaldo C, Mancone C, Tarallo R, Battistelli C, Alonzi T, Weisz A, Tripodi M. The RNA-Binding Protein SYNCRIP Is a Component of the Hepatocyte Exosomal Machinery Controlling MicroRNA Sorting. Cell Reports. 2016; 17(3):799-808. doi: https://doi.org/10.1016/j.celrep.2016.09.031.
52. Temoche-Diaz M M, Shurtleff M J, Nottingham R M, Yao J, Fadadu R P, Lambowitz A M, Schekman R. Distinct mechanisms of microRNA sorting into cancer cell-derived extracellular vesicle subtypes. eLife. 2019; 8:e47544. doi: 10.7554/eLife.47544.
53. Luo X, Jean-Toussaint R, Sacan A, Ajit S K. Differential RNA packaging into small extracellular vesicles by neurons and astrocytes. Cell Communication and Signaling. 2021; 19(1):75. doi: 10.1186/s12964-021-00757-4.
54. Garcia-Martin R, Wang G, Brandão B B, Zanotto T M, Shah S, Kumar Patel S, Schilling B, Kahn C R. MicroRNA sequence codes for small extracellular vesicle release and cellular retention. Nature. 2022; 601(7893):446-51. doi: 10.1038/s41586-021-04234-3.
55. Denti M A, Rosa A, Sthandier O, DeAngelis F G, Bozzoni I. A New Vector, Based on the PolII Promoter of the U1 snRNA Gene, for the Expression of siRNAs in Mammalian Cells. Molecular Therapy. 2004; 10:191-9.
56. Shen J, Xia W, Khotskaya Y B, Huo L, Nakanishi K, Lim S-O, Du Y, Wang Y, Chang W-C, Chen C-H, Hsu J L, Wu Y, Lam Y C, James B P, Liu X, Liu C-G, Patel D J, Hung M-C. EGFR modulates microRNA maturation in response to hypoxia through phosphorylation of AGO2. Nature. 2013; 497(7449):383-7. doi: 10.1038/nature12080.
57. Zeng Y, Sankala H, Zhang X, Graves Paul R. Phosphorylation of Argonaute 2 at serine-387 facilitates its localization to processing bodies. Biochemical Journal. 2008; 413(3):429-36. doi: 10.1042/BJ 20080599% J Biochemical Journal.
58. Yang M, Haase Astrid D, Huang F-K, Coulis G, Rivera Keith D, Dickinson Bryan C, Chang Christopher J, Pappin Darryl J, Neubert Thomas A, Hannon Gregory J, Boivin B, Tonks Nicholas K. Dephosphorylation of Tyrosine 393 in Argonaute 2 by Protein Tyrosine Phosphatase 1B Regulates Gene Silencing in Oncogenic RAS-Induced Senescence. Molecular Cell. 2014; 55(5):782-90. doi: https://doi.org/10.1016/j.molcel.2014.07.018.
59. Shankar S, Pitchiaya S, Malik R, Kothari V, Hosono Y, Yocum Anastasia K, Gundlapalli H, White Y, Firestone A, Cao X, Dhanasekaran Saravana M, Stuckey Jeanne A, Bollag G, Shannon K, Walter Nils G, Kumar-Sinha C, Chinnaiyan Arul M. KRAS Engages AGO2 to Enhance Cellular Transformation. Cell Reports. 2016; 14(6):1448-61. doi: https://doi.org/10.1016/j.celrep.2016.01.034.
60. Martins-Marques T, Costa M C, Catarino S, Simoes I, Aasen T, Enguita F J, Girao H. Cx43-mediated sorting of miRNAs into extracellular vesicles 2022; 23(7):e54312. doi: https://doi.org/10.15252/embr.202154312.
61. Kramer M F. Stem-Loop RT-qPCR for miRNAs2011; 95(1):15.0.1-.0. doi: https://doi.org/10.1002/0471142727.mb1510s95.
62. Characterization of Complete Particles (VSV-G/SIN-GFP) and Empty Particles (VSV-G/EMPTY) in Human Immunodeficiency Virus Type 1-Based Lentiviral Products for Gene Therapy: Potential Applications for Improvement of Product Quality and Safety 2008; 19(5):475-86. doi: 10.1089/hum.2007.119. PubM ed PMID: 18412516.
63. Wright J F. AAV Empty Capsids: For Better or for Worse? Molecular Therapy. 2014; 22(1):1-2. doi: https://doi.org/10.1038/mt.2013.268.
64. Albanese M, Chen Y-F A, Hüls C, Gärtner K, Tagawa T, Mejias-Perez E, Keppler O T, Göbel C, Zeidler R, Shein M, Schütz A K, Hammerschmidt W. MicroRNAs are minor constituents of extracellular vesicles that are rarely delivered to target cells. PLOS Genetics. 2021; 17(12):e1009951. doi: 10.1371/journal.pgen.1009951.
65. Shrivastava S, Ray RM, Holguin L, Echavarria L, Grepo N, Scott T A, Burnett J, Morris K V. Exosome-mediated stable epigenetic repression of HIV-1. Nature Communications. 2021; 12(1):5541. doi: 10.1038/s41467-021-25839-2.
66. Kim H, Kim M, Im S-K, Fang S. Mouse Cre-LoxP system: general principles to determine tissue-specific roles of target genes. lar. 2018; 34(4):147-59. doi: 10.5625/lar.2018.34.4.147.
67. Scott T A, Supramaniam A, Idris A, Cardoso A A, Shrivastava S, Kelly G, Grepo N A, Soemardy C, Ray R M, McMillan N A J, Morris K V. Engineered extracellular vesicles directed to the spike protein inhibit SARS-CoV-2. Molecular Therapy—Methods & Clinical Development. 2022; 24:355-66. doi: 10.1016/j.omtm.2022.01.015.
68. Segel M, Lash B, Song J, Ladha A, Liu C C, Jin X, Mekhedov S L, Macrae R K, Koonin E V, Zhang F. Mammalian retrovirus-like protein PEG10 packages its own mRNA and can be pseudotyped for mRNA delivery 2021; 373(6557):882-9. doi: doi:10.1126/science.abg6155.
69. Zhu W, Zhou B, Zhao C, Ba Z, Xu H, Yan X, Liu W, Zhu B, Wang L, Ren C. Myoferlin, a multifunctional protein in normal cells, has novel and key roles in various cancers 2019; 23(11):7180-9. doi: https://doi.org/10.1111/jcmm.14648.
70. Scott T A, O'Meally D, Grepo N A, Soemardy C, Lazar D C, Zheng Y, Weinberg M S, Planelles V, Morris K V. Broadly active zinc finger protein-guided transcriptional activation of HIV-1. Molecular Therapy—Methods & Clinical Development. 2021; 20:18-29. doi: https://doi.org/10.1016/j.omtm.2020.10.018.

### EXAMPLE 3 REFERENCES

1. Kojima, R., Bojar, D., Rizzi, G., Hamri, G. C.-E., El-Baba, M. D., Saxena, P., Auslander, S., Tan, K. R. and Fussenegger, M. (2018) Designer exosomes produced by implanted cells intracerebrally deliver therapeutic cargo for Parkinson's disease treatment. Nature Communications, 9, 1305.
2. Scott, T., Soemardy, C. and Morris, K. V. (2020) Development of a Facile Approach for Generating Chemically Modified CRISPR/Cas9 RNA. Molecular Therapy—Nucleic Acids, 19, 1176-1185.
3. Dooley, K., McConnell, R. E., Xu, K., Lewis, N. D., Haupt, S., Youniss, M. R., Martin, S., Sia, C. L., McCoy, C., Moniz, R. J. et al. (2021) A versatile platform for generating engineered extracellular vesicles with defined therapeutic properties. Molecular Therapy, 29, 1729-1743.

TABLE 1
shRNA-GFP sequences

| | | SEQ ID | shRNA Sequence | SEQ ID |
|---|---|---|---|---|
| shRNA | Sequence (5′-3′) | NO | (5′-3′) | NO |
| U6-miR451- | GAGGGCCTATTTCCCATGATTCCTTCATAT | 1 | GAUCUUACUGACUGCC | 42 |
| GFP | TTGCATATACGATACAAGGCTGTTAGAGA | | AGGGCACUUGGGAAU | |
| | GATAATTGGAATTAATTTGACTGTAAACA | | GGCAAGGAUGAACUUC | |
| | CAAAGATATTAGTACAAAATACGTGACG | | AGGGUCAGCUUGCGU | |
| | TAGAAAGTAATAATTTCTTGGGTAGTTTG | | UGACCCUGAAGUUCAU | |
| | CAGTTTTAAAATTATGTTTTAAAATGGAC | | UCUUGCUAUACCCAGA | |
| | TATCATATGCTTACCGTAACTTGAAAGTA | | AAACGUGCCUU | |
| | TTTCGATTTCTTGGCTTTATATATCTTGTG | | | |
| | GAAAGGACGAAGATCCGATCTTACTGACT | | | |
| | GCCAGGGCACTTGGGAATGGCAAGGATG | | | |
| | AACTTCAGGGTCAGCTTGCGTTGACCCTG | | | |
| | AAGTTCATTCTTGCTATACCCAGAAAACGT | | | |
| | GCCTTTTTT | | | |
| U6-miR451- | GAGGGCCTATTTCCCATGATTCCTTCATAT | 2 | GCCAGGGCACUUGGGA | 43 |
| GFP-2 | TTGCATATACGATACAAGGCTGTTAGAGA | | AUGGCAAGGAUGAACU | |
| | GATAATTGGAATTAATTTGACTGTAAACA | | UCAGGGUCAGCUUGCG | |
| | CAAAGATATTAGTACAAAATACGTGACG | | UUGACCCUGAAGUUCA | |
| | TAGAAAGTAATAATTTCTTGGGTAGTTTG | | CUCUUGCUAUACCCAG | |
| | CAGTTTTAAAATTATGTTTTAAAATGGAC | | AAAACGUGCCUU | |
| | TATCATATGCTTACCGTAACTTGAAAGTA | | | |
| | TTTCGATTTCTTGGCTTTATATATCTTGTG | | | |
| | GAAAGGACGAAGATCCGCCAGGGCACTT | | | |
| | GGGAATGGCAAGGATGAACTTCAGGGTC | | | |
| | AGCTTGCGTTGACCCTGAAGTTCACTCTTG | | | |
| | CTATACCCAGAAAACGTGCCTTTTTT | | | |
| U6-miR451- | GAGGGCCTATTTCCCATGATTCCTTCATAT | 3 | GAUGAACUUCAGGGUC | 44 |
| GFP-3 | TTGCATATACGATACAAGGCTGTTAGAGA | | AGCUUGGCUGACCCUG | |
| | GATAATTGGAATTAATTTGACTGTAAACA | | AAGUUCAUCUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCGATGAACTTCAG | | | |
| | GGTCAGCTTGGCTGACCCTGAAGTTCATCT | | | |
| | TTTTT | | | |
| U6-miR-16- | GAGGGCCTATTTCCCATGATTCCTTCATAT | 4 | AUGAACUUCAGGGUCA | 45 |
| GFP | TTGCATATACGATACAAGGCTGTTAGAGA | | GCUUGCUUAAGAUUC | |
| | GATAATTGGAATTAATTTGACTGTAAACA | | UAAAAUUAUGUAAGC | |
| | CAAAGATATTAGTACAAAATACGTGACG | | UGUCCUCUGAAGUUCA | |
| | TAGAAAGTAATAATTTCTTGGGTAGTTTG | | UU | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCTTAAGATTCTAAAATTATGT | | | |
| | AAGCTGTCCTCTGAAGTTCATTTTTTT | | | |
| U6-miR-16- | GAGGGCCTATTTCCCATGATTCCTTCATAT | 5 | GUCAGCAGUGCCUAUG | 46 |
| GFPb | TTGCATATACGATACAAGGCTGTTAGAGA | | AACUUCAGGGUCAGCU | |
| | GATAATTGGAATTAATTTGACTGTAAACA | | UGCUUAAGAUUCUAAA | |
| | CAAAGATATTAGTACAAAATACGTGACG | | AUUAUGUAAGCUGAC | |
| | TAGAAAGTAATAATTTCTTGGGTAGTTTG | | GCUCGAAGUUCAUAGU | |
| | CAGTTTTAAAATTATGTTTTAAAATGGAC | | AAGGUUGACUU | |
| | TATCATATGCTTACCGTAACTTGAAAGTA | | | |
| | TTTCGATTTCTTGGCTTTATATATCTTGTG | | | |
| | GAAAGGACGAAGATCCGTCAGCAGTGCC | | | |
| | TATGAACTTCAGGGTCAGCTTGCTTAAGAT | | | |
| | TCTAAAATTATGTAAGCTGACGCTCGAAG | | | |
| | TTCATAGTAAGGTTGACTTTTTTT | | | |
| U6-Dicersh- | GAGGGCCTATTTCCCATGATTCCTTCATAT | 6 | GCAGAUGAACUUCAGG | 47 |
| GFP | TTGCATATACGATACAAGGCTGTTAGAGA | | GUCAGCUUGGCUGACC | |
| | GATAATTGGAATTAATTTGACTGTAAACA | | CUGAAGUUCAUCUGCU | |
| | CAAAGATATTAGTACAAAATACGTGACG | | U | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCGCAGATGAACTT | | | |
| | CAGGGTCAGCTTGGCTGACCCTGAAGTTC | | | |
| | ATCTGCTTTTTT | | | |
| U6-AgoSh- | GAGGGCCTATTTCCCATGATTCCTTCATAT | 7 | AUGAACUUCAGGGUCA | 48 |
| GFPa | TTGCATATACGATACAAGGCTGTTAGAGA | | GCUUGGCUGACCCUGA | |
| | GATAATTGGAATTAATTTGACTGTAAACA | | AGUUCAUCUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGGCTGACCCTGAAGTTCATCTT | | | |
| | TTTT | | | |
| U6-AgoSh- | GAGGGCCTATTTCCCATGATTCCTTCATAT | 8 | AUGAACUUCAGGGUCA | 49 |
| GFPb | TTGCATATACGATACAAGGCTGTTAGAGA | | GCUUGCGCUGACCCUG | |
| | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCCTGAAGTTCACTT | | | |
| | TTTTT | | | |
| U6-shAgo- | GAGGGCCTATTTCCCATGATTCCTTCATAT | 9 | AUGAACUUCAGGGUCA | 50 |
| GFPc | TTGCATATACGATACAAGGCTGTTAGAGA | | GCUUGCGCUGACCCUG | |
| | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCCTGAAGTTCACTT | | | |
| | TTTTT | | | |
| U6-shAgo- | GAGGGCCTATTTCCCATGATTCCTTCATAT | 10 | AUGAACUUCAGGGUCA | 51 |
| GFPd | TTGCATATACGATACAAGGCTGTTAGAGA | | GCUUGCGCUGACCCUG | |
| | GATAATTGGAATTAATTTGACTGTAAACA | | AAGUUCACUCUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCCTGAAGTTCACTC | | | |
| | TTTTTT | | | |
| U6-AgoSh- | GAGGGCCTATTTCCCATGATTCCTTCATAT | 11 | AUGAACUUCAGGGUCA | 52 |
| GFPb-5′A- | TTGCATATACGATACAAGGCTGTTAGAGA | | GCUUGCGCUGACCCUG | |
| 3′A | GATAATTGGAATTAATTTGACTGTAAACA | | AAGUUCAAUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCCTGAAGTTCAATT | | | |
| | TTTT | | | |
| U6-AgoSh- | GAGGGCCTATTTCCCATGATTCCTTCATAT | 12 | AUGAACUUCAGGGUCA | 53 |
| GFPb-5′A- | TTGCATATACGATACAAGGCTGTTAGAGA | | GCUUGCGCUGACCCUG | |
| 3′Delta | GATAATTGGAATTAATTTGACTGTAAACA | | AAGUUCAUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCCTGAAGTTCATTT | | | |
| | TTT | | | |
| U6-AgoSh- | GAGGGCCTATTTCCCATGATTCCTTCATAT | 13 | AUGAACUUCAGGGUCA | 54 |
| GFPb-5′A- | TTGCATATACGATACAAGGCTGTTAGAGA | | GCUUGCGCUGACCCUG | |
| 3′G | GATAATTGGAATTAATTTGACTGTAAACA | | AAGUUCAGUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCCTGAAGTTCAGTT | | | |
| | TTTT | | | |
| U6-AgoSh- | GAGGGCCTATTTCCCATGATTCCTTCATAT | 14 | AUGAACUUCAGGGUCA | 55 |
| GFPb-5′A- | TTGCATATACGATACAAGGCTGTTAGAGA | | GCUUGCGCUGACCCUG | |
| 3′T | GATAATTGGAATTAATTTGACTGTAAACA | | AAGUUCAUUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCCTGAAGTTCATTT | | | |
| | TTTT | | | |
| U6-AgoSh- | GAGGGCCTATTTCCCATGATTCCTTCATAT | 15 | GUGAACUUCAGGGUCA | 56 |
| GFPb-5′G- | TTGCATATACGATACAAGGCTGTTAGAGA | | GCUUGCGCUGACCCUG | |
| 3′A | GATAATTGGAATTAATTTGACTGTAAACA | | AAGUUCAAUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCGTGAACTTCAGG | | | |
| | GTCAGCTTGCGCTGACCCTGAAGTTCAATT | | | |
| | TTTT | | | |
| U6-AgoSh- | GAGGGCCTATTTCCCATGATTCCTTCATAT | 16 | GUGAACUUCAGGGUCA | 57 |
| GFPb-5′G- | TTGCATATACGATACAAGGCTGTTAGAGA | | GCUUGCGCUGACCCUG | |
| 3′C | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCGTGAACTTCAGG | | | |
| | GTCAGCTTGCGCTGACCCTGAAGTTCACTT | | | |
| | TTTT | | | |
| U6-AgoSh- | GAGGGCCTATTTCCCATGATTCCTTCATAT | 17 | GUGAACUUCAGGGUCA | 58 |
| GFPb-5′G- | TTGCATATACGATACAAGGCTGTTAGAGA | | GCUUGCGCUGACCCUG | |
| 3′delta | GATAATTGGAATTAATTTGACTGTAAACA | | AAGUUCAUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCGTGAACTTCAGG | | | |
| | GTCAGCTTGCGCTGACCCTGAAGTTCATTT | | | |
| | TTT | | | |
| U6-AgoSh- | GAGGGCCTATTTCCCATGATTCCTTCATAT | 18 | GUGAACUUCAGGGUCA | 59 |
| GFPb-5′G- | TTGCATATACGATACAAGGCTGTTAGAGA | | GCUUGCGCUGACCCUG | |
| 3′G | GATAATTGGAATTAATTTGACTGTAAACA | | AAGUUCAGUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCGTGAACTTCAGG | | | |
| | GTCAGCTTGCGCTGACCCTGAAGTTCAGTT | | | |
| | TTTT | | | |
| U6-AgoSh- | GAGGGCCTATTTCCCATGATTCCTTCATAT | 19 | GUGAACUUCAGGGUCA | 60 |
| GFPb-5′G- | TTGCATATACGATACAAGGCTGTTAGAGA | | GCUUGCGCUGACCCUG | |
| 3′T | GATAATTGGAATTAATTTGACTGTAAACA | | AAGUUCAUUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCGTGAACTTCAGG | | | |
| | GTCAGCTTGCGCTGACCCTGAAGTTCATTT | | | |
| | TTTT | | | |
| U6-AgoSh- | GAGGGCCTATTTCCCATGATTCCTTCATAT | 20 | GUGAACUUCAGGGUCA | 61 |
| GFPb-5′G | TTGCATATACGATACAAGGCTGTTAGAGA | | GCUUGCGCUGACCCUG | |
| | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCGTGAACTTCAGG | | | |
| | GTCAGCTTGCGCTGACCCTGAAGTTCACTT | | | |
| | TTTT | | | |
| AgoSh- | GAGGGCCTATTTCCCATGATTCCTTCATAT | 21 | GUGAACUUCAGGGUCA | 62 |
| GFPd-5′G | TTGCATATACGATACAAGGCTGTTAGAGA | | GCUUGCGCUGACCCUG | |
| | GATAATTGGAATTAATTTGACTGTAAACA | | AAGUUCACUCUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCGTGAACTTCAGG | | | |
| | GTCAGCTTGCGCTGACCCTGAAGTTCACTC | | | |
| | TTTTTT | | | |
| U6-shAGo- | GAGGGCCTATTTCCCATGATTCCTTCATAT | 22 | AUGAACUUCAGGGUCA | 63 |
| GFPb-8A | TTGCATATACGATACAAGGCTGTTAGAGA | | GCUUGCGCUGACCCUA | |
| | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCCTAAAGTTCACTT | | | |
| | TTTT | | | |
| U6-shAgo- | GAGGGCCTATTTCCCATGATTCCTTCATAT | 23 | AUGAACUUCAGGGUCA | 64 |
| GFPb-9A | TTGCATATACGATACAAGGCTGTTAGAGA | | GCUUGCGCUGACCCAG | |
| | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCCAGAAGTTCACTT | | | |
| | TTTT | | | |
| U6-AgoSh- | GAGGGCCTATTTCCCATGATTCCTTCATAT | 24 | AUGAACUUCAGGGUCA | 65 |
| GFPb-10-A | TTGCATATACGATACAAGGCTGTTAGAGA | | GCUUGCGCUGACCAUG | |
| | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCATGAAGTTCACTT | | | |
| | TTTT | | | |
| U6-AgoSh- | GAGGGCCTATTTCCCATGATTCCTTCATAT | 25 | AUGAACUUCAGGGUCA | 66 |
| GFPb-11-A | TTGCATATACGATACAAGGCTGTTAGAGA | | GCUUGCGCUGACACUG | |
| | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACACTGAAGTTCACTT | | | |
| | TTTT | | | |
| U6-shAgo- | GAGGGCCTATTTCCCATGATTCCTTCATAT | 26 | AUGAACUUCAGGGUCA | 67 |
| GFP-9A-10A | TTGCATATACGATACAAGGCTGTTAGAGA | | GCUUGCGCUGACCAAG | |
| | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACCAAGAAGTTCACT | | | |
| | TTTTT | | | |
| U6-AgoSh- | GAGGGCCTATTTCCCATGATTCCTTCATAT | 27 | AUGAACUUCAGGGUCA | 68 |
| GFPb-10-11- | TTGCATATACGATACAAGGCTGTTAGAGA | | GCUUGCGCUGACAAUG | |
| A | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACAATGAAGTTCACTT | | | |
| | TTTT | | | |
| U6-shAgo- | GAGGGCCTATTTCCCATGATTCCTTCATAT | 28 | AUGAACUUCAGGGUCA | 69 |
| GFP-9A- | TTGCATATACGATACAAGGCTGTTAGAGA | | GCUUGCGCUGACAAAG | |
| 10A-11A | GATAATTGGAATTAATTTGACTGTAAACA | | AAGUUCACUU | |
| | CAAAGATATTAGTACAAAATACGTGACG | | | |
| | TAGAAAGTAATAATTTCTTGGGTAGTTTG | | | |
| | CAGTTTTAAAATTATGTTTTAAAATGGAC | | | |
| | TATCATATGCTTACCGTAACTTGAAAGTA | | | |
| | TTTCGATTTCTTGGCTTTATATATCTTGTG | | | |
| | GAAAGGACGAAGATCCATGAACTTCAGG | | | |
| | GTCAGCTTGCGCTGACAAAGAAGTTCACT | | | |
| | TTTTT | | | |
| tRNAlys3- | GCCCGGATAGCTCAGTCGGTAGAGCATC | 29 | AUGAACUUCAGGGUCA | 70 |
| Agosh-GFPb | AGACTTTTAATCTGAGGGTCCAGGGTTCA | | GCUUGCGCUGACCCUG | |
| | AGTCCCTGTTCGGGCGATGAACTTCAGGG | | AAGUUCACUU | |
| | TCAGCTTGCGCTGACCCTGAAGTTCACTTT | | | |
| | TTT | | | |
| U1-Pol II- | CTAAGGACCAGCTTCTTTGGGAGAGAAC | 30 | AUGAACUUCAGGGUCA | 71 |
| Ago2- | AGACGCAGGGGGGGGAGGGAAAAAGGG | | GCUUGCGCUGACCCUG | |
| shGFPb | AGAGGCAGACGTCACTTCCCCTTGGCGGC | | AAGUUCAC | |
| | TCTGGCAGCAGATTGGTCGGTTGAGTGG | | | |
| | CAGAAAGGCAGACGGGGACTGGGCAAG | | | |
| | GCACTGTCGGTGACATCACGGACAGGGC | | | |
| | GACTTCTATGTAGATGAGGCAGCGCAGA | | | |
| | GGCTGCTGCTTCGCCACTTGCTGCTTCGCC | | | |
| | ACGAAGGAGTTCCCGTGCCCTGGGAGCG | | | |
| | GGTTCAGGACCGCGGATCGGAAGTGAGA | | | |
| | ATCCCAGCTGTGTGTCAGGGCTGGAAAG | | | |
| | GGCTCGGGAGTGCGCGGGGCAAGTGACC | | | |
| | GTGTGTGTAAAGAGTGAGGCGTATGAGG | | | |
| | CTGTGTCGGGGCAGAGCCCGAAGATCAT | | | |
| | GAACTTCAGGGTCAGCTTGCGCTGACCCT | | | |
| | GAAGTTCACactttctggagtttcaaaagtagac | | | |
| U1-Pol II- | CTAAGGACCAGCTTCTTTGGGAGAGAAC | 31 | AUGAACUUCAGGGUCA | 72 |
| Ago2- | AGACGCAGGGGGGGGAGGGAAAAAGGG | | GCUUGCGCUGACCCUG | |
| shGFPb-T | AGAGGCAGACGTCACTTCCCCTTGGCGGC | | AAGUUCACU | |
| | TCTGGCAGCAGATTGGTCGGTTGAGTGG | | | |
| | CAGAAAGGCAGACGGGGACTGGGCAAG | | | |
| | GCACTGTCGGTGACATCACGGACAGGGC | | | |
| | GACTTCTATGTAGATGAGGCAGCGCAGA | | | |
| | GGCTGCTGCTTCGCCACTTGCTGCTTCGCC | | | |
| | ACGAAGGAGTTCCCGTGCCCTGGGAGCG | | | |
| | GGTTCAGGACCGCGGATCGGAAGTGAGA | | | |
| | ATCCCAGCTGTGTGTCAGGGCTGGAAAG | | | |
| | GGCTCGGGAGTGCGCGGGGCAAGTGACC | | | |
| | GTGTGTGTAAAGAGTGAGGCGTATGAGG | | | |
| | CTGTGTCGGGGCAGAGCCCGAAGATCAT | | | |
| | GAACTTCAGGGTCAGCTTGCGCTGACCCT | | | |
| | GAAGTTCACTactttctggagtttcaaaagtagac | | | |
| U1-Pol II- | CTAAGGACCAGCTTCTTTGGGAGAGAAC | 32 | AUGAACUUCAGGGUCA | 73 |
| Ago2- | AGACGCAGGGGGGGGAGGGAAAAAGGG | | GCUUGCGCUGACCCUG | |
| shGFPb-TT | AGAGGCAGACGTCACTTCCCCTTGGCGGC | | AAGUUCACUU | |
| | TCTGGCAGCAGATTGGTCGGTTGAGTGG | | | |
| | CAGAAAGGCAGACGGGGACTGGGCAAG | | | |
| | GCACTGTCGGTGACATCACGGACAGGGC | | | |
| | GACTTCTATGTAGATGAGGCAGCGCAGA | | | |
| | GGCTGCTGCTTCGCCACTTGCTGCTTCGCC | | | |
| | ACGAAGGAGTTCCCGTGCCCTGGGAGCG | | | |
| | GGTTCAGGACCGCGGATCGGAAGTGAGA | | | |
| | ATCCCAGCTGTGTGTCAGGGCTGGAAAG | | | |
| | GGCTCGGGAGTGCGCGGGGCAAGTGACC | | | |
| | GTGTGTGTAAAGAGTGAGGCGTATGAGG | | | |
| | CTGTGTCGGGGCAGAGCCCGAAGATCAT | | | |
| | GAACTTCAGGGTCAGCTTGCGCTGACCCT | | | |
| | GAAGTTCACTTactttctggagtttcaaaagtagac | | | |
| U1-Pol II- | CTAAGGACCAGCTTCTTTGGGAGAGAAC | 33 | AUGAACUUCAGGGUCA | 74 |
| Ago2- | AGACGCAGGGGGGGGAGGGAAAAAGGG | | GCUUGCGCUGACCCUG | |
| shGFPb-TTT | AGAGGCAGACGTCACTTCCCCTTGGCGGC | | AAGUUCACUUU | |
| | TCTGGCAGCAGATTGGTCGGTTGAGTGG | | | |
| | CAGAAAGGCAGACGGGGACTGGGCAAG | | | |
| | GCACTGTCGGTGACATCACGGACAGGGC | | | |
| | GACTTCTATGTAGATGAGGCAGCGCAGA | | | |
| | GGCTGCTGCTTCGCCACTTGCTGCTTCGCC | | | |
| | ACGAAGGAGTTCCCGTGCCCTGGGAGCG | | | |
| | GGTTCAGGACCGCGGATCGGAAGTGAGA | | | |
| | ATCCCAGCTGTGTGTCAGGGCTGGAAAG | | | |
| | GGCTCGGGAGTGCGCGGGGCAAGTGACC | | | |
| | GTGTGTGTAAAGAGTGAGGCGTATGAGG | | | |
| | CTGTGTCGGGGCAGAGCCCGAAGATCAT | | | |
| | GAACTTCAGGGTCAGCTTGCGCTGACCCT | | | |
| | GAAGTTCACTTTactttctggagtttcaaaagtag | | | |
| | ac | | | |
| H1-shAgo- | TGCAATATTTGCATGTCGCTATGTGTTCTG | 34 | AUGAACUUCAGGGUCA | 75 |
| shGFPb | GGAAATCACCATAAACGTGAAATGTCTTT | | GCUUGCGCUGACCCUG | |
| | GGATTTGGGAATCTTATAAGTTCTGTATG | | AAGUUCACUU | |
| | AGACCACTCAGATCCATGAACTTCAGGGT | | | |
| | CAGCTTGCGCTGACCCTGAAGTTCACTTTT | | | |
| | TT | | | |
| tRNAval- | GTTTTCGTAGTGTAGTGGTTATCACGTGT | 35 | AUGAACUUCAGGGUCA | 76 |
| shAgo-GFPb | GCTTCACACGCACAAGGTCCCCGGTTCGA | | GCUUGCGCUGACCCUG | |
| | ACCCGGGCGAAAACAATGAACTTCAGGG | | AAGUUCACUU | |
| | TCAGCTTGCGCTGACCCTGAAGTTCACTTT | | | |
| | TTT | | | |
| tRNAser- | GAAAATGACTTTGCCACGCTTAGCATGTG | 36 | AUGAACUUCAGGGUCA | 77 |
| shAgo-GFPb | ACGAGGTGGCCGAGTGGTTAAGGCGATG | | GCUUGCGCUGACCCUG | |
| | GACTGCTAATCCATTGTGCTCTGCACGCG | | AAGUUCACUU | |
| | TGGGTTCGAATCCCATCCTCGTCGATGAA | | | |
| | CTTCAGGGTCAGCTTGCGCTGACCCTGAA | | | |
| | GTTCACTTTTTT | | | |
| tRNAgly- | GCATGGGTGGTTCAGTGGTAGAATTCTC | 37 | AUGAACUUCAGGGUCA | 78 |
| shAgo-GFPb | GCCTGCCACGCGGGAGGCCCGGGTTCGA | | GCUUGCGCUGACCCUG | |
| | TTCCCGGCCCATGCAATGAACTTCAGGGT | | AAGUUCACUU | |
| | CAGCTTGCGCTGACCCTGAAGTTCACTTTT | | | |
| | TT | | | |
| tRNAGln- | GGTTCCATGGTGTAATGGTTAGCACTCTG | 38 | AUGAACUUCAGGGUCA | 79 |
| shAgo-GFPb | GACTCTGAATCCAGCGATCCGAGTTCAAA | | GCUUGCGCUGACCCUG | |
| | TCTCGGTGGAACCTATGAACTTCAGGGTC | | AAGUUCACUU | |
| | AGCTTGCGCTGACCCTGAAGTTCACTTTTT | | | |
| | T | | | |
| tRNAPro- | GGCTCGTTGGTCTAGGGGTATGATTCTCG | 39 | AUGAACUUCAGGGUCA | 80 |
| shAgo-GFPb | CTTAGGGTGCGAGAGGTCCCGGGTTCAA | | GCUUGCGCUGACCCUG | |
| | ATCCCGGACGAGCCCATGAACTTCAGGGT | | AAGUUCACUU | |
| | CAGCTTGCGCTGACCCTGAAGTTCACTTTT | | | |
| | TT | | | |
| tRNALeu- | GTCAGGATGGCCGAGTGGTCTAAGGCGC | 40 | AUGAACUUCAGGGUCA | 81 |
| shAgo-GFPb | TGCGTTCAGGTCGCAGTCTACTCTGTAGG | | GCUUGCGCUGACCCUG | |
| | CGTGGGTTCGAATCCCACTTCTGACAATG | | AAGUUCACUU | |
| | AACTTCAGGGTCAGCTTGCGCTGACCCTG | | | |
| | AAGTTCACTTTTTT | | | |
| tRNAAsn- | GCCTCCGTGGCGCAATTGGTTAGCGCGTT | 41 | AUGAACUUCAGGGUCA | 82 |
| shAgo-GFPb | CGGCTGTTAACCGAAAGGTTGGTGGTTC | | GCUUGCGCUGACCCUG | |
| | GAGTCCACCCGGGGGCGATGAACTTCAG | | AAGUUCACUU | |
| | GGTCAGCTTGCGCTGACCCTGAAGTTCAC | | | |
| | TTTTTT | | | |

Bold: small RNA promoter; underlined: Pol III Poly T transcription terminator; lower case: U1 Pol II 3′ Box transcription terminator; italics: predicted UU added on during Pol III transcription termination.

TABLE 2
A go-shG FP including an exosome-specific RNA motif

| | | | | | RNA- |
|---|---|---|---|---|---|
| | | | | | Binding |
| | | SEQ | shRNA | | Protein of |
| | | ID | Sequence | SEQ ID | the Exo- |
| shRNA | Sequence (5′-3′) | NO | (5′-3′) | NO | Motif |
| U6- | GAGGGCCTATTTCCCATGATTCCTTCATATT | 83 | AUGAACUUC | 97 | SYNCRIP |
| AgoSh- | TGCATATACGATACAAGGCTGTTAGAGAG | | AGGGUCAGC | | (NCBI |
| GFPb- | ATAATTGGAATTAATTTGACTGTAAACACA | | UUGCGCUGA | | Gene: |
| hExo | AAGATATTAGTACAAAATACGTGACGTAG | | CCCUGAAGU | | 10492) |
| | AAAGTAATAATTTCTTGGGTAGTTTGCAGT | | UCACGGCUG | | |
| | TTTAAAATTATGTTTTAAAATGGACTATCA | | UU | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACGGCTGTTTTTT | | | | |
| U6- | GAGGGCCTATTTCCCATGATTCCTTCATATT | 84 | AUGAACUUC | 98 | hnRNPA2B1 |
| AgoSh- | TGCATATACGATACAAGGCTGTTAGAGAG | | AGGGUCAGC | | (NCBI |
| GFPb- | ATAATTGGAATTAATTTGACTGTAAACACA | | UUGCGCUGA | | Gene: |
| ExoMotif- | AAGATATTAGTACAAAATACGTGACGTAG | | CCCUGAAGU | | 3181) |
| 1 | AAAGTAATAATTTCTTGGGTAGTTTGCAGT | | UCACGGAGU | | |
| | TTTAAAATTATGTTTTAAAATGGACTATCA | | U | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACGGAGTTTTTT | | | | |
| U6- | GAGGGCCTATTTCCCATGATTCCTTCATATT | 85 | AUGAACUUC | 99 | hnRNPA2B1 |
| AgoSh- | TGCATATACGATACAAGGCTGTTAGAGAG | | AGGGUCAGC | | (NCBI |
| GFPb- | ATAATTGGAATTAATTTGACTGTAAACACA | | UUGCGCUGA | | Gene: |
| ExoMotif- | AAGATATTAGTACAAAATACGTGACGTAG | | CCCUGAAGU | | 3181) |
| 2 | AAAGTAATAATTTCTTGGGTAGTTTGCAGT | | UCACCCCUU | | |
| | TTTAAAATTATGTTTTAAAATGGACTATCA | | | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACCCCTTTTTTT | | | | |
| U6- | GAGGGCCTATTTCCCATGATTCCTTCATATT | 86 | AUGAACUUC | 100 | |
| AgoSh- | TGCATATACGATACAAGGCTGTTAGAGAG | | AGGGUCAGC | | |
| GFPb- | ATAATTGGAATTAATTTGACTGTAAACACA | | UUGCGCUGA | | |
| ExoCore- | AAGATATTAGTACAAAATACGTGACGTAG | | CCCUGAAGU | | |
| BAT | AAAGTAATAATTTCTTGGGTAGTTTGCAGT | | UCACCAUGU | | |
| | TTTAAAATTATGTTTTAAAATGGACTATCA | | U | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACCATGTTTTTT | | | | |
| U6- | GAGGGCCTATTTCCCATGATTCCTTCATATT | 87 | AUGAACUUC | 101 | |
| AgoSh- | TGCATATACGATACAAGGCTGTTAGAGAG | | AGGGUCAGC | | |
| GFPb- | ATAATTGGAATTAATTTGACTGTAAACACA | | UUGCGCUGA | | |
| ExoCore- | AAGATATTAGTACAAAATACGTGACGTAG | | CCCUGAAGU | | |
| 3T3-L1 | AAAGTAATAATTTCTTGGGTAGTTTGCAGT | | UCACGUGCU | | |
| | TTTAAAATTATGTTTTAAAATGGACTATCA | | U | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACGTGCTTTTTT | | | | |
| U6- | GAGGGCCTATTTCCCATGATTCCTTCATATT | 88 | AUGAACUUC | 102 | |
| AgoSh- | TGCATATACGATACAAGGCTGTTAGAGAG | | AGGGUCAGC | | |
| GFPb- | ATAATTGGAATTAATTTGACTGTAAACACA | | UUGCGCUGA | | |
| ExoCore- | AAGATATTAGTACAAAATACGTGACGTAG | | CCCUGAAGU | | |
| AML12 | AAAGTAATAATTTCTTGGGTAGTTTGCAGT | | UCACCCCCUU | | |
| | TTTAAAATTATGTTTTAAAATGGACTATCA | | | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACCCCCTTTTTT | | | | |
| U6- | GAGGGCCTATTTCCCATGATTCCTTCATATT | 89 | AUGAACUUC | 103 | Fus (NCBI |
| AgoSh- | TGCATATACGATACAAGGCTGTTAGAGAG | | AGGGUCAGC | | Gene: |
| GFPb- | ATAATTGGAATTAATTTGACTGTAAACACA | | UUGCGCUGA | | 2521), |
| ExoCore- | AAGATATTAGTACAAAATACGTGACGTAG | | CCCUGAAGU | | Alyref |
| SVEC | AAAGTAATAATTTCTTGGGTAGTTTGCAGT | | UCACCGGGU | | (NCBI |
| | TTTAAAATTATGTTTTAAAATGGACTATCA | | U | | Gene: |
| | TATGCTTACCGTAACTTGAAAGTATTTCGA | | | | 10189) |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACCGGGTTTTTT | | | | |
| U6- | GAGGGCCTATTTCCCATGATTCCTTCATATT | 90 | AUGAACUUC | 104 | |
| AgoSh- | TGCATATACGATACAAGGCTGTTAGAGAG | | AGGGUCAGC | | |
| GFPb- | ATAATTGGAATTAATTTGACTGTAAACACA | | UUGCGCUGA | | |
| ExoExt- | AAGATATTAGTACAAAATACGTGACGTAG | | CCCUGAAGU | | |
| BAT | AAAGTAATAATTTCTTGGGTAGTTTGCAGT | | UCACUGUGU | | |
| | TTTAAAATTATGTTTTAAAATGGACTATCA | | U | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACTGTGTTTTTTT | | | | |
| U6- | GAGGGCCTATTTCCCATGATTCCTTCATATT | 91 | AUGAACUUC | 105 | |
| AgoSh- | TGCATATACGATACAAGGCTGTTAGAGAG | | AGGGUCAGC | | |
| GFPb- | ATAATTGGAATTAATTTGACTGTAAACACA | | UUGCGCUGA | | |
| ExoExt- | AAGATATTAGTACAAAATACGTGACGTAG | | CCCUGAAGU | | |
| C2C12 | AAAGTAATAATTTCTTGGGTAGTTTGCAGT | | UCACUGUGU | | |
| | TTTAAAATTATGTTTTAAAATGGACTATCA | | GUU | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACTGTGTGTTTTT | | | | |
| | TT | | | | |
| U6- | GAGGGCCTATTTCCCATGATTCCTTCATATT | 92 | AUGAACUUC | 106 | |
| AgoSh- | TGCATATACGATACAAGGCTGTTAGAGAG | | AGGGUCAGC | | |
| GFPb- | ATAATTGGAATTAATTTGACTGTAAACACA | | UUGCGCUGA | | |
| ExoExt- | AAGATATTAGTACAAAATACGTGACGTAG | | CCCUGAAGU | | |
| AML12 | AAAGTAATAATTTCTTGGGTAGTTTGCAGT | | UCACCCGGAG | | |
| | TTTAAAATTATGTTTTAAAATGGACTATCA | | UU | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACCCGGAGTTTT | | | | |
| | TT | | | | |
| U6- | GAGGGCCTATTTCCCATGATTCCTTCATATT | 93 | AUGAACUUC | 107 | Fus (NCBI |
| AgoSh- | TGCATATACGATACAAGGCTGTTAGAGAG | | AGGGUCAGC | | Gene: |
| GFPb- | ATAATTGGAATTAATTTGACTGTAAACACA | | UUGCGCUGA | | 2521), |
| ExoExt- | AAGATATTAGTACAAAATACGTGACGTAG | | CCCUGAAGU | | Alyref |
| SVEC | AAAGTAATAATTTCTTGGGTAGTTTGCAGT | | UCACCGGGA | | (NCBI |
| | TTTAAAATTATGTTTTAAAATGGACTATCA | | GUU | | Gene: |
| | TATGCTTACCGTAACTTGAAAGTATTTCGA | | | | 10189) |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACCGGGAGTTTT | | | | |
| | TT | | | | |
| U6- | GAGGGCCTATTTCCCATGATTCCTTCATATT | 94 | AUGAACUUC | 108 | |
| AgoSh- | TGCATATACGATACAAGGCTGTTAGAGAG | | AGGGUCAGC | | |
| GFPb- | ATAATTGGAATTAATTTGACTGTAAACACA | | UUGCGCUGA | | |
| ExoNeu | AAGATATTAGTACAAAATACGTGACGTAG | | CCCUGAAGU | | |
| | AAAGTAATAATTTCTTGGGTAGTTTGCAGT | | UCACCAGUA | | |
| | TTTAAAATTATGTTTTAAAATGGACTATCA | | GUU | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACCAGTAGTTTT | | | | |
| | TT | | | | |
| U6- | GAGGGCCTATTTCCCATGATTCCTTCATATT | 95 | AUGAACUUC | 109 | |
| AgoSh- | TGCATATACGATACAAGGCTGTTAGAGAG | | AGGGUCAGC | | |
| GFPb- | ATAATTGGAATTAATTTGACTGTAAACACA | | UUGCGCUGA | | |
| ExoAstro | AAGATATTAGTACAAAATACGTGACGTAG | | CCCUGAAGU | | |
| | AAAGTAATAATTTCTTGGGTAGTTTGCAGT | | UCACGUACU | | |
| | TTTAAAATTATGTTTTAAAATGGACTATCA | | U | | |
| | TATGCTTACCGTAACTTGAAAGTATTTCGA | | | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACGTACTTTTTT | | | | |
| U6- | GAGGGCCTATTTCCCATGATTCCTTCATATT | 96 | AUGAACUUC | 110 | U1 snRNPA |
| Agosh- | TGCATATACGATACAAGGCTGTTAGAGAG | | AGGGUCAGC | | (Gene |
| GFPb- | ATAATTGGAATTAATTTGACTGTAAACACA | | UUGCGCUGA | | ID: 26871) |
| U1aRBS | AAGATATTAGTACAAAATACGTGACGTAG | | CCCUGAAGU | | |
| | AAAGTAATAATTTCTTGGGTAGTTTGCAGT | | UCACAAUCCA | | |
| | TTTAAAATTATGTTTTAAAATGGACTATCA | | UUGCACUCC | | |
| | TATGCTTACCGTAACTTGAAAGTATTTCGA | | GGAUUUAUU | | |
| | TTTCTTGGCTTTATATATCTTGTGGAAAGG | | | | |
| | ACGAAGATCCATGAACTTCAGGGTCAGCTT | | | | |
| | GCGCTGACCCTGAAGTTCACAATCCATTGC | | | | |
| | ACTCCGGATTTATTTTTT | | | | |

Bold: small RNA promoter; underlined: Pol III Poly T transcription terminator; lower case: U1 Pol II 3′ Box transcription terminator; italics: predicted UU added on during Pol III transcription termination; bold italics: exosome-specific RNA motif (e.g. exo-motif)

TABLE 3
A go-shRNA targeted to specific genes

| | | SEQ ID | shRNA Sequence | SEQ ID |
|---|---|---|---|---|
| shRNA | Sequence (5′-3′) | NO | (5′-3′) | NO |
| U6-AgoSh- | GAGGGCCTATTTCCCATGATTCCTTCATATTTGCAT | 111 | AAGCGUAGAGUC | 132 |
| STAT3-1 | ATACGATACAAGGCTGTTAGAGAGATAATTGGAA | | ACACUUGCAAAA | |
| | TTAATTTGACTGTAAACACAAAGATATTAGTACAA | | GUGUGACUCUA | |
| | AATACGTGACGTAGAAAGTAATAATTTCTTGGGTA | | CGCUCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAAGCGTAGAGTCACACTTGCAAAAGTGTGACTCT | | | |
| | ACGCTCTTTTTT | | | |
| U6-AgoSh- | GAGGGCCTATTTCCCATGATTCCTTCATATTTGCAT | 112 | AUCUUGACUCUC | 133 |
| STAT3-2 | ATACGATACAAGGCTGTTAGAGAGATAATTGGAA | | AAUCCAAGGGUG | |
| | TTAATTTGACTGTAAACACAAAGATATTAGTACAA | | GAUUGAGAGUC | |
| | AATACGTGACGTAGAAAGTAATAATTTCTTGGGTA | | AAGACUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCATCTTGACTCTCAATCCAAGGGTGGATTGAGAGT | | | |
| | CAAGACTTTTTT | | | |
| U6-AgoSh- | GAGGGCCTATTTCCCATGATTCCTTCATATTTGCAT | 113 | ACUGCUGGUCAA | 134 |
| STAT3-3 | ATACGATACAAGGCTGTTAGAGAGATAATTGGAA | | UCUCUCCCAGGA | |
| | TTAATTTGACTGTAAACACAAAGATATTAGTACAA | | GAGAUUGACCAG | |
| | AATACGTGACGTAGAAAGTAATAATTTCTTGGGTA | | CAGCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCACTGCTGGTCAATCTCTCCCAGGAGAGATTGACC | | | |
| | AGCAGCTTTTTT | | | |
| U6-AgoSh- | GAGGGCCTATTTCCCATGATTCCTTCATATTTGCAT | 114 | ACUGCUUGAUU | 135 |
| STAT3-4 | ATACGATACAAGGCTGTTAGAGAGATAATTGGAA | | CUUCGUAGAUU | |
| | TTAATTTGACTGTAAACACAAAGATATTAGTACAA | | UACGAAGAAUCA | |
| | AATACGTGACGTAGAAAGTAATAATTTCTTGGGTA | | AGCAGCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCACTGCTTGATTCTTCGTAGATTTACGAAGAATCA | | | |
| | AGCAGCTTTTTT | | | |
| U6-AgoSh- | GAGGGCCTATTTCCCATGATTCCTTCATATTTGCAT | 115 | AUCUCCAUUGGC | 136 |
| STAT3-5 | ATACGATACAAGGCTGTTAGAGAGATAATTGGAA | | UUCUCAAGAUU | |
| | TTAATTTGACTGTAAACACAAAGATATTAGTACAA | | GAGAAGCCAAUG | |
| | AATACGTGACGTAGAAAGTAATAATTTCTTGGGTA | | GAGACUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCATCTCCATTGGCTTCTCAAGATTGAGAAGCCAAT | | | |
| | GGAGACTTTTTT | | | |
| U6-AgoSh- | GAGGGCCTATTTCCCATGATTCCTTCATATTTGCAT | 116 | AGUAACUGUUG | 137 |
| HPV16-E6- | ATACGATACAAGGCTGTTAGAGAGATAATTGGAA | | CUUGCAGUACAC | |
| 1 | TTAATTTGACTGTAAACACAAAGATATTAGTACAA | | UGCAAGCAACAG | |
| | AATACGTGACGTAGAAAGTAATAATTTCTTGGGTA | | UUACCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAGTAACTGTTGCTTGCAGTACACTGCAAGCAACA | | | |
| | GTTACCTTTTTT | | | |
| U6-AgoSh- | GAGGGCCTATTTCCCATGATTCCTTCATATTTGCAT | 117 | AGUACACACAUU | 138 |
| HPV16-E6- | ATACGATACAAGGCTGTTAGAGAGATAATTGGAA | | CUAAUAUUAUU | |
| 2 | TTAATTTGACTGTAAACACAAAGATATTAGTACAA | | AUUAGAAUGUG | |
| | AATACGTGACGTAGAAAGTAATAATTTCTTGGGTA | | UGUACCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAGTACACACATTCTAATATTATTATTAGAATGTGT | | | |
| | GTACCTTTTTT | | | |
| U6-AgoSh- | GAGGGCCTATTTCCCATGATTCCTTCATATTTGCAT | 118 | AUGUAUAGUUG | 139 |
| HPV16-E6- | ATACGATACAAGGCTGTTAGAGAGATAATTGGAA | | UUUGCAGCUCUC | |
| 3 | TTAATTTGACTGTAAACACAAAGATATTAGTACAA | | UGCAAACAACUA | |
| | AATACGTGACGTAGAAAGTAATAATTTCTTGGGTA | | UACACUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCATGTATAGTTGTTTGCAGCTCTCTGCAAACAACT | | | |
| | ATACACTTTTTT | | | |
| U6-AgoSh- | GAGGGCCTATTTCCCATGATTCCTTCATATTTGCAT | 119 | AGUUGUUUGCA | 140 |
| HPV16-E6- | ATACGATACAAGGCTGTTAGAGAGATAATTGGAA | | GCUCUGUGCAU | |
| 4 | TTAATTTGACTGTAAACACAAAGATATTAGTACAA | | ACAGAGCUGCAA | |
| | AATACGTGACGTAGAAAGTAATAATTTCTTGGGTA | | ACAACCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAGTTGTTTGCAGCTCTGTGCATACAGAGCTGCAA | | | |
| | ACAACCTTTTTT | | | |
| U6-AgoSh- | GAGGGCCTATTTCCCATGATTCCTTCATATTTGCAT | 120 | ACCUCACGUCGC | 141 |
| HPV16-E6- | ATACGATACAAGGCTGTTAGAGAGATAATTGGAA | | AGUAACUGUUG | |
| 5 | TTAATTTGACTGTAAACACAAAGATATTAGTACAA | | UUACUGCGACG | |
| | AATACGTGACGTAGAAAGTAATAATTTCTTGGGTA | | UGAGGCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCACCTCACGTCGCAGTAACTGTTGTTACTGCGACG | | | |
| | TGAGGCTTTTTT | | | |
| U6-AgoSh- | GAGGGCCTATTTCCCATGATTCCTTCATATTTGCAT | 121 | AAGCGUAGAGUC | 142 |
| HPV16-E7- | ATACGATACAAGGCTGTTAGAGAGATAATTGGAA | | ACACUUGCAAAA | |
| 1 | TTAATTTGACTGTAAACACAAAGATATTAGTACAA | | GUGUGACUCUA | |
| | AATACGTGACGTAGAAAGTAATAATTTCTTGGGTA | | CGCUCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAAGCGTAGAGTCACACTTGCAAAAGTGTGACTCT | | | |
| | ACGCTCTTTTTT | | | |
| U6-AgoSh- | GAGGGCCTATTTCCCATGATTCCTTCATATTTGCAT | 122 | AUUCCUAGUGU | 143 |
| HPV16-E7- | ATACGATACAAGGCTGTTAGAGAGATAATTGGAA | | GCCCAUUAACAA | |
| 2 | TTAATTTGACTGTAAACACAAAGATATTAGTACAA | | AUGGGCACACUA | |
| | AATACGTGACGTAGAAAGTAATAATTTCTTGGGTA | | GGAACUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCATTCCTAGTGTGCCCATTAACAAATGGGCACACT | | | |
| | AGGAACTTTTTT | | | |
| U6-AgoSh- | GAGGGCCTATTTCCCATGATTCCTTCATATTTGCAT | 123 | AUGGGCUCUGU | 144 |
| HPV16-E7- | ATACGATACAAGGCTGTTAGAGAGATAATTGGAA | | CCGGUUCUGCU | |
| 3 | TTAATTTGACTGTAAACACAAAGATATTAGTACAA | | GAACCGGACAGA | |
| | AATACGTGACGTAGAAAGTAATAATTTCTTGGGTA | | GCCCACUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCATGGGCTCTGTCCGGTTCTGCTGAACCGGACAG | | | |
| | AGCCCACTTTTTT | | | |
| U6-AgoSh- | GAGGGCCTATTTCCCATGATTCCTTCATATTTGCAT | 124 | AGUUGUCUCUG | 145 |
| HPV16-E7- | ATACGATACAAGGCTGTTAGAGAGATAATTGGAA | | GUUGCAAAUCU | |
| 4 | TTAATTTGACTGTAAACACAAAGATATTAGTACAA | | UUGCAACCAGAG | |
| | AATACGTGACGTAGAAAGTAATAATTTCTTGGGTA | | ACAACCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAGTTGTCTCTGGTTGCAAATCTTTGCAACCAGAG | | | |
| | ACAACCTTTTTT | | | |
| U6-AgoSh- | GAGGGCCTATTTCCCATGATTCCTTCATATTTGCAT | 125 | ACGUGUGUGCU | 146 |
| HPV16-E7- | ATACGATACAAGGCTGTTAGAGAGATAATTGGAA | | UUGUACGCACAC | |
| 5 | TTAATTTGACTGTAAACACAAAGATATTAGTACAA | | GUACAAAGCACA | |
| | AATACGTGACGTAGAAAGTAATAATTTCTTGGGTA | | CACGCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCACGTGTGTGCTTTGTACGCACACGTACAAAGCAC | | | |
| | ACACGCTTTTTT | | | |
| U6-AgoSh- | GAGGGCCTATTTCCCATGATTCCTTCATATTTGCAT | 126 | AGUCGUGCUGC | 147 |
| GFPb-2 | ATACGATACAAGGCTGTTAGAGAGATAATTGGAA | | UUCAUGUGGUC | |
| | TTAATTTGACTGTAAACACAAAGATATTAGTACAA | | ACAUGAAGCAGC | |
| | AATACGTGACGTAGAAAGTAATAATTTCTTGGGTA | | ACGACCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAGTCGTGCTGCTTCATGTGGTCACATGAAGCAGC | | | |
| | ACGACCTTTTTT | | | |
| U6-AgoSh- | GAGGGCCTATTTCCCATGATTCCTTCATATTTGCAT | 127 | AUGCCCUUCAGC | 148 |
| GFPb-3 | ATACGATACAAGGCTGTTAGAGAGATAATTGGAA | | UCGAUGCGGUC | |
| | TTAATTTGACTGTAAACACAAAGATATTAGTACAA | | AUCGAGCUGAAG | |
| | AATACGTGACGTAGAAAGTAATAATTTCTTGGGTA | | GGCACUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCATGCCCTTCAGCTCGATGCGGTCATCGAGCTGAA | | | |
| | GGGCACTTTTTT | | | |
| U6-AgoSh- | GAGGGCCTATTTCCCATGATTCCTTCATATTTGCAT | 128 | AGCUUCUUCUGC | 149 |
| Fluc1 | ATACGATACAAGGCTGTTAGAGAGATAATTGGAA | | ACAUUCAGGAGA | |
| | TTAATTTGACTGTAAACACAAAGATATTAGTACAA | | AUGUGCAGAAG | |
| | AATACGTGACGTAGAAAGTAATAATTTCTTGGGTA | | AAGCCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAGCTTCTTCTGCACATTCAGGAGAATGTGCAGAA | | | |
| | GAAGCCTTTTTT | | | |
| U6-AgoSh- | GAGGGCCTATTTCCCATGATTCCTTCATATTTGCAT | 129 | AGUGGCUGGUC | 150 |
| Fluc2 | ATACGATACAAGGCTGTTAGAGAGATAATTGGAA | | ACAAAGGUAUAC | |
| | TTAATTTGACTGTAAACACAAAGATATTAGTACAA | | CUUUGUGACCA | |
| | AATACGTGACGTAGAAAGTAATAATTTCTTGGGTA | | GCCACCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAGTGGCTGGTCACAAAGGTATACCTTTGTGACCA | | | |
| | GCCACCTTTTTT | | | |
| U6-AgoSh- | GAGGGCCTATTTCCCATGATTCCTTCATATTTGCAT | 130 | AGUUCUCAGAGC | 151 |
| Fluc3 | ATACGATACAAGGCTGTTAGAGAGATAATTGGAA | | ACACCACAAUUG | |
| | TTAATTTGACTGTAAACACAAAGATATTAGTACAA | | GUGUGCUCUGA | |
| | AATACGTGACGTAGAAAGTAATAATTTCTTGGGTA | | GAACCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAGTTCTCAGAGCACACCACAATTGGTGTGCTCTG | | | |
| | AGAACCTTTTTT | | | |
| U6-AgoSh- | GAGGGCCTATTTCCCATGATTCCTTCATATTTGCAT | 131 | AGGAAAGUCCCC | 152 |
| 362 (HIV) | ATACGATACAAGGCTGTTAGAGAGATAATTGGAA | | AGCGGAAAGUUC | |
| | TTAATTTGACTGTAAACACAAAGATATTAGTACAA | | CGCUGGGGACU | |
| | AATACGTGACGTAGAAAGTAATAATTTCTTGGGTA | | UUCCCUU | |
| | GTTTGCAGTTTTAAAATTATGTTTTAAAATGGACTA | | | |
| | TCATATGCTTACCGTAACTTGAAAGTATTTCGATTT | | | |
| | CTTGGCTTTATATATCTTGTGGAAAGGACGAAGAT | | | |
| | CCAGGAAAGTCCCCAGCGGAAAGTTCCGCTGGGGA | | | |
| | CTTTCCCTTTTTT | | | |

Bold: small RNA Promoter; underlined: Pol III Poly T transcription terminator; lower case: U1 Pol II 3′ Box transcription terminator; italics: predicted UU added on during Pol III transcription termination.

TABLE 4
Exosome-specific RNA motifs

| | | Exo-Motif |
|---|---|---|
| RNA | Motif Sequence | binding protien |
| miR-451 | CUC | MEX3C-1 |
| Exo-motif | GGAG, CCCU | hnRNPA2B1 |
| hExo-motif | GGCU | SYNCRIP |
| A stro and Neuro-motif | GUAC, CAGUAG | Unknown |
| LA motif | UGGA or PolyU | LA protein |
| Poly uridylation motif | Poly U | Unknown |
| Core and Extended Exo-Motif | UGUGU, UGUGUGU, CCGGAG, | ALYREF, Fus |
| | CGGGAG, CAGUAG CAUG, GUGC, | |
| | CCCC, CGGG | |

TABLE 5
Small RNA Promoters

| | | SEQ ID |
|---|---|---|
| Promoter | Sequence | NO |
| U6 | GAGGGCCTATTTCCCATGATTCCTTCATATTTGCATATACGATACAAGGCTGTTAGAGAG | 160 |
| | ATAATTGGAATTAATTTGACTGTAAACACAAAGATATTAGTACAAAATACGTGACGTAG | |
| | AAAGTAATAATTTCTTGGGTAGTTTGCAGTTTTAAAATTATGTTTTAAAATGGACTATCA | |
| | TATGCTTACCGTAACTTGAAAGTATTTCGATTTCTTGGCTTTATATATCTTGTGGAAAGG | |
| | ACGAAGATCC | |
| mU6 | GATCCGACGCCGCCATCTCTAGGCCCGCGCCGGCCCCCTCGCACAGACTTGTGGGAGA | 161 |
| | AGCTCGGCTACTCCCCTGCCCCGGTTAATTTGCATATAATATTTCCTAGTAACTATAGAG | |
| | GCTTAATGTGCGATAAAAGACAGATAATCTGTTCTTTTTAATACTAGCTACATTTTACAT | |
| | GATAGGCTTGGATTTCTATAAGAGATACAAATACTAAATTATTATTTTAAAAAACAGCAC | |
| | AAAAGGAAACTCACCCTAACTGTAAAGTAATTGTGTGTTTTGAGACTATAAATATCCCTT | |
| | GGAGAAAAGCCTTGTT | |
| H1 | TGCAATATTTGCATGTCGCTATGTGTTCTGGGAAATCACCATAAACGTGAAATGTCTTTG | 162 |
| | GATTTGGGAATCTTATAAGTTCTGTATGAGACCACTCAGATCC | |
| 7SK | CTGCAGTATTTAGCATGCCCCACCCATCTGCAAGGCATTCTGGATAGTGTCAAAACAGC | 163 |
| | CGGAAATCAAGTCCGTTTATCTCAAACTTTAGCATTTTGGGAATAAATGATATTTGCTAT | |
| | GCTGGTTAAATTAGATTTTAGTTAAATTTCCTGCTGAAGCTCTAGTACGATAAGTAACTT | |
| | GACCTAAGTGTAAAGTTGAGATTTCCTTCAGGTTTATATAGCTTGTGCGCCGCCTGGGT | |
| | ACCTC | |
| U1 | CTAAGGACCAGCTTCTTTGGGAGAGAACAGACGCAGGGGGGGGAGGGAAAAAGGGA | 164 |
| | GAGGCAGACGTCACTTCCCCTTGGCGGCTCTGGCAGCAGATTGGTCGGTTGAGTGGCA | |
| | GAAAGGCAGACGGGGACTGGGCAAGGCACTGTCGGTGACATCACGGACAGGGCGACT | |
| | TCTATGTAGATGAGGCAGCGCAGAGGCTGCTGCTTCGCCACTTGCTGCTTCGCCACGAA | |
| | GGAGTTCCCGTGCCCTGGGAGCGGGTTCAGGACCGCGGATCGGAAGTGAGAATCCCA | |
| | GCTGTGTGTCAGGGCTGGAAAGGGCTCGGGAGTGCGCGGGGCAAGTGACCGTGTGTG | |
| | TAAAGAGTGAGGCGTATGAGGCTGTGTCGGGGCAGAGCCCGAAGATC | |
| T7 | TAATACGACTCACTATAGGG | 165 |
| SP6 | ATTTAGGTGACACTATAGA | 166 |
| T3 | AATTAACCCTCACTAAAGG | 167 |
| tRNAlys3 | GCCCGGATAGCTCAGTCGGTAGAGCATCAGACTTTTAATCTGAGGGTCCAGGGTTCAA | 168 |
| | GTCCCTGTTCGGGCG | |
| tRNAval | GTTTTCGTAGTGTAGTGGTTATCACGTGTGCTTCACACGCACAAGGTCCCCGGTTCGAA | 169 |
| | CCCGGGCGAAAACA | |
| tRNAser | GAAAATGACTTTGCCACGCTTAGCATGTGACGAGGTGGCCGAGTGGTTAAGGCGATGG | 170 |
| | ACTGCTAATCCATTGTGCTCTGCACGCGTGGGTTCGAATCCCATCCTCGTCG | |
| tRNAgly | GCATGGGTGGTTCAGTGGTAGAATTCTCGCCTGCCACGCGGGAGGCCCGGGTTCGATT | 171 |
| | CCCGGCCCATGCA | |
| tRNAGln | GGTTCCATGGTGTAATGGTTAGCACTCTGGACTCTGAATCCAGCGATCCGAGTTCAAAT | 172 |
| | CTCGGTGGAACCT | |
| tRNAPro | GGCTCGTTGGTCTAGGGGTATGATTCTCGCTTAGGGTGCGAGAGGTCCCGGGTTCAAA | 173 |
| | TCCCGGACGAGCCC | |
| tRNALeu | GTCAGGATGGCCGAGTGGTCTAAGGCGCTGCGTTCAGGTCGCAGTCTACTCTGTAGGC | 174 |
| | GTGGGTTCGAATCCCACTTCTGACA | |
| tRNAAsn | GCCTCCGTGGCGCAATTGGTTAGCGCGTTCGGCTGTTAACCGAAAGGTTGGTGGTTCG | 175 |
| | AGTCCACCCGGGGGCG | |

TABLE 6
Primer and G-block sequences

| | | SEQ ID |
|---|---|---|
| Oligomer/G-block | Sequence (5′-3′) | NO |
| pSI-GFP sense (+) | TCGAGTACGGCAAGCTGACCCTGAAGTTCATCTGCACGC | 176 |
| pSI-GFP sense (−) | GGCCGCGTGCAGATGAACTTCAGGGTCAGCTTGCCGTAC | 177 |
| pSI-GFP anti-sense (+) | TCGAGGTGCAGATGAACTTCAGGGTCAGCTTGCCGTAGC | 178 |
| pSI-GFP anti-sense (−) | GGCCGCTACGGCAAGCTGACCCTGAAGTTCATCTGCACC | 179 |
| pSI-362 sense (+) | TCGAGGGGACTTTCCGCTGGGGACTTTCCAGGGC | 180 |
| pSI-362 sense (−) | GGCCGCCCTGGAAAGTCCCCAGCGGAAAGTCCCC | 181 |
| pSI-362 anti-sense (+) | TCGAGCCTGGAAAGTCCCCAGCGGAAAGTCCCGC | 182 |
| pSI-362 anti-sense (−) | GGCCGCGGGACTTTCCGCTGGGGACTTTCCAGGC | 183 |
| HPV-E6/E7 gBlock | CAGTAATTCTAGGCGATCGCTCGAGATGCACCAAAAGAGAACTGCAATG | 184 |
| | TTTCAGGACCCACAGGAGCGACCCAGAAAGTTACCACAGTTATGCACAG | |
| | AGCTGCAAACAACTATACATGATATAATATTAGAATGTGTGTACTGCAAG | |
| | CAACAGTTACTGCGACGTGAGGTATATGACTTTGCTTTTCGGGATTTATG | |
| | CATAGTATATAGAGATGGGAATCCATATGCTGTATGTGATAAATGTTTAA | |
| | AGTTTTATTCTAAAATTAGTGAGTATAGACATTATTGTTATAGTTTGTATG | |
| | GAACAACATTAGAACAGCAATACAACAAACCGTTGTGTGATTTGTTAATT | |
| | AGGTGTATTAACTGTCAAAAGCCACTGTGTCCTGAAGAAAAGCAAAGAC | |
| | ATCTGGACAAAAAGCAAAGATTCCATAATATAAGGGGTCGGTGGACCGG | |
| | TCGATGTATGTCTTGTTGCAGATCATCAAGAACACGTAGAGAAACCCAGC | |
| | TGTAATCATGCATGGAGATACACCTACATTGCATGAATATATGTTAGATT | |
| | TGCAACCAGAGACAACTGATCTCTACTGTTATGAGCAATTAAATGACAGC | |
| | TCAGAGGAGGAGGATGAAATAGATGGTCCAGCTGGACAAGCAGAACCG | |
| | GACAGAGCCCATTACAATATTGTAACCTTTTGTTGCAAGTGTGACTCTAC | |
| | GCTTCGGTTGTGCGTACAAAGCACACACGTAGACATTCGTACTTTGGAAG | |
| | ACCTGTTAATGGGCACACTAGGAATTGTGTGCCCCATCTGTTCTCAGAAA | |
| | CCATAAGCGGCCGCTGGCCGCAATAAAATAT | |
| KRAS | CGACTCACTATAGGCTAGCCTCGAGGCCACCATGTACCCATATGACGTGC | 185 |
| | CCGATTACGCCGGCAGCGGCACTGAATATAAACTTGTGGTAGTTGGAGC | |
| | TGGTGGCGTAGGCAAGAGTGCCTTGACGATACAGCTAATTCAGAATCAT | |
| | TTTGTGGACGAATATGATCCAACAATAGAGGATTCCTACAGGAAGCAAG | |
| | TAGTAATTGATGGAGAAACCTGTCTCTTGGATATTCTCGACACAGCAGGT | |
| | CAAGAGGAGTACAGTGCAATGAGGGACCAGTACATGAGGACTGGGGAG | |
| | GGCTTTCTTTGTGTATTTGCCATAAATAATACTAAATCATTTGAAGATATT | |
| | CACCATTATAGAGAACAAATTAAAAGAGTTAAGGACTCTGAAGATGTAC | |
| | CTATGGTCCTAGTAGGAAATAAATGTGATTTGCCTTCTAGAACAGTAGAC | |
| | ACAAAACAGGCTCAGGACTTAGCAAGAAGTTATGGAATTCCTTTTATTGA | |
| | AACATCAGCAAAGACAAGACAGGGTGTTGATGATGCCTTCTATACATTA | |
| | GTTCGAGAAATTCGAAAACATAAAGAAAAGATGAGCAAAGATGGTAAA | |
| | AAGAAGAAAAAGAAGTCAAAGACAAAGTGTGTAATTATGTAATAGTAAG | |
| | CGGCCGCTTCCCTTTAGTGAGG | |
| HRAS | CGACTCACTATAGGCTAGCCTCGAGGCCACCATGTACCCATATGACGTGC | 186 |
| | CCGATTACGCCGGCAGCGGCACGGAATATAAGCTGGTGGTGGTGGGCG | |
| | CCGGCGGTGTGGGCAAGAGTGCGCTGACCATCCAGCTGATCCAGAACCA | |
| | TTTTGTGGACGAATACGACCCCACTATAGAGGATTCCTACCGGAAGCAG | |
| | GTGGTCATTGATGGGGAGACGTGCCTGTTGGACATCCTGGATACCGCCG | |
| | GCCAGGAGGAGTACAGCGCCATGCGGGACCAGTACATGCGCACCGGGG | |
| | AGGGCTTCCTGTGTGTGTTTGCCATCAACAACACCAAGTCTTTTGAGGAC | |
| | ATCCACCAGTACAGGGAGCAGATCAAACGGGTGAAGGACTCGGATGAC | |
| | GTGCCCATGGTGCTGGTGGGGAACAAGTGTGACCTGGCTGCACGCACTG | |
| | TGGAATCTCGGCAGGCTCAGGACCTCGCCCGAAGCTACGGCATCCCCTA | |
| | CATCGAGACCTCGGCCAAGACCCGGCAGGGAGTGGAGGATGCCTTCTAC | |
| | ACGTTGGTGCGTGAGATCCGGCAGCACAAGCTGCGGAAGCTGAACCCTC | |
| | CTGATGAGAGTGGCCCCGGCTGCATGAGCTGCAAGTGTGTGCTCTCCTG | |
| | ATAGTAAGCGGCCGCTTCCCTTTAGTGAGG | |
| NRAS | CGACTCACTATAGGCTAGCCTCGAGGCCACCATGTACCCATATGACGTGC | 187 |
| | CCGATTACGCCGGCAGCGGCACTGAGTACAAACTGGTGGTGGTTGGAGC | |
| | AGGTGGTGTTGGGAAAAGCGCACTGACAATCCAGCTAATCCAGAACCAC | |
| | TTTGTAGATGAATATGATCCCACCATAGAGGATTCTTACAGAAAACAAGT | |
| | GGTTATAGATGGTGAAACCTGTTTGTTGGACATACTGGATACAGCTGGA | |
| | CAAGAAGAGTACAGTGCCATGAGAGACCAATACATGAGGACAGGCGAA | |
| | GGCTTCCTCTGTGTATTTGCCATCAATAATAGCAAGTCATTTGCGGATATT | |
| | AACCTCTACAGGGAGCAGATTAAGCGAGTAAAAGACTCGGATGATGTAC | |
| | CTATGGTGCTAGTGGGAAACAAGTGTGATTTGCCAACAAGGACAGTTGA | |
| | TACAAAACAAGCCCACGAACTGGCCAAGAGTTACGGGATTCCATTCATTG | |
| | AAACCTCAGCCAAGACCAGACAGGGTGTTGAAGATGCTTTTTACACACTG | |
| | GTAAGAGAAATACGCCAGTACCGAATGAAAAAACTCAACAGCAGTGATG | |
| | ATGGGACTCAGGGTTGTATGGGATTGCCATGTGTGGTGATGTAATAGTA | |
| | AGCGGCCGCTTCCCTTTAGTGAGG | |
| PTPN1 | CGACTCACTATAGGCTAGCCTCGAGGCCACCATGTACCCATATGACGTGC | 188 |
| | CCGATTACGCCGGCAGCGGCGAGATGGAAAAGGAGTTCGAGCAGATCG | |
| | ACAAGTCCGGGAGCTGGGCGGCCATTTACCAGGATATCCGACATGAAGC | |
| | CAGTGACTTCCCATGTAGAGTGGCCAAGCTTCCTAAGAACAAAAACCGA | |
| | AATAGGTACAGAGACGTCAGTCCCTTTGACCATAGTCGGATTAAACTACA | |
| | TCAAGAAGATAATGACTATATCAACGCTAGTTTGATAAAAATGGAAGAA | |
| | GCCCAAAGGAGTTACATTCTTACCCAGGGCCCTTTGCCTAACACATGCGG | |
| | TCACTTTTGGGAGATGGTGTGGGAGCAGAAAAGCAGGGGTGTCGTCAT | |
| | GCTCAACAGAGTGATGGAGAAAGGTTCGTTAAAATGCGCACAATACTGG | |
| | CCACAAAAAGAAGAAAAAGAGATGATCTTTGAAGACACAAATTTGAAAT | |
| | TAACATTGATCTCTGAAGATATCAAGTCATATTATACAGTGCGACAGCTA | |
| | GAATTGGAAAACCTTACAACCCAAGAAACTCGGGAGATCTTACATTTCCA | |
| | CTATACCACATGGCCTGACTTTGGAGTCCCTGAATCACCAGCCTCATTCTT | |
| | GAACTTTCTTTTCAAAGTCCGAGAGTCAGGGTCACTCAGCCCGGAGCACG | |
| | GGCCCGTTGTGGTGCACTGCAGTGCAGGCATCGGCAGGTCTGGAACCTT | |
| | CTGTCTGGCTGATACCTGCCTCTTGCTGATGGACAAGAGGAAAGACCCTT | |
| | CTTCCGTTGATATCAAGAAAGTGCTGTTAGAAATGAGGAAGTTTCGGAT | |
| | GGGGCTGATCCAGACAGCCGACCAGCTGCGCTTCTCCTACCTGGCTGTG | |
| | ATCGAAGGTGCCAAATTCATCATGGGGGACTCTTCCGTGCAGGATCAGT | |
| | GGAAGGAGCTTTCCCACGAGGACCTGGAGCCCCCACCCGAGCATATCCC | |
| | CCCACCTCCCCGGCCACCCAAACGAATCCTGGAGCCACACAATGGGAAAT | |
| | GCAGGGAGTTCTTCCCAAATCACCAGTGGGTGAAGGAAGAGACCCAGG | |
| | AGGATAAAGACTGCCCCATCAAGGAAGAAAAAGGAAGCCCCTTAAATGC | |
| | CGCACCCTACGGCATCGAAAGCATGAGTCAAGACACTGAAGTTAGAAGT | |
| | CGGGTCGTGGGGGGAAGTCTTCGAGGTGCCCAGGCTGCCTCCCCAGCCA | |
| | AAGGGGAGCCGTCACTGCCCGAGAAGGACGAGGACCATGCACTGAGTT | |
| | ACTGGAAGCCCTTCCTGGTCAACATGTGCGTGGCTACGGTCCTCACGGCC | |
| | GGCGCTTACCTCTGCTACAGGTTCCTGTTCAACAGCAACACATAGTAGTA | |
| | AGCGGCCGCTTCCCTTTAGTGAGG | |
| KRAS-S17N | CGACTCACTATAGGCTAGCCTCGAGGCCACCATGTACCCATATGACGTGC | 189 |
| | CCGATTACGCCGGCAGCGGCACTGAATATAAACTTGTGGTAGTTGGAGC | |
| | TGGTGGCGTAGGCAAGAATGCCTTGACGATACAGCTAATTCAGAATCAT | |
| | TTTGTGGACGAATATGATCCAACAATAGAGGATTCCTACAGGAAGCAAG | |
| | TAGTAATTGATGGAGAAACCTGTCTCTTGGATATTCTCGACACAGCAGGT | |
| | CAAGAGGAGTACAGTGCAATGAGGGACCAGTACATGAGGACTGGGGAG | |
| | GGCTTTCTTTGTGTATTTGCCATAAATAATACTAAATCATTTGAAGATATT | |
| | CACCATTATAGAGAACAAATTAAAAGAGTTAAGGACTCTGAAGATGTAC | |
| | CTATGGTCCTAGTAGGAAATAAATGTGATTTGCCTTCTAGAACAGTAGAC | |
| | ACAAAACAGGCTCAGGACTTAGCAAGAAGTTATGGAATTCCTTTTATTGA | |
| | AACATCAGCAAAGACAAGACAGGGTGTTGATGATGCCTTCTATACATTA | |
| | GTTCGAGAAATTCGAAAACATAAAGAAAAGATGAGCAAAGATGGTAAA | |
| | AAGAAGAAAAAGAAGTCAAAGACAAAGTGTGTAATTATGTAATAGTAAG | |
| | CGGCCGCTTCCCTTTAGTGAGG | |
| HRAS-S17N | CGACTCACTATAGGCTAGCCTCGAGGCCACCATGTACCCATATGACGTGC | 190 |
| | CCGATTACGCCGGCAGCGGCACGGAATATAAGCTGGTGGTGGTGGGCG | |
| | CCGGCGGTGTGGGCAAGAACGCGCTGACCATCCAGCTGATCCAGAACCA | |
| | TTTTGTGGACGAATACGACCCCACTATAGAGGATTCCTACCGGAAGCAG | |
| | GTGGTCATTGATGGGGAGACGTGCCTGTTGGACATCCTGGATACCGCCG | |
| | GCCAGGAGGAGTACAGCGCCATGCGGGACCAGTACATGCGCACCGGGG | |
| | AGGGCTTCCTGTGTGTGTTTGCCATCAACAACACCAAGTCTTTTGAGGAC | |
| | ATCCACCAGTACAGGGAGCAGATCAAACGGGTGAAGGACTCGGATGAC | |
| | GTGCCCATGGTGCTGGTGGGGAACAAGTGTGACCTGGCTGCACGCACTG | |
| | TGGAATCTCGGCAGGCTCAGGACCTCGCCCGAAGCTACGGCATCCCCTA | |
| | CATCGAGACCTCGGCCAAGACCCGGCAGGGAGTGGAGGATGCCTTCTAC | |
| | ACGTTGGTGCGTGAGATCCGGCAGCACAAGCTGCGGAAGCTGAACCCTC | |
| | CTGATGAGAGTGGCCCCGGCTGCATGAGCTGCAAGTGTGTGCTCTCCTG | |
| | ATAGTAAGCGGCCGCTTCCCTTTAGTGAGG | |
| HRAS-G12D | CGACTCACTATAGGCTAGCCTCGAGGCCACCATGTACCCATATGACGTGC | 191 |
| | CCGATTACGCCGGCAGCGGCACGGAATATAAGCTGGTGGTGGTGGGCG | |
| | CCGACGGTGTGGGCAAGAGTGCGCTGACCATCCAGCTGATCCAGAACCA | |
| | TTTTGTGGACGAATACGACCCCACTATAGAGGATTCCTACCGGAAGCAG | |
| | GTGGTCATTGATGGGGAGACGTGCCTGTTGGACATCCTGGATACCGCCG | |
| | GCCAGGAGGAGTACAGCGCCATGCGGGACCAGTACATGCGCACCGGGG | |
| | AGGGCTTCCTGTGTGTGTTTGCCATCAACAACACCAAGTCTTTTGAGGAC | |
| | ATCCACCAGTACAGGGAGCAGATCAAACGGGTGAAGGACTCGGATGAC | |
| | GTGCCCATGGTGCTGGTGGGGAACAAGTGTGACCTGGCTGCACGCACTG | |
| | TGGAATCTCGGCAGGCTCAGGACCTCGCCCGAAGCTACGGCATCCCCTA | |
| | CATCGAGACCTCGGCCAAGACCCGGCAGGGAGTGGAGGATGCCTTCTAC | |
| | ACGTTGGTGCGTGAGATCCGGCAGCACAAGCTGCGGAAGCTGAACCCTC | |
| | CTGATGAGAGTGGCCCCGGCTGCATGAGCTGCAAGTGTGTGCTCTCCTG | |
| | ATAGTAAGCGGCCGCTTCCCTTTAGTGAGG | |
| HRAS-G12V | CGACTCACTATAGGCTAGCCTCGAGGCCACCATGTACCCATATGACGTGC | 192 |
| | CCGATTACGCCGGCAGCGGCACGGAATATAAGCTGGTGGTGGTGGGCG | |
| | CCGTGGGTGTGGGCAAGAGTGCGCTGACCATCCAGCTGATCCAGAACCA | |
| | TTTTGTGGACGAATACGACCCCACTATAGAGGATTCCTACCGGAAGCAG | |
| | GTGGTCATTGATGGGGAGACGTGCCTGTTGGACATCCTGGATACCGCCG | |
| | GCCAGGAGGAGTACAGCGCCATGCGGGACCAGTACATGCGCACCGGGG | |
| | AGGGCTTCCTGTGTGTGTTTGCCATCAACAACACCAAGTCTTTTGAGGAC | |
| | ATCCACCAGTACAGGGAGCAGATCAAACGGGTGAAGGACTCGGATGAC | |
| | GTGCCCATGGTGCTGGTGGGGAACAAGTGTGACCTGGCTGCACGCACTG | |
| | TGGAATCTCGGCAGGCTCAGGACCTCGCCCGAAGCTACGGCATCCCCTA | |
| | CATCGAGACCTCGGCCAAGACCCGGCAGGGAGTGGAGGATGCCTTCTAC | |
| | ACGTTGGTGCGTGAGATCCGGCAGCACAAGCTGCGGAAGCTGAACCCTC | |
| | CTGATGAGAGTGGCCCCGGCTGCATGAGCTGCAAGTGTGTGCTCTCCTG | |
| | ATAGTAAGCGGCCGCTTCCCTTTAGTGAGG | |
| HNRNPA2B1 | CGACTCACTATAGGCTAGCCTCGAGGCCACCATGGAGAAGACCCTGGAG | 193 |
| | ACCGTGCCCCTGGAGAGAAAGAAGAGAGAGAAGGAGCAGTTCAGAAAG | |
| | CTGTTCATCGGCGGCCTGAGCTTCGAGACCACCGAGGAGAGCCTGAGAA | |
| | ACTACTACGAGCAGTGGGGCAAGCTGACCGACTGCGTGGTGATGAGAG | |
| | ACCCCGCCAGCAAGAGAAGCAGAGGCTTCGGCTTCGTGACCTTCAGCAG | |
| | CATGGCCGAGGTGGACGCCGCCATGGCCGCCAGACCCCACAGCATCGAC | |
| | GGCAGAGTGGTGGAGCCCAAGAGAGCCGTGGCCAGAGAGGAGAGCGG | |
| | CAAGCCCGGCGCCCACGTGACCGTGAAGAAGCTGTTCGTGGGCGGCATC | |
| | AAGGAGGACACCGAGGAGCACCACCTGAGAGACTACTTCGAGGAGTAC | |
| | GGCAAGATCGACACCATCGAGATCATCACCGACAGACAGAGCGGCAAG | |
| | AAGAGAGGCTTCGGCTTCGTGACCTTCGACGACCACGACCCCGTGGACA | |
| | AGATCGTGCTGCAGAAGTACCACACCATCAACGGCCACAACGCCGAGGT | |
| | GAGAAAGGCCCTGAGCAGACAGGAGATGCAGGAGGTGCAGAGCAGCA | |
| | GAAGCGGCAGAGGCGGCAACTTCGGCTTCGGCGACAGCAGAGGCGGCG | |
| | GCGGCAACTTCGGCCCCGGCCCCGGCAGCAACTTCAGAGGCGGCAGCG | |
| | ACGGCTACGGCAGCGGCAGAGGCTTCGGCGACGGCTACAACGGCTACG | |
| | GCGGCGGCCCCGGCGGCGGCAACTTCGGCGGCAGCCCCGGCTACGGCG | |
| | GCGGCAGAGGCGGCTACGGCGGCGGCGGCCCCGGCTACGGCAACCAGG | |
| | GCGGCGGCTACGGCGGCGGCTACGACAACTACGGCGGCGGCAACTACG | |
| | GCAGCGGCAACTACAACGACTTCGGCAACTACAACCAGCAGCCCAGCAA | |
| | CTACGGCCCCATGAAGAGCGGCAACTTCGGCGGCAGCAGAAACATGGG | |
| | CGGCCCCTACGGCGGCGGCAACTACGGCCCCGGCGGCAGCGGCGGCAG | |
| | CGGCGGCTACGGCGGCAGAAGCAGATACGGCAGCGGCGAGCAGAAGCT | |
| | GATCTCAGAGGAGGACCTGTAGTAAGCGGCCGCTTCCCTTTAGTGAGG | |
| SYNCRIP | CGACTCACTATAGGCTAGCCTCGAGGCCACCATGGCCACCGAGCACGTG | 194 |
| | AACGGCAACGGCACCGAGGAGCCCATGGACACCACCAGCGCCGTGATCC | |
| | ACAGCGAGAACTTCCAGACCCTGCTGGACGCCGGCCTGCCCCAGAAGGT | |
| | GGCCGAGAAGCTGGACGAGATCTACGTGGCCGGCCTGGTGGCCCACAG | |
| | CGACCTGGACGAGAGAGCCATCGAGGCCCTGAAGGAGTTCAACGAGGA | |
| | CGGCGCCCTGGCCGTGCTGCAGCAGTTCAAGGACAGCGACCTGAGCCAC | |
| | GTGCAGAACAAGAGCGCCTTCCTGTGCGGCGTGATGAAGACCTACAGAC | |
| | AGAGAGAGAAGCAGGGCACCAAGGTGGCCGACAGCAGCAAGGGCCCC | |
| | GACGAGGCCAAGATCAAGGCCCTGCTGGAGAGAACCGGCTACACCCTG | |
| | GACGTGACCACCGGCCAGAGAAAGTACGGCGGCCCCCCCCCCGACAGC | |
| | GTGTACAGCGGCCAGCAGCCCAGCGTGGGCACCGAGATCTTCGTGGGCA | |
| | AGATCCCCAGAGACCTGTTCGAGGACGAGCTGGTGCCCCTGTTCGAGAA | |
| | GGCCGGCCCCATCTGGGACCTGAGACTGATGATGGACCCCCTGACCGGC | |
| | CTGAACAGAGGCTACGCCTTCGTGACCTTCTGCACCAAGGAGGCCGCCC | |
| | AGGAGGCCGTGAAGCTGTACAACAACCACGAGATCAGAAGCGGCAAGC | |
| | ACATCGGCGTGTGCATCAGCGTGGCCAACAACAGACTGTTCGTGGGCAG | |
| | CATCCCCAAGAGCAAGACCAAGGAGCAGATCCTGGAGGAGTTCAGCAA | |
| | GGTGACCGAGGGCCTGACCGACGTGATCCTGTACCACCAGCCCGACGAC | |
| | AAGAAGAAGAACAGAGGCTTCTGCTTCCTGGAGTACGAGGACCACAAGA | |
| | CCGCCGCCCAGGCCAGAAGAAGACTGATGAGCGGCAAGGTGAAGGTGT | |
| | GGGGCAACGTGGGCACCGTGGAGTGGGCCGACCCCATCGAGGACCCCG | |
| | ACCCCGAGGTGATGGCCAAGGTGAAGGTGCTGTTCGTGAGAAACCTGGC | |
| | CAACACCGTGACCGAGGAGATCCTGGAGAAGGCCTTCAGCCAGTTCGGC | |
| | AAGCTGGAGAGAGTGAAGAAGCTGAAGGACTACGCCTTCATCCACTTCG | |
| | ACGAGAGAGACGGCGCCGTGAAGGCCATGGAGGAGATGAACGGCAAG | |
| | GACCTGGAGGGCGAGAACATCGAGATCGTGTTCGCCAAGCCCCCCGACC | |
| | AGAAGAGAAAGGAGAGAAAGGCCCAGAGACAGGCCGCCAAGAACCAG | |
| | ATGTACGACGACTACTACTACTACGGCCCCCCCCACATGCCCCCCCCCACC | |
| | AGAGGCAGAGGCAGAGGCGGCAGAGGCGGCTACGGCTACCCCCCCGAC | |
| | TACTACGGCTACGAGGACTACTACGACTACTACGGCTACGACTACCACAA | |
| | CTACAGAGGCGGCTACGAGGACCCCTACTACGGCTACGAGGACTTCCAG | |
| | GTGGGCGCCAGAGGCAGAGGCGGCAGAGGCGCCAGAGGCGCCGCCCC | |
| | CAGCAGAGGCAGAGGCGCCGCCCCCCCCAGAGGCAGAGCCGGCTACAG | |
| | CCAGAGAGGCGGCCCCGGCAGCGCCAGAGGCGTGAGAGGCGCCAGAG | |
| | GCGGCGCCCAGCAGCAGAGAGGCAGAGGCGTGAGAGGCGCCAGAGGC | |
| | GGCAGAGGCGGCAACGTGGGCGGCAAGAGAAAGGCCGACGGCTACAA | |
| | CCAGCCCGACAGCAAGAGAAGACAGACCAACAACCAGAACTGGGGCAG | |
| | CCAGCCCATCGCCCAGCAGCCCCTGCAGGGCGGCGACCACAGCGGCAAC | |
| | TACGGCTACAAGAGCGAGAACCAGGAGTTCTACCAGGACACCTTCGGCC | |
| | AGCAGTGGAAGGGCAGCGGCGAGCAGAAGCTGATCTCAGAGGAGGAC | |
| | CTGTAGTAAGCGGCCGCTTCCCTTTAGTGAGG | |
| LA PROTEIN | CGACTCACTATAGGCTAGCCTCGAGGCCACCATGGCCGAGAACGGCGAC | 195 |
| | AACGAGAAGATGGCCGCCCTGGAGGCCAAGATCTGCCACCAGATCGAGT | |
| | ACTACTTCGGCGACTTCAACCTGCCCAGAGACAAGTTCCTGAAGGAGCA | |
| | GATCAAGCTGGACGAGGGCTGGGTGCCCCTGGAGATCATGATCAAGTTC | |
| | AACAGACTGAACAGACTGACCACCGACTTCAACGTGATCGTGGAGGCCC | |
| | TGAGCAAGAGCAAGGCCGAGCTGATGGAGATCAGCGAGGACAAGACCA | |
| | AGATCAGAAGAAGCCCCAGCAAGCCCCTGCCCGAGGTGACCGACGAGTA | |
| | CAAGAACGACGTGAAGAACAGAAGCGTGTACATCAAGGGCTTCCCCACC | |
| | GACGCCACCCTGGACGACATCAAGGAGTGGCTGGAGGACAAGGGCCAG | |
| | GTGCTGAACATCCAGATGAGAAGAACCCTGCACAAGGCCTTCAAGGGCA | |
| | GCATCTTCGTGGTGTTCGACAGCATCGAGAGCGCCAAGAAGTTCGTGGA | |
| | GACCCCCGGCCAGAAGTACAAGGAGACCGACCTGCTGATCCTGTTCAAG | |
| | GACGACTACTTCGCCAAGAAGAACGAGGAGAGAAAGCAGAACAAGGTG | |
| | GAGGCCAAGCTGAGAGCCAAGCAGGAGCAGGAGGCCAAGCAGAAGCT | |
| | GGAGGAGGACGCCGAGATGAAGAGCCTGGAGGAGAAGATCGGCTGCC | |
| | TGCTGAAGTTCAGCGGCGACCTGGACGACCAGACCTGCAGAGAGGACCT | |
| | GCACATCCTGTTCAGCAACCACGGCGAGATCAAGTGGATCGACTTCGTG | |
| | AGAGGCGCCAAGGAGGGCATCATCCTGTTCAAGGAGAAGGCCAAGGAG | |
| | GCCCTGGGCAAGGCCAAGGACGCCAACAACGGCAACCTGCAGCTGAGA | |
| | AACAAGGAGGTGACCTGGGAGGTGCTGGAGGGCGAGGTGGAGAAGGA | |
| | GGCCCTGAAGAAGATCATCGAGGACCAGCAGGAGAGCCTGAACAAGTG | |
| | GAAGAGCAAGGGCAGAAGATTCAAGGGCAAGGGCAAGGGCAACAAGG | |
| | CCGCCCAGCCCGGCAGCGGCAAGGGCAAGGTGCAGTTCCAGGGCAAGA | |
| | AGACCAAGTTCGCCAGCGACGACGAGCACGACGAGCACGACGAGAACG | |
| | GCGCCACCGGCCCCGTGAAGAGAGCCAGAGAGGAGACCGACAAGGAG | |
| | GAGCCCGCCAGCAAGCAGCAGAAGACCGAGAACGGCGCCGGCGACCAG | |
| | GGCAGCGGCGAGCAGAAGCTGATCTCAGAGGAGGACCTGTAGTAAGCG | |
| | GCCGCTTCCCTTTAGTGAGG | |
| ALYREF | CGACTCACTATAGGCTAGCCTCGAGGCCACCATGGCCGACAAGATGGAC | 196 |
| | ATGAGCCTGGACGACATCATCAAGCTGAACAGAAGCCAGAGAGGCGGC | |
| | AGAGGCGGCGGCAGAGGCAGAGGCAGAGCCGGCAGCCAGGGCGGCAG | |
| | AGGCGGCGGCGCCCAGGCCGCCGCCAGAGTGAACAGAGGCGGCGGCCC | |
| | CATCAGAAACAGACCCGCCATCGCCAGAGGCGCCGCCGGCGGCGGCGG | |
| | CAGAAACAGACCCGCCCCCTACAGCAGACCCAAGCAGCTGCCCGACAAG | |
| | TGGCAGCACGACCTGTTCGACAGCGGCTTCGGCGGCGGCGCCGGCGTG | |
| | GAGACCGGCGGCAAGCTGCTGGTGAGCAACCTGGACTTCGGCGTGAGC | |
| | GACGCCGACATCCAGGAGCTGTTCGCCGAGTTCGGCACCCTGAAGAAGG | |
| | CCGCCGTGCACTACGACAGAAGCGGCAGAAGCCTGGGCACCGCCGACG | |
| | TGCACTTCGAGAGAAAGGCCGACGCCCTGAAGGCCATGAAGCAGTACAA | |
| | CGGCGTGCCCCTGGACGGCAGACCCATGAACATCCAGCTGGTGACCAGC | |
| | CAGATCGACGCCCAGAGAAGACCCGCCCAGAGCGTGAACAGAGGCGGC | |
| | ATGACCAGAAACAGAGGCGCCGGCGGCTTCGGCGGCGGCGGCGGCACC | |
| | AGAAGAGGCACCAGAGGCGGCGCCAGAGGCAGAGGCAGAGGCGCCGG | |
| | CAGAAACAGCAAGCAGCAGCTGAGCGCCGAGGAGCTGGACGCCCAGCT | |
| | GGACGCCTACAACGCCAGAATGGACACCAGCGGCAGCGGCGAGCAGAA | |
| | GCTGATCTCAGAGGAGGACCTGTAGTAAGCGGCCGCTTCCCTTTAGTGA | |
| | GG | |
| FUS | CGACTCACTATAGGCTAGCCTCGAGGCCACCATGGCCAGCAACGACTAC | 197 |
| | ACCCAGCAGGCCACCCAGAGCTACGGCGCCTACCCCACCCAGCCCGGCC | |
| | AGGGCTACAGCCAGCAGAGCAGCCAGCCCTACGGCCAGCAGAGCTACA | |
| | GCGGCTACAGCCAGAGCACCGACACCAGCGGCTACGGCCAGAGCAGCT | |
| | ACAGCAGCTACGGCCAGAGCCAGAACACCGGCTACGGCACCCAGAGCAC | |
| | CCCCCAGGGCTACGGCAGCACCGGCGGCTACGGCAGCAGCCAGAGCAG | |
| | CCAGAGCAGCTACGGCCAGCAGAGCAGCTACCCCGGCTACGGCCAGCA | |
| | GCCCGCCCCCAGCAGCACCAGCGGCAGCTACGGCAGCAGCAGCCAGAG | |
| | CAGCAGCTACGGCCAGCCCCAGAGCGGCAGCTACAGCCAGCAGCCCAGC | |
| | TACGGCGGCCAGCAGCAGAGCTACGGCCAGCAGCAGAGCTACAACCCCC | |
| | CCCAGGGCTACGGCCAGCAGAACCAGTACAACAGCAGCAGCGGCGGCG | |
| | GCGGCGGCGGCGGCGGCGGCGGCAACTACGGCCAGGACCAGAGCAGC | |
| | ATGAGCAGCGGCGGCGGCAGCGGCGGCGGCTACGGCAACCAGGACCA | |
| | GAGCGGCGGCGGCGGCAGCGGCGGCTACGGCCAGCAGGACAGAGGCG | |
| | GCAGAGGCAGAGGCGGCAGCGGCGGCGGCGGCGGCGGCGGCGGCGG | |
| | CGGCTACAACAGAAGCAGCGGCGGCTACGAGCCCAGAGGCAGAGGCGG | |
| | CGGCAGAGGCGGCAGAGGCGGCATGGGGGGCAGCGACAGAGGCGGCT | |
| | TCAACAAGTTCGGCGGCCCCAGAGACCAGGGCAGCAGACACGACAGCG | |
| | AGCAGGACAACAGCGACAACAACACCATCTTCGTGCAGGGCCTGGGCGA | |
| | GAACGTGACCATCGAGAGCGTGGCCGACTACTTCAAGCAGATCGGCATC | |
| | ATCAAGACCAACAAGAAGACCGGCCAGCCCATGATCAACCTGTACACCG | |
| | ACAGAGAGACCGGCAAGCTGAAGGGCGAGGCCACCGTGAGCTTCGACG | |
| | ACCCCCCCAGCGCCAAGGCCGCCATCGACTGGTTCGACGGCAAGGAGTT | |
| | CAGCGGCAACCCCATCAAGGTGAGCTTCGCCACCAGAAGAGCCGACTTC | |
| | AACAGAGGCGGCGGCAACGGCAGAGGCGGCAGAGGCAGAGGCGGCCC | |
| | CATGGGCAGAGGCGGCTACGGCGGCGGCGGCAGCGGCGGCGGCGGCA | |
| | GAGGCGGCTTCCCCAGCGGCGGCGGCGGCGGCGGCGGCCAGCAGAGA | |
| | GCCGGCGACTGGAAGTGCCCCAACCCCACCTGCGAGAACATGAACTTCA | |
| | GCTGGAGAAACGAGTGCAACCAGTGCAAGGCCCCCAAGCCCGACGGCC | |
| | CCGGCGGCGGCCCCGGCGGCAGCCACATGGGCGGCAACTACGGCGACG | |
| | ACAGAAGAGGCGGCAGAGGCGGCTACGACAGAGGCGGCTACAGAGGC | |
| | AGAGGCGGCGACAGAGGCGGCTTCAGAGGCGGCAGAGGCGGCGGCGA | |
| | CAGAGGCGGCTTCGGCCCCGGCAAGATGGACAGCAGAGGCGAGCACAG | |
| | ACAGGACAGAAGAGAGAGACCCTACGGCAGCGGCGAGCAGAAGCTGAT | |
| | CTCAGAGGAGGACCTGTAGTAAGCGGCCGCTTCCCTTTAGTGAGG | |
| MEX3C-1 | CGACTCACTATAGGCTAGCCTCGAGGCCACCATGCCCAGCGGCAGCAGC | 198 |
| | GCCGCCCTGGCCCTGGCCGCCGCCCCCGCCCCCCTGCCCCAGCCCCCCCC | |
| | CCCCCCCCCCCCCCCCCCCCCCCCCCTGCCCCCCCCCAGCGGCGGCCCCGA | |
| | GCTGGAGGGCGACGGCCTGCTGCTGAGAGAGAGACTGGCCGCCCTGGG | |
| | CCTGGACGACCCCAGCCCCGCCGAGCCCGGCGCCCCCGCCCTGAGAGCC | |
| | CCCGCCGCCGCCGCCCAGGGCCAGGCCAGAAGAGCCGCCGAGCTGAGC | |
| | CCCGAGGAGAGAGCCCCCCCCGGCAGACCCGGCGCCCCCGAGGCCGCC | |
| | GAGCTGGAGCTGGAGGAGGACGAGGAGGAGGGCGAGGAGGCCGAGCT | |
| | GGACGGCGACCTGCTGGAGGAGGAGGAGCTGGAGGAGGCCGAGGAGG | |
| | AGGACAGAAGCAGCCTGCTGCTGCTGAGCCCCCCCGCCGCCACCGCCAG | |
| | CCAGACCCAGCAGATCCCCGGCGGCAGCCTGGGCAGCGTGCTGCTGCCC | |
| | GCCGCCAGATTCGACGCCAGAGAGGCCGCCGCCGCCGCCGCCGCCGCCG | |
| | GCGTGCTGTACGGCGGCGACGACGCCCAGGGCATGATGGCCGCCATGCT | |
| | GAGCCACGCCTACGGCCCCGGCGGCTGCGGCGCCGCCGCCGCCGCCCTG | |
| | AACGGCGAGCAGGCCGCCCTGCTGAGAAGAAAGAGCGTGAACACCACC | |
| | GAGTGCGTGCCCGTGCCCAGCAGCGAGCACGTGGCCGAGATCGTGGGC | |
| | AGACAGGGCTGCAAGATCAAGGCCCTGAGAGCCAAGACCAACACCTACA | |
| | TCAAGACCCCCGTGAGAGGCGAGGAGCCCATCTTCGTGGTGACCGGCAG | |
| | AAAGGAGGACGTGGCCATGGCCAAGAGAGAGATCCTGAGCGCCGCCGA | |
| | GCACTTCAGCATGATCAGAGCCAGCAGAAACAAGAACGGCCCCGCCCTG | |
| | GGCGGCCTGAGCTGCAGCCCCAACCTGCCCGGCCAGACCACCGTGCAGG | |
| | TGAGAGTGCCCTACAGAGTGGTGGGCCTGGTGGTGGGCCCCAAGGGCG | |
| | CCACCATCAAGAGAATCCAGCAGCAGACCCACACCTACATCGTGACCCCC | |
| | AGCAGAGACAAGGAGCCCGTGTTCGAGGTGACCGGCATGCCCGAGAAC | |
| | GTGGACAGAGCCAGAGAGGAGATCGAGATGCACATCGCCATGAGAACC | |
| | GGCAACTACATCGAGCTGAACGAGGAGAACGACTTCCACTACAACGGCA | |
| | CCGACGTGAGCTTCGAGGGCGGCACCCTGGGCAGCGCCTGGCTGAGCA | |
| | GCAACCCCGTGCCCCCCAGCAGAGCCAGAATGATCAGCAACTACAGAAA | |
| | CGACAGCAGCAGCAGCCTGGGCAGCGGCAGCACCGACAGCTACTTCGG | |
| | CAGCAACAGACTGGCCGACTTCAGCCCCACCAGCCCCTTCAGCACCGGCA | |
| | ACTTCTGGTTCGGCGACACCCTGCCCAGCGTGGGCAGCGAGGACCTGGC | |
| | CGTGGACAGCCCCGCCTTCGACAGCCTGCCCACCAGCGCCCAGACCATCT | |
| | GGACCCCCTTCGAGCCCGTGAACCCCCTGAGCGGCTTCGGCAGCGACCC | |
| | CAGCGGCAACATGAAGACCCAGAGAAGAGGCAGCCAGCCCAGCACCCC | |
| | CAGACTGAGCCCCACCTTCCCCGAGAGCATCGAGCACCCCCTGGCCAGA | |
| | AGAGTGAGAAGCGACCCCCCCAGCACCGGCAACCACGTGGGCCTGCCCA | |
| | TCTACATCCCCGCCTTCAGCAACGGCACCAACAGCTACAGCAGCAGCAAC | |
| | GGCGGCAGCACCAGCAGCAGCCCCCCCGAGAGCAGAAGAAAGCACGAC | |
| | TGCGTGATCTGCTTCGAGAACGAGGTGATCGCCGCCCTGGTGCCCTGCG | |
| | GCCACAACCTGTTCTGCATGGAGTGCGCCAACAAGATCTGCGAGAAGAG | |
| | AACCCCCAGCTGCCCCGTGTGCCAGACCGCCGTGACCCAGGCCATCCAG | |
| | ATCCACAGCGGCAGCGGCGAGCAGAAGCTGATCTCAGAGGAGGACCTG | |
| | TAGTAAGCGGCCGCTTCCCTTTAGTGAGG | |
| CD63 | CGACTCACTATAGGCTAGCCTCGAGGCCACCATGGCGGTGGAAGGAGG | 199 |
| | AATGAAATGTGTGAAGTTCTTGCTCTACGTCCTCCTGCTGGCCTTTTGCGC | |
| | CTGTGCAGTGGGACTGATTGCCGTGGGTGTCGGGGCACAGCTTGTCCTG | |
| | AGTCAGACCATAATCCAGGGGGCTACCCCTGGCTCTCTGTTGCCAGTGGT | |
| | CATCATCGCAGTGGGTGTCTTCCTCTTCCTGGTGGCTTTTGTGGGCTGCT | |
| | GCGGGGCCTGCAAGGAGAACTATTGTCTTATGATCACGTTTGCCATCTTT | |
| | CTGTCTCTTATCATGTTGGTGGAGGTGGCCGCAGCCATTGCTGGCTATGT | |
| | GTTTAGAGATAAGGTGATGTCAGAGTTTAATAACAACTTCCGGCAGCAG | |
| | ATGGAGAATTACCCGAAAAACAACCACACTGCTTCGATCCTGGACAGGA | |
| | TGCAGGCAGATTTTAAGTGCTGTGGGGCTGCTAACTACACAGATTGGGA | |
| | GAAAATCCCTTCCATGTCGAAGAACCGAGTCCCCGACTCCTGCTGCATTA | |
| | ATGTTACTGTGGGCTGTGGGATTAATTTCAACGAGAAGGCGATCCATAA | |
| | GGAGGGCTGTGTGGAGAAGATTGGGGGCTGGCTGAGGAAAAATGTGCT | |
| | GGTGGTAGCTGCAGCAGCCCTTGGAATTGCTTTTGTCGAGGTTTTGGGA | |
| | ATTGTCTTTGCCTGCTGCCTCGTGAAGAGTATCAGAAGTGGCTACGAGGT | |
| | GATGTAGTAAGCGGCCGCTTCCCTTTAGTGAGG | |
| CD63-U1a | CGACTCACTATAGGCTAGCCTCGAGGCCACCATGGCGGTGGAAGGAGG | 200 |
| | AATGAAATGTGTGAAGTTCTTGCTCTACGTCCTCCTGCTGGCCTTTTGCGC | |
| | CTGTGCAGTGGGACTGATTGCCGTGGGTGTCGGGGCACAGCTTGTCCTG | |
| | AGTCAGACCATAATCCAGGGGGCTACCCCTGGCTCTCTGTTGCCAGTGGT | |
| | CATCATCGCAGTGGGTGTCTTCCTCTTCCTGGTGGCTTTTGTGGGCTGCT | |
| | GCGGGGCCTGCAAGGAGAACTATTGTCTTATGATCACGTTTGCCATCTTT | |
| | CTGTCTCTTATCATGTTGGTGGAGGTGGCCGCAGCCATTGCTGGCTATGT | |
| | GTTTAGAGATAAGGTGATGTCAGAGTTTAATAACAACTTCCGGCAGCAG | |
| | ATGGAGAATTACCCGAAAAACAACCACACTGCTTCGATCCTGGACAGGA | |
| | TGCAGGCAGATTTTAAGTGCTGTGGGGCTGCTAACTACACAGATTGGGA | |
| | GAAAATCCCTTCCATGTCGAAGAACCGAGTCCCCGACTCCTGCTGCATTA | |
| | ATGTTACTGTGGGCTGTGGGATTAATTTCAACGAGAAGGCGATCCATAA | |
| | GGAGGGCTGTGTGGAGAAGATTGGGGGCTGGCTGAGGAAAAATGTGCT | |
| | GGTGGTAGCTGCAGCAGCCCTTGGAATTGCTTTTGTCGAGGTTTTGGGA | |
| | ATTGTCTTTGCCTGCTGCCTCGTGAAGAGTATCAGAAGTGGCTACGAGGT | |
| | GATGGAATTCGGCGGAGGCGGGTCCATGGCAGTTCCCGAGACCCGCCCT | |
| | AACCACACTATTTATATCAACAACCTCAATGAGAAGATCAAGAAGGATGA | |
| | GCTAAAAAAGTCCCTGTACGCCATCTTCTCCCAGTTTGGCCAGATCCTGG | |
| | ATATCCTGGTATCACGGAGCCTGAAGATGAGGGGCCAGGCCTTTGTCAT | |
| | CTTCAAGGAGGTCAGCAGCGCCACCAACGCCCTGCGCTCCATGCAGGGT | |
| | TTCCCTTTCTATGACAAACCTATGCGTATCCAGTATGCCAAGACCGACTCA | |
| | GATATCATTGCCAAGATGAAATAGTAAGCGGCCGCTTCCCTTTAGTGAGG | |
| Nluc-Ago2 | GATTACAAGGATGACGATGACAAGCTTGTCTTCACACTCGAAGATTTCGT | 201 |
| | TGGGGACTGGCGACAGACAGCCGGCTACAACCTGGACCAAGTCCTTGAA | |
| | CAGGGAGGTGTGTCCAGTTTGTTTCAGAATCTCGGGGTGTCCGTAACTCC | |
| | GATCCAAAGGATTGTCCTGAGCGGTGAAAATGGGCTGAAGATCGACATC | |
| | CATGTCATCATCCCGTATGAAGGTCTGAGCGGCGACCAAATGGGCCAGA | |
| | TCGAAAAAATTTTTAAGGTGGTGTACCCTGTGGATGATCATCACTTTAAG | |
| | GTGATCCTGCACTATGGCACACTGGTAATCGACGGGGTTACGCCGAACA | |
| | TGATCGACTATTTCGGACGGCCGTATGAAGGCATCGCCGTGTTCGACGG | |
| | CAAAAAGATCACTGTAACAGGGACCCTGTGGAACGGCAACAAAATTATC | |
| | GACGAGCGCCTGATCAACCCCGACGGCTCCCTGCTGTTCCGAGTAACCAT | |
| | CAACGGAGTGACCGGCTGGCGGCTGTGCGAACGCATTCTGGCGGGAGG | |
| | TGGATCCATGCACCCATTCCAGTG | |
| U6-F | GAGGGCCTATTTCCCATGATT | 202 |
| Cas13d (CasRx) Dicer- | AAAAAAAAAATGGGTCCTTTCTTTGGACTGCGTTTCAAACCCCGACCAGT | 203 |
| crRNA-R | TGGTAGGGGTACGGTGTTTCGTCCTTTCCACAA | |
| MQ-F Amp seq | ACACTCTTTCCCTACACGACGCTCTTCCGATCTTATGACCATGATTACGCC | 204 |
| | AAGCTTGGTACCGAGCTCGGATCCACTAGTAACGGCCGCCAGTGTGCTG | |
| | GAATTCGGCTTAAGCAGTGGTATCAACGCAGAGTAC | |
| AS-1 AmpEz R | GACTGGAGTTCAGACGTGTGCTCTTCCGATCTATGAACTTCAGGGTCAGC | 205 |
| shGFP qPCR F 18 nt | ATGAACTTCAGGGTCAGC | 206 |
| shGFP qPCR F 19 nt | ATGAACTTCAGGGTCAGCT | 207 |
| shGFP qPCR F 20 nt | ATGAACTTCAGGGTCAGCTT | 208 |
| shGFP qPCR F 21 nt | ATGAACTTCAGGGTCAGCTTG | 209 |
| shGFP qPCR F 22 nt | ATGAACTTCAGGGTCAGCTTGC | 210 |
| siGFP RNA 18 nt | AUGAACUUCAGGGUCAGC | 211 |
| hDICER1 qPCR F | AATATCAGGTTGAACTGCTTGA | 212 |
| hDICER1 qPCR R | TGCAATAAATGTCTTCCCTGAG | 213 |
| GAPDH F | CTCTGCTCCTCCTGTTCGAC | 214 |
| GAPDH R | TTAAAAGCAGCCCTGGTGAC | 215 |

TABLE 7
Amino acid sequences ot RAS proteins and mutants

| Name | SEQ ID NO | Amino acids (N-C) |
|---|---|---|
| KRAS | 217 | MTEYKLVVVGAGGVGKSALTIQLIQNHFVDEYDPTIEDSYRKQVVIDG |
| | | ETCLLDILDTAGQEEYSAMRDQYMRTGEGFLCVFAINNTKSFEDIHHY |
| | | REQIKRVKDSEDVPMVLVGNKCDLPSRTVDTKQAQDLARSYGIPFIETS |
| | | AKTRQGVDDAFYTLVREIRKHKEKMSKDGKKKKKKSKTKCVIM |
| KRAS-S17N | 218 | MTEYKLVVVGAGGVGKNALTIQLIQNHFVDEYDPTIEDSYRKQVVIDG |
| | | ETCLLDILDTAGQEEYSAMRDQYMRTGEGFLCVFAINNTKSFEDIHHY |
| | | REQIKRVKDSEDVPMVLVGNKCDLPSRTVDTKQAQDLARSYGIPFIETS |
| | | AKTRQGVDDAFYTLVREIRKHKEKMSKDGKKKKKKSKTKCVIM |
| KRAS-G12D | 219 | MTEYKLVVVGADGVGKSALTIQLIQNHFVDEYDPTIEDSYRKQVVIDGE |
| | | TCLLDILDTAGQEEYSAMRDQYMRTGEGFLCVFAINNTKSFEDIHHYR |
| | | EQIKRVKDSEDVPMVLVGNKCDLPSRTVDTKQAQDLARSYGIPFIETSA |
| | | KTRQGVDDAFYTLVREIRKHKEKMSKDGKKKKKKSKTKCVIM |
| KRAS-G12V | 220 | MTEYKLVVVGAVGVGKSALTIQLIQNHFVDEYDPTIEDSYRKQVVIDGE |
| | | TCLLDILDTAGQEEYSAMRDQYMRTGEGFLCVFAINNTKSFEDIHHYR |
| | | EQIKRVKDSEDVPMVLVGNKCDLPSRTVDTKQAQDLARSYGIPFIETSA |
| | | KTRQGVDDAFYTLVREIRKHKEKMSKDGKKKKKKSKTKCVIM |
| HRAS | 221 | MTEYKLVVVGAGGVGKSALTIQLIQNHFVDEYDPTIEDSYRKQVVIDG |
| | | ETCLLDILDTAGQEEYSAMRDQYMRTGEGFLCVFAINNTKSFEDIHQY |
| | | REQIKRVKDSDDVPMVLVGNKCDLAARTVESRQAQDLARSYGIPYIET |
| | | SAKTRQGVEDAFYTLVREIRQHKLRKLNPPDESGPGCMSCKCVLS |
| HRAS-S17N | 222 | MTEYKLVVVGAGGVGKNALTIQLIQNHFVDEYDPTIEDSYRKQVVIDG |
| | | ETCLLDILDTAGQEEYSAMRDQYMRTGEGFLCVFAINNTKSFEDIHQY |
| | | REQIKRVKDSDDVPMVLVGNKCDLAARTVESRQAQDLARSYGIPYIET |
| | | SAKTRQGVEDAFYTLVREIRQHKLRKLNPPDESGPGCMSCKCVLS |
| HRAS-G12D | 223 | MTEYKLVVVGADGVGKSALTIQLIQNHFVDEYDPTIEDSYRKQVVIDGE |
| | | TCLLDILDTAGQEEYSAMRDQYMRTGEGFLCVFAINNTKSFEDIHQYR |
| | | EQIKRVKDSDDVPMVLVGNKCDLAARTVESRQAQDLARSYGIPYIETS |
| | | AKTRQGVEDAFYTLVREIRQHKLRKLNPPDESGPGCMSCKCVLS |
| HRAS-G12V | 224 | MTEYKLVVVGAVGVGKSALTIQLIQNHFVDEYDPTIEDSYRKQVVIDGE |
| | | TCLLDILDTAGQEEYSAMRDQYMRTGEGFLCVFAINNTKSFEDIHQYR |
| | | EQIKRVKDSDDVPMVLVGNKCDLAARTVESRQAQDLARSYGIPYIETS |
| | | AKTRQGVEDAFYTLVREIRQHKLRKLNPPDESGPGCMSCKCVLS |
| NRAS | 225 | MTEYKLVVVGAGGVGKSALTIQLIQNHFVDEYDPTIEDSYRKQVVIDG |
| | | ETCLLDILDTAGQEEYSAMRDQYMRTGEGFLCVFAINNSKSFADINLYR |
| | | EQIKRVKDSDDVPMVLVGNKCDLPTRTVDTKQAHELAKSYGIPFIETSA |
| | | KTRQGVEDAFYTLVREIRQYRMKKLNSSDDGTQGCMGLPCVVM |
| NRAS-S17N | 226 | MTEYKLVVVGAGGVGKNALTIQLIQNHFVDEYDPTIEDSYRKQVVIDG |
| | | ETCLLDILDTAGQEEYSAMRDQYMRTGEGFLCVFAINNSKSFADINLYR |
| | | EQIKRVKDSDDVPMVLVGNKCDLPTRTVDTKQAHELAKSYGIPFIETSA |
| | | KTRQGVEDAFYTLVREIRQYRMKKLNSSDDGTQGCMGLPCVVM |
| NRAS-G12D | 227 | MTEYKLVVVGADGVGKSALTIQLIQNHFVDEYDPTIEDSYRKQVVIDGE |
| | | TCLLDILDTAGQEEYSAMRDQYMRTGEGFLCVFAINNSKSFADINLYRE |
| | | QIKRVKDSDDVPMVLVGNKCDLPTRTVDTKQAHELAKSYGIPFIETSAK |
| | | TRQGVEDAFYTLVREIRQYRMKKLNSSDDGTQGCMGLPCVVM |
| NRAS-G12V | 228 | MTEYKLVVVGAVGVGKSALTIQLIQNHFVDEYDPTIEDSYRKQVVIDGE |
| | | TCLLDILDTAGQEEYSAMRDQYMRTGEGFLCVFAINNSKSFADINLYRE |
| | | QIKRVKDSDDVPMVLVGNKCDLPTRTVDTKQAHELAKSYGIPFIETSAK |
| | | TRQGVEDAFYTLVREIRQYRMKKLNSSDDGTQGCMGLPCVVM |

TABLE 8
ShV PS4B and CHMP4C shRNA sequences
Bold text indicates the small RNA promoter (H1) and underlined is the Pol III
Poly T transcription terminator.

| | SEQ ID | | SEQ ID | |
|---|---|---|---|---|
| | NO | Sequence (5′-3′) | NO | shRNA |
| ShVPS4B | 229 | ATATTTGCATGTCGCTATGTGTTCTGG | 231 | GCGAAGAUUUGA |
| | | GAAATCACCATAAACGTGAAATGTCT | | GAAAUGAGUUAU |
| | | TTGGATTTGGGAATCTTATAAGTTCTG | | CAAGAGUAAUUCG |
| | | TATGAGACCACTCAGATCCGCGAAGA | | UUUCUCAAAUCU |
| | | TTTGAGAAATGAGTTATCAAGAGTAAT | | UCGC |
| | | TCGTTTCTCAAATCTTCGCTTTTTT | | |
| shCHMP | 230 | ATATTTGCATGTCGCTATGTGTTCTGGGAAATC | 232 | GCAGAAUAAGCGA |
| 4C_sh3 | | ACCATAAACGTGAAATGTCTTTGGATTTGGGA | | GUUGCGUUAUCA |
| | | ATCTTATAAGTTCTGTATGAGACCACTCAGATC | | AGAGUAAUGCAGC |
| | | CGCAGAATAAGCGAGTTGCGTTATCAAGAGTA | | UCGCUUAUUCUG |
| | | ATGCAGCTCGCTTATTCTGCTTTTTT | | C |

ADDITIONAL SEQUENCES

pLenti-E1falpa-GFP-Fluc-IRES-Puro (SEQ ID NO: 153)
Bold: EF-1 core/5′ LTR (truncated) promoter; bold/italic: eGFP; italic: Fluc;
underlined: IRES; underlined/bold: puromycin resistance gene
GGATCTGCGATCGCTCCGGTGCCCGTCAGTGGGCAGAGCGCACATCGCCCACAGT
CCCCGAGAAGTTGGGGGGAGGGGTCGGCAATTGAACCGGTGCCTAGAGAAGGTG
GCGCGGGGTAAACTGGGAAAGTGATGTCGTGTACTGGCTCCGCCTTTTTCCCGAG
GGTGGGGGAGAACCGTATATAAGTGCAGTAGTCGCCGTGAACGTTCTTTTTCGCA
ACGGGTTTGCCGCCAGAACACAGCTGAAGCTTCGAGGGGCTCGCATCTCTCCTTCAC
GCGCCCGCCGCCCTACCTGAGGCCGCCATCCACGCCGGTTGAGTCGCGTTCTGCCGCCT
CCCGCCTGTGGTGCCTCCTGAACTGCGTCCGCCGTCTAGGTAAGTTTAAAGCTCAGGTC
GAGACCGGGCCTTTGTCCGGCGCTCCCTTGGAGCCTACCTAGACTCAGCCGGCTCTCCA
CGCTTTGCCTGACCCTGCTTGCTCAACTCTACGTCTTTGTTTCGTTTTCTGTTCTGCGCCG
TTACAGATCCAAGCTGTGACCGGCGCCTACCAAGTTTGTACAAAAAAGCAGGCTGCCA
CCATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGCCAC
CTACGGCAAGCTGACCCTGAAGTTCATCTGCACCACCGGCAAGCTGCCCGTGCCCTGGCC
CACCCTCGTGACCACCCTGACCTACGGCGTGCAGTGCTTCAGCCGCTACCCCGACCACAT
GAAGCAGCACGACTTCTTCAAGTCCGCCATGCCCGAAGGCTACGTCCAGGAGCGCACCAT
CTTCTTCAAGGACGACGGCAACTACAAGACCCGCGCCGAGGTGAAGTTCGAGGGCGACA
CCCTGGTGAACCGCATCGAGCTGAAGGGCATCGACTTCAAGGAGGACGGCAACATCCTG
GGGCACAAGCTGGAGTACAACTACAACAGCCACAACGTCTATATCATGGCCGACAAGCAG
AAGAACGGCATCAAGGTGAACTTCAAGATCCGCCACAACATCGAGGACGGCAGCGTGCA
GCTCGCCGACCACTACCAGCAGAACACCCCCATCGGCGACGGCCCCGTGCTGCTGCCCG
ACAACCACTACCTGAGCACCCAGTCCGCCCTGAGCAAAGACCCCAACGAGAAGCGCGAT
CACATGGTCCTGCTGGAGTTCGTGACCGCCGCCGGGATCACTCTCGGCATGGACGAGCT
GTACAAGGGAGGAGGAATGGAGGATGCCAAGAATATTAAGAAAGGCCCTGCCCCATTCTACC
CTCTGGAAGATGGCACTGCTGGTGAGCAACTGCACAAGGCCATGAAGAGGTATGCCCTGGTC
CCTGGCACCATTGCCTTCACTGATGCTCACATTGAGGTGGACATCACCTATGCTGAATACTTT
GAGATGTCTGTGAGGCTGGCAGAAGCCATGAAAAGATATGGACTGAACACCAACCACAGGAT
TGTGGTGTGCTCTGAGAACTCTCTCCAGTTCTTCATGCCTGTGTTAGGAGCCCTGTTCATTGG
AGTGGCTGTGGCCCCTGCCAATGACATCTACAATGAGAGAGAGCTCCTGAACAGCATGGGCA
TCAGCCAGCCAACTGTGGTCTTTGTGAGCAAGAAGGGCCTGCAAAAGATCCTGAATGTGCAG
AAGAAGCTGCCCATCATCCAGAAGATCATCATCATGGACAGCAAGACTGACTACCAGGGCTTC
CAGAGCATGTATACCTTTGTGACCAGCCACTTACCCCCTGGCTTCAATGAGTATGACTTTGTG
CCTGAGAGCTTTGACAGGGACAAGACCATTGCTCTGATTATGAACAGCTCTGGCTCCACTGGA
CTGCCCAAAGGTGTGGCTCTGCCCCACAGAACTGCTTGTGTGAGATTCAGCCATGCCAGAGA
CCCCATCTTTGGCAACCAGATCATCCCTGACACTGCCATCCTGTCTGTGGTTCCATTCCATCA
TGGCTTTGGCATGTTCACAACACTGGGGTACCTGATCTGTGGCTTCAGAGTGGTGCTGATGTA
TAGGTTTGAGGAGGAGCTGTTTCTGAGGAGCCTACAAGACTACAAGATCCAGTCTGCCCTGC
TGGTGCCCACTCTGTTCAGCTTCTTTGCCAAGAGCACCCTCATTGACAAGTATGACCTGAGCA
ACCTGCATGAGATTGCCTCTGGAGGAGCACCCCTGAGCAAGGAGGTGGGTGAGGCTGTGGC
AAAGAGGTTCCATCTCCCAGGAATCAGACAGGGCTATGGCCTGACTGAGACCACCTCTGCCA
TCCTCATCACCCCTGAAGGAGATGACAAGCCTGGTGCTGTGGGCAAGGTGGTTCCCTTTTTT
GAGGCCAAGGTGGTGGACCTGGACACTGGCAAGACCCTGGGAGTGAACCAGAGGGGTGAG
CTGTGTGTGAGGGGTCCCATGATCATGTCTGGCTATGTGAACAACCCTGAGGCCACCAATGC
CCTGATTGACAAGGATGGCTGGCTGCACTCTGGTGACATTGCCTACTGGGATGAGGATGAGC
ACTTTTTCATTGTGGACAGGCTGAAGAGCCTCATCAAGTACAAAGGCTACCAAGTGGCACCTG
CTGAGCTAGAGAGCATCCTGCTCCAGCACCCCAACATCTTTGATGCTGGTGTGGCTGGCCTG
CCTGATGATGATGCTGGAGAGCTGCCTGCTGCTGTTGTGGTTCTGGAGCATGGAAAGACCAT
GACTGAGAAGGAGATTGTGGACTATGTGGCCAGTCAGGTGACCACTGCCAAGAAGCTGAGG
GGAGGTGTGGTGTTTGTGGATGAGGTGCCAAAGGGTCTGACTGGCAAGCTGGATGCCAGAA
AGATCAGAGAGATCCTGATCAAGGCCAAGAAGGGTGGCAAATGAACCCAGCTTTCTTGTACAA
AGTGGGCCCCTCTCCCTCCCCCCCCCCTAACGTTACTGGCCGAAGCCGCTTGGAATAAGGCC
GGTGTGCGTTTGTCTATATGTTATTTTCCACCATATTGCCGTCTTTTGGCAATGTGAGGGCCCG
GAAACCTGGCCCTGTCTTCTTGACGAGCATTCCTAGGGGTCTTTCCCCTCTCGCCAAAGGAAT
GCAAGGTCTGTTGAATGTCGTGAAGGAAGCAGTTCCTCTGGAAGCTTCTTGAAGACAAACAAC
GTCTGTAGCGACCCTTTGCAGGCAGCGGAACCCCCCACCTGGCGACAGGTGCCTCTGCGGC
CAAAAGCCACGTGTATAAGATACACCTGCAAAGGCGGCACAACCCCAGTGCCACGTTGTGAG
TTGGATAGTTGTGGAAAGAGTCAAATGGCTCTCCTCAAGCGTATTCAACAAGGGGCTGAAGGA
TGCCCAGAAGGTACCCCATTGTATGGGATCTGATCTGGGGCCTCGGTGCACATGCTTTACAT
GTGTTTAGTCGAGGTTAAAAAAACGTCTAGGCCCCCCGAACCACGGGGACGTGGTTTTCCTTT
GAAAAACACGATGATAATATGGCCACAACCATGACCGAGTACAAGCCCACGGTGCGCCTCGC
CACCCGCGACGACGTCCCCAGGGCCGTACGCACCCTCGCCGCCGCGTTCGCCGACTACCC
CGCCACGCGCCACACCGTCGATCCGGACCGCCACATCGAGCGGGTCACCGAGCTGCAAGAA
CTCTTCCTCACGCGCGTCGGGCTCGACATCGGCAAGGTGTGGGTCGCGGACGACGGCGCC
GCGGTGGCGGTCTGGACCACGCCGGAGAGCGTCGAAGCGGGGGCGGTGTTCGCCGAGATC
GGCCCGCGCATGGCCGAGTTGAGCGGTTCCCGGCTGGCCGCGCAGCAACAGATGGAAGGC
CTCCTGGCGCCGCACCGGCCCAAGGAGCCCGCGTGGTTCCTGGCCACCGTCGGCGTCTCG
CCCGACCACCAGGGCAAGGGTCTGGGCAGCGCCGTCGTGCTCCCCGGAGTGGAGGCGGCC
GAGCGCGCCGGGGTGCCCGCCTTCCTGGAGACCTCCGCGCCCCGCAACCTCCCCTTCTACG
AGCGGCTCGGCTTCACCGTCACCGCCGACGTCGAGGTGCCCGAAGGACCGCGCACCTGGT
GCATGACCCGCAAGCCCGGTGCCTGAACCCAGCTTTCTTGTACAAAGTGGGCCCCTCTCC
CTCCCCCCCCCCTAACGTTACTGGCCGAAGCCGCTTGGAATAAGGCCGGTGTGCGTTTG
TCTATATGTTATTTTCCACCATATTGCCGTCTTTTGGCAATGTGAGGGCCCGGAAACCTG
GCCCTGTCTTCTTGACGAGCATTCCTAGGGGTCTTTCCCCTCTCGCCAAAGGAATGCAA
GGTCTGTTGAATGTCGTGAAGGAAGCAGTTCCTCTGGAAGCTTCTTGAAGACAAACAAC
GTCTGTAGCGACCCTTTGCAGGCAGCGGAACCCCCCACCTGGCGACAGGTGCCTCTGCG
GCCAAAAGCCACGTGTATAAGATACACCTGCAAAGGCGGCACAACCCCAGTGCCACGT
TGTGAGTTGGATAGTTGTGGAAAGAGTCAAATGGCTCTCCTCAAGCGTATTCAACAAGG
GGCTGAAGGATGCCCAGAAGGTACCCCATTGTATGGGATCTGATCTGGGGCCTCGGTG
CACATGCTTTACATGTGTTTAGTCGAGGTTAAAAAAACGTCTAGGCCCCCCGAACCACG
GGGACGTGGTTTTCCTTTGAAAAACACGATGATAATATGGCCACAACCATGACCGAGT
ACAAGCCCACGGTGCGCCTCGCCACCCGCGACGACGTCCCCAGGGCCGTACGCAC
CCTCGCCGCCGCGTTCGCCGACTACCCCGCCACGCGCCACACCGTCGATCCGGAC
CGCCACATCGAGCGGGTCACCGAGCTGCAAGAACTCTTCCTCACGCGCGTCGGGC
TCGACATCGGCAAGGTGTGGGTCGCGGACGACGGCGCCGCGGTGGCGGTCTGGA
CCACGCCGGAGAGCGTCGAAGCGGGGGCGGTGTTCGCCGAGATCGGCCCGCGCA
TGGCCGAGTTGAGCGGTTCCCGGCTGGCCGCGCAGCAACAGATGGAAGGCCTCCT
GGCGCCGCACCGGCCCAAGGAGCCCGCGTGGTTCCTGGCCACCGTCGGCGTCTC
GCCCGACCACCAGGGCAAGGGTCTGGGCAGCGCCGTCGTGCTCCCCGGAGTGGA
GGCGGCCGAGCGCGCCGGGGTGCCCGCCTTCCTGGAGACCTCCGCGCCCCGCAA
CCTCCCCTTCTACGAGCGGCTCGGCTTCACCGTCACCGCCGACGTCGAGGTGCCC
GAAGGACCGCGCACCTGGTGCATGACCCGCAAGCCCGGTGCCTGA
Nluc-Ago2 DNA sequence (SEQ ID NO: 154)
Bold: Flag-tag; italic: Nluc; underline: linker; bold/underline: A go2
ATGGACTACAAAGACCATGACGGTGATTATAAAGATCATGACATCGATTACAAGGA
TGACGATGACAAGCTTGTCTTCACACTCGAAGATTTCGTTGGGGACTGGCGACAGACAGC
CGGCTACAACCTGGACCAAGTCCTTGAACAGGGAGGTGTGTCCAGTTTGTTTCAGAATCTCG
GGGTGTCCGTAACTCCGATCCAAAGGATTGTCCTGAGCGGTGAAAATGGGCTGAAGATCGAC
ATCCATGTCATCATCCCGTATGAAGGTCTGAGCGGCGACCAAATGGGCCAGATCGAAAAAATT
TTTAAGGTGGTGTACCCTGTGGATGATCATCACTTTAAGGTGATCCTGCACTATGGCACACTG
GTAATCGACGGGGTTACGCCGAACATGATCGACTATTTCGGACGGCCGTATGAAGGCATCGC
CGTGTTCGACGGCAAAAAGATCACTGTAACAGGGACCCTGTGGAACGGCAACAAAATTATCG
ACGAGCGCCTGATCAACCCCGACGGCTCCCTGCTGTTCCGAGTAACCATCAACGGAGTGACC
GGCTGGCGGCTGTGCGAACGCATTCTGGCGGGAGGTGGATCCATGCACCCATTCCAGT
GGTGTAACGCACTTGCACCTCCTGCGCCGCCGCCCCCCATCCAAGGATATGCCTT
CAAGCCTCCACCTAGACCCGACTTTGGGACCTCCGGGAGAACAATCAAATTACAG
GCCAATTTCTTCGAAATGGACATCCCCAAAATTGACATCTATCATTATGAATTGGA
TATCAAGCCAGAGAAGTGCCCGAGGAGAGTTAACAGGGAAATCGTGGAACACATG
GTCCAGCACTTTAAAACACAGATCTTTGGGGATCGGAAGCCCGTGTTTGACGGCA
GGAAGAATCTATACACAGCCATGCCCCTTCCGATTGGGAGGGACAAGGTGGAGCT
GGAGGTCACGCTGCCAGGAGAAGGCAAGGATCGCATCTTCAAGGTGTCCATCAAG
TGGGTGTCCTGCGTGAGCTTGCAGGCGTTACACGATGCACTTTCAGGGCGGCTGC
CCAGCGTCCCTTTTGAGACGATCCAGGCCCTGGACGTGGTCATGAGGCACTTGCC
ATCCATGAGGTACACCCCCGTGGGCCGCTCCTTCTTCACCGCGTCCGAAGGCTGC
TCTAACCCTCTTGGCGGGGGCCGAGAAGTGTGGTTTGGCTTCCATCAGTCCGTCC
GGCCTTCTCTCTGGAAAATGATGCTGAATATTGATGTGTCAGCAACAGCGTTTTAC
AAGGCACAGCCAGTAATCGAGTTTGTTTGTGAAGTTTTGGATTTTAAAAGTATTGA
AGAACAACAAAAACCTCTGACAGATTCCCAAAGGGTAAAGTTTACCAAAGAAATTA
AAGGTCTAAAGGTGGAGATAACGCACTGTGGGCAGATGAAGAGGAAGTACCGTGT
CTGCAATGTGACCCGGCGGCCCGCCAGTCACCAAACATTCCCGCTGCAGCAGGAG
AGCGGGCAGACGGTGGAGTGCACGGTGGCCCAGTATTTCAAGGACAGGCACAAG
TTGGTTCTGCGCTACCCCCACCTCCCATGTTTACAAGTCGGACAGGAGCAGAAAC
ACACCTACCTTCCCCTGGAGGTCTGTAACATTGTGGCAGGACAAAGATGTATTAAA
AAATTAACGGACAATCAGACCTCAACCATGATCAGAGCAACTGCTATGTCGGCGC
CCGATCGGCAAGAAGAGATTAGCAAATTGATGCGAAGTGCAAGTTTCAACACAGA
TCCATACGTCCGTGAATTTGGAATCATGGTCAAAGATGAGATGACAGACGTGACT
GGGCGGGTGCTGCAGCCGCCCTCCATCCTCTACGGGGGCAGGAATAAAGCTATTG
CGACCCCTGTCCAGGGCGTCTGGGACATGCGGAACAAGCAGTTCCACACGGGCAT
CGAGATCAAGGTGTGGGCCATTGCGTGCTTCGCCCCCCAGCGCCAGTGCACGGAA
GTCCATCTGAAGTCCTTCACAGAGCAGCTCAGAAAGATCTCGAGAGACGCTGGCA
TGCCCATCCAGGGCCAGCCGTGCTTCTGCAAATACGCGCAGGGGGCGGACAGCGT
GGAGCCCATGTTCCGGCACCTGAAGAACACGTATGCGGGCCTGCAGCTGGTGGTG
GTCATCCTGCCCGGCAAGACGCCCGTGTACGCCGAGGTCAAGCGCGTGGGAGAC
ACGGTGCTGGGGATGGCCACGCAGTGCGTGCAGATGAAGAACGTGCAGAGGACC
ACGCCACAGACCCTGTCCAACCTCTGCCTGAAGATCAACGTCAAGCTGGGAGGCG
TGAACAACATCCTGCTGCCCCAGGGCAGGCCGCCGGTGTTCCAGCAGCCCGTCAT
CTTTCTGGGAGCAGACGTCACTCACCCCCCCGCCGGGGATGGGAAGAAGCCCTCC
ATTGCCGCCGTGGTGGGCAGCATGGACGCCCACCCCAATCGCTACTGCGCCACCG
TGCGCGTGCAGCAGCACCGGCAGGAGATCATACAAGACCTGGCCGCCATGGTCCG
CGAGCTCCTCATCCAGTTCTACAAGTCCACGCGCTTCAAGCCCACCCGCATCATCT
TCTACCGCGACGGTGTCTCTGAAGGCCAGTTCCAGCAGGTTCTCCACCACGAGTT
GCTGGCCATCCGTGAGGCCTGTATCAAGCTAGAAAAAGACTACCAGCCCGGGATC
ACCTTCATCGTGGTGCAGAAGAGGCACCACACCCGGCTCTTCTGCACTGACAAGA
ACGAGCGGGTTGGGAAAAGTGGAAACATTCCAGCAGGCACGACTGTGGACACGA
AAATCACCCACCCCACCGAGTTCGACTTCTACCTGTGTAGTCACGCTGGCATCCAG
GGGACAAGCAGGCCTTCGCACTATCACGTCCTCTGGGACGACAATCGTTTCTCCT
CTGATGAGCTGCAGATCCTAACCTACCAGCTGTGTCACACCTACGTGCGCTGCAC
ACGCTCCGTGTCCATCCCAGCGCCAGCATACTACGCTCACCTGGTGGCCTTCCGG
GCCAGGTACCACCTGGTGGATAAGGAACATGACAGTGCTGAAGGAAGCCATACCT
CTGGGCAGAGTAACGGGCGAGACCACCAAGCACTGGCCAAGGCGGTCCAGGTTC
ACCAAGACACTCTGCGCACCATGTACTTTGCTTAA
Nluc-Ago2 amino acid sequence (SEQ ID NO: 155)
Bold: Flag-tag; italic: Nluc; underline: linker; underline/bold: A go2
MDYKDHDGDYKDHDIDYKDDDDKLVFTLEDFVGDWRQTAGYNLDQVLEQGGVSSLFQNL
GVSVTPIQRIVLSGENGLKIDIHVIIPYEGLSGDQMGQIEKIFKVVYPVDDHHFKVILHYGTLVIDG
VTPNMIDYFGRPYEGIAVFDGKKITVTGTLWNGNKIIDERLINPDGSLLFRVTINGVTGWRLCERIL
AGGGSMHPFQWCNALAPPAPPPPIQGYAFKPPPRPDFGTSGRTIKLQANFFEMDIPKIDI
YHYELDIKPEKCPRRVNREIVEHMVQHFKTQIFGDRKPVFDGRKNLYTAMPLPIGRD
KVELEVTLPGEGKDRIFKVSIKWVSCVSLQALHDALSGRLPSVPFETIQALDVVMRHL
PSMRYTPVGRSFFTASEGCSNPLGGGREVWFGFHQSVRPSLWKMMLNIDVSATAFYK
AQPVIEFVCEVLDFKSIEEQQKPLTDSQRVKFTKEIKGLKVEITHCGQMKRKYRVCNV
TRRPASHQTFPLQQESGQTVECTVAQYFKDRHKLVLRYPHLPCLQVGQEQKHTYLPL
EVCNIVAGQRCIKKLTDNQTSTMIRATAMSAPDRQEEISKLMRSASFNTDPYVREFGI
MVKDEMTDVTGRVLQPPSILYGGRNKAIATPVQGVWDMRNKQFHTGIEIKVWAIACF
APQRQCTEVHLKSFTEQLRKISRDAGMPIQGQPCFCKYAQGADSVEPMFRHLKNTYA
GLQLVVVILPGKTPVYAEVKRVGDTVLGMATQCVQMKNVQRTTPQTLSNLCLKINV
KLGGVNNILLPQGRPPVFQQPVIFLGADVTHPPAGDGKKPSIAAVVGSMDAHPNRYCA
TVRVQQHRQEIIQDLAAMVRELLIQFYKSTRFKPTRIIFYRDGVSEGQFQQVLHHELL
AIREACIKLEKDYQPGITFIVVQKRHHTRLFCTDKNERVGKSGNIPAGTTVDTKITHPT
EFDFYLCSHAGIQGTSRPSHYHVLWDDNRFSSDELQILTYQLCHTYVRCTRSVSIPAPA
YYAHLVAFRARYHLVDKEHDSAEGSHTSGQSNGRDHQALAKAVQVHQDTLRTMYFA
A go2 (position 387 bold/underline) (SEQ ID NO: 156)
MHPFQWCNALAPPAPPPPIQGYAFKPPPRPDFGTSGRTIKLQANFFEMDIPKIDIYHYELDIKP
EKCPRRVNREIVEHMVQHFKTQIFGDRKPVFDGRKNLYTAMPLPIGRDKVELEVTLPGEGK
DRIFKVSIKWVSCVSLQALHDALSGRLPSVPFETIQALDVVMRHLPSMRYTPVGRSFFTASE
GCSNPLGGGREVWFGFHQSVRPSLWKMMLNIDVSATAFYKAQPVIEFVCEVLDFKSIEEQQ
KPLTDSQRVKFTKEIKGLKVEITHCGQMKRKYRVCNVTRRPASHQTFPLQQESGQTVECTV
AQYFKDRHKLVLRYPHLPCLQVGQEQKHTYLPLEVCNIVAGQRCIKKLTDNQTSTMIRATA
MSAPDRQEEISKLMRSASFNTDPYVREFGIMVKDEMTDVTGRVLQPPSILYGGRNKAIATP
VQGVWDMRNKQFHTGIEIKVWAIACFAPQRQCTEVHLKSFTEQLRKISRDAGMPIQGQPCF
CKYAQGADSVEPMFRHLKNTYAGLQLVVVILPGKTPVYAEVKRVGDTVLGMATQCVQM
KNVQRTTPQTLSNLCLKINVKLGGVNNILLPQGRPPVFQQPVIFLGADVTHPPAGDGKKPSI
AAVVGSMDAHPNRYCATVRVQQHRQEIIQDLAAMVRELLIQFYKSTRFKPTRIIFYRDGVS
EGQFQQVLHHELLAIREACIKLEKDYQPGITFIVVQKRHHTRLFCTDKNERVGKSGNIPAGT
TVDTKITHPTEFDFYLCSHAGIQGTSRPSHYHVLWDDNRFSSDELQILTYQLCHTYVRCTRS
VSIPAPAYYAHLVAFRARYHLVDKEHDSAEGSHTSGQSNGRDHQALAKAVQVHQDTLRT
MYFA
GALA peptide (SEQ ID NO: 157)
WEAALAEALAEALAEHLAEALAEALEALAA
EALA peptide (SEQ ID NO: 158)
AALAEALAEALAEALAEALAEALAAAAGGC
KALA peptide (SEQ ID NO: 159)
WEAKLAKALAKALAKHLAKALAKALKACEA
A dapter sequence (SEQ ID NO: 216)
GCTGACCCTGAAGTTCAT

1. A method for making an extracellular vesicle comprising a small interfering RNA (siRNA) nucleic acid, the method comprising:
i) transfecting a cell with a nucleic acid encoding an argonaute 2 (AGO2) protein and a nucleic acid encoding a short hairpin RNA (shRNA) nucleic acid, and
ii) culturing the cell under conditions conducive for the cell to express said AGO2 protein and said shRNA nucleic acid, thereby forming said extracellular vesicle comprising said siRNA nucleic acid.

2. The method of claim 1, further comprising isolating said extracellular vesicle comprising said siRNA nucleic acid from said cell.

3. The method of claim 1, wherein said shRNA nucleic acid is a dicer-independent shRNA nucleic acid.

4. The method of claim 1, wherein said shRNA nucleic acid comprises a stem loop comprising a stem sequence no more than about 19 base pairs in length.

5.-7. (canceled)

8. The method of claim 1, wherein said shRNA nucleic acid comprises an unmatched base pair.

9. The method of claim 1, wherein said shRNA nucleic acid comprises an overhang sequence on the 3′ end.

10. The method of claim 9, wherein said overhang sequence comprises an exosome-specific RNA motif.

11.-15. (canceled)

16. The method of claim 1, wherein said siRNA nucleic acid is a single-stranded RNA (ssRNA).

17.-19. (canceled)

20. The method of claim 16, wherein said siRNA nucleic acid comprises a loop sequence of said shRNA stem loop.

21.-25. (canceled)

26. The method of claim 1, wherein step i) further comprises transfecting said cell with a nucleic acid encoding a fusogen protein.

27.-30. (canceled)

31. The method of claim 1, wherein step i) further comprises transfecting said cell with a nucleic acid encoding a fusion protein comprising:
a) an extracellular vesicle membrane-associated protein, and
b) a targeting protein or a detectable moiety.

32.-36. (canceled)

37. The method of claim 1, wherein the cell comprises a nucleic acid encoding a Charged Multivesicular Body Protein 4C (CHMP4C) inhibitor, a nucleic acid encoding a Vacuolar Protein Sorting 4 Homolog B (VPS4B) inhibitor, or a combination thereof.

38.-41. (canceled)

42. The method of claim 1, wherein the cell comprises a nucleic acid encoding an Ago2 sorting factor.

43. (canceled)

44. (canceled)

45. The method of claim 1, wherein said cell is a neural stem cell (NSC), a mesenchymal stem cell (MSC), an induced pluripotent stem cell (iPSC), an embryonic stem cell (ESC), an immune cell, or an epithelial cell.

46. (canceled)

47. A cell comprising an argonaute 2 (AGO2) protein and an extracellular vesicle comprising a small interfering (siRNA) nucleic acid.

48.-72. (canceled)

73. A cell stably transfected with a nucleic acid encoding an argonaute 2 (AGO2) protein and a nucleic acid encoding a short hairpin RNA (shRNA) nucleic acid.

74. The cell of claim 73, wherein said shRNA nucleic acid is a dicer-independent shRNA nucleic acid.

75. The cell of claim 73, wherein said shRNA nucleic acid comprises a stem loop comprising a stem sequence no more than about 19 base pairs in length.

76.-78. (canceled)

79. The cell of claim 73, wherein said shRNA nucleic acid comprises an unmatched base pair or an overhang sequence on the 3′ end.

80.-100. (canceled)

101. The cell of claim 73, wherein said cell is a neural stem cell (NSC), a mesenchymal stem cell (MSC), an induced pluripotent stem cell (iPSC), an embryonic stem cell (ESC), an immune cell, or an epithelial cell.

102. (canceled)


SEQUENCE LISTING (232 sequences)

SEQ ID NO: 1 - DNA, 358 bp, synthetic construct
  source 1..358: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgatcttactgactgccagggcacttgggaatggcaaggatgaacttcagggtcagcttgcgttgaccctgaagttcattcttgctatacccagaaaacgtgcctttttt

SEQ ID NO: 2 - DNA, 345 bp, synthetic construct
  source 1..345: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgccagggcacttgggaatggcaaggatgaacttcagggtcagcttgcgttgaccctgaagttcactcttgctatacccagaaaacgtgcctttttt

SEQ ID NO: 3 - DNA, 296 bp, synthetic construct
  source 1..296: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgatgaacttcagggtcagcttggctgaccctgaagttcatctttttt

SEQ ID NO: 4 - DNA, 318 bp, synthetic construct
  source 1..318: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcttaagattctaaaattatgtaagctgtcctctgaagttcattttttt

SEQ ID NO: 5 - DNA, 344 bp, synthetic construct
  source 1..344: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgtcagcagtgcctatgaacttcagggtcagcttgcttaagattctaaaattatgtaagctgacgctcgaagttcatagtaaggttgacttttttt

SEQ ID NO: 6 - DNA, 302 bp, synthetic construct
  source 1..302: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgcagatgaacttcagggtcagcttggctgaccctgaagttcatctgctttttt

SEQ ID NO: 7 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttggctgaccctgaagttcatctttttt

SEQ ID NO: 8 - DNA, 296 bp, synthetic construct
  source 1..296: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcacttttttt

SEQ ID NO: 9 - DNA, 296 bp, synthetic construct; identical to SEQ ID NO: 8
  source 1..296: mol_type = other DNA

SEQ ID NO: 10 - DNA, 297 bp, synthetic construct
  source 1..297: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcactctttttt

SEQ ID NO: 11 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcaatttttt

SEQ ID NO: 12 - DNA, 294 bp, synthetic construct
  source 1..294: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcatttttt

SEQ ID NO: 13 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcagtttttt

SEQ ID NO: 14 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcattttttt

SEQ ID NO: 15 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgtgaacttcagggtcagcttgcgctgaccctgaagttcaatttttt

SEQ ID NO: 16 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgtgaacttcagggtcagcttgcgctgaccctgaagttcactttttt

SEQ ID NO: 17 - DNA, 294 bp, synthetic construct
  source 1..294: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgtgaacttcagggtcagcttgcgctgaccctgaagttcatttttt

SEQ ID NO: 18 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgtgaacttcagggtcagcttgcgctgaccctgaagttcagtttttt

SEQ ID NO: 19 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgtgaacttcagggtcagcttgcgctgaccctgaagttcattttttt

SEQ ID NO: 20 - DNA, 295 bp, synthetic construct; identical to SEQ ID NO: 16
  source 1..295: mol_type = other DNA

SEQ ID NO: 21 - DNA, 297 bp, synthetic construct
  source 1..297: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccgtgaacttcagggtcagcttgcgctgaccctgaagttcactctttttt

SEQ ID NO: 22 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctaaagttcactttttt

SEQ ID NO: 23 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgacccagaagttcactttttt

SEQ ID NO: 24 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccatgaagttcactttttt

SEQ ID NO: 25 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgacactgaagttcactttttt

SEQ ID NO: 26 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccaagaagttcactttttt

SEQ ID NO: 27 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgacaatgaagttcactttttt

SEQ ID NO: 28 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgacaaagaagttcactttttt

SEQ ID NO: 29 - DNA, 119 bp, synthetic construct
  source 1..119: mol_type = other DNA
gcccggatagctcagtcggtagagcatcagacttttaatctgagggtccagggttcaagtccctgttcgggcgatgaacttcagggtcagcttgcgctgaccctgaagttcactttttt

SEQ ID NO: 30 - DNA, 456 bp, synthetic construct
  source 1..456: mol_type = other DNA
ctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaaggagttcccgtgccctgggagcgggttcaggaccgcggatcggaagtgagaatcccagctgtgtgtcagggctggaaagggctcgggagtgcgcggggcaagtgaccgtgtgtgtaaagagtgaggcgtatgaggctgtgtcggggcagagcccgaagatcatgaacttcagggtcagcttgcgctgaccctgaagttcacactttctggagtttcaaaagtagac

SEQ ID NO: 31 - DNA, 457 bp, synthetic construct
  source 1..457: mol_type = other DNA
ctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaaggagttcccgtgccctgggagcgggttcaggaccgcggatcggaagtgagaatcccagctgtgtgtcagggctggaaagggctcgggagtgcgcggggcaagtgaccgtgtgtgtaaagagtgaggcgtatgaggctgtgtcggggcagagcccgaagatcatgaacttcagggtcagcttgcgctgaccctgaagttcactactttctggagtttcaaaagtagac

SEQ ID NO: 32 - DNA, 458 bp, synthetic construct
  source 1..458: mol_type = other DNA
ctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaaggagttcccgtgccctgggagcgggttcaggaccgcggatcggaagtgagaatcccagctgtgtgtcagggctggaaagggctcgggagtgcgcggggcaagtgaccgtgtgtgtaaagagtgaggcgtatgaggctgtgtcggggcagagcccgaagatcatgaacttcagggtcagcttgcgctgaccctgaagttcacttactttctggagtttcaaaagtagac

SEQ ID NO: 33 - DNA, 459 bp, synthetic construct
  source 1..459: mol_type = other DNA
ctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaaggagttcccgtgccctgggagcgggttcaggaccgcggatcggaagtgagaatcccagctgtgtgtcagggctggaaagggctcgggagtgcgcggggcaagtgaccgtgtgtgtaaagagtgaggcgtatgaggctgtgtcggggcagagcccgaagatcatgaacttcagggtcagcttgcgctgaccctgaagttcactttactttctggagtttcaaaagtagac

SEQ ID NO: 34 - DNA, 149 bp, synthetic construct
  source 1..149: mol_type = other DNA
tgcaatatttgcatgtcgctatgtgttctgggaaatcaccataaacgtgaaatgtctttggatttgggaatcttataagttctgtatgagaccactcagatccatgaacttcagggtcagcttgcgctgaccctgaagttcactttttt

SEQ ID NO: 35 - DNA, 119 bp, synthetic construct
  source 1..119: mol_type = other DNA
gttttcgtagtgtagtggttatcacgtgtgcttcacacgcacaaggtccccggttcgaacccgggcgaaaacaatgaacttcagggtcagcttgcgctgaccctgaagttcactttttt

SEQ ID NO: 36 - DNA, 156 bp, synthetic construct
  source 1..156: mol_type = other DNA
gaaaatgactttgccacgcttagcatgtgacgaggtggccgagtggttaaggcgatggactgctaatccattgtgctctgcacgcgtgggttcgaatcccatcctcgtcgatgaacttcagggtcagcttgcgctgaccctgaagttcactttttt

SEQ ID NO: 37 - DNA, 117 bp, synthetic construct
  source 1..117: mol_type = other DNA
gcatgggtggttcagtggtagaattctcgcctgccacgcgggaggcccgggttcgattcccggcccatgcaatgaacttcagggtcagcttgcgctgaccctgaagttcactttttt

SEQ ID NO: 38 - DNA, 118 bp, synthetic construct
  source 1..118: mol_type = other DNA
ggttccatggtgtaatggttagcactctggactctgaatccagcgatccgagttcaaatctcggtggaacctatgaacttcagggtcagcttgcgctgaccctgaagttcactttttt

SEQ ID NO: 39 - DNA, 118 bp, synthetic construct
  source 1..118: mol_type = other DNA
ggctcgttggtctaggggtatgattctcgcttagggtgcgagaggtcccgggttcaaatcccggacgagcccatgaacttcagggtcagcttgcgctgaccctgaagttcactttttt

SEQ ID NO: 40 - DNA, 129 bp, synthetic construct
  source 1..129: mol_type = other DNA
gtcaggatggccgagtggtctaaggcgctgcgttcaggtcgcagtctactctgtaggcgtgggttcgaatcccacttctgacaatgaacttcagggtcagcttgcgctgaccctgaagttcactttttt

SEQ ID NO: 41 - DNA, 120 bp, synthetic construct
  source 1..120: mol_type = other DNA
gcctccgtggcgcaattggttagcgcgttcggctgttaaccgaaaggttggtggttcgagtccacccgggggcgatgaacttcagggtcagcttgcgctgaccctgaagttcactttttt

SEQ ID NO: 42 - RNA, 105 nt, synthetic construct
  source 1..105: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 9: mod_base = OTHER; note = Uracil
  modified_base 13: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 40: mod_base = OTHER; note = Uracil
  modified_base 45: mod_base = OTHER; note = Uracil
  modified_base 46: mod_base = OTHER; note = Uracil
  modified_base 52: mod_base = OTHER; note = Uracil
  modified_base 57: mod_base = OTHER; note = Uracil
  modified_base 58: mod_base = OTHER; note = Uracil
  modified_base 62: mod_base = OTHER; note = Uracil
  modified_base 63: mod_base = OTHER; note = Uracil
  modified_base 69: mod_base = OTHER; note = Uracil
  modified_base 74: mod_base = OTHER; note = Uracil
  modified_base 75: mod_base = OTHER; note = Uracil
  modified_base 78: mod_base = OTHER; note = Uracil
  modified_base 79: mod_base = OTHER; note = Uracil
  modified_base 81: mod_base = OTHER; note = Uracil
  modified_base 82: mod_base = OTHER; note = Uracil
  modified_base 85: mod_base = OTHER; note = Uracil
  modified_base 87: mod_base = OTHER; note = Uracil
  modified_base 100: mod_base = OTHER; note = Uracil
  modified_base 104: mod_base = OTHER; note = Uracil
  modified_base 105: mod_base = OTHER; note = Uracil
gatcttactgactgccagggcacttgggaatggcaaggatgaacttcagggtcagcttgcgttgaccctgaagttcattcttgctatacccagaaaacgtgcctt

SEQ ID NO: 43 - RNA, 92 nt, synthetic construct
  source 1..92: mol_type = other RNA
  modified_base 11: mod_base = OTHER; note = Uracil
  modified_base 12: mod_base = OTHER; note = Uracil
  modified_base 18: mod_base = OTHER; note = Uracil
  modified_base 27: mod_base = OTHER; note = Uracil
  modified_base 32: mod_base = OTHER; note = Uracil
  modified_base 33: mod_base = OTHER; note = Uracil
  modified_base 39: mod_base = OTHER; note = Uracil
  modified_base 44: mod_base = OTHER; note = Uracil
  modified_base 45: mod_base = OTHER; note = Uracil
  modified_base 49: mod_base = OTHER; note = Uracil
  modified_base 50: mod_base = OTHER; note = Uracil
  modified_base 56: mod_base = OTHER; note = Uracil
  modified_base 61: mod_base = OTHER; note = Uracil
  modified_base 62: mod_base = OTHER; note = Uracil
  modified_base 66: mod_base = OTHER; note = Uracil
  modified_base 68: mod_base = OTHER; note = Uracil
  modified_base 69: mod_base = OTHER; note = Uracil
  modified_base 72: mod_base = OTHER; note = Uracil
  modified_base 74: mod_base = OTHER; note = Uracil
  modified_base 87: mod_base = OTHER; note = Uracil
  modified_base 91: mod_base = OTHER; note = Uracil
  modified_base 92: mod_base = OTHER; note = Uracil
gccagggcacttgggaatggcaaggatgaacttcagggtcagcttgcgttgaccctgaagttcactcttgctatacccagaaaacgtgcctt

SEQ ID NO: 44 - RNA, 43 nt, synthetic construct
  source 1..43: mol_type = other RNA
  modified_base 3: mod_base = OTHER; note = Uracil
  modified_base 8: mod_base = OTHER; note = Uracil
  modified_base 9: mod_base = OTHER; note = Uracil
  modified_base 15: mod_base = OTHER; note = Uracil
  modified_base 20: mod_base = OTHER; note = Uracil
  modified_base 21: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 40: mod_base = OTHER; note = Uracil
  modified_base 42: mod_base = OTHER; note = Uracil
  modified_base 43: mod_base = OTHER; note = Uracil
gatgaacttcagggtcagcttggctgaccctgaagttcatctt

SEQ ID NO: 45 - RNA, 64 nt, synthetic construct
  source 1..64: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 23: mod_base = OTHER; note = Uracil
  modified_base 24: mod_base = OTHER; note = Uracil
  modified_base 29: mod_base = OTHER; note = Uracil
  modified_base 30: mod_base = OTHER; note = Uracil
  modified_base 32: mod_base = OTHER; note = Uracil
  modified_base 37: mod_base = OTHER; note = Uracil
  modified_base 38: mod_base = OTHER; note = Uracil
  modified_base 40: mod_base = OTHER; note = Uracil
  modified_base 42: mod_base = OTHER; note = Uracil
  modified_base 47: mod_base = OTHER; note = Uracil
  modified_base 49: mod_base = OTHER; note = Uracil
  modified_base 52: mod_base = OTHER; note = Uracil
  modified_base 54: mod_base = OTHER; note = Uracil
  modified_base 59: mod_base = OTHER; note = Uracil
  modified_base 60: mod_base = OTHER; note = Uracil
  modified_base 63: mod_base = OTHER; note = Uracil
  modified_base 64: mod_base = OTHER; note = Uracil
atgaacttcagggtcagcttgcttaagattctaaaattatgtaagctgtcctctgaagttcatt

SEQ ID NO: 46 - RNA, 90 nt, synthetic construct
  source 1..90: mol_type = other RNA
  modified_base 2: mod_base = OTHER; note = Uracil
  modified_base 9: mod_base = OTHER; note = Uracil
  modified_base 13: mod_base = OTHER; note = Uracil
  modified_base 15: mod_base = OTHER; note = Uracil
  modified_base 20: mod_base = OTHER; note = Uracil
  modified_base 21: mod_base = OTHER; note = Uracil
  modified_base 27: mod_base = OTHER; note = Uracil
  modified_base 32: mod_base = OTHER; note = Uracil
  modified_base 33: mod_base = OTHER; note = Uracil
  modified_base 36: mod_base = OTHER; note = Uracil
  modified_base 37: mod_base = OTHER; note = Uracil
  modified_base 42: mod_base = OTHER; note = Uracil
  modified_base 43: mod_base = OTHER; note = Uracil
  modified_base 45: mod_base = OTHER; note = Uracil
  modified_base 50: mod_base = OTHER; note = Uracil
  modified_base 51: mod_base = OTHER; note = Uracil
  modified_base 53: mod_base = OTHER; note = Uracil
  modified_base 55: mod_base = OTHER; note = Uracil
  modified_base 60: mod_base = OTHER; note = Uracil
  modified_base 66: mod_base = OTHER; note = Uracil
  modified_base 72: mod_base = OTHER; note = Uracil
  modified_base 73: mod_base = OTHER; note = Uracil
  modified_base 76: mod_base = OTHER; note = Uracil
  modified_base 79: mod_base = OTHER; note = Uracil
  modified_base 84: mod_base = OTHER; note = Uracil
  modified_base 85: mod_base = OTHER; note = Uracil
  modified_base 89: mod_base = OTHER; note = Uracil
  modified_base 90: mod_base = OTHER; note = Uracil
gtcagcagtgcctatgaacttcagggtcagcttgcttaagattctaaaattatgtaagctgacgctcgaagttcatagtaaggttgactt

SEQ ID NO: 47 - RNA, 49 nt, synthetic construct
  source 1..49: mol_type = other RNA
  modified_base 6: mod_base = OTHER; note = Uracil
  modified_base 11: mod_base = OTHER; note = Uracil
  modified_base 12: mod_base = OTHER; note = Uracil
  modified_base 18: mod_base = OTHER; note = Uracil
  modified_base 23: mod_base = OTHER; note = Uracil
  modified_base 24: mod_base = OTHER; note = Uracil
  modified_base 28: mod_base = OTHER; note = Uracil
  modified_base 34: mod_base = OTHER; note = Uracil
  modified_base 39: mod_base = OTHER; note = Uracil
  modified_base 40: mod_base = OTHER; note = Uracil
  modified_base 43: mod_base = OTHER; note = Uracil
  modified_base 45: mod_base = OTHER; note = Uracil
  modified_base 48: mod_base = OTHER; note = Uracil
  modified_base 49: mod_base = OTHER; note = Uracil
gcagatgaacttcagggtcagcttggctgaccctgaagttcatctgctt

SEQ ID NO: 48 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 24: mod_base = OTHER; note = Uracil
  modified_base 30: mod_base = OTHER; note = Uracil
  modified_base 35: mod_base = OTHER; note = Uracil
  modified_base 36: mod_base = OTHER; note = Uracil
  modified_base 39: mod_base = OTHER; note = Uracil
  modified_base 41: mod_base = OTHER; note = Uracil
  modified_base 42: mod_base = OTHER; note = Uracil
atgaacttcagggtcagcttggctgaccctgaagttcatctt

SEQ ID NO: 49 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
atgaacttcagggtcagcttgcgctgaccctgaagttcactt

SEQ ID NO: 50 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 51 - RNA, 44 nt, synthetic construct
  source 1..44: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 43: mod_base = OTHER; note = Uracil
  modified_base 44: mod_base = OTHER; note = Uracil
atgaacttcagggtcagcttgcgctgaccctgaagttcactctt

SEQ ID NO: 52 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
atgaacttcagggtcagcttgcgctgaccctgaagttcaatt

SEQ ID NO: 53 - RNA, 41 nt, synthetic construct
  source 1..41: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 40: mod_base = OTHER; note = Uracil
  modified_base 41: mod_base = OTHER; note = Uracil
atgaacttcagggtcagcttgcgctgaccctgaagttcatt

SEQ ID NO: 54 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
atgaacttcagggtcagcttgcgctgaccctgaagttcagtt

SEQ ID NO: 55 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 40: mod_base = OTHER; note = Uracil
  modified_base 41: mod_base = OTHER; note = Uracil
  modified_base 42: mod_base = OTHER; note = Uracil
atgaacttcagggtcagcttgcgctgaccctgaagttcattt

SEQ ID NO: 56 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
gtgaacttcagggtcagcttgcgctgaccctgaagttcaatt

SEQ ID NO: 57 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
gtgaacttcagggtcagcttgcgctgaccctgaagttcactt

SEQ ID NO: 58 - RNA, 41 nt, synthetic construct
  source 1..41: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 40: mod_base = OTHER; note = Uracil
  modified_base 41: mod_base = OTHER; note = Uracil
gtgaacttcagggtcagcttgcgctgaccctgaagttcatt

SEQ ID NO: 59 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
gtgaacttcagggtcagcttgcgctgaccctgaagttcagtt

SEQ ID NO: 60 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 40: mod_base = OTHER; note = Uracil
  modified_base 41: mod_base = OTHER; note = Uracil
  modified_base 42: mod_base = OTHER; note = Uracil
gtgaacttcagggtcagcttgcgctgaccctgaagttcattt

SEQ ID NO: 61 - RNA, 42 nt, synthetic construct; identical to SEQ ID NO: 57
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 62 - RNA, 44 nt, synthetic construct
  source 1..44: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 43: mod_base = OTHER; note = Uracil
  modified_base 44: mod_base = OTHER; note = Uracil
gtgaacttcagggtcagcttgcgctgaccctgaagttcactctt

SEQ ID NO: 63 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
atgaacttcagggtcagcttgcgctgaccctaaagttcactt

SEQ ID NO: 64 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
atgaacttcagggtcagcttgcgctgacccagaagttcactt

SEQ ID NO: 65 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
atgaacttcagggtcagcttgcgctgaccatgaagttcactt

SEQ ID NO: 66 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
atgaacttcagggtcagcttgcgctgacactgaagttcactt

SEQ ID NO: 67 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
atgaacttcagggtcagcttgcgctgaccaagaagttcactt

SEQ ID NO: 68 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
atgaacttcagggtcagcttgcgctgacaatgaagttcactt

SEQ ID NO: 69 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
atgaacttcagggtcagcttgcgctgacaaagaagttcactt

SEQ ID NO: 70 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 71 - RNA, 40 nt, synthetic construct
  source 1..40: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
atgaacttcagggtcagcttgcgctgaccctgaagttcac

SEQ ID NO: 72 - RNA, 41 nt, synthetic construct
  source 1..41: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
atgaacttcagggtcagcttgcgctgaccctgaagttcact

SEQ ID NO: 73 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 74 - RNA, 43 nt, synthetic construct
  source 1..43: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
  modified_base 43: mod_base = OTHER; note = Uracil
atgaacttcagggtcagcttgcgctgaccctgaagttcacttt

SEQ ID NO: 75 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 76 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 77 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 78 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 79 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 80 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 81 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 82 - RNA, 42 nt, synthetic construct; identical to SEQ ID NO: 49
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil

SEQ ID NO: 83 - DNA, 300 bp, synthetic construct
  source 1..300: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcacggctgtttttt

SEQ ID NO: 84 - DNA, 299 bp, synthetic construct
  source 1..299: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcacggagtttttt

SEQ ID NO: 85 - DNA, 299 bp, synthetic construct
  source 1..299: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcaccccttttttt

SEQ ID NO: 86 - DNA, 299 bp, synthetic construct
  source 1..299: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcaccatgtttttt

SEQ ID NO: 87 - DNA, 299 bp, synthetic construct
  source 1..299: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcacgtgctttttt

SEQ ID NO: 88 - DNA, 299 bp, synthetic construct
  source 1..299: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcaccccctttttt

SEQ ID NO: 89 - DNA, 299 bp, synthetic construct
  source 1..299: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcaccgggtttttt

SEQ ID NO: 90 - DNA, 300 bp, synthetic construct
  source 1..300: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcactgtgttttttt

SEQ ID NO: 91 - DNA, 302 bp, synthetic construct
  source 1..302: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcactgtgtgttttttt

SEQ ID NO: 92 - DNA, 301 bp, synthetic construct
  source 1..301: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcacccggagtttttt

SEQ ID NO: 93 - DNA, 301 bp, synthetic construct
  source 1..301: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcaccgggagtttttt

SEQ ID NO: 94 - DNA, 301 bp, synthetic construct
  source 1..301: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcaccagtagtttttt

SEQ ID NO: 95 - DNA, 299 bp, synthetic construct
  source 1..299: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcacgtactttttt

SEQ ID NO: 96 - DNA, 317 bp, synthetic construct
  source 1..317: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgaacttcagggtcagcttgcgctgaccctgaagttcacaatccattgcactccggatttatttttt

SEQ ID NO: 97 - RNA, 47 nt, synthetic construct
  source 1..47: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 44: mod_base = OTHER; note = Uracil
  modified_base 46: mod_base = OTHER; note = Uracil
  modified_base 47: mod_base = OTHER; note = Uracil
atgaacttcagggtcagcttgcgctgaccctgaagttcacggctgtt

SEQ ID NO: 98 - RNA, 46 nt, synthetic construct
  source 1..46: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 45: mod_base = OTHER; note = Uracil
  modified_base 46: mod_base = OTHER; note = Uracil
atgaacttcagggtcagcttgcgctgaccctgaagttcacggagtt

SEQ ID NO: 99 - RNA, 45 nt, synthetic construct
  source 1..45: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 44: mod_base = OTHER; note = Uracil
  modified_base 45: mod_base = OTHER; note = Uracil
atgaacttcagggtcagcttgcgctgaccctgaagttcacccctt

SEQ ID NO: 100 - RNA, 46 nt, synthetic construct
  source 1..46: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
atgaacttcagggtcagcttgcgctgaccctgaagttcaccatgtt

SEQ ID NO: 101 - RNA, 46 nt, synthetic construct
  source 1..46: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 42: mod_base = OTHER; note = Uracil
  modified_base 45: mod_base = OTHER; note = Uracil
  modified_base 46: mod_base = OTHER; note = Uracil
atgaacttcagggtcagcttgcgctgaccctgaagttcacgtgctt

SEQ ID NO: 102 - RNA, 46 nt, synthetic construct
  source 1..46: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 45: mod_base = OTHER; note = Uracil
  modified_base 46: mod_base = OTHER; note = Uracil
atgaacttcagggtcagcttgcgctgaccctgaagttcaccccctt

SEQ ID NO: 103 - RNA, 46 nt, synthetic construct
  source 1..46: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 45: mod_base = OTHER; note = Uracil
  modified_base 46: mod_base = OTHER; note = Uracil
atgaacttcagggtcagcttgcgctgaccctgaagttcaccgggtt

SEQ ID NO: 104 - RNA, 46 nt, synthetic construct
  source 1..46: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
atgaacttcagggtcagcttgcgctgaccctgaagttcactgtgtt

SEQ ID NO: 105 - RNA, 48 nt, synthetic construct
  source 1..48: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 43: mod_base = OTHER; note = Uracil
  modified_base 45: mod_base = OTHER; note = Uracil
  modified_base 47: mod_base = OTHER; note = Uracil
  modified_base 48: mod_base = OTHER; note = Uracil
atgaacttcagggtcagcttgcgctgaccctgaagttcactgtgtgtt

SEQ ID NO: 106 - RNA, 48 nt, synthetic construct
  source 1..48: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 47: mod_base = OTHER; note = Uracil
  modified_base 48: mod_base = OTHER; note = Uracil
atgaacttcagggtcagcttgcgctgaccctgaagttcacccggagtt

SEQ ID NO: 107 - RNA, 48 nt, synthetic construct
  source 1..48: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 47: mod_base = OTHER; note = Uracil
  modified_base 48: mod_base = OTHER; note = Uracil
atgaacttcagggtcagcttgcgctgaccctgaagttcaccgggagtt

SEQ ID NO: 108 - RNA, 48 nt, synthetic construct
  source 1..48: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 44: mod_base = OTHER; note = Uracil
  modified_base 47: mod_base = OTHER; note = Uracil
  modified_base 48: mod_base = OTHER; note = Uracil
atgaacttcagggtcagcttgcgctgaccctgaagttcaccagtagtt

SEQ ID NO: 109 - RNA, 46 nt, synthetic construct
  source 1..46: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 42: mod_base = OTHER; note = Uracil
  modified_base 45: mod_base = OTHER; note = Uracil
  modified_base 46: mod_base = OTHER; note = Uracil
atgaacttcagggtcagcttgcgctgaccctgaagttcacgtactt

SEQ ID NO: 110 - RNA, 64 nt, synthetic construct
  source 1..64: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 19: mod_base = OTHER; note = Uracil
  modified_base 20: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 43: mod_base = OTHER; note = Uracil
  modified_base 47: mod_base = OTHER; note = Uracil
  modified_base 48: mod_base = OTHER; note = Uracil
  modified_base 53: mod_base = OTHER; note = Uracil
  modified_base 59: mod_base = OTHER; note = Uracil
  modified_base 60: mod_base = OTHER; note = Uracil
  modified_base 61: mod_base = OTHER; note = Uracil
  modified_base 63: mod_base = OTHER; note = Uracil
  modified_base 64: mod_base = OTHER; note = Uracil
atgaacttcagggtcagcttgcgctgaccctgaagttcacaatccattgcactccggatttatt

SEQ ID NO: 111 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccaagcgtagagtcacacttgcaaaagtgtgactctacgctctttttt

SEQ ID NO: 112 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatcttgactctcaatccaagggtggattgagagtcaagactttttt

SEQ ID NO: 113 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccactgctggtcaatctctcccaggagagattgaccagcagctttttt

SEQ ID NO: 114 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccactgcttgattcttcgtagatttacgaagaatcaagcagctttttt

SEQ ID NO: 115 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatctccattggcttctcaagattgagaagccaatggagactttttt

SEQ ID NO: 116 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccagtaactgttgcttgcagtacactgcaagcaacagttacctttttt

SEQ ID NO: 117 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccagtacacacattctaatattattattagaatgtgtgtacctttttt

SEQ ID NO: 118 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgtatagttgtttgcagctctctgcaaacaactatacactttttt

SEQ ID NO: 119 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccagttgtttgcagctctgtgcatacagagctgcaaacaacctttttt

SEQ ID NO: 120 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccacctcacgtcgcagtaactgttgttactgcgacgtgaggctttttt

SEQ ID NO: 121 - DNA, 295 bp, synthetic construct; identical to SEQ ID NO: 111
  source 1..295: mol_type = other DNA

SEQ ID NO: 122 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccattcctagtgtgcccattaacaaatgggcacactaggaactttttt

SEQ ID NO: 123 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgggctctgtccggttctgctgaaccggacagagcccactttttt

SEQ ID NO: 124 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccagttgtctctggttgcaaatctttgcaaccagagacaacctttttt

SEQ ID NO: 125 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccacgtgtgtgctttgtacgcacacgtacaaagcacacacgctttttt

SEQ ID NO: 126 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccagtcgtgctgcttcatgtggtcacatgaagcagcacgacctttttt

SEQ ID NO: 127 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccatgcccttcagctcgatgcggtcatcgagctgaagggcactttttt

SEQ ID NO: 128 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccagcttcttctgcacattcaggagaatgtgcagaagaagcctttttt

SEQ ID NO: 129 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccagtggctggtcacaaaggtatacctttgtgaccagccacctttttt

SEQ ID NO: 130 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccagttctcagagcacaccacaattggtgtgctctgagaacctttttt

SEQ ID NO: 131 - DNA, 295 bp, synthetic construct
  source 1..295: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatccaggaaagtccccagcggaaagttccgctggggactttccctttttt

SEQ ID NO: 132 - RNA, 42 nt, synthetic construct
  source 1..42: mol_type = other RNA
  modified_base 6: mod_base = OTHER; note = Uracil
  modified_base 11: mod_base = OTHER; note = Uracil
  modified_base 17: mod_base = OTHER; note = Uracil
  modified_base 18: mod_base = OTHER; note = Uracil
  modified_base 26: mod_base = OTHER; note = Uracil
  modified_base 28: mod_base = OTHER; note = Uracil
  modified_base 32: mod_base = OTHER; note = Uracil
  modified_base 34: mod_base = OTHER; note = Uracil
  modified_base 39: mod_base = OTHER; note = Uracil
  modified_base 41: mod_base = OTHER; note = Uracil
  modified_base 42: mod_base = OTHER; note = Uracil
aagcgtagagtcacacttgcaaaagtgtgactctacgctctt

SEQ ID NO: 133 - RNA, 42 nt, synthetic construct
  source 1..42: mol_type = other RNA
  modified_base 2: mod_base = OTHER; note = Uracil
  modified_base 4: mod_base = OTHER; note = Uracil
  modified_base 5: mod_base = OTHER; note = Uracil
  modified_base 9: mod_base = OTHER; note = Uracil
  modified_base 11: mod_base = OTHER; note = Uracil
  modified_base 15: mod_base = OTHER; note = Uracil
  modified_base 23: mod_base = OTHER; note = Uracil
  modified_base 27: mod_base = OTHER; note = Uracil
  modified_base 28: mod_base = OTHER; note = Uracil
  modified_base 34: mod_base = OTHER; note = Uracil
  modified_base 41: mod_base = OTHER; note = Uracil
  modified_base 42: mod_base = OTHER; note = Uracil
atcttgactctcaatccaagggtggattgagagtcaagactt

SEQ ID NO: 134 - RNA, 42 nt, synthetic construct
  source 1..42: mol_type = other RNA
  modified_base 3: mod_base = OTHER; note = Uracil
  modified_base 6: mod_base = OTHER; note = Uracil
  modified_base 9: mod_base = OTHER; note = Uracil
  modified_base 13: mod_base = OTHER; note = Uracil
  modified_base 15: mod_base = OTHER; note = Uracil
  modified_base 17: mod_base = OTHER; note = Uracil
  modified_base 29: mod_base = OTHER; note = Uracil
  modified_base 30: mod_base = OTHER; note = Uracil
  modified_base 41: mod_base = OTHER; note = Uracil
  modified_base 42: mod_base = OTHER; note = Uracil
actgctggtcaatctctcccaggagagattgaccagcagctt

SEQ ID NO: 135 - RNA, 42 nt, synthetic construct
  source 1..42: mol_type = other RNA
  modified_base 3: mod_base = OTHER; note = Uracil
  modified_base 6: mod_base = OTHER; note = Uracil
  modified_base 7: mod_base = OTHER; note = Uracil
  modified_base 10: mod_base = OTHER; note = Uracil
  modified_base 11: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 21: mod_base = OTHER; note = Uracil
  modified_base 22: mod_base = OTHER; note = Uracil
  modified_base 23: mod_base = OTHER; note = Uracil
  modified_base 32: mod_base = OTHER; note = Uracil
  modified_base 41: mod_base = OTHER; note = Uracil
  modified_base 42: mod_base = OTHER; note = Uracil
actgcttgattcttcgtagatttacgaagaatcaagcagctt

SEQ ID NO: 136 - RNA, 42 nt, synthetic construct
  source 1..42: mol_type = other RNA
  modified_base 2: mod_base = OTHER; note = Uracil
  modified_base 4: mod_base = OTHER; note = Uracil
  modified_base 8: mod_base = OTHER; note = Uracil
  modified_base 9: mod_base = OTHER; note = Uracil
  modified_base 13: mod_base = OTHER; note = Uracil
  modified_base 14: mod_base = OTHER; note = Uracil
  modified_base 16: mod_base = OTHER; note = Uracil
  modified_base 22: mod_base = OTHER; note = Uracil
  modified_base 23: mod_base = OTHER; note = Uracil
  modified_base 34: mod_base = OTHER; note = Uracil
  modified_base 41: mod_base = OTHER; note = Uracil
  modified_base 42: mod_base = OTHER; note = Uracil
atctccattggcttctcaagattgagaagccaatggagactt

SEQ ID NO: 137 - RNA, 42 nt, synthetic construct
  source 1..42: mol_type = other RNA
  modified_base 3: mod_base = OTHER; note = Uracil
  modified_base 7: mod_base = OTHER; note = Uracil
  modified_base 9: mod_base = OTHER; note = Uracil
  modified_base 10: mod_base = OTHER; note = Uracil
  modified_base 13: mod_base = OTHER; note = Uracil
  modified_base 14: mod_base = OTHER; note = Uracil
  modified_base 19: mod_base = OTHER; note = Uracil
  modified_base 24: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
agtaactgttgcttgcagtacactgcaagcaacagttacctt

SEQ ID NO: 138 - RNA, 42 nt, synthetic construct
  source 1..42: mol_type = other RNA
  modified_base 3: mod_base = OTHER; note = Uracil
  modified_base 11: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 23: mod_base = OTHER; note = Uracil
  modified_base 25: mod_base = OTHER; note = Uracil
  modified_base 26: mod_base = OTHER; note = Uracil
  modified_base 31: mod_base = OTHER; note = Uracil
  modified_base 33: mod_base = OTHER; note = Uracil
  modified_base 35: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
agtacacacattctaatattattattagaatgtgtgtacctt

SEQ ID NO: 139 - RNA, 42 nt, synthetic construct
  source 1..42: mol_type = other RNA
  modified_base 2: mod_base = OTHER; note = Uracil
  modified_base 4: mod_base = OTHER; note = Uracil
  modified_base 6: mod_base = OTHER; note = Uracil
  modified_base 9: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 20: mod_base = OTHER; note = Uracil
  modified_base 22: mod_base = OTHER; note = Uracil
  modified_base 24: mod_base = OTHER; note = Uracil
  modified_base 34: mod_base = OTHER; note = Uracil
  modified_base 36: mod_base = OTHER; note = Uracil
  modified_base 41: mod_base = OTHER; note = Uracil
  modified_base 42: mod_base = OTHER; note = Uracil
atgtatagttgtttgcagctctctgcaaacaactatacactt

SEQ ID NO: 140 - RNA, 42 nt, synthetic construct
  source 1..42: mol_type = other RNA
  modified_base 3: mod_base = OTHER; note = Uracil
  modified_base 4: mod_base = OTHER; note = Uracil
  modified_base 6: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
  modified_base 16: mod_base = OTHER; note = Uracil
  modified_base 18: mod_base = OTHER; note = Uracil
  modified_base 22: mod_base = OTHER; note = Uracil
  modified_base 30: mod_base = OTHER; note = Uracil
  modified_base 41: mod_base = OTHER; note = Uracil
  modified_base 42: mod_base = OTHER; note = Uracil
agttgtttgcagctctgtgcatacagagctgcaaacaacctt

SEQ ID NO: 141 - RNA, 42 nt, synthetic construct
  source 1..42: mol_type = other RNA
  modified_base 4: mod_base = OTHER; note = Uracil
  modified_base 9: mod_base = OTHER; note = Uracil
  modified_base 15: mod_base = OTHER; note = Uracil
  modified_base 19: mod_base = OTHER; note = Uracil
  modified_base 21: mod_base = OTHER; note = Uracil
  modified_base 22: mod_base = OTHER; note = Uracil
  modified_base 24: mod_base = OTHER; note = Uracil
  modified_base 25: mod_base = OTHER; note = Uracil
  modified_base 28: mod_base = OTHER; note = Uracil
  modified_base 35: mod_base = OTHER; note = Uracil
  modified_base 41: mod_base = OTHER; note = Uracil
  modified_base 42: mod_base = OTHER; note = Uracil
acctcacgtcgcagtaactgttgttactgcgacgtgaggctt

SEQ ID NO: 142 - RNA, 42 nt, synthetic construct; identical to SEQ ID NO: 132
  source 1..42: mol_type = other RNA
  modified_base 6: mod_base = OTHER; note = Uracil
  modified_base 11: mod_base = OTHER; note = Uracil
  modified_base 17: mod_base = OTHER; note = Uracil
  modified_base 18: mod_base = OTHER; note = Uracil
  modified_base 26: mod_base = OTHER; note = Uracil
  modified_base 28: mod_base = OTHER; note = Uracil
  modified_base 32: mod_base = OTHER; note = Uracil
  modified_base 34: mod_base = OTHER; note = Uracil
  modified_base 39: mod_base = OTHER; note = Uracil
  modified_base 41: mod_base = OTHER; note = Uracil
  modified_base 42: mod_base = OTHER; note = Uracil

SEQ ID NO: 143 - RNA, 42 nt, synthetic construct
  source 1..42: mol_type = other RNA
  modified_base 2: mod_base = OTHER; note = Uracil
  modified_base 3: mod_base = OTHER; note = Uracil
  modified_base 6: mod_base = OTHER; note = Uracil
  modified_base 9: mod_base = OTHER; note = Uracil
  modified_base 11: mod_base = OTHER; note = Uracil
  modified_base 17: mod_base = OTHER; note = Uracil
  modified_base 18: mod_base = OTHER; note = Uracil
  modified_base 25: mod_base = OTHER; note = Uracil
  modified_base 34: mod_base = OTHER; note = Uracil
  modified_base 41: mod_base = OTHER; note = Uracil
  modified_base 42: mod_base = OTHER; note = Uracil
attcctagtgtgcccattaacaaatgggcacactaggaactt

SEQ ID NO: 144 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 9: mod_base = OTHER; note = Uracil
  modified_base 11: mod_base = OTHER; note = Uracil
  modified_base 16: mod_base = OTHER; note = Uracil
  modified_base 17: mod_base = OTHER; note = Uracil
  modified_base 19: mod_base = OTHER; note = Uracil
  modified_base 22: mod_base = OTHER; note = Uracil
  modified_base 41: mod_base = OTHER; note = Uracil
  modified_base 42: mod_base = OTHER; note = Uracil
atgggctctgtccggttctgctgaaccggacagagcccactt

SEQ ID NO: 145 - RNA, 42 nt, synthetic construct
  source 1..42: mol_type = other RNA
  modified_base 3: mod_base = OTHER; note = Uracil
  modified_base 4: mod_base = OTHER; note = Uracil
  modified_base 6: mod_base = OTHER; note = Uracil
  modified_base 8: mod_base = OTHER; note = Uracil
  modified_base 10: mod_base = OTHER; note = Uracil
  modified_base 13: mod_base = OTHER; note = Uracil
  modified_base 14: mod_base = OTHER; note = Uracil
  modified_base 20: mod_base = OTHER; note = Uracil
  modified_base 22: mod_base = OTHER; note = Uracil
  modified_base 23: mod_base = OTHER; note = Uracil
  modified_base 24: mod_base = OTHER; note = Uracil
  modified_base 41: mod_base = OTHER; note = Uracil
  modified_base 42: mod_base = OTHER; note = Uracil
agttgtctctggttgcaaatctttgcaaccagagacaacctt

SEQ ID NO: 146 - RNA, 42 nt, synthetic construct
  source 1..42: mol_type = other RNA
  modified_base 4: mod_base = OTHER; note = Uracil
  modified_base 6: mod_base = OTHER; note = Uracil
  modified_base 8: mod_base = OTHER; note = Uracil
  modified_base 11: mod_base = OTHER; note = Uracil
  modified_base 12: mod_base = OTHER; note = Uracil
  modified_base 13: mod_base = OTHER; note = Uracil
  modified_base 15: mod_base = OTHER; note = Uracil
  modified_base 25: mod_base = OTHER; note = Uracil
  modified_base 41: mod_base = OTHER; note = Uracil
  modified_base 42: mod_base = OTHER; note = Uracil
acgtgtgtgctttgtacgcacacgtacaaagcacacacgctt

SEQ ID NO: 147 - RNA, 42 nt, synthetic construct
  source 1..42: mol_type = other RNA
  modified_base 3: mod_base = OTHER; note = Uracil
  modified_base 6: mod_base = OTHER; note = Uracil
  modified_base 9: mod_base = OTHER; note = Uracil
  modified_base 12: mod_base = OTHER; note = Uracil
  modified_base 13: mod_base = OTHER; note = Uracil
  modified_base 16: mod_base = OTHER; note = Uracil
  modified_base 18: mod_base = OTHER; note = Uracil
  modified_base 21: mod_base = OTHER; note = Uracil
  modified_base 26: mod_base = OTHER; note = Uracil
  modified_base 41: mod_base = OTHER; note = Uracil
  modified_base 42: mod_base = OTHER; note = Uracil
agtcgtgctgcttcatgtggtcacatgaagcagcacgacctt

SEQ ID NO: 148 - RNA, 42 nt, synthetic construct
  source 1..42: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 13: mod_base = OTHER; note = Uracil
  modified_base 17: mod_base = OTHER; note = Uracil
  modified_base 22: mod_base = OTHER; note = Uracil
  modified_base 25: mod_base = OTHER; note = Uracil
  modified_base 31: mod_base = OTHER; note = Uracil
  modified_base 41: mod_base = OTHER; note = Uracil
  modified_base 42: mod_base = OTHER; note = Uracil
atgcccttcagctcgatgcggtcatcgagctgaagggcactt

SEQ ID NO: 149 - RNA, 42 nt, synthetic construct
  source 1..42: mol_type = other RNA
  modified_base 4: mod_base = OTHER; note = Uracil
  modified_base 5: mod_base = OTHER; note = Uracil
  modified_base 7: mod_base = OTHER; note = Uracil
  modified_base 8: mod_base = OTHER; note = Uracil
  modified_base 10: mod_base = OTHER; note = Uracil
  modified_base 16: mod_base = OTHER; note = Uracil
  modified_base 17: mod_base = OTHER; note = Uracil
  modified_base 26: mod_base = OTHER; note = Uracil
  modified_base 28: mod_base = OTHER; note = Uracil
  modified_base 41: mod_base = OTHER; note = Uracil
  modified_base 42: mod_base = OTHER; note = Uracil
agcttcttctgcacattcaggagaatgtgcagaagaagcctt

SEQ ID NO: 150 - RNA, 42 nt, synthetic construct
  source 1..42: mol_type = other RNA
  modified_base 3: mod_base = OTHER; note = Uracil
  modified_base 7: mod_base = OTHER; note = Uracil
  modified_base 10: mod_base = OTHER; note = Uracil
  modified_base 19: mod_base = OTHER; note = Uracil
  modified_base 21: mod_base = OTHER; note = Uracil
  modified_base 25: mod_base = OTHER; note = Uracil
  modified_base 26: mod_base = OTHER; note = Uracil
  modified_base 27: mod_base = OTHER; note = Uracil
  modified_base 29: mod_base = OTHER; note = Uracil
  modified_base 41: mod_base = OTHER; note = Uracil
  modified_base 42: mod_base = OTHER; note = Uracil
agtggctggtcacaaaggtatacctttgtgaccagccacctt

SEQ ID NO: 151 - RNA, 42 nt, synthetic construct
  source 1..42: mol_type = other RNA
  modified_base 3: mod_base = OTHER; note = Uracil
  modified_base 4: mod_base = OTHER; note = Uracil
  modified_base 6: mod_base = OTHER; note = Uracil
  modified_base 22: mod_base = OTHER; note = Uracil
  modified_base 23: mod_base = OTHER; note = Uracil
  modified_base 26: mod_base = OTHER; note = Uracil
  modified_base 28: mod_base = OTHER; note = Uracil
  modified_base 31: mod_base = OTHER; note = Uracil
  modified_base 33: mod_base = OTHER; note = Uracil
  modified_base 41: mod_base = OTHER; note = Uracil
  modified_base 42: mod_base = OTHER; note = Uracil
agttctcagagcacaccacaattggtgtgctctgagaacctt

SEQ ID NO: 152 - RNA, 42 nt, synthetic construct
  source 1..42: mol_type = other RNA
  modified_base 8: mod_base = OTHER; note = Uracil
  modified_base 22: mod_base = OTHER; note = Uracil
  modified_base 23: mod_base = OTHER; note = Uracil
  modified_base 28: mod_base = OTHER; note = Uracil
  modified_base 35: mod_base = OTHER; note = Uracil
  modified_base 36: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
aggaaagtccccagcggaaagttccgctggggactttccctt

SEQ ID NO: 153 - DNA, 5368 bp, synthetic construct
  source 1..5368: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctaccaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaggaggaatggaggatgccaagaatattaagaaaggccctgccccattctaccctctggaagatggcactgctggtgagcaactgcacaaggccatgaagaggtatgccctggtccctggcaccattgccttcactgatgctcacattgaggtggacatcacctatgctgaatactttgagatgtctgtgaggctggcagaagccatgaaaagatatggactgaacaccaaccacaggattgtggtgtgctctgagaactctctccagttcttcatgcctgtgttaggagccctgttcattggagtggctgtggcccctgccaatgacatctacaatgagagagagctcctgaacagcatgggcatcagccagccaactgtggtctttgtgagcaagaagggcctgcaaaagatcctgaatgtgcagaagaagctgcccatcatccagaagatcatcatcatggacagcaagactgactaccagggcttccagagcatgtatacctttgtgaccagccacttaccccctggcttcaatgagtatgactttgtgcctgagagctttgacagggacaagaccattgctctgattatgaacagctctggctccactggactgcccaaaggtgtggctctgccccacagaactgcttgtgtgagattcagccatgccagagaccccatctttggcaaccagatcatccctgacactgccatcctgtctgtggttccattccatcatggctttggcatgttcacaacactggggtacctgatctgtggcttcagagtggtgctgatgtataggtttgaggaggagctgtttctgaggagcctacaagactacaagatccagtctgccctgctggtgcccactctgttcagcttctttgccaagagcaccctcattgacaagtatgacctgagcaacctgcatgagattgcctctggaggagcacccctgagcaaggaggtgggtgaggctgtggcaaagaggttccatctcccaggaatcagacagggctatggcctgactgagaccacctctgccatcctcatcacccctgaaggagatgacaagcctggtgctgtgggcaaggtggttcccttttttgaggccaaggtggtggacctggacactggcaagaccctgggagtgaaccagaggggtgagctgtgtgtgaggggtcccatgatcatgtctggctatgtgaacaaccctgaggccaccaatgccctgattgacaaggatggctggctgcactctggtgacattgcctactgggatgaggatgagcactttttcattgtggacaggctgaagagcctcatcaagtacaaaggctaccaagtggcacctgctgagctagagagcatcctgctccagcaccccaacatctttgatgctggtgtggctggcctgcctgatgatgatgctggagagctgcctgctgctgttgtggttctggagcatggaaagaccatgactgagaaggagattgtggactatgtggccagtcaggtgaccactgccaagaagctgaggggaggtgtggtgtttgtggatgaggtgccaaagggtctgactggcaagctggatgccagaaagatcagagagatcctgatcaaggccaagaagggtggcaaatgaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 154 - DNA, 3177 bp, synthetic construct
  source 1..3177: mol_type = other DNA
atggactacaaagaccatgacggtgattataaagatcatgacatcgattacaaggatgacgatgacaagcttgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaggtggatccatgcacccattccagtggtgtaacgcacttgcacctcctgcgccgccgccccccatccaaggatatgccttcaagcctccacctagacccgactttgggacctccgggagaacaatcaaattacaggccaatttcttcgaaatggacatccccaaaattgacatctatcattatgaattggatatcaagccagagaagtgcccgaggagagttaacagggaaatcgtggaacacatggtccagcactttaaaacacagatctttggggatcggaagcccgtgtttgacggcaggaagaatctatacacagccatgccccttccgattgggagggacaaggtggagctggaggtcacgctgccaggagaaggcaaggatcgcatcttcaaggtgtccatcaagtgggtgtcctgcgtgagcttgcaggcgttacacgatgcactttcagggcggctgcccagcgtcccttttgagacgatccaggccctggacgtggtcatgaggcacttgccatccatgaggtacacccccgtgggccgctccttcttcaccgcgtccgaaggctgctctaaccctcttggcgggggccgagaagtgtggtttggcttccatcagtccgtccggccttctctctggaaaatgatgctgaatattgatgtgtcagcaacagcgttttacaaggcacagccagtaatcgagtttgtttgtgaagttttggattttaaaagtattgaagaacaacaaaaacctctgacagattcccaaagggtaaagtttaccaaagaaattaaaggtctaaaggtggagataacgcactgtgggcagatgaagaggaagtaccgtgtctgcaatgtgacccggcggcccgccagtcaccaaacattcccgctgcagcaggagagcgggcagacggtggagtgcacggtggcccagtatttcaaggacaggcacaagttggttctgcgctacccccacctcccatgtttacaagtcggacaggagcagaaacacacctaccttcccctggaggtctgtaacattgtggcaggacaaagatgtattaaaaaattaacggacaatcagacctcaaccatgatcagagcaactgctatgtcggcgcccgatcggcaagaagagattagcaaattgatgcgaagtgcaagtttcaacacagatccatacgtccgtgaatttggaatcatggtcaaagatgagatgacagacgtgactgggcgggtgctgcagccgccctccatcctctacgggggcaggaataaagctattgcgacccctgtccagggcgtctgggacatgcggaacaagcagttccacacgggcatcgagatcaaggtgtgggccattgcgtgcttcgccccccagcgccagtgcacggaagtccatctgaagtccttcacagagcagctcagaaagatctcgagagacgctggcatgcccatccagggccagccgtgcttctgcaaatacgcgcagggggcggacagcgtggagcccatgttccggcacctgaagaacacgtatgcgggcctgcagctggtggtggtcatcctgcccggcaagacgcccgtgtacgccgaggtcaagcgcgtgggagacacggtgctggggatggccacgcagtgcgtgcagatgaagaacgtgcagaggaccacgccacagaccctgtccaacctctgcctgaagatcaacgtcaagctgggaggcgtgaacaacatcctgctgccccagggcaggccgccggtgttccagcagcccgtcatctttctgggagcagacgtcactcacccccccgccggggatgggaagaagccctccattgccgccgtggtgggcagcatggacgcccaccccaatcgctactgcgccaccgtgcgcgtgcagcagcaccggcaggagatcatacaagacctggccgccatggtccgcgagctcctcatccagttctacaagtccacgcgcttcaagcccacccgcatcatcttctaccgcgacggtgtctctgaaggccagttccagcaggttctccaccacgagttgctggccatccgtgaggcctgtatcaagctagaaaaagactaccagcccgggatcaccttcatcgtggtgcagaagaggcaccacacccggctcttctgcactgacaagaacgagcgggttgggaaaagtggaaacattccagcaggcacgactgtggacacgaaaatcacccaccccaccgagttcgacttctacctgtgtagtcacgctggcatccaggggacaagcaggccttcgcactatcacgtcctctgggacgacaatcgtttctcctctgatgagctgcagatcctaacctaccagctgtgtcacacctacgtgcgctgcacacgctccgtgtccatcccagcgccagcatactacgctcacctggtggccttccgggccaggtaccacctggtggataaggaacatgacagtgctgaaggaagccatacctctgggcagagtaacgggcgagaccaccaagcactggccaaggcggtccaggttcaccaagacactctgcgcaccatgtactttgcttaa

SEQ ID NO: 155 - protein, 1058 aa, synthetic construct
MDYKDHDGDYKDHDIDYKDDDDKLVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGGSMHPFQWCNALAPPAPPPPIQGYAFKPPPRPDFGTSGRTIKLQANFFEMDIPKIDIYHYELDIKPEKCPRRVNREIVEHMVQHFKTQIFGDRKPVFDGRKNLYTAMPLPIGRDKVELEVTLPGEGKDRIFKVSIKWVSCVSLQALHDALSGRLPSVPFETIQALDVVMRHLPSMRYTPVGRSFFTASEGCSNPLGGGREVWFGFHQSVRPSLWKMMLNIDVSATAFYKAQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQRCIKKLTDNQTSTMIRATAMSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFA

SEQ ID NO: 156 - protein, 860 aa, synthetic construct
MHPFQWCNALAPPAPPPPIQGYAFKPPPRPDFGTSGRTIKLQANFFEMDIPKIDIYHYELDIKPEKCPRRVNREIVEHMVQHFKTQIFGDRKPVFDGRKNLYTAMPLPIGRDKVELEVTLPGEGKDRIFKVSIKWVSCVSLQALHDALSGRLPSVPFETIQALDVVMRHLPSMRYTPVGRSFFTASEGCSNPLGGGREVWFGFHQSVRPSLWKMMLNIDVSATAFYKAQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQRCIKKLTDNQTSTMIRATAMSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFA

SEQ ID NO: 157 - protein, 30 aa, synthetic construct
WEAALAEALAEALAEHLAEALAEALEALAA

SEQ ID NO: 158 - protein, 30 aa, synthetic construct
AALAEALAEALAEALAEALAEALAAAAGGC

SEQ ID NO: 159 - protein, 30 aa, synthetic construct
WEAKLAKALAKALAKHLAKALAKALKACEA

SEQ ID NO: 160 - DNA, 249 bp, synthetic construct
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaagatcc

SEQ ID NO: 161 - DNA, 314 bp, synthetic construct
  source 1..314: mol_type = other DNA
gatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtt

SEQ ID NO: 162 - DNA, 103 bp, synthetic construct
  source 1..103: mol_type = other DNA
tgcaatatttgcatgtcgctatgtgttctgggaaatcaccataaacgtgaaatgtctttggatttgggaatcttataagttctgtatgagaccactcagatcc

SEQ ID NO: 163 - DNA, 243 bp, synthetic construct
  source 1..243: mol_type = other DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 164 - DNA, 391 bp, synthetic construct
  source 1..391: mol_type = other DNA
ctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaaggagttcccgtgccctgggagcgggttcaggaccgcggatcggaagtgagaatcccagctgtgtgtcagggctggaaagggctcgggagtgcgcggggcaagtgaccgtgtgtgtaaagagtgaggcgtatgaggctgtgtcggggcagagcccgaagatc

SEQ ID NO: 165 - DNA, 20 bp, synthetic construct
  source 1..20: mol_type = other DNA
taatacgactcactataggg

SEQ ID NO: 166 - DNA, 19 bp, synthetic construct
  source 1..19: mol_type = other DNA
atttaggtgacactataga

SEQ ID NO: 167 - DNA, 19 bp, synthetic construct
  source 1..19: mol_type = other DNA
aattaaccctcactaaagg

SEQ ID NO: 168 - DNA, 73 bp, synthetic construct
  source 1..73: mol_type = other DNA
gcccggatagctcagtcggtagagcatcagacttttaatctgagggtccagggttcaagtccctgttcgggcg

SEQ ID NO: 169 - DNA, 73 bp, synthetic construct
  source 1..73: mol_type = other DNA
gttttcgtagtgtagtggttatcacgtgtgcttcacacgcacaaggtccccggttcgaacccgggcgaaaaca

SEQ ID NO: 170 - DNA, 110 bp, synthetic construct
  source 1..110: mol_type = other DNA
gaaaatgactttgccacgcttagcatgtgacgaggtggccgagtggttaaggcgatggactgctaatccattgtgctctgcacgcgtgggttcgaatcccatcctcgtcg

SEQ ID NO: 171 - DNA, 71 bp, synthetic construct
  source 1..71: mol_type = other DNA
gcatgggtggttcagtggtagaattctcgcctgccacgcgggaggcccgggttcgattcccggcccatgca

SEQ ID NO: 172 - DNA, 72 bp, synthetic construct
  source 1..72: mol_type = other DNA
ggttccatggtgtaatggttagcactctggactctgaatccagcgatccgagttcaaatctcggtggaacct

SEQ ID NO: 173 - DNA, 72 bp, synthetic construct
  source 1..72: mol_type = other DNA
ggctcgttggtctaggggtatgattctcgcttagggtgcgagaggtcccgggttcaaatcccggacgagccc

SEQ ID NO: 174 - DNA, 83 bp, synthetic construct
  source 1..83: mol_type = other DNA
gtcaggatggccgagtggtctaaggcgctgcgttcaggtcgcagtctactctgtaggcgtgggttcgaatcccacttctgaca

SEQ ID NO: 175 - DNA, 74 bp, synthetic construct
  source 1..74: mol_type = other DNA
gcctccgtggcgcaattggttagcgcgttcggctgttaaccgaaaggttggtggttcgagtccacccgggggcg

SEQ ID NO: 176 - DNA, 39 bp, synthetic construct
  source 1..39: mol_type = other DNA
tcgagtacggcaagctgaccctgaagttcatctgcacgc

SEQ ID NO: 177 - DNA, 39 bp, synthetic construct
  source 1..39: mol_type = other DNA
ggccgcgtgcagatgaacttcagggtcagcttgccgtac

SEQ ID NO: 178 - DNA, 39 bp, synthetic construct
  source 1..39: mol_type = other DNA
tcgaggtgcagatgaacttcagggtcagcttgccgtagc

SEQ ID NO: 179 - DNA, 39 bp, synthetic construct
  source 1..39: mol_type = other DNA
ggccgctacggcaagctgaccctgaagttcatctgcacc

SEQ ID NO: 180 - DNA, 34 bp, synthetic construct
  source 1..34: mol_type = other DNA
tcgaggggactttccgctggggactttccagggc

SEQ ID NO: 181 - DNA, 34 bp, synthetic construct
  source 1..34: mol_type = other DNA
ggccgccctggaaagtccccagcggaaagtcccc

SEQ ID NO: 182 - DNA, 34 bp, synthetic construct
  source 1..34: mol_type = other DNA
tcgagcctggaaagtccccagcggaaagtcccgc

SEQ ID NO: 183 - DNA, 34 bp, synthetic construct
  source 1..34: mol_type = other DNA
ggccgcgggactttccgctggggactttccaggc

SEQ ID NO: 184 - DNA, 826 bp, synthetic construct
  source 1..826: mol_type = other DNA
cagtaattctaggcgatcgctcgagatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataagcggccgctggccgcaataaaatat

SEQ ID NO: 185 - DNA, 663 bp, synthetic construct
  source 1..663: mol_type = other DNA
cgactcactataggctagcctcgaggccaccatgtacccatatgacgtgcccgattacgccggcagcggcactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaatagtaagcggccgcttccctttagtgagg

SEQ ID NO: 186 - DNA, 666 bp, synthetic construct
  source 1..666: mol_type = other DNA
cgactcactataggctagcctcgaggccaccatgtacccatatgacgtgcccgattacgccggcagcggcacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgatagtaagcggccgcttccctttagtgagg

SEQ ID NO: 187 - DNA, 666 bp, synthetic construct
  source 1..666: mol_type = other DNA
cgactcactataggctagcctcgaggccaccatgtacccatatgacgtgcccgattacgccggcagcggcactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaatagtaagcggccgcttccctttagtgagg

SEQ ID NO: 188 - DNA, 1404 bp, synthetic construct
  source 1..1404: mol_type = other DNA
cgactcactataggctagcctcgaggccaccatgtacccatatgacgtgcccgattacgccggcagcggcgagatggaaaaggagttcgagcagatcgacaagtccgggagctgggcggccatttaccaggatatccgacatgaagccagtgacttcccatgtagagtggccaagcttcctaagaacaaaaaccgaaataggtacagagacgtcagtccctttgaccatagtcggattaaactacatcaagaagataatgactatatcaacgctagtttgataaaaatggaagaagcccaaaggagttacattcttacccagggccctttgcctaacacatgcggtcacttttgggagatggtgtgggagcagaaaagcaggggtgtcgtcatgctcaacagagtgatggagaaaggttcgttaaaatgcgcacaatactggccacaaaaagaagaaaaagagatgatctttgaagacacaaatttgaaattaacattgatctctgaagatatcaagtcatattatacagtgcgacagctagaattggaaaaccttacaacccaagaaactcgggagatcttacatttccactataccacatggcctgactttggagtccctgaatcaccagcctcattcttgaactttcttttcaaagtccgagagtcagggtcactcagcccggagcacgggcccgttgtggtgcactgcagtgcaggcatcggcaggtctggaaccttctgtctggctgatacctgcctcttgctgatggacaagaggaaagacccttcttccgttgatatcaagaaagtgctgttagaaatgaggaagtttcggatggggctgatccagacagccgaccagctgcgcttctcctacctggctgtgatcgaaggtgccaaattcatcatgggggactcttccgtgcaggatcagtggaaggagctttcccacgaggacctggagcccccacccgagcatatccccccacctccccggccacccaaacgaatcctggagccacacaatgggaaatgcagggagttcttcccaaatcaccagtgggtgaaggaagagacccaggaggataaagactgccccatcaaggaagaaaaaggaagccccttaaatgccgcaccctacggcatcgaaagcatgagtcaagacactgaagttagaagtcgggtcgtggggggaagtcttcgaggtgcccaggctgcctccccagccaaaggggagccgtcactgcccgagaaggacgaggaccatgcactgagttactggaagcccttcctggtcaacatgtgcgtggctacggtcctcacggccggcgcttacctctgctacaggttcctgttcaacagcaacacatagtagtaagcggccgcttccctttagtgagg

SEQ ID NO: 189 - DNA, 663 bp, synthetic construct
  source 1..663: mol_type = other DNA
cgactcactataggctagcctcgaggccaccatgtacccatatgacgtgcccgattacgccggcagcggcactgaatataaacttgtggtagttggagctggtggcgtaggcaagaatgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaatagtaagcggccgcttccctttagtgagg

SEQ ID NO: 190 - DNA, 666 bp, synthetic construct
  source 1..666: mol_type = other DNA
cgactcactataggctagcctcgaggccaccatgtacccatatgacgtgcccgattacgccggcagcggcacggaatataagctggtggtggtgggcgccggcggtgtgggcaagaacgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgatagtaagcggccgcttccctttagtgagg

SEQ ID NO: 191 - DNA, 666 bp, synthetic construct
  source 1..666: mol_type = other DNA
cgactcactataggctagcctcgaggccaccatgtacccatatgacgtgcccgattacgccggcagcggcacggaatataagctggtggtggtgggcgccgacggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgatagtaagcggccgcttccctttagtgagg

SEQ ID NO: 192 - DNA, 666 bp, synthetic construct
  source 1..666: mol_type = other DNA
cgactcactataggctagcctcgaggccaccatgtacccatatgacgtgcccgattacgccggcagcggcacggaatataagctggtggtggtgggcgccgtgggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgatagtaagcggccgcttccctttagtgagg

SEQ ID NO: 193 - DNA, 1158 bp, synthetic construct
  source 1..1158: mol_type = other DNA
cgactcactataggctagcctcgaggccaccatggagaagaccctggagaccgtgcccctggagagaaagaagagagagaaggagcagttcagaaagctgttcatcggcggcctgagcttcgagaccaccgaggagagcctgagaaactactacgagcagtggggcaagctgaccgactgcgtggtgatgagagaccccgccagcaagagaagcagaggcttcggcttcgtgaccttcagcagcatggccgaggtggacgccgccatggccgccagaccccacagcatcgacggcagagtggtggagcccaagagagccgtggccagagaggagagcggcaagcccggcgcccacgtgaccgtgaagaagctgttcgtgggcggcatcaaggaggacaccgaggagcaccacctgagagactacttcgaggagtacggcaagatcgacaccatcgagatcatcaccgacagacagagcggcaagaagagaggcttcggcttcgtgaccttcgacgaccacgaccccgtggacaagatcgtgctgcagaagtaccacaccatcaacggccacaacgccgaggtgagaaaggccctgagcagacaggagatgcaggaggtgcagagcagcagaagcggcagaggcggcaacttcggcttcggcgacagcagaggcggcggcggcaacttcggccccggccccggcagcaacttcagaggcggcagcgacggctacggcagcggcagaggcttcggcgacggctacaacggctacggcggcggccccggcggcggcaacttcggcggcagccccggctacggcggcggcagaggcggctacggcggcggcggccccggctacggcaaccagggcggcggctacggcggcggctacgacaactacggcggcggcaactacggcagcggcaactacaacgacttcggcaactacaaccagcagcccagcaactacggccccatgaagagcggcaacttcggcggcagcagaaacatgggcggcccctacggcggcggcaactacggccccggcggcagcggcggcagcggcggctacggcggcagaagcagatacggcagcggcgagcagaagctgatctcagaggaggacctgtagtaagcggccgcttccctttagtgagg

SEQ ID NO: 194 - DNA, 1968 bp, synthetic construct
  source 1..1968: mol_type = other DNA
cgactcactataggctagcctcgaggccaccatggccaccgagcacgtgaacggcaacggcaccgaggagcccatggacaccaccagcgccgtgatccacagcgagaacttccagaccctgctggacgccggcctgccccagaaggtggccgagaagctggacgagatctacgtggccggcctggtggcccacagcgacctggacgagagagccatcgaggccctgaaggagttcaacgaggacggcgccctggccgtgctgcagcagttcaaggacagcgacctgagccacgtgcagaacaagagcgccttcctgtgcggcgtgatgaagacctacagacagagagagaagcagggcaccaaggtggccgacagcagcaagggccccgacgaggccaagatcaaggccctgctggagagaaccggctacaccctggacgtgaccaccggccagagaaagtacggcggccccccccccgacagcgtgtacagcggccagcagcccagcgtgggcaccgagatcttcgtgggcaagatccccagagacctgttcgaggacgagctggtgcccctgttcgagaaggccggccccatctgggacctgagactgatgatggaccccctgaccggcctgaacagaggctacgccttcgtgaccttctgcaccaaggaggccgcccaggaggccgtgaagctgtacaacaaccacgagatcagaagcggcaagcacatcggcgtgtgcatcagcgtggccaacaacagactgttcgtgggcagcatccccaagagcaagaccaaggagcagatcctggaggagttcagcaaggtgaccgagggcctgaccgacgtgatcctgtaccaccagcccgacgacaagaagaagaacagaggcttctgcttcctggagtacgaggaccacaagaccgccgcccaggccagaagaagactgatgagcggcaaggtgaaggtgtggggcaacgtgggcaccgtggagtgggccgaccccatcgaggaccccgaccccgaggtgatggccaaggtgaaggtgctgttcgtgagaaacctggccaacaccgtgaccgaggagatcctggagaaggccttcagccagttcggcaagctggagagagtgaagaagctgaaggactacgccttcatccacttcgacgagagagacggcgccgtgaaggccatggaggagatgaacggcaaggacctggagggcgagaacatcgagatcgtgttcgccaagccccccgaccagaagagaaaggagagaaaggcccagagacaggccgccaagaaccagatgtacgacgactactactactacggccccccccacatgcccccccccaccagaggcagaggcagaggcggcagaggcggctacggctacccccccgactactacggctacgaggactactacgactactacggctacgactaccacaactacagaggcggctacgaggacccctactacggctacgaggacttccaggtgggcgccagaggcagaggcggcagaggcgccagaggcgccgcccccagcagaggcagaggcgccgccccccccagaggcagagccggctacagccagagaggcggccccggcagcgccagaggcgtgagaggcgccagaggcggcgcccagcagcagagaggcagaggcgtgagaggcgccagaggcggcagaggcggcaacgtgggcggcaagagaaaggccgacggctacaaccagcccgacagcaagagaagacagaccaacaaccagaactggggcagccagcccatcgcccagcagcccctgcagggcggcgaccacagcggcaactacggctacaagagcgagaaccaggagttctaccaggacaccttcggccagcagtggaagggcagcggcgagcagaagctgatctcagaggaggacctgtagtaagcggccgcttccctttagtgagg

SEQ ID NO: 195 - DNA, 1323 bp, synthetic construct
  source 1..1323: mol_type = other DNA
cgactcactataggctagcctcgaggccaccatggccgagaacggcgacaacgagaagatggccgccctggaggccaagatctgccaccagatcgagtactacttcggcgacttcaacctgcccagagacaagttcctgaaggagcagatcaagctggacgagggctgggtgcccctggagatcatgatcaagttcaacagactgaacagactgaccaccgacttcaacgtgatcgtggaggccctgagcaagagcaaggccgagctgatggagatcagcgaggacaagaccaagatcagaagaagccccagcaagcccctgcccgaggtgaccgacgagtacaagaacgacgtgaagaacagaagcgtgtacatcaagggcttccccaccgacgccaccctggacgacatcaaggagtggctggaggacaagggccaggtgctgaacatccagatgagaagaaccctgcacaaggccttcaagggcagcatcttcgtggtgttcgacagcatcgagagcgccaagaagttcgtggagacccccggccagaagtacaaggagaccgacctgctgatcctgttcaaggacgactacttcgccaagaagaacgaggagagaaagcagaacaaggtggaggccaagctgagagccaagcaggagcaggaggccaagcagaagctggaggaggacgccgagatgaagagcctggaggagaagatcggctgcctgctgaagttcagcggcgacctggacgaccagacctgcagagaggacctgcacatcctgttcagcaaccacggcgagatcaagtggatcgacttcgtgagaggcgccaaggagggcatcatcctgttcaaggagaaggccaaggaggccctgggcaaggccaaggacgccaacaacggcaacctgcagctgagaaacaaggaggtgacctgggaggtgctggagggcgaggtggagaaggaggccctgaagaagatcatcgaggaccagcaggagagcctgaacaagtggaagagcaagggcagaagattcaagggcaagggcaagggcaacaaggccgcccagcccggcagcggcaagggcaaggtgcagttccagggcaagaagaccaagttcgccagcgacgacgagcacgacgagcacgacgagaacggcgccaccggccccgtgaagagagccagagaggagaccgacaaggaggagcccgccagcaagcagcagaagaccgagaacggcgccggcgaccagggcagcggcgagcagaagctgatctcagaggaggacctgtagtaagcggccgcttccctttagtgagg

SEQ ID NO: 196 - DNA, 870 bp, synthetic construct
  source 1..870: mol_type = other DNA
cgactcactataggctagcctcgaggccaccatggccgacaagatggacatgagcctggacgacatcatcaagctgaacagaagccagagaggcggcagaggcggcggcagaggcagaggcagagccggcagccagggcggcagaggcggcggcgcccaggccgccgccagagtgaacagaggcggcggccccatcagaaacagacccgccatcgccagaggcgccgccggcggcggcggcagaaacagacccgccccctacagcagacccaagcagctgcccgacaagtggcagcacgacctgttcgacagcggcttcggcggcggcgccggcgtggagaccggcggcaagctgctggtgagcaacctggacttcggcgtgagcgacgccgacatccaggagctgttcgccgagttcggcaccctgaagaaggccgccgtgcactacgacagaagcggcagaagcctgggcaccgccgacgtgcacttcgagagaaaggccgacgccctgaaggccatgaagcagtacaacggcgtgcccctggacggcagacccatgaacatccagctggtgaccagccagatcgacgcccagagaagacccgcccagagcgtgaacagaggcggcatgaccagaaacagaggcgccggcggcttcggcggcggcggcggcaccagaagaggcaccagaggcggcgccagaggcagaggcagaggcgccggcagaaacagcaagcagcagctgagcgccgaggagctggacgcccagctggacgcctacaacgccagaatggacaccagcggcagcggcgagcagaagctgatctcagaggaggacctgtagtaagcggccgcttccctttagtgagg

SEQ ID NO: 197 - DNA, 1677 bp, synthetic construct
  source 1..1677: mol_type = other DNA
cgactcactataggctagcctcgaggccaccatggccagcaacgactacacccagcaggccacccagagctacggcgcctaccccacccagcccggccagggctacagccagcagagcagccagccctacggccagcagagctacagcggctacagccagagcaccgacaccagcggctacggccagagcagctacagcagctacggccagagccagaacaccggctacggcacccagagcaccccccagggctacggcagcaccggcggctacggcagcagccagagcagccagagcagctacggccagcagagcagctaccccggctacggccagcagcccgcccccagcagcaccagcggcagctacggcagcagcagccagagcagcagctacggccagccccagagcggcagctacagccagcagcccagctacggcggccagcagcagagctacggccagcagcagagctacaaccccccccagggctacggccagcagaaccagtacaacagcagcagcggcggcggcggcggcggcggcggcggcggcaactacggccaggaccagagcagcatgagcagcggcggcggcagcggcggcggctacggcaaccaggaccagagcggcggcggcggcagcggcggctacggccagcaggacagaggcggcagaggcagaggcggcagcggcggcggcggcggcggcggcggcggcggctacaacagaagcagcggcggctacgagcccagaggcagaggcggcggcagaggcggcagaggcggcatgggcggcagcgacagaggcggcttcaacaagttcggcggccccagagaccagggcagcagacacgacagcgagcaggacaacagcgacaacaacaccatcttcgtgcagggcctgggcgagaacgtgaccatcgagagcgtggccgactacttcaagcagatcggcatcatcaagaccaacaagaagaccggccagcccatgatcaacctgtacaccgacagagagaccggcaagctgaagggcgaggccaccgtgagcttcgacgacccccccagcgccaaggccgccatcgactggttcgacggcaaggagttcagcggcaaccccatcaaggtgagcttcgccaccagaagagccgacttcaacagaggcggcggcaacggcagaggcggcagaggcagaggcggccccatgggcagaggcggctacggcggcggcggcagcggcggcggcggcagaggcggcttccccagcggcggcggcggcggcggcggccagcagagagccggcgactggaagtgccccaaccccacctgcgagaacatgaacttcagctggagaaacgagtgcaaccagtgcaaggcccccaagcccgacggccccggcggcggccccggcggcagccacatgggcggcaactacggcgacgacagaagaggcggcagaggcggctacgacagaggcggctacagaggcagaggcggcgacagaggcggcttcagaggcggcagaggcggcggcgacagaggcggcttcggccccggcaagatggacagcagaggcgagcacagacaggacagaagagagagaccctacggcagcggcgagcagaagctgatctcagaggaggacctgtagtaagcggccgcttccctttagtgagg

SEQ ID NO: 198 - DNA, 2076 bp, synthetic construct
  source 1..2076: mol_type = other DNA
cgactcactataggctagcctcgaggccaccatgcccagcggcagcagcgccgccctggccctggccgccgcccccgcccccctgccccagcccccccccccccccccccccccccccccccccctgcccccccccagcggcggccccgagctggagggcgacggcctgctgctgagagagagactggccgccctgggcctggacgaccccagccccgccgagcccggcgcccccgccctgagagcccccgccgccgccgcccagggccaggccagaagagccgccgagctgagccccgaggagagagccccccccggcagacccggcgcccccgaggccgccgagctggagctggaggaggacgaggaggagggcgaggaggccgagctggacggcgacctgctggaggaggaggagctggaggaggccgaggaggaggacagaagcagcctgctgctgctgagcccccccgccgccaccgccagccagacccagcagatccccggcggcagcctgggcagcgtgctgctgcccgccgccagattcgacgccagagaggccgccgccgccgccgccgccgccggcgtgctgtacggcggcgacgacgcccagggcatgatggccgccatgctgagccacgcctacggccccggcggctgcggcgccgccgccgccgccctgaacggcgagcaggccgccctgctgagaagaaagagcgtgaacaccaccgagtgcgtgcccgtgcccagcagcgagcacgtggccgagatcgtgggcagacagggctgcaagatcaaggccctgagagccaagaccaacacctacatcaagacccccgtgagaggcgaggagcccatcttcgtggtgaccggcagaaaggaggacgtggccatggccaagagagagatcctgagcgccgccgagcacttcagcatgatcagagccagcagaaacaagaacggccccgccctgggcggcctgagctgcagccccaacctgcccggccagaccaccgtgcaggtgagagtgccctacagagtggtgggcctggtggtgggccccaagggcgccaccatcaagagaatccagcagcagacccacacctacatcgtgacccccagcagagacaaggagcccgtgttcgaggtgaccggcatgcccgagaacgtggacagagccagagaggagatcgagatgcacatcgccatgagaaccggcaactacatcgagctgaacgaggagaacgacttccactacaacggcaccgacgtgagcttcgagggcggcaccctgggcagcgcctggctgagcagcaaccccgtgccccccagcagagccagaatgatcagcaactacagaaacgacagcagcagcagcctgggcagcggcagcaccgacagctacttcggcagcaacagactggccgacttcagccccaccagccccttcagcaccggcaacttctggttcggcgacaccctgcccagcgtgggcagcgaggacctggccgtggacagccccgccttcgacagcctgcccaccagcgcccagaccatctggacccccttcgagcccgtgaaccccctgagcggcttcggcagcgaccccagcggcaacatgaagacccagagaagaggcagccagcccagcacccccagactgagccccaccttccccgagagcatcgagcaccccctggccagaagagtgagaagcgacccccccagcaccggcaaccacgtgggcctgcccatctacatccccgccttcagcaacggcaccaacagctacagcagcagcaacggcggcagcaccagcagcagcccccccgagagcagaagaaagcacgactgcgtgatctgcttcgagaacgaggtgatcgccgccctggtgccctgcggccacaacctgttctgcatggagtgcgccaacaagatctgcgagaagagaacccccagctgccccgtgtgccagaccgccgtgacccaggccatccagatccacagcggcagcggcgagcagaagctgatctcagaggaggacctgtagtaagcggccgcttccctttagtgagg

SEQ ID NO: 199 - DNA, 774 bp, synthetic construct
  source 1..774: mol_type = other DNA
cgactcactataggctagcctcgaggccaccatggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgtagtaagcggccgcttccctttagtgagg

SEQ ID NO: 200 - DNA, 1089 bp, synthetic construct
  source 1..1089: mol_type = other DNA
cgactcactataggctagcctcgaggccaccatggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatggaattcggcggaggcgggtccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaatagtaagcggccgcttccctttagtgagg

SEQ ID NO: 201 - DNA, 566 bp, synthetic construct
  source 1..566: mol_type = other DNA
gattacaaggatgacgatgacaagcttgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaggtggatccatgcacccattccagtg

SEQ ID NO: 202 - DNA, 21 bp, synthetic construct
  source 1..21: mol_type = other DNA
gagggcctatttcccatgatt

SEQ ID NO: 203 - DNA, 83 bp, synthetic construct
  source 1..83: mol_type = other DNA
aaaaaaaaaatgggtcctttctttggactgcgtttcaaaccccgaccagttggtaggggtacggtgtttcgtcctttccacaa

SEQ ID NO: 204 - DNA, 136 bp, synthetic construct
  source 1..136: mol_type = other DNA
acactctttccctacacgacgctcttccgatcttatgaccatgattacgccaagcttggtaccgagctcggatccactagtaacggccgccagtgtgctggaattcggcttaagcagtggtatcaacgcagagtac

SEQ ID NO: 205 - DNA, 50 bp, synthetic construct
  source 1..50: mol_type = other DNA
gactggagttcagacgtgtgctcttccgatctatgaacttcagggtcagc

SEQ ID NO: 206 - DNA, 18 bp, synthetic construct
  source 1..18: mol_type = other DNA
atgaacttcagggtcagc

SEQ ID NO: 207 - DNA, 19 bp, synthetic construct
  source 1..19: mol_type = other DNA
atgaacttcagggtcagct

SEQ ID NO: 208 - DNA, 20 bp, synthetic construct
  source 1..20: mol_type = other DNA
atgaacttcagggtcagctt

SEQ ID NO: 209 - DNA, 21 bp, synthetic construct
  source 1..21: mol_type = other DNA
atgaacttcagggtcagcttg

SEQ ID NO: 210 - DNA, 22 bp, synthetic construct
  source 1..22: mol_type = other DNA
atgaacttcagggtcagcttgc

SEQ ID NO: 211 - RNA, 18 nt, synthetic construct
  source 1..18: mol_type = other RNA
  modified_base 2: mod_base = OTHER; note = Uracil
  modified_base 7: mod_base = OTHER; note = Uracil
  modified_base 8: mod_base = OTHER; note = Uracil
  modified_base 14: mod_base = OTHER; note = Uracil
atgaacttcagggtcagc

SEQ ID NO: 212 - DNA, 22 bp, synthetic construct
  source 1..22: mol_type = other DNA
aatatcaggttgaactgcttga

SEQ ID NO: 213 - DNA, 22 bp, synthetic construct
  source 1..22: mol_type = other DNA
tgcaataaatgtcttccctgag

SEQ ID NO: 214 - DNA, 20 bp, synthetic construct
  source 1..20: mol_type = other DNA
ctctgctcctcctgttcgac

SEQ ID NO: 215 - DNA, 20 bp, synthetic construct
  source 1..20: mol_type = other DNA
ttaaaagcagccctggtgac

SEQ ID NO: 216 - DNA, 18 bp, synthetic construct
  source 1..18: mol_type = other DNA
gctgaccctgaagttcat

SEQ ID NO: 217 - protein, 188 aa, Homo sapiens [sequence omitted]

SEQ ID NO: 218 - protein, 188 aa, synthetic construct
MTEYKLVVVGAGGVGKNALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 219 - protein, 188 aa, synthetic construct [sequence omitted]

SEQ ID NO: 220 - protein, 188 aa, synthetic construct
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 221 - protein, 189 aa, synthetic construct
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 222 - protein, 189 aa, synthetic construct
MTEYKLVVVGAGGVGKNALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 223 - protein, 189 aa, synthetic construct
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 224 - protein, 189 aa, synthetic construct [sequence omitted]

SEQ ID NO: 225 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 226 - protein, 189 aa, synthetic construct
MTEYKLVVVGAGGVGKNALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 227 - protein, 189 aa, synthetic construct [sequence omitted]

SEQ ID NO: 228 - protein, 189 aa, synthetic construct
MTEYKLVVVGAVGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 229 - DNA, 158 bp, synthetic construct
  source 1..158: mol_type = other DNA
atatttgcatgtcgctatgtgttctgggaaatcaccataaacgtgaaatgtctttggatttgggaatcttataagttctgtatgagaccactcagatccgcgaagatttgagaaatgagttatcaagagtaattcgtttctcaaatcttcgctttttt

SEQ ID NO: 230 - DNA, 156 bp, synthetic construct
  source 1..156: mol_type = other DNA
atatttgcatgtcgctatgtgttctgggaaatcaccataaacgtgaaatgtctttggatttgggaatcttataagttctgtatgagaccactcagatccgcagaataagcgagttgcgttatcaagagtaatgcagctcgcttattctgctttttt

SEQ ID NO: 231 - RNA, 53 nt, synthetic construct
  source 1..53: mol_type = other RNA
  modified_base 8: mod_base = OTHER; note = Uracil
  modified_base 9: mod_base = OTHER; note = Uracil
  modified_base 10: mod_base = OTHER; note = Uracil
  modified_base 17: mod_base = OTHER; note = Uracil
  modified_base 21: mod_base = OTHER; note = Uracil
  modified_base 22: mod_base = OTHER; note = Uracil
  modified_base 24: mod_base = OTHER; note = Uracil
  modified_base 31: mod_base = OTHER; note = Uracil
  modified_base 34: mod_base = OTHER; note = Uracil
  modified_base 35: mod_base = OTHER; note = Uracil
  modified_base 38: mod_base = OTHER; note = Uracil
  modified_base 39: mod_base = OTHER; note = Uracil
  modified_base 40: mod_base = OTHER; note = Uracil
  modified_base 42: mod_base = OTHER; note = Uracil
  modified_base 47: mod_base = OTHER; note = Uracil
  modified_base 49: mod_base = OTHER; note = Uracil
  modified_base 50: mod_base = OTHER; note = Uracil
gcgaagatttgagaaatgagttatcaagagtaattcgtttctcaaatcttcgc

SEQ ID NO: 232 - RNA, 51 nt, synthetic construct
  source 1..51: mol_type = other RNA
  modified_base 7: mod_base = OTHER; note = Uracil
  modified_base 15: mod_base = OTHER; note = Uracil
  modified_base 16: mod_base = OTHER; note = Uracil
  modified_base 20: mod_base = OTHER; note = Uracil
  modified_base 21: mod_base = OTHER; note = Uracil
  modified_base 23: mod_base = OTHER; note = Uracil
  modified_base 30: mod_base = OTHER; note = Uracil
  modified_base 33: mod_base = OTHER; note = Uracil
  modified_base 39: mod_base = OTHER; note = Uracil
  modified_base 43: mod_base = OTHER; note = Uracil
  modified_base 44: mod_base = OTHER; note = Uracil
  modified_base 46: mod_base = OTHER; note = Uracil
  modified_base 47: mod_base = OTHER; note = Uracil
  modified_base 49: mod_base = OTHER; note = Uracil
gcagaataagcgagttgcgttatcaagagtaatgcagctcgcttattctgc